(19) United States
(12) Patent Application Publication
(10) Pub. No.: US 2026/0035387 A1
(43) Pub. Date: Feb. 5, 2026

(54) COMPOUNDS FOR TREATING HUNTINGTON'S DISEASE

(71) Applicant: BIOGEN MA INC., Cambridge, MA (US)

(72) Inventors: Magnus Pfaffenbach, Chelsea, MA (US); Eric Stefan, Watertown, MA (US); Daniel R. Smith, Dorchester, MA (US); Philippe Bolduc, Weymouth, MA (US); Nupur Bansal, Medfield, MA (US); Chaofan Xu, Newton, MA (US); Emily Anne Peterson, Belmont, MA (US)

(21) Appl. No.: 19/099,325

(22) PCT Filed: Jul. 28, 2023
(86) PCT No.: PCT/US2023/028923

§ 371 Date: Jan. 28, 2025

Related U.S. Application Data
(60) Provisional application No. 63/393,496, filed on Jul. 29, 2022.

(51) Int. Cl. C07D 519/00; A61K 31/437; A61K 31/4545; A61K 31/496; A61K 31/4985; A61K 31/519; A61P 25/28; C07D 495/04

(52) U.S. Cl. CPC C07D 519/00; A61K 31/437; A61K 31/4545; A61K 31/496; A61K 31/4985; A61K 31/519; A61P 25/28; C07D 495/04

(57) ABSTRACT

The present disclosure provides a compound of Formula (I′), or a pharmaceutically acceptable salt thereof and its use in, e.g. treating a condition, disease, or disorder in which lowering mutant huntingtin protein (“mHTT”) in a subject is of therapeutic benefit, specifically in treating Huntington disease (“HD”). This disclosure also features a composition containing the same as well as methods of using and making the same.

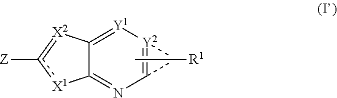

### RELATED APPLICATION

This application claims the benefit of the filing date, under 35 U.S.C. § 119(e), of U.S. Provisional Application No. 63/393,496, filed on Jul. 29, 2022, the entire contents of which are incorporated here by reference.

### BACKGROUND

Huntington's disease (HD) is an autosomal dominant progressive neurodegenerative disorder, which has a prevalence of between three and seven individuals per 100,000 worldwide. HD is caused by cytosine-adenine-guanine (CAG) repeat expansions in the huntingtin (HTT) gene resulting in the production of a ubiquitously expressed pathogenic mutant HTT (mHTT) protein. Mutant huntingtin contains an abnormally long polyglutamine (polyQ) sequence that corresponds to the CAG genetic expansion; the protein exhibits toxic properties that cause dysfunction and death of neurons. The disease is characterized by motor, cognitive, psychiatric and functional capacity decline.

Some research progresses are being made in identifying HTT protein-lowering therapies using multiple tools, including ribonucleic acid (RNA) interference using short interfering RNAs, short-hairpin RNAs, or microRNAs and antisense oligonucleotides (“ASO”) causing translational repression or messenger RNA (mRNA) degradation. However, these therapies require either surgical delivery of a viral vector for chronic HTT transcript lowering by RNAi, or repeated infusions into the cerebral spinal fluid (“CSF”) by lumbar puncture for ASOs in the clinic.

More recently, a small molecule compound platform, which modulates RNA expression, i.e. splicing correction, is under development. NVS-SMT (LMI070), now called branaplam, is a pyridazine derivative. It is reported that branaplam lowers mHTT protein levels in HD patient cells, in an HD mouse model and in blood samples from Spinal Muscular Atrophy (SMA) Type I patients dosed orally for SMA (NCT02268552). See Keller, C. etc., An Orally Available, Brain Penetrant, Small Molecule Lowers Huntingtin Levels by Enhancing Pseudoexon Inclusion, Nature Communications, (2022) 13:1150.

However, there are no approved disease-modifying treatments for HD till now, leaving a high unmet need for medications that can be used for treating or ameliorating HD. Accordingly, there is a need to find disease-modifying therapies for HD (i.e. therapeutic options that can slow disease progression).

### SUMMARY

Described herein are compounds or pharmaceutically acceptable salts thereof, which can be useful in treating HD in a subject.

In one aspect, the present disclosure provides a compound of Formula (I′) or a pharmaceutically acceptable salt thereof:

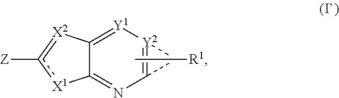

wherein X¹, X², Y¹, Y², Z, and R¹ are as defined herein.

Also provided are pharmaceutical compositions comprising a compound of Formula (I′) or a pharmaceutically acceptable salt thereof and a pharmaceutically acceptable carrier or excipient.

The present disclosure further provides methods of lowering mHTT in a subject, comprising administering to the subject a compound of Formula (I′) or a pharmaceutically acceptable salt thereof.

The present disclosure also provides methods of treating a disease or condition modulated at least in part by mHTT in a subject, comprising administering to the subject a therapeutically effective amount of a compound of Formula (I′) or a pharmaceutically acceptable salt thereof.

The present disclosure further provides a method of treating Huntington disease (“HD”) in a subject in need thereof, comprising administering to the subject an effective amount of (1) a compound of Formula (I′) or a pharmaceutically acceptable salt thereof; or (2) a pharmaceutically acceptable composition comprising a compound of Formula (I′) or a pharmaceutically acceptable salt thereof, and a pharmaceutically acceptable carrier.

In certain embodiments of the methods of the present disclosure, HD can be treated by lowering mHTT level in a subject.

The present disclosure also provides a use of a compound of Formula (I′), a pharmaceutically acceptable salt, or a pharmaceutical composition comprising the same in any of the methods described herein. In one embodiment, provided is a compound of Formula (I′) or a pharmaceutically acceptable salt thereof or a pharmaceutical composition comprising the same for use in any of the methods described herein. In another embodiment, provided is use of a compound of Formula (I′) or a pharmaceutically acceptable salt thereof or a pharmaceutical composition comprising the same for the manufacture of a medicament for any of the methods described herein.

### DETAILED DESCRIPTION

### 1. Compounds

In a first aspect, the present disclosure provides a compound of Formula (I′):

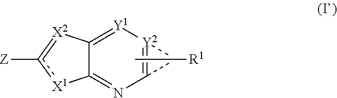

or a pharmaceutically acceptable salt thereof, wherein:
[symbol] is a single bond or double bond, provided the ring containing X₁ and X₂ is a 5-membered heteroaryl ring;

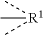

indicates that R¹ is substituted at one of two positions on the 6-membered ring to which the dash lines connect and the other position to which the dash lines connect is unsubstituted;
Z is —C(═O)NR²R³ or —NR²C(═O)R³;
X¹ is S or CH;
X² is N, O or CH;
one of Y¹ and Y² is N and the other is CH;
R¹ is 4 to 12 membered heterocyclyl, 4 to 12 membered carbocyclyl, —NR¹¹R¹², —C₁₋₆alkylene-NR¹³R¹⁴, or —OR¹⁵ wherein
   said 4 to 12 membered carbocyclyl or 4 to 12-membered heterocyclyl represented by R¹ is optionally substituted with one or more R^{A}; wherein
      each R^{A} is independently C₁₋₆alkyl, C₃₋₆cycloalkyl, haloC₁₋₆alkyl, —NR^{a}R^{b}, —C₁₋₃alkylene-NR^{a}R^{b}, —C₃₋₆cycloalkylene-NR^{a}R^{b}, —C(═O)R^{a}, or 4 to 6-membered saturated heterocyclyl; wherein each R^{a} and R^{b} is independently H or C₁₋₆alkyl; wherein said 4 to 6-membered saturated heterocyclyl represented by R^{A} is optionally substituted by one or more C₁₋₆alkyl; R¹¹ is H or C₁₋₆alkyl;
   R¹² is C₁₋₆alkyl, 6 to 10-membered aryl, 4 to 12-membered heterocyclyl, or 5-10 membered heteroaryl; wherein said C₁₋₆alkyl, 6 to 10-membered aryl, 4 to 12-membered heterocyclyl, or 5-10 membered heteroaryl represented by R¹² is optionally substituted by one or more R^{B}; wherein
      R^{B} is halo, C₁₋₆alkyl, —NR^{a}R^{b}, 4 to 6-membered heterocyclyl, or —C₁₋₆ alkylene-4 to 6-membered heterocyclyl; wherein said 4 to 6-membered heterocyclyl represented by R^{B} is optionally substituted by one or more C₁₋₆alkyl;
   R¹³ is H or C₁₋₆alkyl;
   R¹⁴ and R¹⁵ are independently selected from H, C₁₋₆alkyl, or —C₁₋₆alkylene-4-6 membered saturated heterocyclyl;
R² is H or C₁₋₃alkyl;
R³ is 6 to 10 membered aryl or 6 to 10 member heteroaryl, wherein said 6 to 10 membered aryl and 6 to 10 member heteroaryl represented by R³ are optionally substituted by one or more R^{C}; wherein
   R^{C} is halo, —CN, —OH, C₁₋₆alkyl, C₁₋₆haloalkyl, or C₁₋₆alkoxy, or two R^{C} together with the intervening atoms together form 5 to 7 membered heterocyclyl; wherein said 5 to 7 membered heterocyclyl represented by R^{C} is optionally substituted by R^{C1}; where R^{C1} is C₁₋₃alkyl or oxo; and
wherein said heterocyclyl comprises 1-3 heteroatoms independently selected from oxygen, nitrogen, and sulfur; and said heteroaryl comprises 1-4 heteroatoms independently selected from oxygen, nitrogen, and sulfur;
provided that the compound of formula (I′) is not represented by

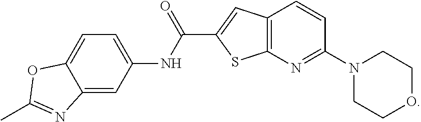 [SMILES: Cc1nc2cc(NC(=O)c3cc4ccc(N5CCOCC5)nc4s3)ccc2o1]

In a first embodiment, the present disclosure provides a compound of Formula (I):

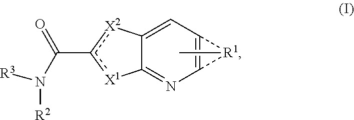

or a pharmaceutically acceptable salt thereof, wherein:
[symbol] is a single bond or double bond, provided the ring containing X₁ and X₂ is a 5-membered heteroaryl ring;

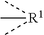

indicates that R¹ is substituted at one of two positions on the pyridyl moiety to which the dash lines connect and the other position to which the dash lines connect is unsubstituted;
X¹ is S or CH;
X² is N, O or CH;
R¹ is 4 to 12 membered heterocyclyl, —NR¹¹R¹², or —C₁₋₆alkylene-NR¹³R¹⁴, wherein
said 4 to 12-membered heterocyclyl represented by R¹ is optionally substituted with one or more R^{A}; wherein
each R^{A} is independently C₁₋₆alkyl, C₃₋₆cycloalkyl, haloC₁₋₆alkyl, —NR^{a}R^{b}, —C₁₋₃alkylene-NR^{a}R^{b}, —C₃₋₆cycloalkylene-NR^{a}R^{b}, —C(═O)R^{a}, or 4 to 6-membered saturated heterocyclyl; wherein each R^{a} and R^{b} is independently H or C₁₋₆alkyl; wherein said 4 to 6-membered saturated heterocyclyl represented by R^{A} is optionally substituted by one or more C₁₋₆alkyl;
R¹¹ is H or C₁₋₆alkyl;
R¹² is C₁₋₆alkyl, 6 to 10-membered aryl, 4 to 12-membered heterocyclyl, or 5-10 membered heteroaryl; wherein said C₁₋₆alkyl, 6 to 10-membered aryl, 4 to 12-membered heterocyclyl, or 5-10 membered heteroaryl represented by R¹² is optionally substituted by one or more R^{B}; wherein
R^{B} is C₁₋₆alkyl, —NR^{a}R^{b}, 4 to 6-membered heterocyclyl, or —C₁₋₆alkylene-4 to 6-membered heterocyclyl; wherein said 4 to 6-membered heterocyclyl represented by R^{B} is optionally substituted by one or more C₁₋₆alkyl;
R¹³ is H or C₁₋₆alkyl;
R¹⁴ is H, C₁₋₆alkyl, or —C₁₋₆alkylene-4-6 membered saturated heterocyclyl;
R² is H or C₁₋₃alkyl;
R³ is 6 to 10 membered aryl or 6 to 10 member heteroaryl, wherein said 6 to 10 membered aryl and 6 to 10 member heteroaryl represented by R³ are optionally substituted by one or more R^{C}; wherein
R^{C} is halo, —CN, —OH, C₁₋₆alkyl, C₁₋₆haloalkyl, or C₁₋₆alkoxy, or two R^{C} together with the intervening atoms together form 5 to 7 membered heterocyclyl; wherein said 5 to 7 membered heterocyclyl represented by R^{C} is optionally substituted by R^{C1}; where R^{C1} is C₁₋₃alkyl or oxo; and
wherein said heterocyclyl comprises 1-3 heteroatoms independently selected from oxygen, nitrogen, and sulfur; and said heteroaryl comprises 1-4 heteroatoms independently selected from oxygen, nitrogen, and sulfur;
provided that the compound of formula (I) is not represented by

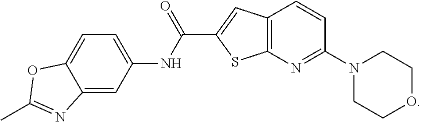 [SMILES: Cc1nc2cc(NC(=O)c3cc4ccc(N5CCOCC5)nc4s3)ccc2o1]

In a second embodiment, the present disclosure provides a compound according to the first aspect or the first embodiment or a pharmaceutically acceptable salt thereof, wherein the compound is represented by Formula (II):

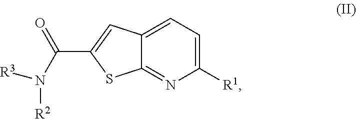

or a pharmaceutically acceptable salt thereof. The definitions of the variables are provided in the first aspect or the first embodiment.

In a third embodiment, the present disclosure provides a compound according to the first aspect or the first embodiment or a pharmaceutically acceptable salt thereof, wherein the compound is represented by Formula (III):

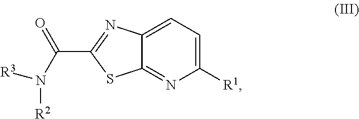

or a pharmaceutically acceptable salt thereof. The definitions of the variables are provided in the first aspect or the first embodiment.

In a fourth embodiment, the present disclosure provides a compound according to the first aspect or the first embodiment or a pharmaceutically acceptable salt thereof, wherein the compound is represented by Formula (IV):

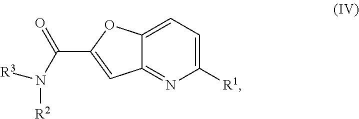

or a pharmaceutically acceptable salt thereof. The definitions of the variables are provided in the first aspect or the first embodiment.

In a fifth embodiment, the present disclosure provides a compound according to the first aspect or the first embodiment or a pharmaceutically acceptable salt thereof, wherein the compound is represented by Formula (V):

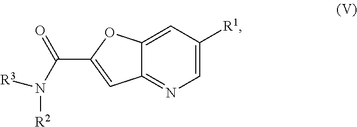

or a pharmaceutically acceptable salt thereof. The definitions of the variables are provided in the first aspect or the first embodiment.

In an alternate fifth embodiment, the present disclosure provides a compound according to the first aspect or the first embodiment or a pharmaceutically acceptable salt thereof, wherein the compound is represented by Formula (VI):

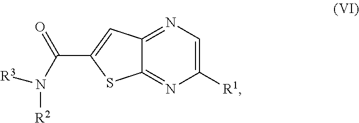

or a pharmaceutically acceptable salt thereof. The definitions of the variables are provided in the first aspect or the first embodiment.

In an alternate fifth embodiment, the present disclosure provides a compound according to the first aspect or the first embodiment or a pharmaceutically acceptable salt thereof, wherein the compound is represented by Formula (VII):

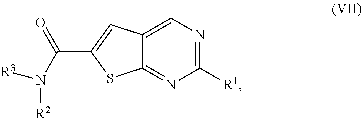

or a pharmaceutically acceptable salt thereof. The definitions of the variables are provided in the first aspect or the first embodiment.

In another alternate fifth embodiment, the present disclosure provides a compound according to the first aspect or the first embodiment or a pharmaceutically acceptable salt thereof, wherein the compound is represented by Formula (VIII):

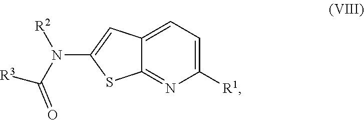

or a pharmaceutically acceptable salt thereof. The definitions of the variables are provided in the first aspect or the first embodiment.

In a sixth embodiment, the present disclosure provides a compound according to the first aspect or any one of the first through fifth embodiments or a pharmaceutically acceptable salt thereof, wherein R² is H. The definitions of the remaining variables are provided in the first aspect or any one of the first through the fifth embodiments or any alternative embodiments described therein.

In a seventh embodiment, the present disclosure provides a compound according to the first aspect or any one of the first through the sixth embodiments or a pharmaceutically acceptable salt thereof, wherein R¹ is a 4 to 12 membered saturated heterocyclyl. The definitions of the remaining variables are provided in the first aspect or any one of the first through the sixth embodiments or any alternative embodiments described therein.

In an eighth embodiment, the present disclosure provides a compound according to the first aspect or any one of the first through seventh embodiments or a pharmaceutically acceptable salt thereof, wherein:
R¹ is a 4 to 12 membered saturated heterocyclyl comprising one or two ring N atoms, provided when said heterocyclyl comprises one ring N atom, it is then optionally substituted with —NR⁷R⁸, —C₁₋₃alkylene-NR⁷R⁸ or —C₃₋₆cycloalkylene-NR⁷R⁸ and optionally further substituted with 1 to 4 R⁹, and when said heterocyclyl comprises two ring N atoms, it is optionally substituted with 1 to 3 R⁹;
R⁷ and R⁸ are each independently H or C₁₋₆alkyl; alternatively R⁷ and R⁸ taken together with N to which they are attached forms a 4 to 6 membered heteterocycle optionally substituted with 1 to 2 C₁₋₆alkyl, wherein said 4 to 6 membered heteterocycle optionally comprises a second hetero atom selected from N and O;
R⁹, for each occurrence, is independently selected from halo, —C(═O)R¹⁰, C₁₋₆alkyl, C₁₋₆haloalkyl, C₁₋₆alkoxyC₁₋₆alkyl, and C₃₋₆cycloalkyl; wherein said C₃₋₆cycloalkyl represented by R⁹ is optionally substituted by one or more substituents independently selected from halo and C₁₋₆alkyl; wherein R¹⁰ is H, C₁₋₃alkyl, or C₃₋₆cycloalkyl. The definitions of the remaining variables are provided in the first aspect or any one of the first through the seventh embodiments or any alternative embodiments described therein.

In an alternative eighth embodiment, the present disclosure provides a compound according to the first aspect or any one of the first through seventh embodiments or a pharmaceutically acceptable salt thereof, wherein:
R¹ is a 4 to 12 membered saturated heterocyclyl comprising one or two ring N atoms, provided when said heterocyclyl comprises one ring N atom, it is then optionally substituted with —NR⁷R⁸, —C₁₋₃alkylene-NR⁷R⁸ or —C₃₋₆cycloalkylene-NR⁷R⁸ and optionally further substituted with 1 to 2 R⁹, and when said heterocyclyl comprises two ring N atoms, it is optionally substituted with 1 to 3 R⁹;
R⁷ and R⁸ are each independently H or C₁₋₆alkyl; alternatively R⁷ and R⁸ taken together with N to which they are attached forms a 4 to 6 membered heteterocycle optionally substituted with 1 to 2 C₁₋₆alkyl, wherein said 4 to 6 membered heteterocycle optionally comprises a second hetero atom selected from N and O;
R⁹, for each occurrence, is independently selected from halo, —C(═O)R¹⁰, C₁₋₆alkyl, C₁₋₆haloalkyl, C₁₋₆alkoxyC₁₋₆alkyl, and C₃₋₆cycloalkyl; wherein said C₃₋₆cycloalkyl represented by R⁹ is optionally substituted by one or more substituents independently selected from halo and C₁₋₆alkyl; wherein R¹⁰ is H, C₁₋₃alkyl, or C₃₋₆cycloalkyl. The definitions of the remaining variables are provided in the first aspect or any one of the first through the seventh embodiments or any alternative embodiments described therein.

In a ninth embodiment, the present disclosure provides a compound according to eighth embodiment or a pharmaceutically acceptable salt thereof, wherein R¹ is a 4 to 12 membered saturated heterocyclyl comprising one ring N atom and is substituted with 1 to 4 R⁹. The definitions of the remaining variables are provided in the eighth embodiment or any alternative embodiments described therein.

In an alternative ninth embodiment, the present disclosure provides a compound according to eighth embodiment or a pharmaceutically acceptable salt thereof, wherein R¹ is selected from pyrrolidinyl, piperidinyl, azabicyclo[3.2.1]octanyl, and azaspiro[3.4]octanyl. The definitions of the remaining variables are provided in the eighth embodiment or any alternative embodiments described therein.

In another alternative ninth embodiment, the present disclosure provides a compound according to eighth embodiment or a pharmaceutically acceptable salt thereof, wherein R¹ is selected from:

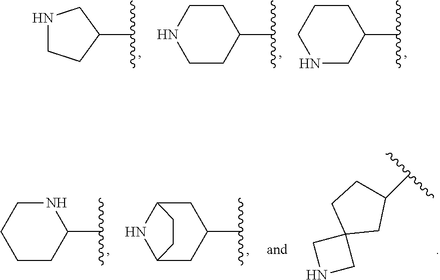

The definitions of the remaining variables are provided in the eighth embodiment or any alternative embodiments described therein.

In a tenth embodiment, the present disclosure provides a compound according to eighth embodiment or a pharmaceutically acceptable salt thereof, wherein R¹ is a 4 to 12 membered saturated heterocyclyl comprising one ring N atom and is substituted with —NR⁷R⁸—C₁₋₃alkylene-NR⁷R⁸ or —C₃₋₆cycloalkylene-NR⁷R⁸ and optionally further substituted with 1 to 2 R⁹. The definitions of the remaining variables are provided in the eighth embodiment or any alternative embodiments described therein.

In an alternative tenth embodiment, the present disclosure provides a compound according to eighth embodiment or a pharmaceutically acceptable salt thereof, wherein R¹ is a 4 to 12 membered saturated heterocyclyl selected from azetidinyl, piperidinyl, pyrrolidinyl, octahydro-1H-isoindolyl, and 3-azabicyclo[3.1.0]hexanyl, each of which is substituted with —NR⁷R⁸, —C₁₋₃alkylene-NR⁷R⁸ or —C₃₋₆cycloalkylene-NR⁷R⁸ and optionally further substituted with 1 to 2 R⁹. The definitions of the remaining variables are provided in the eighth embodiment or any alternative embodiments described therein.

In an eleventh embodiment, the present disclosure provides a compound according to tenth embodiment or a pharmaceutically acceptable salt thereof, wherein R¹ is selected from

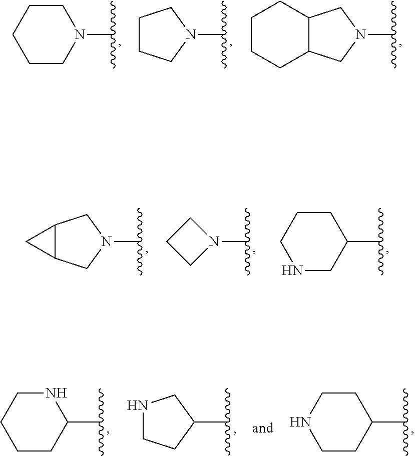

each of which is substituted with —NR⁷R⁸, —C₁₋₃alkylene-NR⁷R⁸ or —C₃₋₆cycloalkylene-NR⁷R⁸ and optionally further substituted with 1 to 2 R⁹. The definitions of the remaining variables are provided in the tenth embodiment or any alternative embodiments described therein.

In an alternataive eleventh embodiment, the present disclosure provides a compound according to tenth embodiment or a pharmaceutically acceptable salt thereof, wherein R¹ is selected from

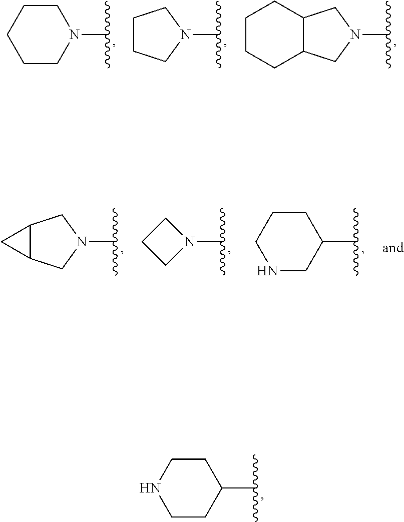

each of which is substituted with —NR⁷R⁸, —C₁₋₃alkylene-NR⁷R⁸ or —C₃₋₆ cycloalkylene-NR⁷R⁸ and optionally further substituted with 1 to 2 R⁹. The definitions of the remaining variables are provided in the tenth embodiment or any alternative embodiments described therein.

In a twelfth embodiment, the present disclosure provides a compound according to the first aspect or any one of the first through the eleventh embodiments or a pharmaceutically acceptable salt thereof, wherein R⁷ and R⁸ are each independently H or C₁₋₃alkyl; alternatively R⁷ and R⁸ taken together are C₂-C₄ alkylene, optionally substituted with 1 or 2 C₁₋₃alkyl. The definitions of the remaining variables are provided in the first aspect or any one of the first through the eleventh embodiments or any alternative embodiments described therein.

In a thirteenth embodiment, the present disclosure provides a compound according to the first aspect or any one of the first through the twelfth embodiments or a pharmaceutically acceptable salt thereof, wherein R⁷ and R⁸ are each independently H, —CH₃ or —CH₂CH₃; alternatively R⁷ and R⁸ taken together are —CH₂CH₂CH₂CH₂—, —CH₂CH₂CH₂— or —CH₂C(CH₃)₂CH₂—. The definitions of the remaining variables are provided in the first aspect or any one of the first through the twelfth embodiments or any alternative embodiments described therein.

In a fourteenth embodiment, the present disclosure provides a compound according to any one of the ninth through the eleventh embodiments or a pharmaceutically acceptable salt thereof, wherein R¹ is selected from a group consisting of

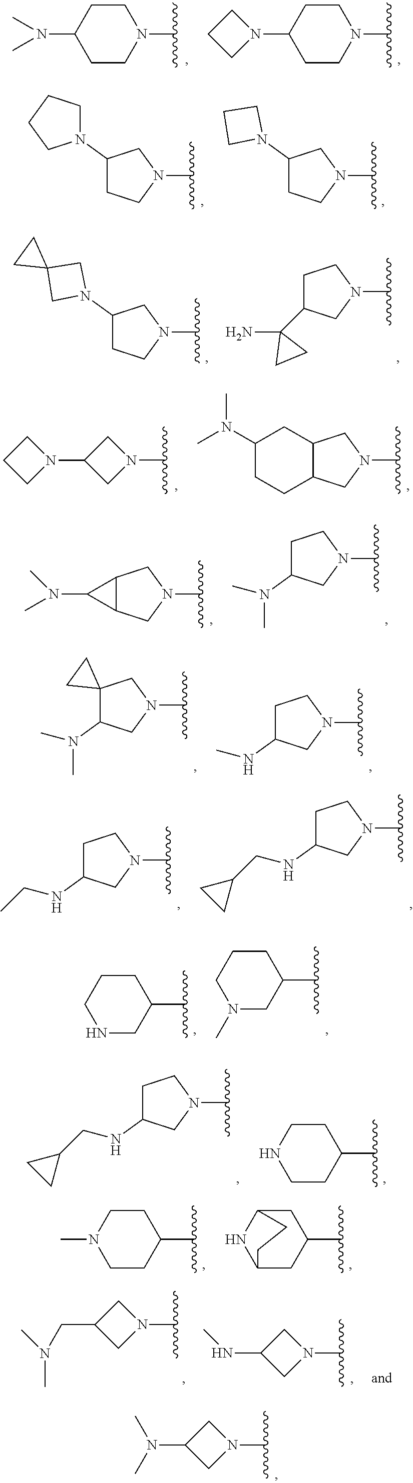

each of which is optionally further substituted with 1 to 2 R⁹. The definitions of the remaining variables are provided in any one of the ninth through the eleventh embodiments or any alternative embodiments described therein.

In an alternative fourteenth embodiment, the present disclosure provides a compound according to any one of the ninth through the eleventh embodiments or a pharmaceutically acceptable salt thereof, wherein R¹ is selected from a group consisting of

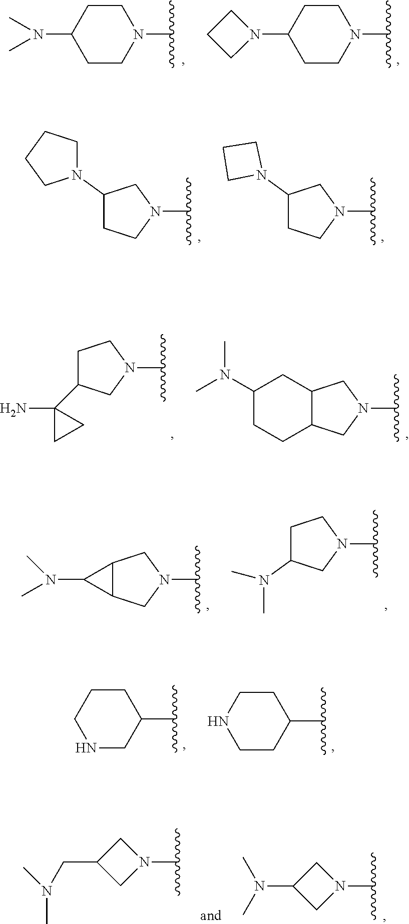

each of which is optionally further substituted with 1 to 2 R⁹. The definitions of the remaining variables are provided in any one of the ninth through the eleventh embodiments or any alternative embodiments described therein.

In a fifteenth embodiment, the present disclosure provides a compound according to the eighth embodiment or a pharmaceutically acceptable salt thereof, wherein R¹ is a 4 to 12 membered saturated heterocyclyl comprising two ring N atoms and is optionally substituted with 1 to 3 R⁹. The definitions of the remaining variables are provided in the eighth embodiment or any alternative embodiments described therein.

In a sixteenth embodiment, the present disclosure provides a compound according to the fifteenth embodiment or a pharmaceutically acceptable salt thereof, the 4 to 12 membered saturated heterocyclyl represented by R¹ is piperazinyl, 4,7-diazaspiro[2.5]octanyl, 3,9-diazaspiro[5.5]undecanyl, 1-oxa-4,9-diazaspiro[5.5]undecanyl, diazabicyclo[2.2.2]octanyl, octahydro-2H-pyrido[4,3-b][1,4]oxazinyl, octahydro-1H-pyrrolo[2,3-c]pyridinyl, 2,5-diazabicyclo[2.2.1]heptanyl, octahydropyrrolo[1,2-a]pyrazinyl, decahydro-1,6-naphthyridinyl, 1,6-diazaspiro[3.4]octanyl, 1,5-diazaspiro[3.4]octanyl, 2λ²,5-diazaspiro[3.4]octanyl, 2λ²,6-diazaspiro[3.4]octanyl, hexahydropyrrolo[3,4-c]pyrrolyl, octahydropyrrolo[3,4-c]pyrrolyl, octahydro-1H-pyrrolo[2,3-c]pyridinyl, octahydropyrrolo[3,4-b]pyrrolyl, 3,6-diazabicyclo[3.2.0]heptanyl, 1,4-diazepanyl, 2,6-diazaspiro[3.5]nonane, 2,6-diazabicyclo[3.2.0]heptanyl, or 1,7-diazaspiro[4.4]nonanyl, each of which is optionally substituted with 1 to 2 R⁹. The definitions of the remaining variables are provided in the fifteenth embodiment or any alternative embodiments described therein

In an alternative sixteenth embodiment, the present disclosure provides a compound according to the fifteenth embodiment or a pharmaceutically acceptable salt thereof, the 4 to 12 membered saturated heterocyclyl represented by R^{r} is piperazinyl, diazabicyclo[2.2.2]octanyl, octahydro-2H-pyrido[4,3-b][1,4]oxazinyl, octahydro-1H-pyrrolo[2,3-c]pyridinyl, 2,5-diazabicyclo[2.2.1]heptanyl, octahydropyrrolo[1,2-a]pyrazinyl, decahydro-1,6-naphthyridinyl, hexahydropyrrolo[3,4-c]pyrrolyl, octahydropyrrolo[3,4-c]pyrrolyl, octahydro-1H-pyrrolo[2,3-c]pyridinyl, octahydropyrrolo[3,4-b]pyrrolyl, 1,4-diazepanyl, or 2,6-diazaspiro[3.5]nonane, each of which is optionally substituted with 1 to 2 R⁹. The definitions of the remaining variables are provided in the fifteenth embodiment or any alternative embodiments described therein.

In a seventeenth embodiment, the present disclosure provides a compound according to the sixteenth embodiment or a pharmaceutically acceptable salt thereof, wherein the 4 to 12 membered saturated heterocyclyl represented by R¹ is:

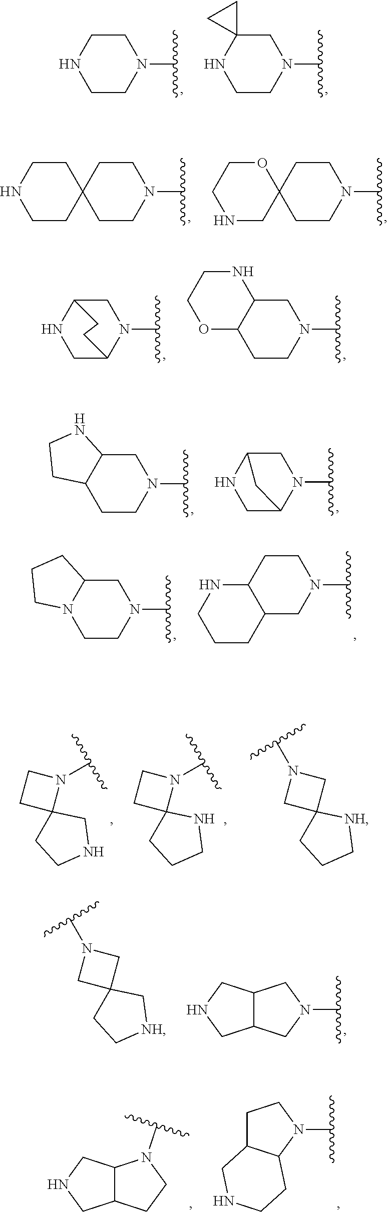

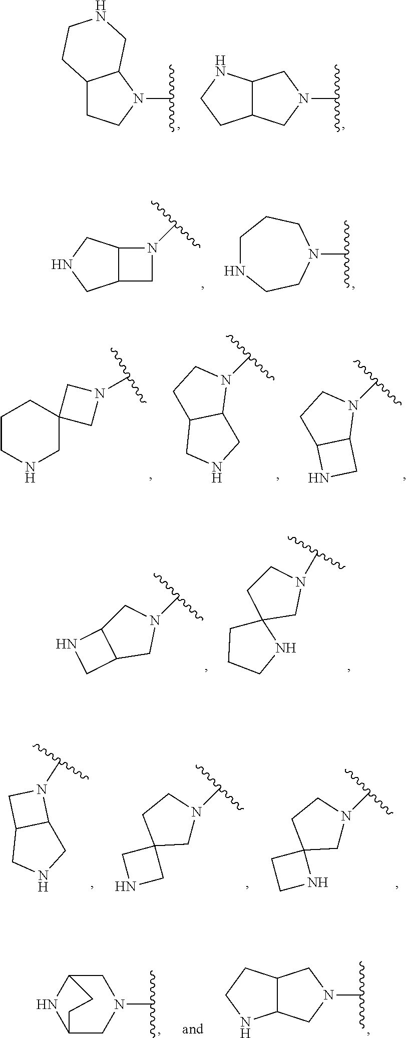

each of which is optionally substituted 1 or 3 R⁹. The definitions of the remaining variables are provided in the sixteenth embodiment or any alternative embodiments described therein.

In an alternative seventeenth embodiment, the present disclosure provides a compound according to the sixteenth embodiment or a pharmaceutically acceptable salt thereof, wherein the 4 to 12 membered saturated heterocyclyl represented by R¹ is:

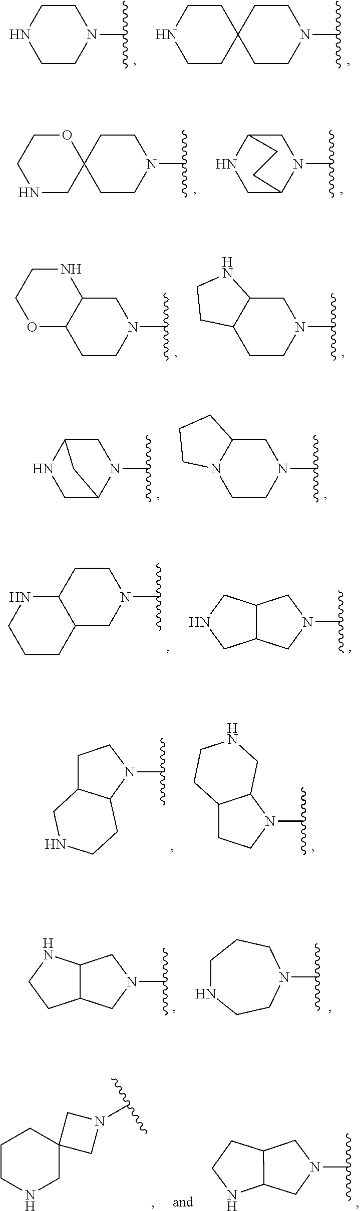

each of which is optionally substituted 1 or 3 R⁹. The definitions of the remaining variables are provided in the sixteenth embodiment or any alternative embodiments described therein.

In an eighteenth embodiment, the present disclosure provides a compound according to the first aspect or any one of the first through the sixth embodiments or a pharmaceutically acceptable salt thereof, wherein R¹ is 4 to 12 membered partially saturated heterocyclyl. The definitions of the remaining variables are provided in the first aspect or any one of the first through the sixth embodiments or any alternative embodiments described therein.

In a nineteenth embodiment, the present disclosure provides a compound according to the eighteenth embodiment or a pharmaceutically acceptable salt thereof, wherein the partially saturated heterocyclyl represented by R¹ is 2,3,4,5-tetrahydro-1H-pyrido[2,3-e][1,4]diazepine, 1,2,3,6-tetrahydropyridinyl, 6-azabicyclo[3.1.1]hept-2-enyl. or 8-azabicyclo[3.2.1]oct-2-enyl. The definitions of the remaining variables are provided in the eighteenth embodiment or any alternative embodiments described therein.

In an alternative nineteenth embodiment, the present disclosure provides a compound according to the eighteenth embodiment or a pharmaceutically acceptable salt thereof, wherein the partially saturated heterocyclyl represented by R¹ is 2,3,4,5-tetrahydro-1H-pyrido[2,3-e][1,4]diazepine, 1,2,3,6-tetrahydropyridinyl or 8-azabicyclo[3.2.1]oct-2-enyl. The definitions of the remaining variables are provided in the eighteenth embodiment or any alternative embodiments described therein.

In a twentieth embodiment, the present disclosure provides a compound according to the eighteenth embodiment or the nineteenth embodiment or a pharmaceutically acceptable salt thereof, wherein the partially saturated heterocyclyl represented by R¹ is selected from a group consisting of:

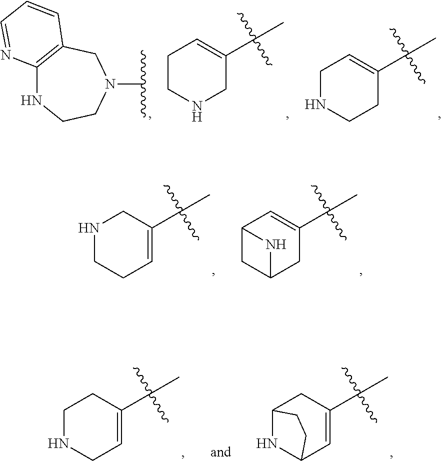

each of which is optionally substituted with 1, 2, 3 or 4 R⁹. The definitions of the remaining variables are provided in the eighteenth embodiment or the nineteenth embodiment or any alternative embodiments described therein.

In an alternative twentieth embodiment, the present disclosure provides a compound according to the eighteenth embodiment or the nineteenth embodiment or a pharmaceutically acceptable salt thereof, wherein the partially saturated heterocyclyl is selected from a group consisting of:

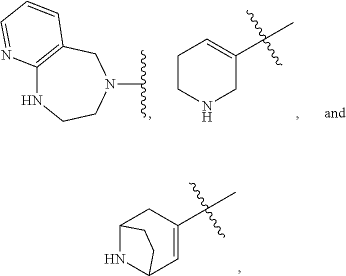

each of which is optionally substituted with 1 or 2 R⁹. The definitions of the remaining variables are provided in the eighteenth embodiment or the nineteenth embodiment or any alternative embodiments described therein.

In a twenty-first embodiment, the present disclosure provides a compound according to the first aspect or any one of the first through the sixth embodiments or a pharmaceutically acceptable salt thereof, wherein R¹ is 4 to 12 membered saturated or partially saturated carbocyclyl substituted with —NR⁷R⁸ and is further optionally substituted with 1 or 2 R⁹. The definitions of the remaining variables are provided in the first aspect or any one of the first through the sixth embodiments or any alternative embodiments described therein.

In an alternative twenty-first embodiment, the present disclosure provides a compound according to the first aspect or any one of the first through the sixth embodiments or a pharmaceutically acceptable salt thereof, wherein R¹ is cyclohexyl or cyclohexenyl, each of which is substituted with —NR⁷R⁸ and is further optionally substituted with 1 or 2 R⁹. The definitions of the remaining variables are provided in the first aspect or any one of the first through the sixth embodiments or any alternative embodiments described therein.

In yet another alternative twenty-first embodiment, the present disclosure provides a compound according to the first aspect or any one of the first through the sixth embodiments or a pharmaceutically acceptable salt thereof, wherein R¹ is selected from and

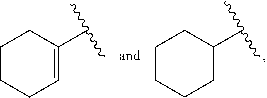 [SMILES: *C1=CCCCC1 ; *C1CCCCC1]

each of which is substituted with —NR⁷R⁸ and is further optionally substituted with 1 or 2 R⁹. The definitions of the remaining variables are provided in the first aspect or any one of the first through the sixth embodiments or any alternative embodiments described therein.

In a twenty-second embodiment, the present disclosure provides a compound according to the twenty-first embodiment or a pharmaceutically acceptable salt thereof, wherein R⁷ and R⁸ are each independently H or C₁₋₃alkyl. The definitions of the remaining variables are provided in the twenty-first embodiment or any alternative embodiments described therein.

In an alternative twenty-second embodiment, the present disclosure provides a compound according to the twenty-first embodiment or a pharmaceutically acceptable salt thereof, wherein R⁷ and R⁸ are each independently H or —CH₃. The definitions of the remaining variables are provided in the twenty-first embodiment or any alternative embodiments described therein.

In a twenty-third embodiment, the present disclosure provides a compound according to the first aspect or any one of the first through the twenty-second embodiments or a pharmaceutically acceptable salt thereof, wherein R⁹, for each occurrence, is independently selected from halo, —C(═O)R¹⁰, C₁₋₄alkyl, C₁₋₄haloalkyl, and C₃₋₆cycloalkyl; wherein said C₃₋₆cycloalkyl represented by R⁹ is optionally substituted by one to three substituents independently selected from F, Cl, and C₁₋₄alkyl; and R¹⁰ is H, C₁₋₂alkyl, C₃₋₄cycloalkyl. The definitions of the remaining variables are provided in the first aspect or any one of the first through the twenty-second embodiments or any alternative embodiments described therein.

In a twenty-fourth embodiment, the present disclosure provides a compound according to the first aspect or any one of the first through the twenty-second embodiments or a pharmaceutically acceptable salt thereof, wherein R⁹, for each occurrence, is independently selected from F, —CH₃, —CH₂CH₃, —C(═O)CH₃, —CH₂CF₃, —CH(CH₃)₂, —CD₃, and cyclopropyl. The definitions of the remaining variables are provided in the first aspect or any one of the first through the twenty-second embodiments or any alternative embodiments described therein.

In an alternative twenty-fourth embodiment, the present disclosure provides a compound according to the first aspect or any one of the first through the twenty-second embodiments or a pharmaceutically acceptable salt thereof, wherein R⁹, for each occurrence, is independently selected from —CH₃, —C(═O)CH₃, —CH₂CF₃, —CH(CH₃)₂, and cyclopropyl. The definitions of the remaining variables are provided in the first aspect or any one of the first through the twenty-second embodiments or any alternative embodiments described therein.

In a twenty-fifth embodiment, the present disclosure provides a compound according to the first aspect or any one of the first through the sixth embodiment or a pharmaceutically acceptable salt thereof, wherein:
R¹ is —NR¹¹R¹²;
R¹¹ is H or C₁₋₆alkyl;
R¹² is C₁₋₆alkyl-NR^{a}R^{b}, phenyl, 4 to 12-membered heterocyclyl comprising at least one ring N atom; wherein said phenyl represented by R¹² is substituted with —NR^{a}R^{b}, Het, or —C₁₋₃alkylene-Het, and Het is a 4 to 6-membered heterocyclyl comprising at least one ring N atom and is optionally substituted with one or two C₁₋₃alkyl; and wherein said 4 to 12-membered heterocyclyl represented by R¹² is optionally substituted by one, two, three, four or five R^{12a}; wherein each R^{12a} is independently C₁₋₃alkyl or halo. The definitions of the remaining variables are provided in the first aspect or any one of the first through the sixth embodiments or any alternative embodiments described therein.

In an alternative twenty-fifth embodiment, the present disclosure provides a compound according to the first aspect or any one of the first through the sixth embodiment or a pharmaceutically acceptable salt thereof, wherein:
R¹ is —NR¹¹R¹²;
R¹¹ is H or C₁₋₆alkyl;
R¹² is C₁₋₆alkyl-NR^{a}R^{b}, phenyl, 4 to 12-membered heterocyclyl comprising at least one ring N atom; wherein said phenyl represented by R¹² is substituted with —NR^{a}R^{b}, Het, or —C₁₋₃alkylene-Het, and Het is a 4 to 6-membered heterocyclyl comprising at least one ring N atom and is optionally substituted with one or two C₁₋₃alkyl; and wherein said 4 to 12-membered heterocyclyl represented by R¹² is optionally substituted by one or two C₁₋₃alkyl. The definitions of the remaining variables are provided in the first aspect or any one of the first through the sixth embodiments or any alternative embodiments described therein.

In a twenty-sixth embodiment, the present disclosure provides a compound according to the first aspect or any one of the first through the sixth embodiments or a pharmaceutically acceptable salt thereof, wherein:
R¹ is —NR¹¹R¹²;
R¹¹ is H or —CH₃;
R¹² is selected from a group consisting of: piperidinyl, hexahydro-1H-pyrrolizinyl, octahydrocyclopenta[c]pyrrolyl, octahydroindolizinyl, isoindolinyl, phenylazetidinyl, 1,2,3,4,5-tetrahydro-1H-benzo[e][1,4]diazepinyl, benzylpyrrolidinyl, and quinuclidinyl, each of which is optionally substituted with one, two, three, four or five R^{12a}; wherein R^{12a} is C₁₋₃alkyl or halo. The definitions of the remaining variables are provided in the first aspect or any one of the first through the sixth embodiments or any alternative embodiments described therein.

In some embodiments, for the compounds according to the twenty-fifth or twenty-sixth embodiment or a pharmaceutically acceptable salt thereof, R^{12a} is methyl or fluoro. The definitions of the remaining variables are provided in twenty-fifth embodiment or twenty-sixth embodiment or any alternative embodiments described therein.

In an alternative twenty-sixth embodiment, the present disclosure provides a compound according to the first aspect or any one of the first through the sixth embodiment, or a pharmaceutically acceptable salt thereof, wherein:
R¹ is —NR¹¹R¹²;
R¹¹ is H or —CH₃;
R¹² is selected from a group consisting of: hexahydro-1H-pyrrolizinyl, octahydrocyclopenta[c]pyrrolyl, octahydroindolizinyl, isoindolinyl, phenylazetidinyl, 1,2,3,4,5-tetrahydro-TH-benzo[e][1,4]diazepinyl, benzylpyrrolidinyl, and quinuclidinyl, each of which is optionally substituted with one or two independently C₁₋₂alkyl. The definitions of the remaining variables are provided in the first aspect or any one of the first through the sixth embodiments or any alternative embodiments described therein.

In a twenty-seventh embodiment, the present disclosure provides a compound according to the first aspect or any one of the first through the sixth embodiment or a pharmaceutically acceptable salt thereof, wherein:
R¹ is —NR¹¹R¹²;
R¹¹ is H or —CH₃;
R¹² is selected from a group consisting of:

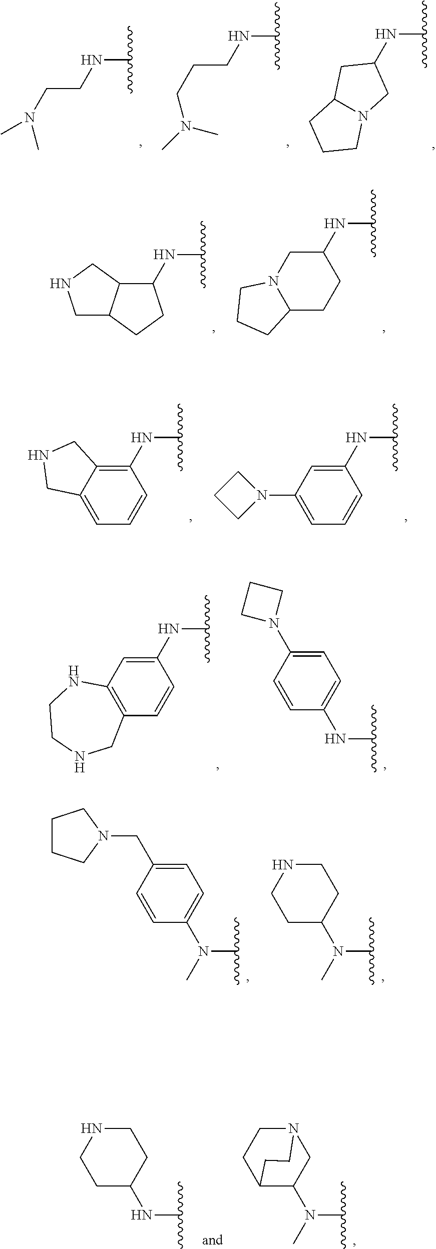

each of which is optionally substituted with one, two, three, four, or five substituents independently selected from, F, —CH₃ and —CH₂CH₃. The definitions of the remaining variables are provided in the first aspect or any one of the first through the sixth embodiments or any alternative embodiments described therein.

In an alternative twenty-seventh embodiment, the present disclosure provides a compound according to the first aspect or any one of the first through the sixth embodiments or a pharmaceutically acceptable salt thereof, wherein:
R¹ is —NR¹¹R¹²;
R¹¹ is H or —CH₃;
R¹² is selected from a group consisting of:

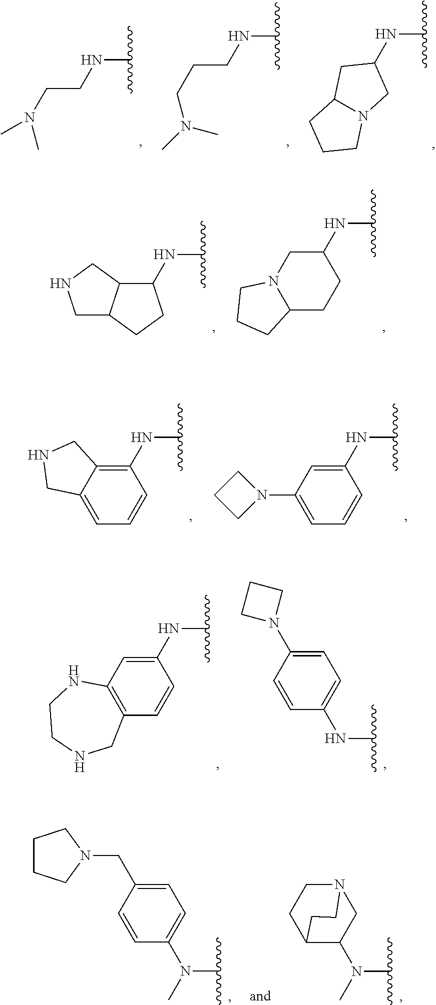

each of which is optionally substituted with one or two substituents independently selected from —CH₃ and —CH₂CH₃. The definitions of the remaining variables are provided in the first aspect or any one of the first through the sixth embodiments or any alternative embodiments described therein.

In a twenty-eighth embodiment, the present disclosure provides a compound according to the first aspect or any one of the first through the sixth embodiment or a pharmaceutically acceptable salt thereof, wherein:
R¹ is —OR¹⁵;
R¹⁵ is C₁₋₆alkyl-NR^{a}R^{b}, phenyl, 4 to 12-membered carbocyclyl, 4 to 12-membered heterocyclyl comprising at least one ring N atom; wherein said phenyl or 4 to 12-membered carbocyclyl represented by R¹⁵ is substituted with —NR^{a}R^{b}, Het, or —C₁₋₃alkylene-Het, and Het is a 4 to 6-membered heterocyclyl comprising at least one ring N atom and is optionally substituted with one or two C₁₋₃alkyl; and wherein said 4 to 12-membered heterocyclyl represented by R¹⁵ is optionally substituted by one or two C₁₋₃alkyl. The definitions of the remaining variables are provided in the first aspect or any one of the first through the sixth or any alternative embodiments described therein.

In some embodiments, for the compounds according to the twenty-eighth embodiment or a pharmaceutically acceptable salt thereof, R¹⁵ is selected from piperidinyl, pyrrolidinyl, 8-azaspiro[4.5]decanyl, and 7-azaspiro[3.5]nonanyl, each of which is optionally substituted with one or two C₁₋₃alkyl or R¹⁵ is cyclopentyl substituted with NR^{a}R^{b}; and R^{a} and R^{b} are each independently H or C₁₋₃alkyl. The definitions of the remaining variables are provided in the twenty-eighth embodiment.

In twenty-ninth embodiment, the present disclosure provides a compound according to the first aspect or any one of the first through the sixth embodiment or a pharmaceutically acceptable salt thereof, wherein:
R¹ is —OR¹⁵;
R¹⁵ is selected from a group consisting of:

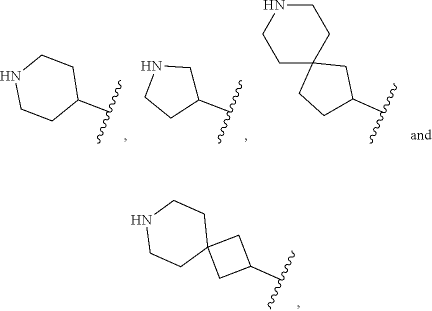

each of which is optionally substituted with one or two substituents independently selected from —CH₃ and —CH₂CH₃; or R¹⁵ is represented by

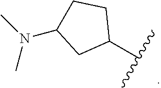 [SMILES: *C1CCC(N(C)C)C1]

The definitions of the remaining variables are provided in the first aspect or any one of the first through the sixth or any alternative embodiments described therein.

In a thirtieth embodiment, the present disclosure provides a compound according to the first aspect or any one of the first through the twenty-ninth embodiments or a pharmaceutically acceptable salt thereof, wherein R³ is a 9-membered bicyclic heteroaryl optionally substituted by one to three R^{C} or a phenyl fused with a 5-membered heterocyclyl optional substituted with one to three R^{C1}. The definitions of the remaining variables are provided in the first aspect or any one of the first through the twenty-ninth embodiments or any alternative embodiments described therein.

In a thirty-first embodiment, the present disclosure provides a compound according to the first aspect or any one of the first through the twenty-ninth embodiments or a pharmaceutically acceptable salt thereof, wherein R³ is selected from a group consisting of indazolyl, imidazopyridinyl, imidazopyridazinyl, imidazopyrazinyl, benzothiazolyl, triazolopyrazinyl, benzooxazolyl, pyrazolopyrimidinyl, and benzothiadiazolyl, each of which is optionally substituted with one to three R^{C} or R³ is 1,3-dihydro-2H-benzo[d]imidazol-2-one or benzo[d]thiazol-2(3H)-one, each of which is optionally substituted with one or two R^{C1}. The definitions of the remaining variables are provided in the first aspect or any one of the first through the twenty-ninth embodiments or any alternative embodiments described therein.

In a thirty-second embodiment, the present disclosure provides a compound according to the thirtieth or thirty-first embodiment or a pharmaceutically acceptable salt thereof, wherein R³ is selected from a group consisting of:

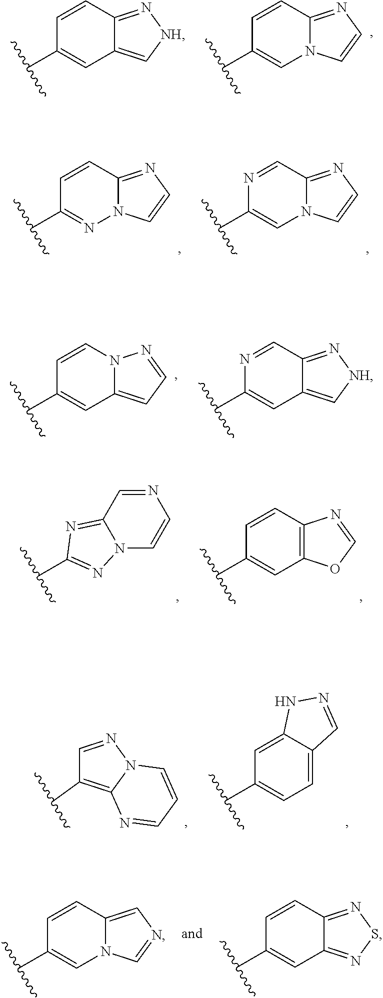

each of which is optionally substituted with one to three R^{C}; or
R³ is

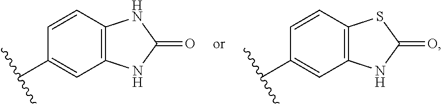 [SMILES: *c1ccc2[nH]c(=O)[nH]c2c1 ; *c1ccc2sc(=O)[nH]c2c1]

each of which is optionally substituted with one or two R^{C1}. The definitions of the remaining variables are provided in the thirtieth or thirty-first embodiment or any alternative embodiments described therein.

In an alternative thirty-second embodiment, the present disclosure provides a compound according to the thirtieth or thirty-first embodiment or a pharmaceutically acceptable salt thereof, wherein R³ is selected from a group consisting of:

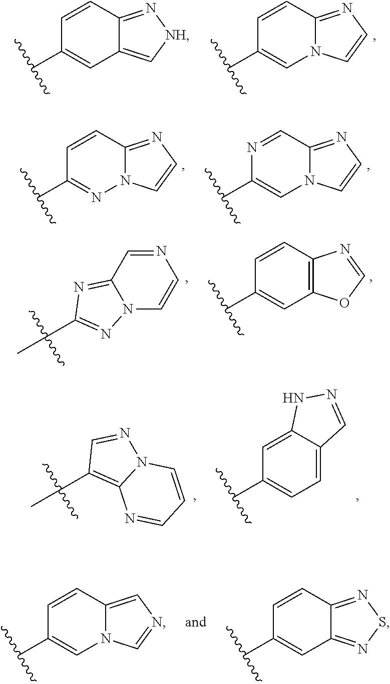

each of which is optionally substituted with one to three R^{C}; or
R³ is

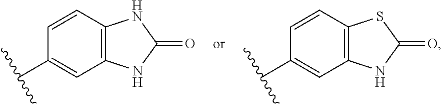 [SMILES: *c1ccc2[nH]c(=O)[nH]c2c1 ; *c1ccc2sc(=O)[nH]c2c1]

each of which is optionally substituted with one or two R^{C1}. The definitions of the remaining variables are provided in the thirtieth or thirty-first embodiment or any alternative embodiments described therein.

In a thirty-third embodiment, the present disclosure provides a compound according to the first aspect or any one of the first through the thirty-second embodiments or a pharmaceutically acceptable salt thereof, wherein R^{C} for each occurrence is independently halo, C₁₋₃alkyl, C₁₋₂haloalkyl, or C₁₋₂alkoxy; and R^{C1} for each occurrence is independently C₁₋₃alkyl. The definitions of the remaining variables are provided in the first aspect or any one of the first through the thirty-second embodiments or any alternative embodiments described therein.

In a thirty-fourth embodiment, the present disclosure provides a compound according to the thirty-third embodiment or a pharmaceutically acceptable salt thereof, wherein R^{C} for each occurrence is independently selected from —F, —CH₃, —CH(CH₃)₂, —CF₃, and —OCH₃; and R^{C1} is —CH₃. The definitions of the remaining variables are provided in the thirty-third embodiment or any alternative embodiments described therein.

In a thirty-fifth embodiment, the present disclosure provides a compound according to the first aspect or the first embodiment or a pharmaceutically acceptable salt thereof, wherein the compound is represented by the following Formula (IIA):

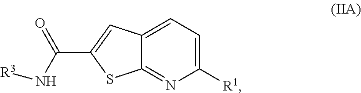

or a pharmaceutically acceptable salt thereof, wherein R¹ is piperazinyl, pyrrolidinyl, diazabicyclo[2.2.1]heptanyl, octahydropyrrolo[3,4-b]pyrrolyl, piperidinyl, 8-azabicyclo[3.2.1]oct-2-enyl or 1,2,3,6-tetrahydropyridinyl, wherein said piperazinyl, pyrrolidinyl, diazabicyclo[2.2.1]heptanyl, octahydropyrrolo[3,4-b]pyrrolyl, piperidinyl, 8-azabicyclo[3.2.1]oct-2-enyl or 1,2,3,6-tetrahydropyridinyl is optionally substituted with 1 to 3 R⁹ and said pyrrolidinyl is optionally substituted with —NR⁷R⁸ or —C₃₋₆cycloalkylene-NR⁷R⁸ and further optionally substituted with 1 or 2 R⁹;
R⁷ and R⁸ are each independently H or C₁₋₄alkyl;
R⁹, for each occurrence, is independently selected from C₁₋₄alkyl and and C₃₋₆cycloalkyl; and
R³ is indazolyl, imidazopyridinyl, imidazopyrazinyl or benzooxazolyl, wherein said indazolyl, imidazopyridinyl, imidazopyrazinyl or benzooxazolyl is optionally substituted with one to two R^{C};
R^{C}, for each occurrence, is independently selected from C₁₋₄alkyl and halo. The definitions of the remaining variables are provided in the first aspect or the first embodiment.

In a thirty-sixth embodiment, the present disclosure provides a compound according to the thirty-fifth embodiment or a pharmaceutically acceptable salt thereof, wherein R is selected from a group consisting of:

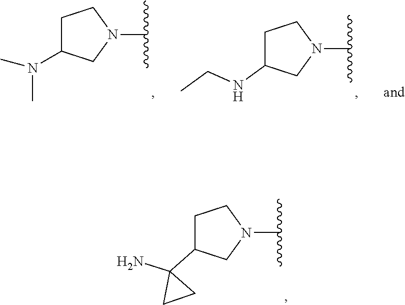

each of which is optionally substituted with 1 or 2 R⁹; or R¹ is selected from a group consisting of

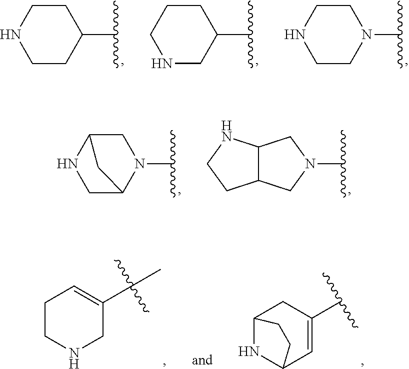

each of which is optionally substituted with 1 to 3 R⁹. The definitions of the remaining variables are provided in the thirty-fifth embodiment or any alternative embodiments described therein.

In a thirty-seventh embodiment, the present disclosure provides a compound according to the thirty-fifth embodiment or the thirty-sixth embodiment or a pharmaceutically acceptable salt thereof, wherein R³ selected from a group consisting of:

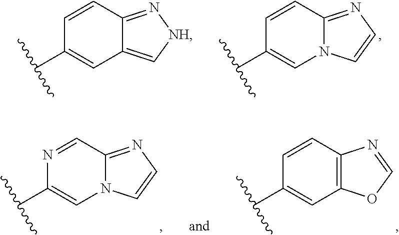 [SMILES: *c1ccc2n[nH]cc2c1 ; *c1ccc2nccn2c1 ; *c1cn2ccnc2cn1 ; *c1ccc2ncoc2c1]

each of which is is optionally substituted with one to two R^{C}. The definitions of the remaining variables are provided in the thirty-fifth embodiment or the thirty-sixth embodiment or any alternative embodiments described therein.

In a thirty-eighth embodiment, the present disclosure provides a compound according to any one of the thirty-fifth through the thirty-seventh embodiments or a pharmaceutically acceptable salt thereof, wherein R⁹, for each occurrence, is independently selected from —CH₃ and cyclopropyl. The definitions of the remaining variables are provided in any one of the thirty-fifth through thirty-seventh embodiments or any alternative embodiments described therein.

In a thirty-ninth embodiment, the present disclosure provides a compound according to any one of the thirty-fifth through the thirty-eighth embodiments or a pharmaceutically acceptable salt thereof, wherein R^{C}, for each occurrence, is independently selected from CH₃ and F. The definitions of the remaining variables are provided in any one of the thirty-fifth through thirty-eighth embodiments or any alternative embodiments described therein.

In a fortieth embodiment, the present disclosure provides a compound according to the first aspect or the first embodiment or a pharmaceutically acceptable salt thereof, wherein the compound is represented by the following formula:

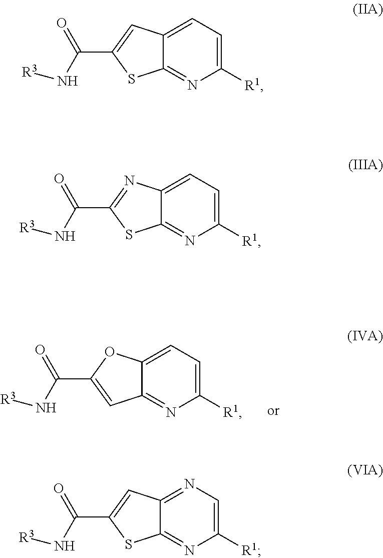

or a pharmaceutically acceptable salt thereof, wherein:
R¹ is piperazinyl, pyrrolidinyl, piperidinyl, diazasprio[4.4]nonanyl, diazabicyclo[3.2.0]heptanyl, or diazaspiro[3.4]octanyl, wherein said piperazinyl, piperidinyl, diazasprio[4.4]nonanyl, diazabicyclo[3.2.0]heptanyl, or diazaspiro[3.4]octanyl is optionally substituted with 1 to 3 R⁹ and said pyrrolidinyl is optionally substituted with —NR⁷R⁸ and is further optionally substituted with 1 or 2 R⁹;
R⁷ and R⁸ are each independently H or C₁₋₄alkyl; or R⁷ and R⁸ together with N atom from which they are attached form a 4 to 6 membered saturated monocyclic heterocyclyl;
R⁹, for each occurrence, is independently C₁₋₃alkyl; and
R³ is indazolyl, pyrazolo[1,5-a]pyridinyl, imidazopyridinyl, or imidazopyrazinyl, wherein said indazolyl, imidazopyridinyl, or imidazopyrazinyl is optionally substituted with one to two R^{C};
R^{C}, for each occurrence, is independently selected from C₁₋₃alkyl, C₁₋₃ haloalkyl, C₁₋₃alkoxy, and halo.

In a forty-first embodiment, for the compound of the fortieth embodiment or a pharmaceutically acceptable salt thereof, R¹ is selected from a group consisting of:

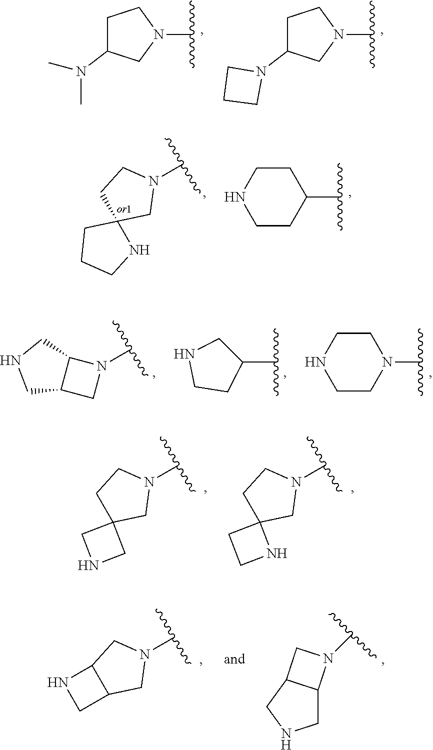

each or which is optionally substituted with 1 or 2 R⁹; and R⁹ for each occurrence is independently C₁₋₃alkyl. The definitions of the remaining variables are provided in fortieth embodiment. In a forty-second embodiment, for the compound of the fortieth or forty-first embodiment or a pharmaceutically acceptable salt thereof, R³ is

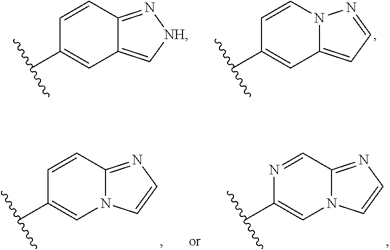 [SMILES: *c1ccc2n[nH]cc2c1 ; *c1ccn2nccc2c1 ; *c1ccc2nccn2c1 ; *c1cn2ccnc2cn1]

each of which is is optionally substituted with one to two R^{C}. The definitions of the remaining variables are provided in fortieth or forty-first embodiment.

In a forty-third embodiment, for the compound of the fortieth, forty-first or forty-second embodiment or a pharmaceutically acceptable salt thereof, R⁹, for each occurrence, is independently selected from —CH₃ and —CH₂CH₃. The definitions of the remaining variables are provided in fortieth, forty-first or forty-second embodiment.

In a forty-fourth embodiment, for the compound of the fortieth, forty-first, forty-second or forty-third embodiment or a pharmaceutically acceptable salt thereof, R^{C}, for each occurrence, is independently selected from F, —CH₃, —OCH₃, and —CHF₂. The definitions of the remaining variables are provided in fortieth, forty-first, forty-second or forty-third embodiment.

In one embodiment, the present disclosure provides a compound selected from Compounds 1-269 described in the Examples section and Table 1, a pharmaceutically acceptable salt, a racemic mixture or a stereoisomer thereof.

TABLE 1

| Cpd ID | Structure | Name |
|---|---|---|
| 1 | 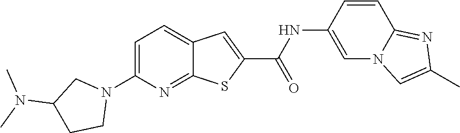 | 6-(3- (dimethylamino)pyrrolidin-1- yl)-N-(2-methylimidazo[1,2- a]pyridin-6-yl)thieno[2,3- b]pyridine-2-carboxamide |
| 2 | 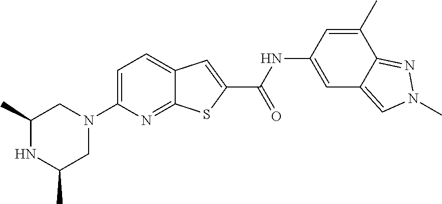 | N-(2,7-dimethyl-2H-indazol- 5-yl)-6-((3S,5R)-3,5- dimethylpiperazin-1- yl)thieno[2,3-b]pyridine-2- carboxamide |
| 3 | 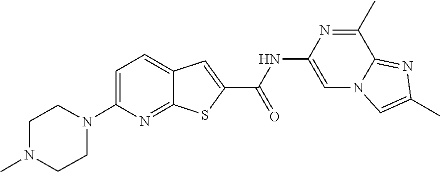 | N-(2,8-dimethylimidazo[1,2- a]pyrazin-6-yl)-6-(4- methylpiperazin-1- yl)thieno[2,3-b]pyridine-2- carboxamide |
| 4 | 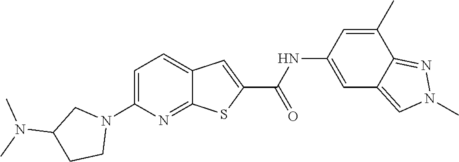 | N-(2,7-dimethyl-2H-indazol- 5-yl)-6-(3- (dimethylamino)pyrrolidin-1- yl)thieno[2,3-b]pyridine-2- carboxamide |
| 5 | 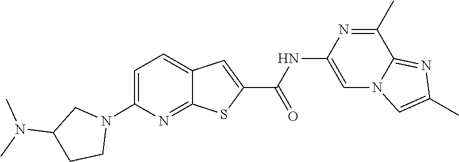 | 6-(3- (dimethylamino)pyrrolidin-1- yl)-N-(2,8- dimethylimidazo[1,2- a]pyrazin-6-yl)thieno[2,3- b]pyridine-2-carboxamide |
| 6 | 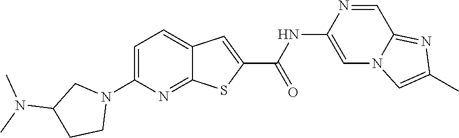 | 6-(3- (dimethylamino)pyrrolidin-1- yl)-N-(2-methylimidazo[1,2- a]pyrazin-6-yl)thieno[2,3- b]pyridine-2-carboxamide |
| 7 | 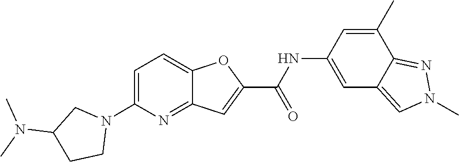 | N-(2,7-dimethyl-2H-indazol- 5-yl)-5-(3- (dimethylamino)pyrrolidin-1- yl)furo[3,2-b]pyridine-2- carboxamide |
| 8 | 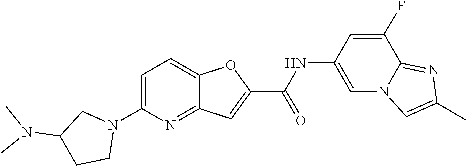 | 5-(3- (dimethylamino)pyrrolidin-1- yl)-N-(8-fluoro-2- methylimidazo[1,2-a]pyridin- 6-yl)furo[3,2-b]pyridine-2- carboxamide |
| 9 | 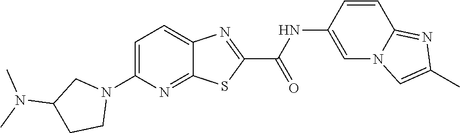 | (R)-5-(3- (dimethylamino)pyrrolidin-1- yl)-N-(2-methylimidazo[1,2- a]pyridin-6-yl)thiazolo[5,4- b]pyridine-2-carboxamide |
| 10 | 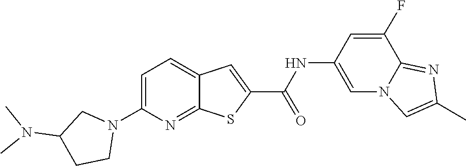 | 6-(3- (dimethylamino)pyrrolidin-1- yl)-N-(8-fluoro-2- methylimidazo[1,2-a]pyridin- 6-yl)thieno[2,3-b]pyridine-2- carboxamide |
| 11 | 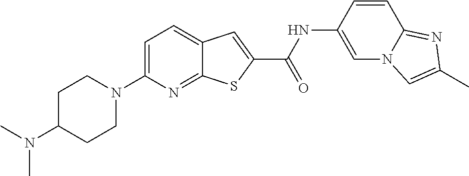 | 6-(4- (dimethylamino)piperidin-1- yl)-N-(2-methylimidazo[1,2- a]pyridin-6-yl)thieno[2,3- b]pyridine-2-carboxamide |
| 12 | 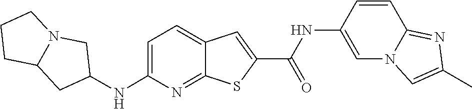 [SMILES: Cc1cn2cc(NC(=O)c3cc4ccc(NC5CC6CCCN6C5)nc4s3)ccc2n1] | 6-((hexahydro-1H-pyrrolizin- 2-yl)amino)-N-(2- methylimidazo[1,2-a]pyridin- 6-yl)thieno[2,3-b]pyridine-2- carboxamide |
| 13 | 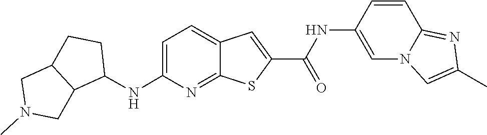 | N-(2-methylimidazo[1,2- a]pyridin-6-yl)-6-((2- methyloctahydrocyclopenta[c] pyrrol-4-yl)amino)thieno[2,3- b]pyridine-2-carboxamide |
| 14 | 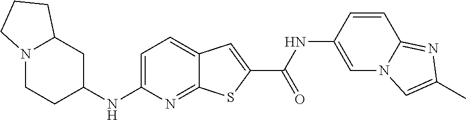 [SMILES: Cc1cn2cc(NC(=O)c3cc4ccc(NC5CCN6CCCC6C5)nc4s3)ccc2n1] | N-(2-methylimidazo[1,2- a]pyridin-6-yl)-6- ((octahydroindolizin-7- yl)amino)thieno[2,3- b]pyridine-2-carboxamide |
| 15 | 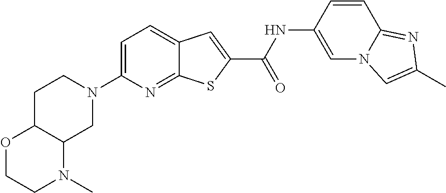 [SMILES: Cc1cn2cc(NC(=O)c3cc4ccc(N5CCC6OCCN(C)C6C5)nc4s3)ccc2n1] | 6-(4-methylhexahydro-2H- pyrido[4,3-b][1,4]oxazin- 6(5H)-yl)-N-(2- methylimidazo[1,2-a]pyridin- 6-yl)thieno[2,3-b]pyridine-2- carboxamide |
| 16 | 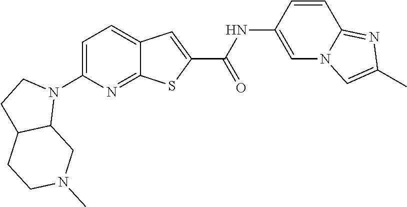 [SMILES: Cc1cn2cc(NC(=O)c3cc4ccc(N5CCC6CCN(C)CC65)nc4s3)ccc2n1] | N-(2-methylimidazo[1,2- a]pyridin-6-yl)-6-(6- methyloctahydro-1H- pyrrolo[2,3-c]pyridin-1- yl)thieno[2,3-b]pyridine-2- carboxamide |
| 17 | 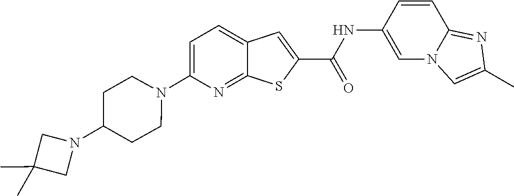 | 6-(4-(3,3-dimethylazetidin-1- yl)piperidin-1-yl)-N-(2- methylimidazo[1,2-a]pyridin- 6-yl)thieno[2,3-b]pyridine-2- carboxamide |
| 18 | 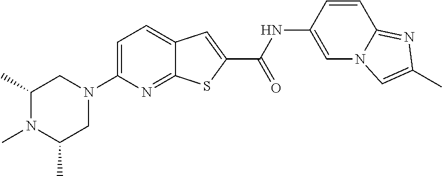 | N-(2-methylimidazo[1,2- a]pyridin-6-yl)-6-((3R,5S)- 3,4,5-trimethylpiperazin-1- yl)thieno[2,3-b]pyridine-2- carboxamide |
| 19 | 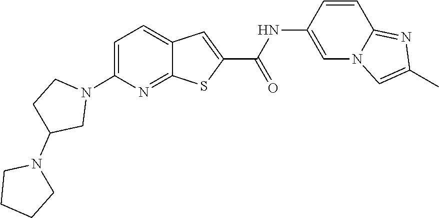 [SMILES: Cc1cn2cc(NC(=O)c3cc4ccc(N5CCC(N6CCCC6)C5)nc4s3)ccc2n1] | 6-([1,3′-bipyrrolidin]-1′-yl)-N- (2-methylimidazo[1,2- a]pyridin-6-yl)thieno[2,3- b]pyridine-2-carboxamide |
| 20 | 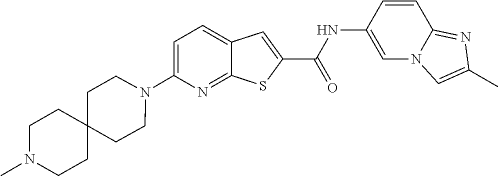 | 6-(9-methyl-3,9- diazaspiro[5.5]undecan-3-yl)- N-(2-methylimidazo[1,2- a]pyridin-6-yl)thieno[2,3- b]pyridine-2-carboxamide |
| 21 | 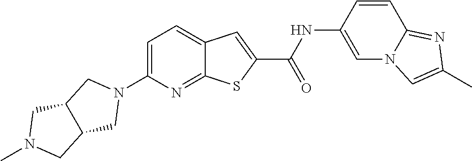 | 6-((3aR,6aS)-5- methylhexahydropyrrolo[3,4- c]pyrrol-2(1H)-yl)-N-(2- methylimidazo[1,2-a]pyridin- 6-yl)thieno[2,3-b]pyridine-2- carboxamide |
| 22 | 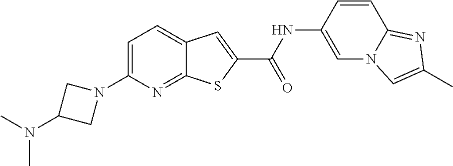 | 6-(3-(dimethylamino)azetidin- 1-yl)-N-(2- methylimidazo[1,2-a]pyridin- 6-yl)thieno[2,3-b]pyridine-2- carboxamide |
| 23 | 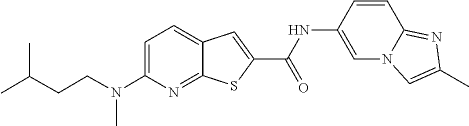 [SMILES: Cc1cn2cc(NC(=O)c3cc4ccc(N(C)CCC(C)C)nc4s3)ccc2n1] | 6-((2- (dimethylamino)ethyl)(methyl) amino)-N-(2- methylimidazo[1,2-a]pyridin- 6-yl)thieno[2,3-b]pyridine-2- carboxamide |
| 24 | 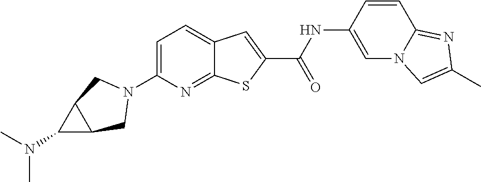 | 6-((1R,5S,6s)-6- (dimethylamino)-3- azabicyclo[3.1.0]hexan-3-yl)- N-(2-methylimidazo[1,2- a]pyridin-6-yl)thieno[2,3- b]pyridine-2-carboxamide |
| 25 | 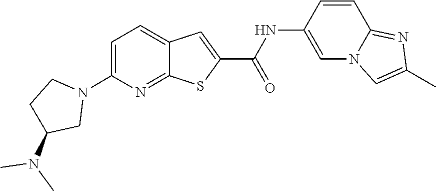 | (R)-6-(3- (dimethylamino)pyrrolidin-1- yl)-N-(2-methylimidazo[1,2- a]pyridin-6-yl)thieno[2,3- b]pyridine-2-carboxamide |
| 26 | 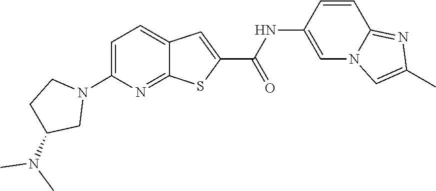 | (S)-6-(3- (dimethylamino)pyrrolidin-1- yl)-N-(2-methylimidazo[1,2- a]pyridin-6-yl)thieno[2,3- b]pyridine-2-carboxamide |
| 27 | 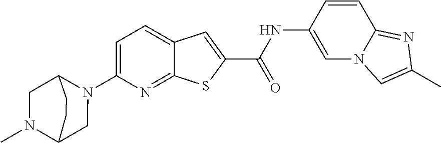 | 6-(5-methyl-2,5- diazabicyclo[2.2.2]octan-2- yl)-N-(2-methylimidazo[1,2- a]pyridin-6-yl)thieno[2,3- b]pyridine-2-carboxamide |
| 28 | 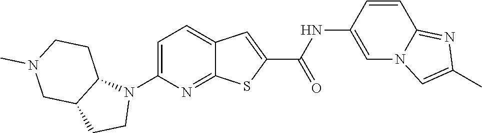 | N-(2-methylimidazo[1,2- a]pyridin-6-yl)-6-((3aS,7aR)- 5-methyloctahydro-1H- pyrrolo[3,2-c]pyridin-1- yl)thieno[2,3-b]pyridine-2- carboxamide |
| 29 | 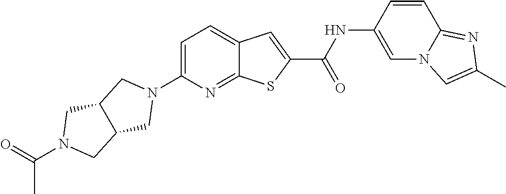 [SMILES: CC(=O)N1C[C@@H]2CN(c3ccc4cc(C(=O)Nc5ccc6nc(C)cn6c5)sc4n3)C[C@@H]2C1] | 6-((3aR,6aS)-5- acetylhexahydropyrrolo[3,4- c]pyrrol-2(1H)-yl)-N-(2- methylimidazo[1,2-a]pyridin- 6-yl)thieno[2,3-b]pyridine-2- carboxamide |
| 30 | 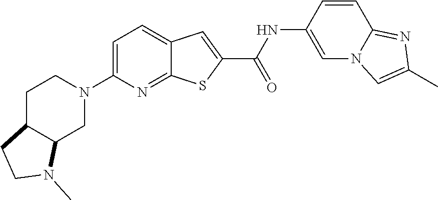 | N-(2-methylimidazo[1,2- a]pyridin-6-yl)-6-((3aR,7aR)- 1-methyloctahydro-6H- pyrrolo[2,3-c]pyridin-6- yl)thieno[2,3-b]pyridine-2- carboxamide |
| 31 | 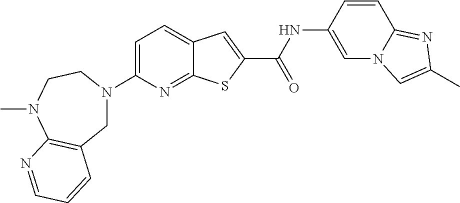 | 6-(1-methyl-1,2,3,5- tetrahydro-4H-pyrido[2,3- e][1,4]diazepin-4-yl)-N-(2- methylimidazo[1,2-a]pyridin- 6-yl)thieno[2,3-b]pyridine-2- carboxamide |
| 32 | 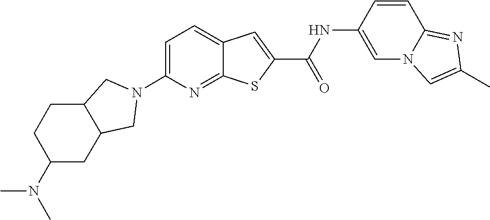 | 6-(5- (dimethylamino)octahydro- 2H-isoindol-2-yl)-N-(2- methylimidazo[1,2-a]pyridin- 6-yl)thieno[2,3-b]pyridine-2- carboxamide |
| 33 | 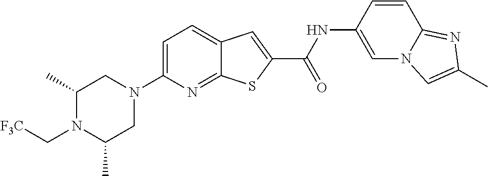 [SMILES: Cc1cn2cc(NC(=O)c3cc4ccc(N5C[C@@H](C)N(CC(F)(F)F)[C@@H](C)C5)nc4s3)ccc2n1] | 6-((3R,5S)-3,5-dimethyl-4- (2,2,2- trifluoroethyl)piperazin-1-yl)- N-(2-methylimidazo[1,2- a]pyridin-6-yl)thieno[2,3- b]pyridine-2-carboxamide |
| 34 | 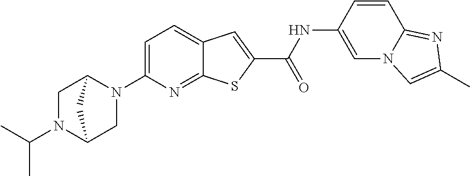 [SMILES: Cc1cn2cc(NC(=O)c3cc4ccc(N5C[C@@H]6C[C@H]5CN6C(C)C)nc4s3)ccc2n1] | 6-((1R,4R)-5-isopropyl-2,5- diazabicyclo[2.2.1]heptan-2- yl)-N-(2-methylimidazo[1,2- a]pyridin-6-yl)thieno[2,3- b]pyridine-2-carboxamide |
| 35 | 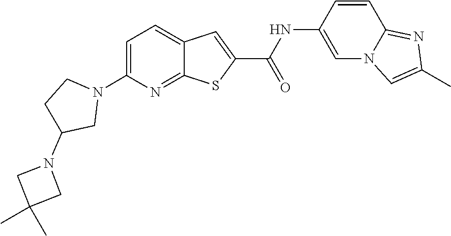 | 6-(3-(3,3-dimethylazetidin-1- yl)pyrrolidin-1-yl)-N-(2- methylimidazo[1,2-a]pyridin- 6-yl)thieno[2,3-b]pyridine-2- carboxamide |
| 36 | 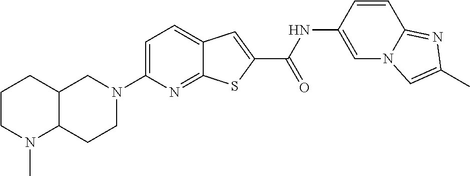 [SMILES: Cc1cn2cc(NC(=O)c3cc4ccc(N5CCC6C(CCCN6C)C5)nc4s3)ccc2n1] | N-(2-methylimidazo[1,2- a]pyridin-6-yl)-6-(1- methyloctahydro-1,6- naphthyridin-6(2H)- yl)thieno[2,3-b]pyridine-2- carboxamide |
| 37 | 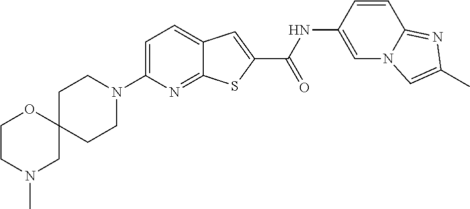 [SMILES: Cc1cn2cc(NC(=O)c3cc4ccc(N5CCC6(CC5)CN(C)CCO6)nc4s3)ccc2n1] | 6-(4-methyl-1-oxa-4,9- diazaspiro[5.5]undecan-9-yl)- N-(2-methylimidazo[1,2- a]pyridin-6-yl)thieno[2,3- b]pyridine-2-carboxamide |
| 38 | 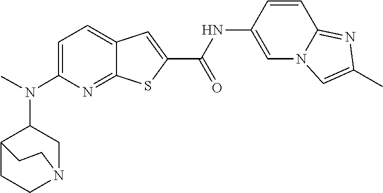 | 6-(methyl(quinuclidin-3- yl)amino)-N-(2- methylimidazo[1,2-a]pyridin- 6-yl)thieno[2,3-b]pyridine-2- carboxamide |
| 39 | 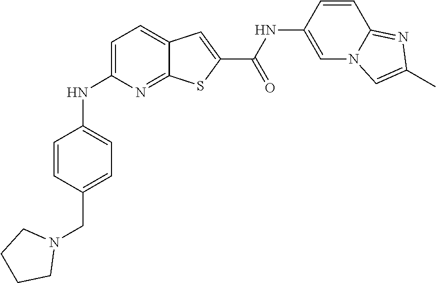 [SMILES: Cc1cn2cc(NC(=O)c3cc4ccc(Nc5ccc(CN6CCCC6)cc5)nc4s3)ccc2n1] | N-(2-methylimidazo[1,2- a]pyridin-6-yl)-6-((4- (pyrrolidin-1- ylmethyl)phenyl)amino)thieno [2,3-b]pyridine-2- carboxamide |
| 40 | 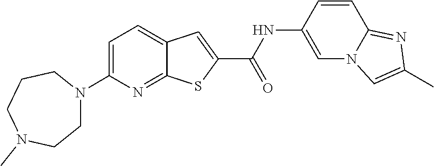 | 6-(4-methyl-1,4-diazepan-1- yl)-N-(2-methylimidazo[1,2- a]pyridin-6-yl)thieno[2,3- b]pyridine-2-carboxamide |
| 41 | 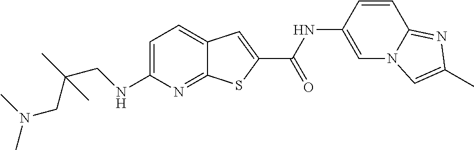 | 6-((3-(dimethylamino)-2,2- dimethylpropyl)amino)-N-(2- methylimidazo[1,2-a]pyridin- 6-yl)thieno[2,3-b]pyridine-2- carboxamide |
| 42 | 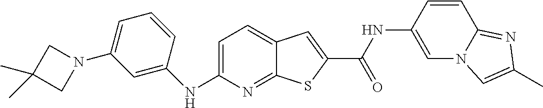 | 6-((3-(3,3-dimethylazetidin-1- yl)phenyl)amino)-N-(2- methylimidazo[1,2-a]pyridin- 6-yl)thieno[2,3-b]pyridine-2- carboxamide |
| 43 | 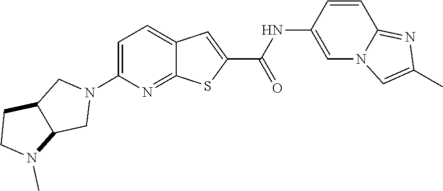 | 6-((3aR,6aR)-1- methylhexahydropyrrolo[3,4- b]pyrrol-5(1H)-yl)-N-(2- methylimidazo[1,2-a]pyridin- 6-yl)thieno[2,3-b]pyridine-2- carboxamide |
| 44 | 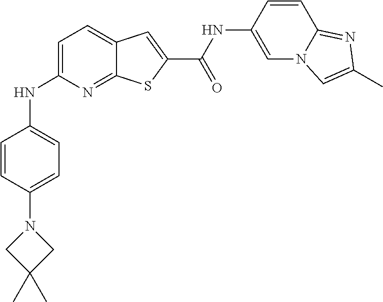 [SMILES: Cc1cn2cc(NC(=O)c3cc4ccc(Nc5ccc(N6CC(C)(C)C6)cc5)nc4s3)ccc2n1] | 6-((4-(3,3-dimethylazetidin-1- yl)phenyl)amino)-N-(2- methylimidazo[1,2-a]pyridin- 6-yl)thieno[2,3-b]pyridine-2- carboxamide |
| 45 | 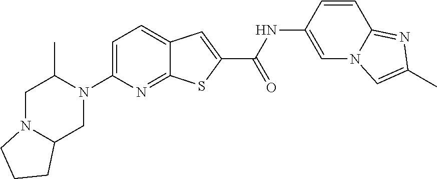 [SMILES: Cc1cn2cc(NC(=O)c3cc4ccc(N5CC6CCCN6CC5C)nc4s3)ccc2n1] | 6-(3- methylhexahydropyrrolo[1,2- a]pyrazin-2(1H)-yl)-N-(2- methylimidazo[1,2-a]pyridin- 6-yl)thieno[2,3-b]pyridine-2- carboxamide |
| 46 | 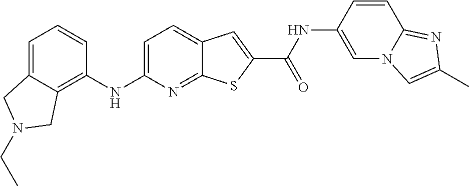 [SMILES: CCN1Cc2cccc(Nc3ccc4cc(C(=O)Nc5ccc6nc(C)cn6c5)sc4n3)c2C1] | 6-((2-ethylisoindolin-4- yl)amino)-N-(2- methylimidazo[1,2-a]pyridin- 6-yl)thieno[2,3-b]pyridine-2- carboxamide |
| 47 | 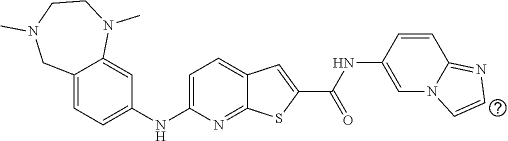 [SMILES: CN1CCN(C)c2cc(Nc3ccc4cc(C(=O)Nc5ccc6nccn6c5)sc4n3)ccc2C1] | 6-((1,4-dimethyl-2,3,4,5- tetrahydro-1H- benzo[e][1,4]diazepin-8- yl)amino)-N-(2- methylimidazo[1,2-a]pyridin- 6-yl)thieno[2,3-b]pyridine-2- carboxamide |
| 48 | 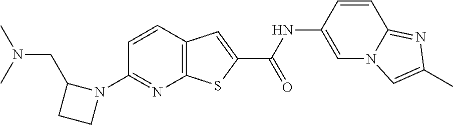 | 6-(2- ((dimethylamino)methyl) azetidin-1-yl)-N-(2- methylimidazo[1,2-a]pyridin- 6-yl)thieno[2,3-b]pyridine-2- carboxamide |
| 49 | 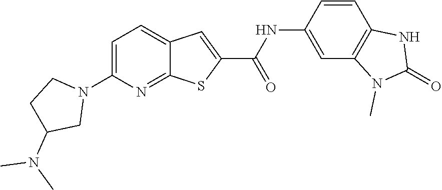 [SMILES: CN(C)C1CCN(c2ccc3cc(C(=O)Nc4ccc5[nH]c(=O)n(C)c5c4)sc3n2)C1] | 6-(3- (dimethylamino)pyrrolidin-1- yl)-N-(3-methyl-2-oxo-2,3- dihydro-1H- benzo[d]imidazol-5- yl)thieno[2,3-b]pyridine-2- carboxamide |
| 50 | 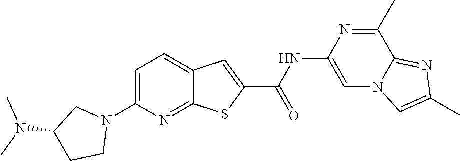 | (S)-6-(3- (dimethylamino)pyrrolidin-1- yl)-N-(2,8- dimethylimidazo[1,2- a]pyrazin-6-yl)thieno[2,3- b]pyridine-2-carboxamide |
| 51 | 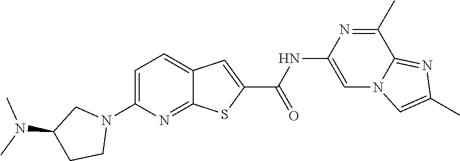 | (R)-6-(3- (dimethylamino)pyrrolidin-1- yl)-N-(2,8- dimethylimidazo[1,2- a]pyrazin-6-yl)thieno[2,3- b]pyridine-2-carboxamide |
| 52 | 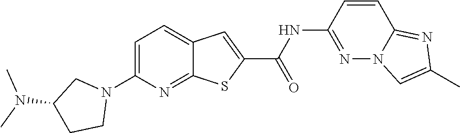 | (S)-6-(3- (dimethylamino)pyrrolidin-1- yl)-N-(2-methylimidazo[1,2- b]pyridazin-6-yl)thieno[2,3- b]pyridine-2-carboxamide |
| 53 | 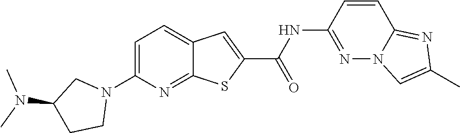 | (R)-6-(3- (dimethylamino)pyrrolidin-1- yl)-N-(2-methylimidazo[1,2- b]pyridazin-6-yl)thieno[2,3- b]pyridine-2-carboxamide |
| 54 | 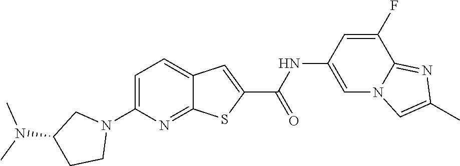 | (S)-6-(3- (dimethylamino)pyrrolidin-1- yl)-N-(8-fluoro-2- methylimidazo[1,2-a]pyridin- 6-yl)thieno[2,3-b]pyridine-2- carboxamide |
| 55 | 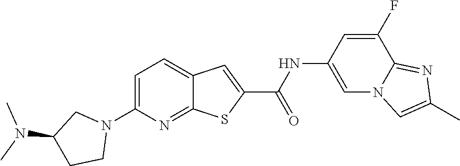 | (R)-6-(3- (dimethylamino)pyrrolidin-1- yl)-N-(8-fluoro-2- methylimidazo[1,2-a]pyridin- 6-yl)thieno[2,3-b]pyridine-2- carboxamide |
| 56 | 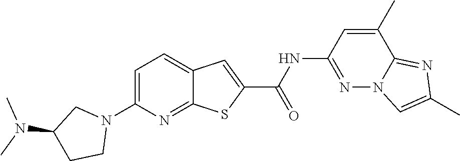 | (R)-6-(3- (dimethylamino)pyrrolidin-1- yl)-N-(2,8- dimethylimidazo[1,2- b]pyridazin-6-yl)thieno[2,3- b]pyridine-2-carboxamide |
| 57 | 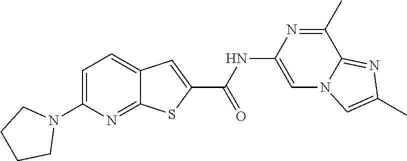 [SMILES: Cc1cn2cc(NC(=O)c3cc4ccc(N5CCCC5)nc4s3)nc(C)c2n1] | N-(2,8-dimethylimidazo[1,2- a]pyrazin-6-yl)-6-(pyrrolidin- 1-yl)thieno[2,3-b]pyridine-2- carboxamide |
| 58 | 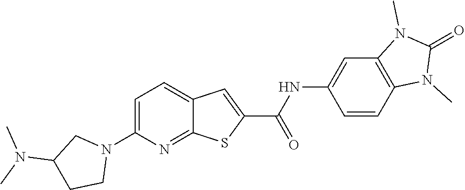 [SMILES: CN(C)C1CCN(c2ccc3cc(C(=O)Nc4ccc5c(c4)n(C)c(=O)n5C)sc3n2)C1] | N-(1,3-dimethyl-2-oxo-2,3- dihydro-1H- benzo[d]imidazol-5-yl)-6-(3- (dimethylamino)pyrrolidin-1- yl)thieno[2,3-b]pyridine-2- carboxamide |
| 59 | 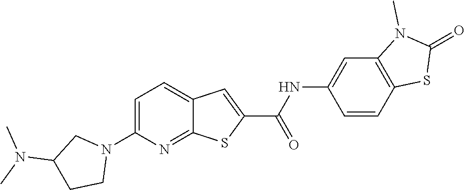 [SMILES: CN(C)C1CCN(c2ccc3cc(C(=O)Nc4ccc5sc(=O)n(C)c5c4)sc3n2)C1] | 6-(3- (dimethylamino)pyrrolidin-1- yl)-N-(3-methyl-2-oxo-2,3- dihydrobenzo[d]thiazol-5- yl)thieno[2,3-b]pyridine-2- carboxamide |
| 60 | 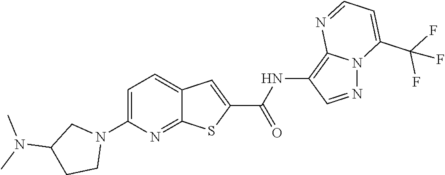 [SMILES: CN(C)C1CCN(c2ccc3cc(C(=O)Nc4cnn5c(C(F)(F)F)ccnc45)sc3n2)C1] | 6-(3- (dimethylamino)pyrrolidin-1- yl)-N-(7- (trifluoromethyl)pyrazolo[1,5- a]pyrimidin-3-yl)thieno[2,3- b]pyridine-2-carboxamide |
| 61 | 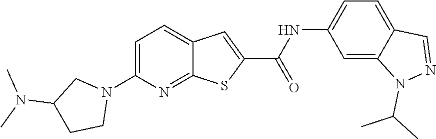 [SMILES: CC(C)n1ncc2ccc(NC(=O)c3cc4ccc(N5CCC(N(C)C)C5)nc4s3)cc21] | 6-(3- (dimethylamino)pyrrolidin-1- yl)-N-(1-isopropyl-1H- indazol-6-yl)thieno[2,3- b]pyridine-2-carboxamide |
| 62 | 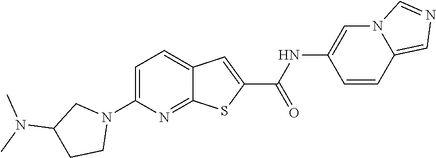 [SMILES: CN(C)C1CCN(c2ccc3cc(C(=O)Nc4ccc5cncn5c4)sc3n2)C1] | 6-(3- (dimethylamino)pyrrolidin-1- yl)-N-(imidazo[1,5-a]pyridin- 6-yl)thieno[2,3-b]pyridine-2- carboxamide |
| 63 | 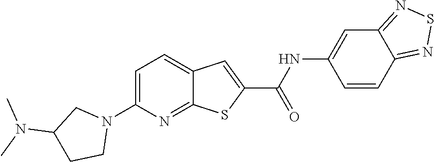 [SMILES: CN(C)C1CCN(c2ccc3cc(C(=O)Nc4ccc5nsnc5c4)sc3n2)C1] | N-(benzo[c][1,2,5]thiadiazol- 5-yl)-6-(3- (dimethylamino)pyrrolidin-1- yl)thieno[2,3-b]pyridine-2- carboxamide |
| 64 | 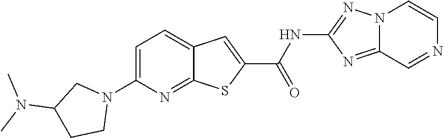 [SMILES: CN(C)C1CCN(c2ccc3cc(C(=O)Nc4nc5cnccn5n4)sc3n2)C1] | N-([1,2,4]triazolo[1,5- a]pyrazin-2-yl)-6-(3- (dimethylamino)pyrrolidin-1- yl)thieno[2,3-b]pyridine-2- carboxamide |
| 65 | 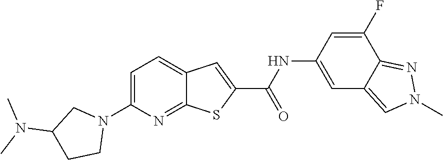 | 6-(3- (dimethylamino)pyrrolidin-1- yl)-N-(7-fluoro-2-methyl-2H- indazol-5-yl)thieno[2,3- b]pyridine-2-carboxamide |
| 66 | 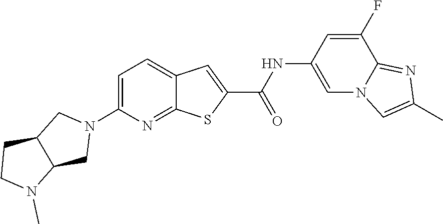 [SMILES: Cc1cn2cc(NC(=O)c3cc4ccc(N5C[C@@H]6CCN(C)[C@@H]6C5)nc4s3)cc(F)c2n1] | N-(8-fluoro-2- methylimidazo[1,2-a]pyridin- 6-yl)-6-((3aS,6aS)-1- methylhexahydropyrrolo[3,4- b]pyrrol-5(1H)-yl)thieno[2,3- b]pyridine-2-carboxamide |
| 67 | 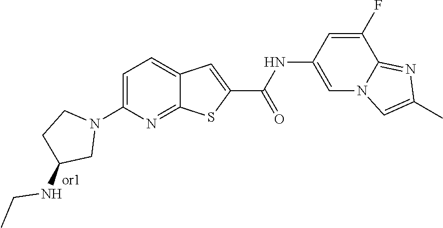 [SMILES: CCN[C@H]1CCN(c2ccc3cc(C(=O)Nc4cc(F)c5nc(C)cn5c4)sc3n2)C1] | rel-(S)-6-(3- (ethylamino)pyrrolidin-1-yl)- N-(8-fluoro-2- methylimidazo[1,2-a]pyridin- 6-yl)thieno[2,3-b]pyridine-2- carboxamide |
| 68 | 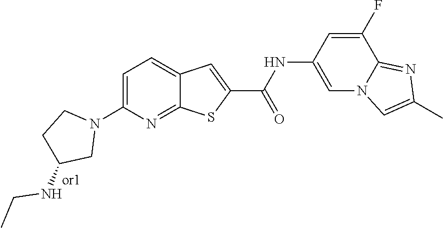 [SMILES: CCN[C@@H]1CCN(c2ccc3cc(C(=O)Nc4cc(F)c5nc(C)cn5c4)sc3n2)C1] | rel-(R)-6-(3- (ethylamino)pyrrolidin-1-yl)- N-(8-fluoro-2- methylimidazo[1,2-a]pyridin- 6-yl)thieno[2,3-b]pyridine-2- carboxamide |
| 69 | 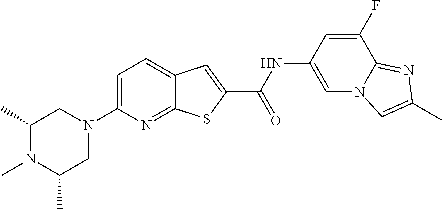 | N-(8-fluoro-2- methylimidazo[1,2-a]pyridin- 6-yl)-6-((3R,5S)-3,4,5- trimethylpiperazin-1- yl)thieno[2,3-b]pyridine-2- carboxamide |
| 70 | 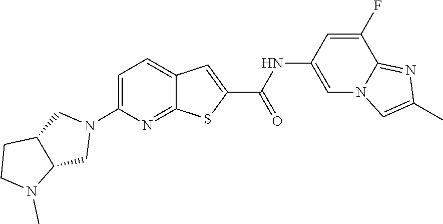 [SMILES: Cc1cn2cc(NC(=O)c3cc4ccc(N5C[C@H]6CCN(C)[C@H]6C5)nc4s3)cc(F)c2n1] | N-(8-fluoro-2- methylimidazo[1,2-a]pyridin- 6-yl)-6-((3aR,6aR)-1- methylhexahydropyrrolo[3,4- b]pyrrol-5(1H)-yl)thieno[2,3- b]pyridine-2-carboxamide |
| 71 | 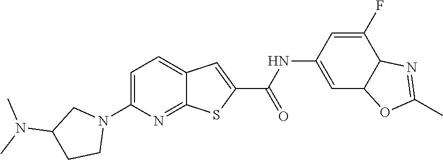 | 6-(3- (dimethylamino)pyrrolidin-1- yl)-N-(4-fluoro-2- methylbenzo[d]oxazol-6- yl)thieno[2,3-b]pyridine-2- carboxamide |
| 72 | 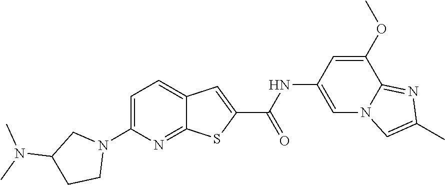 | 6-(3- (dimethylamino)pyrrolidin-1- yl)-N-(8-methoxy-2- methylimidazo[1,2-a]pyridin- 6-yl)thieno[2,3-b]pyridine-2- carboxamide |
| 73 | 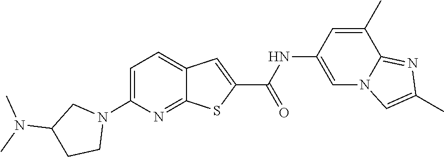 | 6-(3- (dimethylamino)pyrrolidin-1- yl)-N-(2,8- dimethylimidazo[1,2- a]pyridin-6-yl)thieno[2,3- b]pyridine-2-carboxamide |
| 74 | 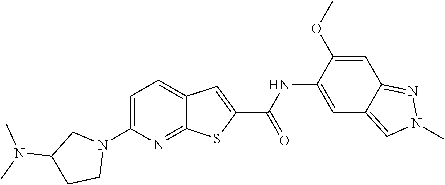 | 6-(3- (dimethylamino)pyrrolidin-1- yl)-N-(6-methoxy-2-methyl- 2H-indazol-5-yl)thieno[2,3- b]pyridine-2-carboxamide |
| 75 | 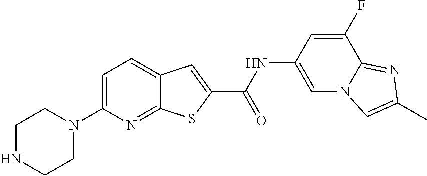 [SMILES: Cc1cn2cc(NC(=O)c3cc4ccc(N5CCNCC5)nc4s3)cc(F)c2n1] | N-(8-fluoro-2- methylimidazo[1,2-a]pyridin- 6-yl)-6-(piperazin-1- yl)thieno[2,3-b]pyridine-2- carboxamide |
| 76 | 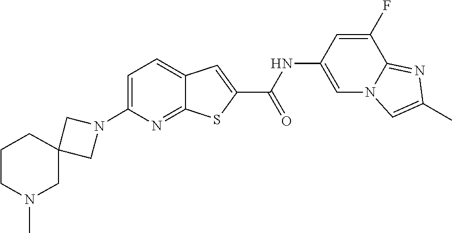 [SMILES: Cc1cn2cc(NC(=O)c3cc4ccc(N5CC6(CCCN(C)C6)C5)nc4s3)cc(F)c2n1] | N-(8-fluoro-2- methylimidazo[1,2-a]pyridin- 6-yl)-6-(6-methyl-2,6- diazaspiro[3.5]nonan-2- yl)thieno[2,3-b]pyridine-2- carboxamide |
| 77 | 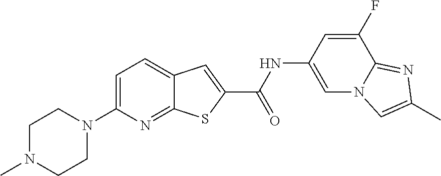 | N-(8-fluoro-2- methylimidazo[1,2-a]pyridin- 6-yl)-6-(4-methylpiperazin-1- yl)thieno[2,3-b]pyridine-2- carboxamide |
| 78 | 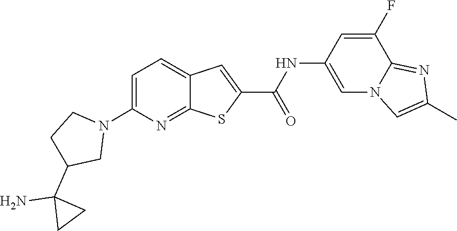 [SMILES: Cc1cn2cc(NC(=O)c3cc4ccc(N5CCC(C6(N)CC6)C5)nc4s3)cc(F)c2n1] | 6-(3-(1- aminocyclopropyl)pyrrolidin- 1-yl)-N-(8-fluoro-2- methylimidazo[1,2-a]pyridin- 6-yl)thieno[2,3-b]pyridine-2- carboxamide |
| 79 | 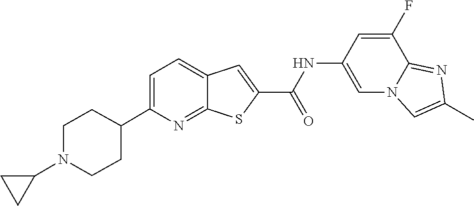 [SMILES: Cc1cn2cc(NC(=O)c3cc4ccc(C5CCN(C6CC6)CC5)nc4s3)cc(F)c2n1] | 6-(4-cyclopropylpiperazin-1- yl)-N-(8-fluoro-2- methylimidazo[1,2-a]pyridin- 6-yl)thieno[2,3-b]pyridine-2- carboxamide |
| 80 | 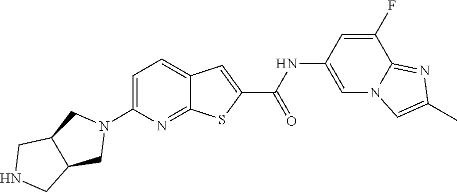 [SMILES: Cc1cn2cc(NC(=O)c3cc4ccc(N5C[C@H]6CNC[C@H]6C5)nc4s3)cc(F)c2n1] | N-(8-fluoro-2- methylimidazo[1,2-a]pyridin- 6-yl)-6-((3aR,6aS)- hexahydropyrrolo[3,4- c]pyrrol-2(1H)-yl)thieno[2,3- b]pyridine-2-carboxamide |
| 81 | 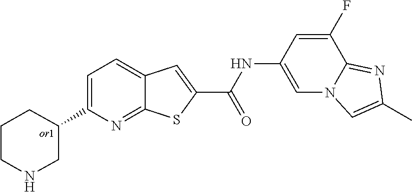 [SMILES: Cc1cn2cc(NC(=O)c3cc4ccc([C@H]5CCCNC5)nc4s3)cc(F)c2n1] | rel-(R)-N-(8-fluoro-2- methylimidazo[1,2-a]pyridin- 6-yl)-6-(piperidin-3- yl)thieno[2,3-b]pyridine-2- carboxamide |
| 82 | 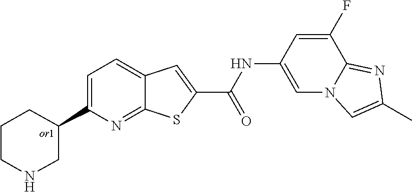 [SMILES: Cc1cn2cc(NC(=O)c3cc4ccc([C@@H]5CCCNC5)nc4s3)cc(F)c2n1] | rel-(S)-N-(8-fluoro-2- methylimidazo[1,2-a]pyridin- 6-yl)-6-(piperidin-3- yl)thieno[2,3-b]pyridine-2- carboxamide |
| 83 | 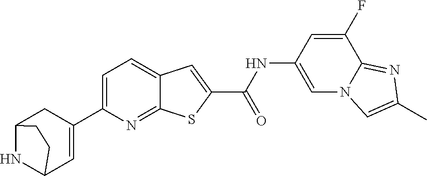 [SMILES: Cc1cn2cc(NC(=O)c3cc4ccc(C5=CC6CCC(C5)N6)nc4s3)cc(F)c2n1] | 6-(8-azabicyclo[3.2.1]oct-2- en-3-yl)-N-(8-fluoro-2- methylimidazo[1,2-a]pyridin- 6-yl)thieno[2,3-b]pyridine-2- carboxamide |
| 84 | 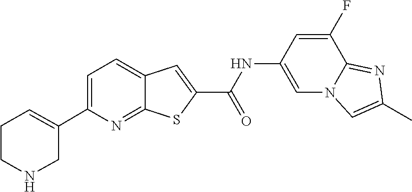 [SMILES: Cc1cn2cc(NC(=O)c3cc4ccc(C5=CCCNC5)nc4s3)cc(F)c2n1] | N-(8-fluoro-2- methylimidazo[1,2-a]pyridin- 6-yl)-6-(1,2,5,6- tetrahydropyridin-3- yl)thieno[2,3-b]pyridine-2- carboxamide |
| 85 | 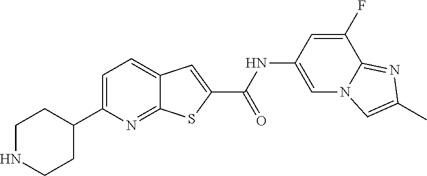 [SMILES: Cc1cn2cc(NC(=O)c3cc4ccc(C5CCNCC5)nc4s3)cc(F)c2n1] | N-(8-fluoro-2- methylimidazo[1,2-a]pyridin- 6-yl)-6-(piperidin-4- yl)thieno[2,3-b]pyridine-2- carboxamide |
| 86 | 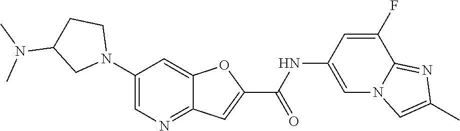 | 6-(3- (dimethylamino)pyrrolidin-1- yl)-N-(8-fluoro-2- methylimidazo[1,2-a]pyridin- 6-yl)furo[3,2-b]pyridine-2- carboxamide |
| 87 | 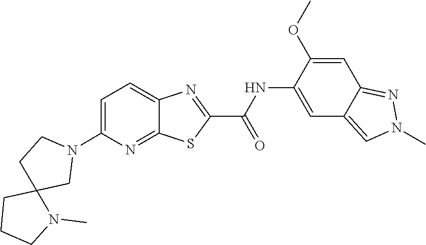 [SMILES: COc1cc2nn(C)cc2cc1NC(=O)c1nc2ccc(N3CCC4(CCCN4C)C3)nc2s1] | N-(6-methoxy-2-methyl-2H- indazol-5-yl)-5-(1-methyl- 1,7-diazaspiro[4.4]nonan-7- yl)thiazolo[5,4-b]pyridine-2- carboxamide |
| 88 | 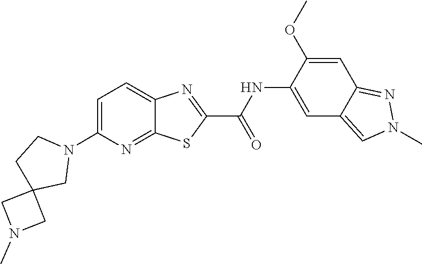 | N-(6-methoxy-2-methyl-2H- indazol-5-yl)-5-(2-methyl- 2,6-diazaspiro[3.4]octan-6- yl)thiazolo[5,4-b]pyridine-2- carboxamide |
| 89 | 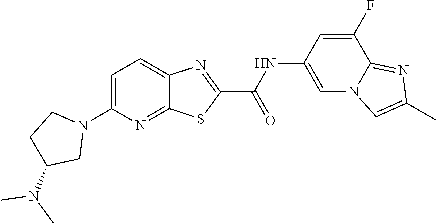 | (R)-5-(3- (dimethylamino)pyrrolidin-1- yl)-N-(8-fluoro-2- methylimidazo[1,2-a]pyridin- 6-yl)thiazolo[5,4-b]pyridine- 2-carboxamide |
| 90 | 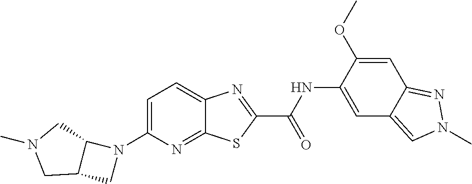 | N-(6-methoxy-2-methyl-2H- indazol-5-yl)-5-((1S,5R)-3- methyl-3,6- diazabicyclo[3.2.0]heptan-6- yl)thiazolo[5,4-b]pyridine-2- carboxamide |
| 91 | 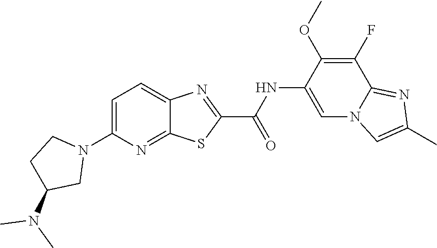 | (S)-5-(3- (dimethylamino)pyrrolidin-1- yl)-N-(8-fluoro-7-methoxy-2- methylimidazo[1,2-a]pyridin- 6-yl)thiazolo[5,4-b]pyridine- 2-carboxamide |
| 92 | 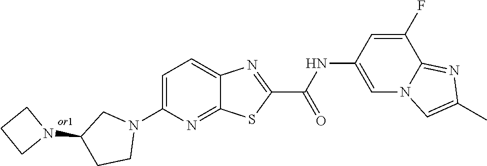 [SMILES: Cc1cn2cc(NC(=O)c3nc4ccc(N5CC[C@@H](N6CCC6)C5)nc4s3)cc(F)c2n1] | (rel-(R)-5-(3-(azetidin-1- yl)pyrrolidin-1-yl)-N-(8- fluoro-2-methylimidazo[1,2- a]pyridin-6-yl)thiazolo[5,4- b]pyridine-2-carboxamide |
| 93 | 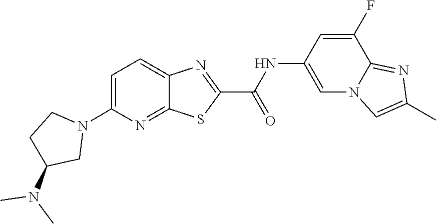 | (S)-5-(3- (dimethylamino)pyrrolidin-1- yl)-N-(8-fluoro-2- methylimidazo[1,2-a]pyridin- 6-yl)thiazolo[5,4-b]pyridine- 2-carboxamide |
| 94 | 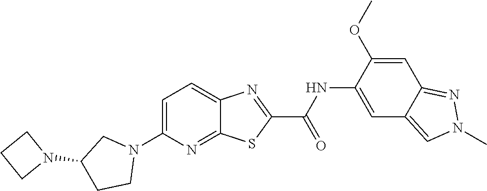 [SMILES: COc1cc2nn(C)cc2cc1NC(=O)c1nc2ccc(N3CC[C@H](N4CCC4)C3)nc2s1] | (S)-5-(3-(azetidin-1- yl)pyrrolidin-1-yl)-N-(6- methoxy-2-methyl-2H- indazol-5-yl)thiazolo[5,4- b]pyridine-2-carboxamide |
| 95 | 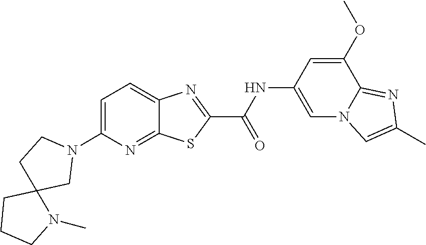 [SMILES: COc1cc(NC(=O)c2nc3ccc(N4CCC5(CCCN5C)C4)nc3s2)cn2cc(C)nc12] | N-(8-methoxy-2- methylimidazo[1,2-a]pyridin- 6-yl)-5-(1-methyl-1,7- diazaspiro[4.4]nonan-7- yl)thiazolo[5,4-b]pyridine-2- carboxamide |
| 96 | 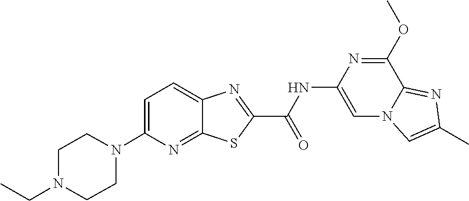 [SMILES: CCN1CCN(c2ccc3nc(C(=O)Nc4cn5cc(C)nc5c(OC)n4)sc3n2)CC1] | 5-(4-ethylpiperazin-1-yl)-N- (8-methoxy-2- methylimidazo[1,2-a]pyrazin- 6-yl)thiazolo[5,4-b]pyridine- 2-carboxamide |
| 97 | 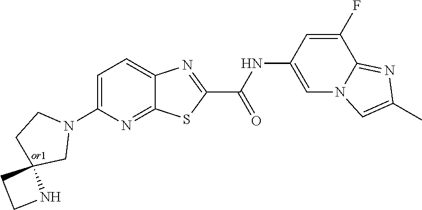 [SMILES: Cc1cn2cc(NC(=O)c3nc4ccc(N5CC[C@]6(CCN6)C5)nc4s3)cc(F)c2n1] | rel-(R)-N-(8-fluoro-2- methylimidazo[1,2-a]pyridin- 6-yl)-5-(1,6- diazaspiro[3.4]octan-6- yl)thiazolo[5,4-b]pyridine-2- carboxamide |
| 98 | 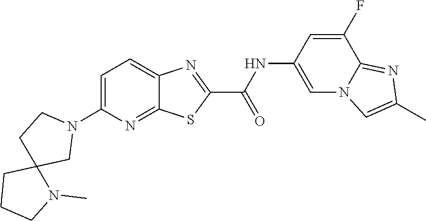 [SMILES: Cc1cn2cc(NC(=O)c3nc4ccc(N5CCC6(CCCN6C)C5)nc4s3)cc(F)c2n1] | N-(8-fluoro-2- methylimidazo[1,2-a]pyridin- 6-yl)-5-(1-methyl-1,7- diazaspiro[4.4]nonan-7- yl)thiazolo[5,4-b]pyridine-2- carboxamide |
| 99 | 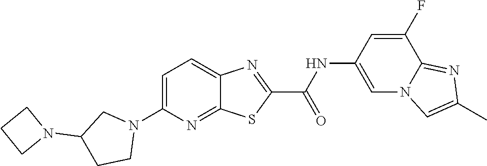 [SMILES: Cc1cn2cc(NC(=O)c3nc4ccc(N5CCC(N6CCC6)C5)nc4s3)cc(F)c2n1] | 5-(3-(azetidin-1-yl)pyrrolidin- 1-yl)-N-(8-fluoro-2- methylimidazo[1,2-a]pyridin- 6-yl)thiazolo[5,4-b]pyridine- 2-carboxamide |
| 100 | 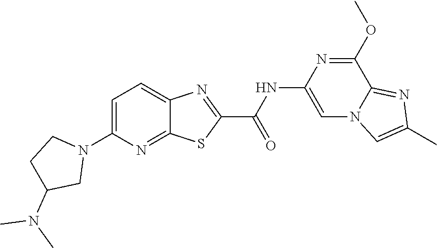 | 5-(3- (dimethylamino)pyrrolidin-1- yl)-N-(8-methoxy-2- methylimidazo[1,2-a]pyrazin- 6-yl)thiazolo[5,4-b]pyridine- 2-carboxamide |
| 101 | 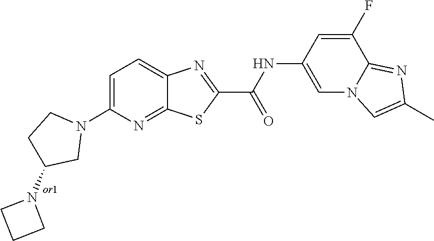 [SMILES: Cc1cn2cc(NC(=O)c3nc4ccc(N5CC[C@@H](N6CCC6)C5)nc4s3)cc(F)c2n1] | rel-(R)-5-(3-(azetidin-1- yl)pyrrolidin-1-yl)-N-(8- fluoro-2-methylimidazo[1,2- a]pyridin-6-yl)thiazolo[5,4- b]pyridine-2-carboxamide |
| 102 | 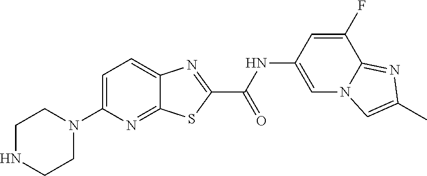 [SMILES: Cc1cn2cc(NC(=O)c3nc4ccc(N5CCNCC5)nc4s3)cc(F)c2n1] | N-(8-fluoro-2- methylimidazo[1,2-a]pyridin- 6-yl)-5-(piperazin-1- yl)thiazolo[5,4-b]pyridine-2- carboxamide |
| 103 | 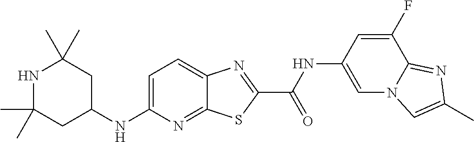 | N-(8-fluoro-2- methylimidazo[1,2-a]pyridin- 6-yl)-5-((2,2,6,6- tetramethylpiperidin-4- yl)amino)thiazolo[5,4- b]pyridine-2-carboxamide |
| 104 | 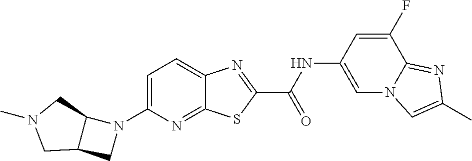 | N-(8-fluoro-2- methylimidazo[1,2-a]pyridin- 6-yl)-5-((1R,5S)-3-methyl- 3,6- diazabicyclo[3.2.0]heptan-6- yl)thiazolo[5,4-b]pyridine-2- carboxamide |
| 105 | 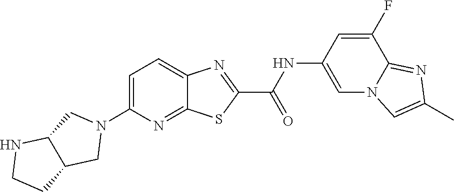 [SMILES: Cc1cn2cc(NC(=O)c3nc4ccc(N5C[C@@H]6CCN[C@@H]6C5)nc4s3)cc(F)c2n1] | N-(8-fluoro-2- methylimidazo[1,2-a]pyridin- 6-yl)-5-((3aS,6aS)- hexahydropyrrolo[3,4- b]pyrrol-5(1H)- yl)thiazolo[5,4-b]pyridine-2- carboxamide |
| 106 | 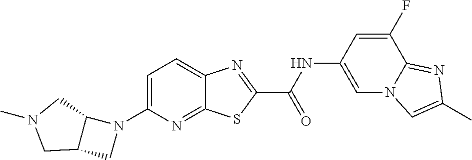 | N-(8-fluoro-2- methylimidazo[1,2-a]pyridin- 6-yl)-5-((1S,5R)-3-methyl- 3,6- diazabicyclo[3.2.0]heptan-6- yl)thiazolo[5,4-b]pyridine-2- carboxamide |
| 107 | 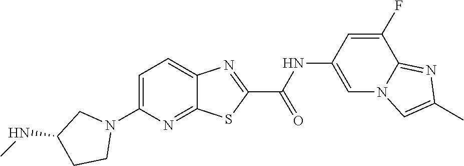 | (S)-N-(8-fluoro-2- methylimidazo[1,2-a]pyridin- 6-yl)-5-(3- (methylamino)pyrrolidin-1- yl)thiazolo[5,4-b]pyridine-2- carboxamide |
| 108 | 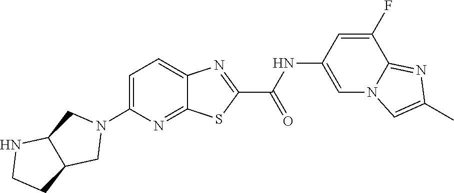 [SMILES: Cc1cn2cc(NC(=O)c3nc4ccc(N5C[C@H]6CCN[C@H]6C5)nc4s3)cc(F)c2n1] | N-(8-fluoro-2- methylimidazo[1,2-a]pyridin- 6-yl)-5-((3aR,6aR)- hexahydropyrrolo[3,4- b]pyrrol-5(1H)- yl)thiazolo[5,4-b]pyridine-2- carboxamide |
| 109 | 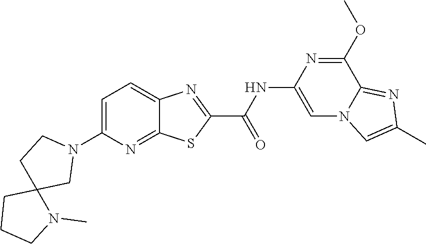 [SMILES: COc1nc(NC(=O)c2nc3ccc(N4CCC5(CCCN5C)C4)nc3s2)cn2cc(C)nc12] | N-(8-methoxy-2- methylimidazo[1,2-a]pyrazin- 6-yl)-5-(1-methyl-1,7- diazaspiro[4.4]nonan-7- yl)thiazolo[5,4-b]pyridine-2- carboxamide |
| 110 | 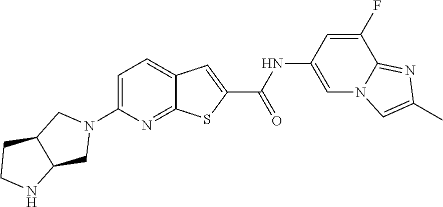 [SMILES: Cc1cn2cc(NC(=O)c3cc4ccc(N5C[C@@H]6CCN[C@@H]6C5)nc4s3)cc(F)c2n1] | N-(8-fluoro-2- methylimidazo[1,2-a]pyridin- 6-yl)-5-((3aS,6aS)- hexahydropyrrolo[3,4- b]pyrrol-5(1H)- yl)thiazolo[5,4-b]pyridine-2- carboxamide |
| 111 | 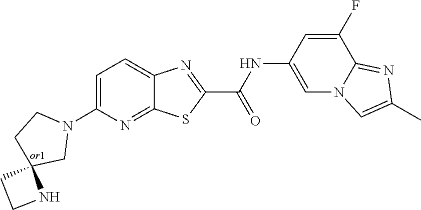 [SMILES: Cc1cn2cc(NC(=O)c3nc4ccc(N5CC[C@@]6(CCN6)C5)nc4s3)cc(F)c2n1] | rel-(R)-N-(8-fluoro-2- methylimidazo[1,2-a]pyridin- 6-yl)-5-(1,6- diazaspiro[3.4]octan-6- yl)thiazolo[5,4-b]pyridine-2- carboxamide |
| 112 | 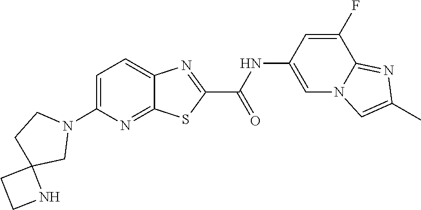 [SMILES: Cc1cn2cc(NC(=O)c3nc4ccc(N5CCC6(CCN6)C5)nc4s3)cc(F)c2n1] | N-(8-fluoro-2- methylimidazo[1,2-a]pyridin- 6-yl)-5-(1,6- diazaspiro[3.4]octan-6- yl)thiazolo[5,4-b]pyridine-2- carboxamide |
| 113 | 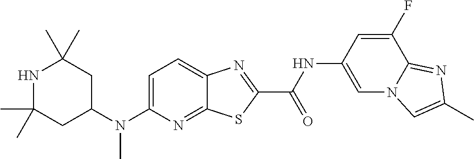 | N-(8-fluoro-2- methylimidazo[1,2-a]pyridin- 6-yl)-5-(methyl(2,2,6,6- tetramethylpiperidin-4- yl)amino)thiazolo[5,4- b]pyridine-2-carboxamide |
| 114 | 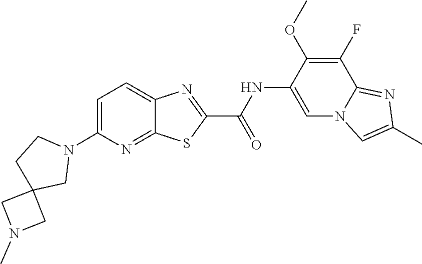 | N-(8-fluoro-7-methoxy-2- methylimidazo[1,2-a]pyridin- 6-yl)-5-(2-methyl-2,6- diazaspiro[3.4]octan-6- yl)thiazolo[5,4-b]pyridine-2- carboxamide |
| 115 | 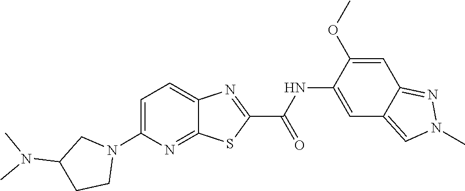 | 5-(3- (dimethylamino)pyrrolidin-1- yl)-N-(6-methoxy-2-methyl- 2H-indazol-5-yl)thiazolo[5,4- b]pyridine-2-carboxamide |
| 116 | 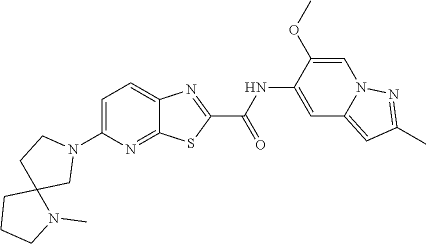 [SMILES: COc1cn2nc(C)cc2cc1NC(=O)c1nc2ccc(N3CCC4(CCCN4C)C3)nc2s1] | N-(6-methoxy-2- methylpyrazolo[1,5-a]pyridin- 5-yl)-5-(1-methyl-1,7- diazaspiro[4.4]nonan-7- yl)thiazolo[5,4-b]pyridine-2- carboxamide |
| 117 | 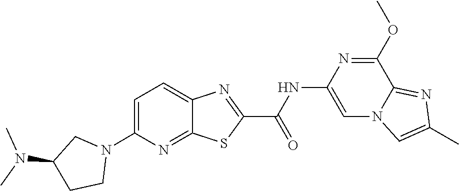 | (R)-5-(3- (dimethylamino)pyrrolidin-1- yl)-N-(8-methoxy-2- methylimidazo[1,2-a]pyrazin- 6-yl)thiazolo[5,4-b]pyridine- 2-carboxamide |
| 118 | 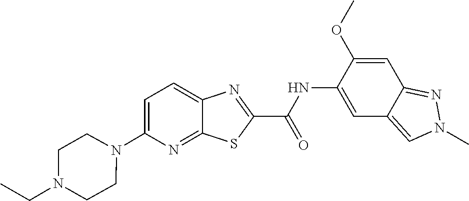 [SMILES: CCN1CCN(c2ccc3nc(C(=O)Nc4cc5cn(C)nc5cc4OC)sc3n2)CC1] | 5-(4-ethylpiperazin-1-yl)-N- (6-methoxy-2-methyl-2H- indazol-5-yl)thiazolo[5,4- b]pyridine-2-carboxamide |
| 119 | 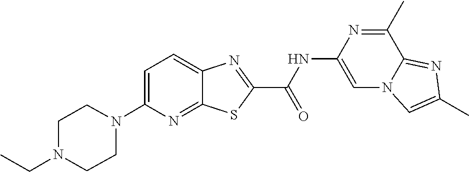 [SMILES: CCN1CCN(c2ccc3nc(C(=O)Nc4cn5cc(C)nc5c(C)n4)sc3n2)CC1] | N-(2,8-dimethylimidazo[1,2- a]pyrazin-6-yl)-5-(4- ethylpiperazin-1- yl)thiazolo[5,4-b]pyridine-2- carboxamide |
| 120 | 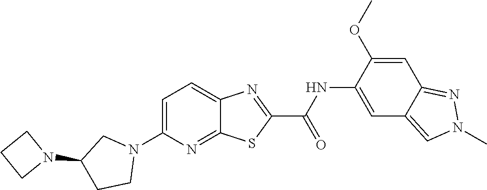 [SMILES: COc1cc2nn(C)cc2cc1NC(=O)c1nc2ccc(N3CC[C@@H](N4CCC4)C3)nc2s1] | (R)-5-(3-(azetidin-1- yl)pyrrolidin-1-yl)-N-(6- methoxy-2-methyl-2H- indazol-5-yl)thiazolo[5,4- b]pyridine-2-carboxamide |
| 121 | 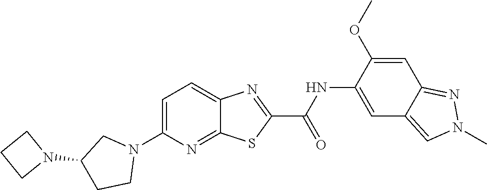 [SMILES: COc1cc2nn(C)cc2cc1NC(=O)c1nc2ccc(N3CC[C@H](N4CCC4)C3)nc2s1] | (S)-5-(3-(azetidin-1- yl)pyrrolidin-1-yl)-N-(6- methoxy-2-methyl-2H- indazol-5-yl)thiazolo[5,4- b]pyridine-2-carboxamide |
| 122 | 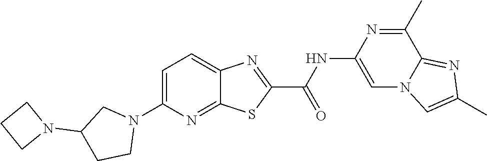 [SMILES: Cc1cn2cc(NC(=O)c3nc4ccc(N5CCC(N6CCC6)C5)nc4s3)nc(C)c2n1] | 5-(3-(azetidin-1-yl)pyrrolidin- 1-yl)-N-(2,8- dimethylimidazo[1,2- a]pyrazin-6-yl)thiazolo[5,4- b]pyridine-2-carboxamide |
| 123 | 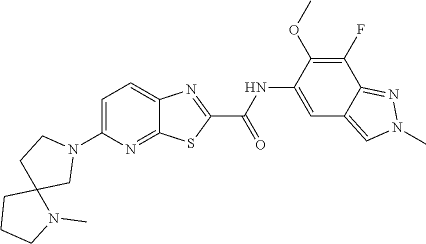 [SMILES: COc1c(NC(=O)c2nc3ccc(N4CCC5(CCCN5C)C4)nc3s2)cc2cn(C)nc2c1F] | N-(7-fluoro-6-methoxy-2- methyl-2H-indazol-5-yl)-5-(1- methyl-1,7- diazaspiro[4.4]nonan-7- yl)thiazolo[5,4-b]pyridine-2- carboxamide |
| 124 | 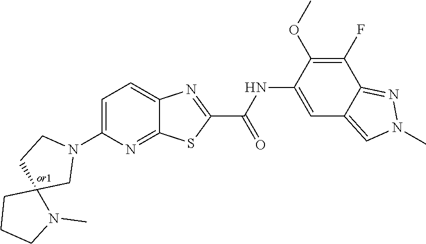 [SMILES: COc1c(NC(=O)c2nc3ccc(N4CC[C@]5(CCCN5C)C4)nc3s2)cc2cn(C)nc2c1F] | rel-(R)-N-(7-fluoro-6- methoxy-2-methyl-2H- indazol-5-yl)-5-(1-methyl- 1,7-diazaspiro[4.4]nonan-7- yl)thiazolo[5,4-b]pyridine-2- carboxamide |
| 125 | 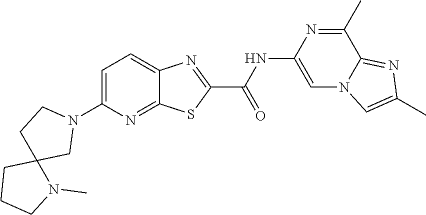 [SMILES: Cc1cn2cc(NC(=O)c3nc4ccc(N5CCC6(CCCN6C)C5)nc4s3)nc(C)c2n1] | N-(2,8-dimethylimidazo[1,2- a]pyrazin-6-yl)-5-(1-methyl- 1,7-diazaspiro[4.4]nonan-7- yl)thiazolo[5,4-b]pyridine-2- carboxamide |
| 126 | 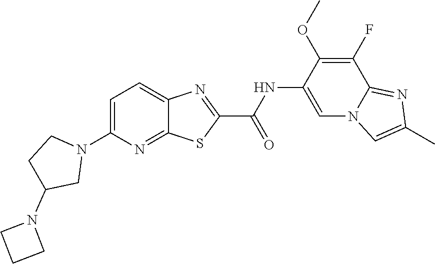 [SMILES: COc1c(NC(=O)c2nc3ccc(N4CCC(N5CCC5)C4)nc3s2)cn2cc(C)nc2c1F] | 5-(3-(azetidin-1-yl)pyrrolidin- 1-yl)-N-(8-fluoro-7-methoxy- 2-methylimidazo[1,2- a]pyridin-6-yl)thiazolo[5,4- b]pyridine-2-carboxamide |
| 127 | 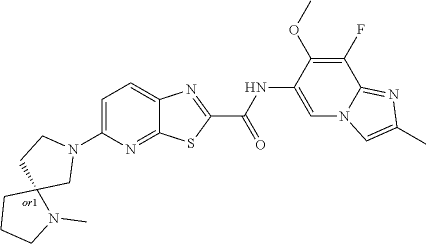 [SMILES: COc1c(NC(=O)c2nc3ccc(N4CC[C@]5(CCCN5C)C4)nc3s2)cn2cc(C)nc2c1F] | rel-(R)-N-(8-fluoro-7- methoxy-2- methylimidazo[1,2-a]pyridin- 6-yl)-5-(1-methyl-1,7- diazaspiro[4.4]nonan-7- yl)thiazolo[5,4-b]pyridine-2- carboxamide |
| 128 | 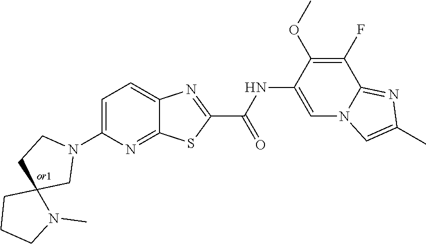 [SMILES: COc1c(NC(=O)c2nc3ccc(N4CC[C@@]5(CCCN5C)C4)nc3s2)cn2cc(C)nc2c1F] | rel-(R)-N-(8-fluoro-7- methoxy-2- methylimidazo[1,2-a]pyridin- 6-yl)-5-(1-methyl-1,7- diazaspiro[4.4]nonan-7- yl)thiazolo[5,4-b]pyridine-2- carboxamide |
| 129 | 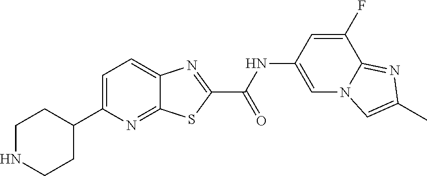 [SMILES: Cc1cn2cc(NC(=O)c3nc4ccc(C5CCNCC5)nc4s3)cc(F)c2n1] | N-(8-fluoro-2- methylimidazo[1,2-a]pyridin- 6-yl)-5-(piperidin-4- yl)thiazolo[5,4-b]pyridine-2- carboxamide |
| 130 | 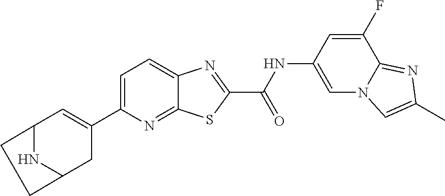 [SMILES: Cc1cn2cc(NC(=O)c3nc4ccc(C5=CC6CCC(C5)N6)nc4s3)cc(F)c2n1] | 5-(8-azabicyclo[3.2.1]oct-2- en-3-yl)-N-(8-fluoro-2- methylimidazo[1,2-a]pyridin- 6-yl)thiazolo[5,4-b]pyridine- 2-carboxamide |
| 131 | 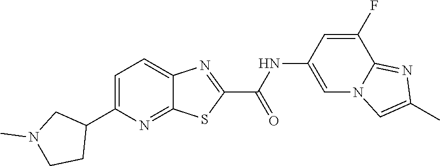 | N-(8-fluoro-2- methylimidazo[1,2-a]pyridin- 6-yl)-5-(1-methylpyrrolidin-3- yl)thiazolo[5,4-b]pyridine-2- carboxamide |
| 132 | 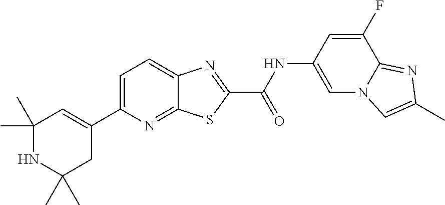 | N-(8-fluoro-2- methylimidazo[1,2-a]pyridin- 6-yl)-5-(2,2,6,6-tetramethyl- 1,2,3,6-tetrahydropyridin-4- yl)thiazolo[5,4-b]pyridine-2- carboxamide |
| 133 | 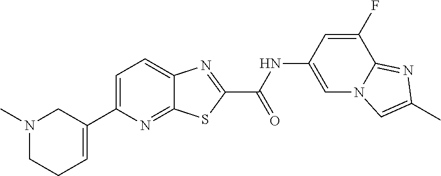 | N-(8-fluoro-2- methylimidazo[1,2-a]pyridin- 6-yl)-5-(1-methyl-1,2,5,6- tetrahydropyridin-3- yl)thiazolo[5,4-b]pyridine-2- carboxamide |
| 134 | 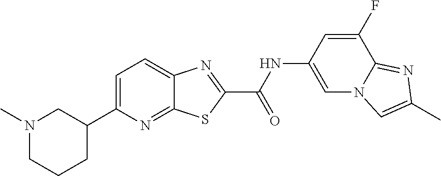 | N-(8-fluoro-2- methylimidazo[1,2-a]pyridin- 6-yl)-5-(1-methylpiperidin-3- yl)thiazolo[5,4-b]pyridine-2- carboxamide |
| 135 | 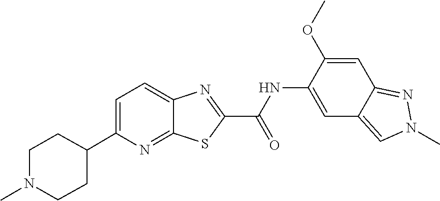 | N-(6-methoxy-2-methyl-2H- indazol-5-yl)-5-(1- methylpiperidin-4- yl)thiazolo[5,4-b]pyridine-2- carboxamide |
| 136 | 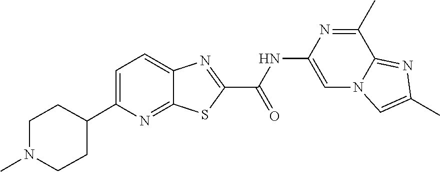 | N-(2,8-dimethylimidazo[1,2- a]pyrazin-6-yl)-5-(1- methylpiperidin-4- yl)thiazolo[5,4-b]pyridine-2- carboxamide |
| 137 | 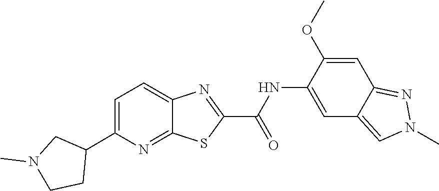 | N-(6-methoxy-2-methyl-2H- indazol-5-yl)-5-(1- methylpyrrolidin-3- yl)thiazolo[5,4-b]pyridine-2- carboxamide |
| 138 | 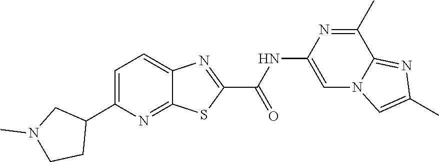 | N-(2,8-dimethylimidazo[1,2- a]pyrazin-6-yl)-5-(1- methylpyrrolidin-3- yl)thiazolo[5,4-b]pyridine-2- carboxamide |
| 139 | 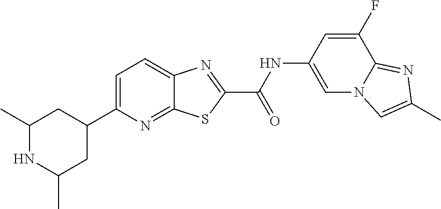 | 5-(2,6-dimethylpiperidin-4- yl)-N-(8-fluoro-2- methylimidazo[1,2-a]pyridin- 6-yl)thiazolo[5,4-b]pyridine- 2-carboxamide |
| 140 | 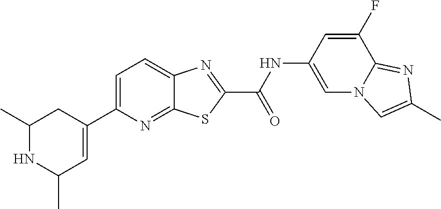 | 5-(2,6-dimethyl-1,2,3,6- tetrahydropyridin-4-yl)-N-(8- fluoro-2-methylimidazo[1,2- a]pyridin-6-yl)thiazolo[5,4- b]pyridine-2-carboxamide |
| 141 | 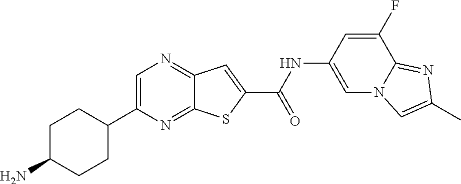 | 3-(4-aminocyclohexyl)-N-(8- fluoro-2-methylimidazo[1,2- a]pyridin-6-yl)thieno[2,3- b]pyrazine-6-carboxamide |
| 142 | 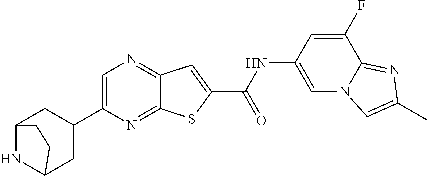 [SMILES: Cc1cn2cc(NC(=O)c3cc4ncc(C5CC6CCC(C5)N6)nc4s3)cc(F)c2n1] | 3-(8-azabicyclo[3.2.1]octan- 3-yl)-N-(8-fluoro-2- methylimidazo[1,2-a]pyridin- 6-yl)thieno[2,3-b]pyrazine-6- carboxamide |
| 143 | 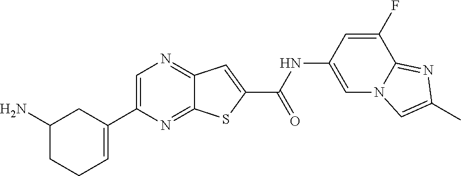 [SMILES: Cc1cn2cc(NC(=O)c3cc4ncc(C5=CCCC(N)C5)nc4s3)cc(F)c2n1] | 3-(5-aminocyclohex-1-en-1- yl)-N-(8-fluoro-2- methylimidazo[1,2-a]pyridin- 6-yl)thieno[2,3-b]pyrazine-6- carboxamide |
| 144 | 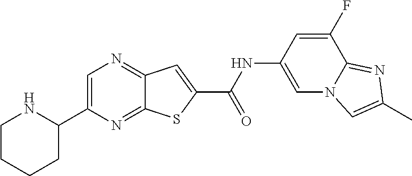 [SMILES: Cc1cn2cc(NC(=O)c3cc4ncc(C5CCCCN5)nc4s3)cc(F)c2n1] | N-(8-fluoro-2- methylimidazo[1,2-a]pyridin- 6-yl)-3-(piperidin-2- yl)thieno[2,3-b]pyrazine-6- carboxamide |
| 145 | 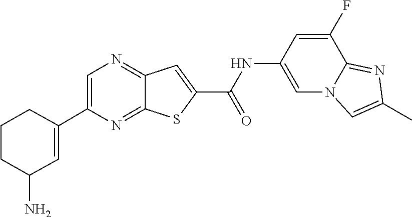 [SMILES: Cc1cn2cc(NC(=O)c3cc4ncc(C5=CC(N)CCC5)nc4s3)cc(F)c2n1] | 3-(3-aminocyclohex-1-en-1- yl)-N-(8-fluoro-2- methylimidazo[1,2-a]pyridin- 6-yl)thieno[2,3-b]pyrazine-6- carboxamide |
| 146 | 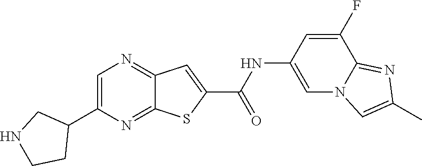 [SMILES: Cc1cn2cc(NC(=O)c3cc4ncc(C5CCNC5)nc4s3)cc(F)c2n1] | N-(8-fluoro-2- methylimidazo[1,2-a]pyridin- 6-yl)-3-(pyrrolidin-3- yl)thieno[2,3-b]pyrazine-6- carboxamide |
| 147 | 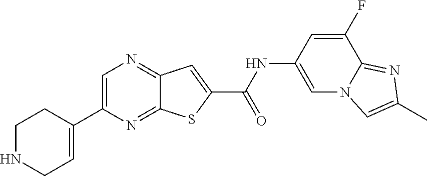 [SMILES: Cc1cn2cc(NC(=O)c3cc4ncc(C5=CCNCC5)nc4s3)cc(F)c2n1] | N-(8-fluoro-2- methylimidazo[1,2-a]pyridin- 6-yl)-3-(1,2,3,6- tetrahydropyridin-4- yl)thieno[2,3-b]pyrazine-6- carboxamide |
| 148 | 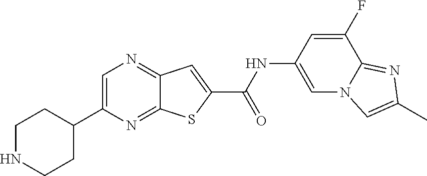 [SMILES: Cc1cn2cc(NC(=O)c3cc4ncc(C5CCNCC5)nc4s3)cc(F)c2n1] | N-(8-fluoro-2- methylimidazo[1,2-a]pyridin- 6-yl)-3-(piperidin-4- yl)thieno[2,3-b]pyrazine-6- carboxamide |
| 149 | 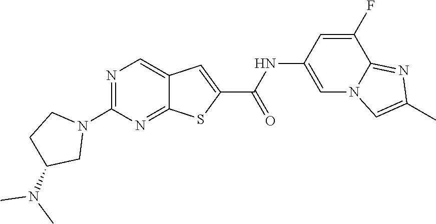 | (R)-2-(3- (dimethylamino)pyrrolidin-1- yl)-N-(8-fluoro-2- methylimidazo[1,2-a]pyridin- 6-yl)thieno[2,3-d]pyrimidine- 6-carboxamide |
| 150 | 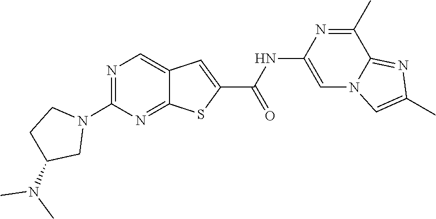 | (R)-2-(3- (dimethylamino)pyrrolidin-1- yl)-N-(2,8- dimethylimidazo[1,2- a]pyrazin-6-yl)thieno[2,3- d]pyrimidine-6-carboxamide |
| 151 | 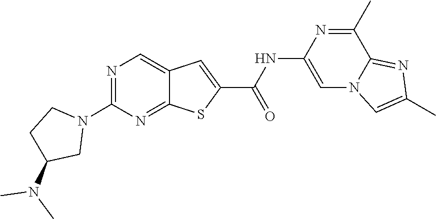 | (S)-2-(3- (dimethylamino)pyrrolidin-1- yl)-N-(2,8- dimethylimidazo[1,2- a]pyrazin-6-yl)thieno[2,3- d]pyrimidine-6-carboxamide |
| 152 | 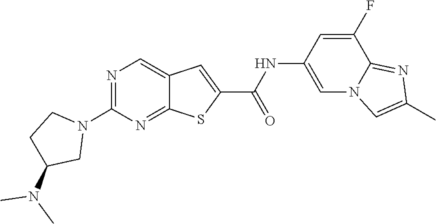 | (S)-2-(3- (dimethylamino)pyrrolidin-1- yl)-N-(8-fluoro-2- methylimidazo[1,2-a]pyridin- 6-yl)thieno[2,3-d]pyrimidine- 6-carboxamide |
| 153 | 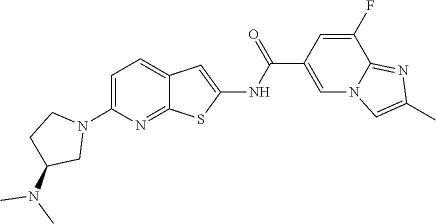 | (S)-N-(6-(3- (dimethylamino)pyrrolidin-1- yl)thieno[2,3-b]pyridin-2-yl)- 8-fluoro-2- methylimidazo[1,2- a]pyridine-6-carboxamide |
| 154 | 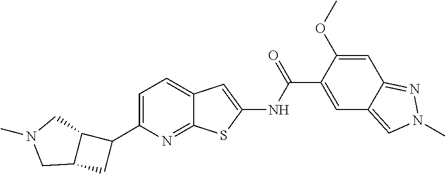 | 6-methoxy-2-methyl-N-(6- ((1S,5R)-3-methyl-3,6- diazabicyclo[3.2.0]heptan-6- yl)thieno[2,3-b]pyridin-2-yl)- 2H-indazole-5-carboxamide |
| 155 | 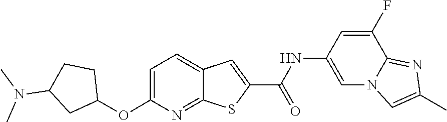 | 6-((3- (dimethylamino)cyclopentyl) oxy)-N-(8-fluoro-2- methylimidazo[1,2-a]pyridin- 6-yl)thieno[2,3-b]pyridine-2- carboxamide |
| 156 | 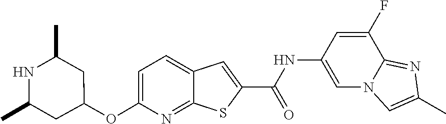 | 6-(((2S,6R)-2,6- dimethylpiperidin-4-yl)oxy)- N-(8-fluoro-2- methylimidazo[1,2-a]pyridin- 6-yl)thieno[2,3-b]pyridine-2- carboxamide |
| 157 | 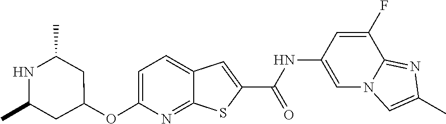 | 6-(((2R,6R)-2,6- dimethylpiperidin-4-yl)oxy)- N-(8-fluoro-2- methylimidazo[1,2-a]pyridin- 6-yl)thieno[2,3-b]pyridine-2- carboxamide |
| 158 | 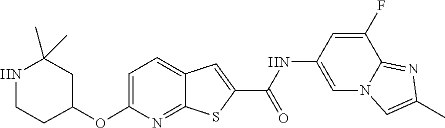 [SMILES: Cc1cn2cc(NC(=O)c3cc4ccc(OC5CCNC(C)(C)C5)nc4s3)cc(F)c2n1] | 6-((2,2-dimethylpiperidin-4- yl)oxy)-N-(8-fluoro-2- methylimidazo[1,2-a]pyridin- 6-yl)thieno[2,3-b]pyridine-2- carboxamide |
| 159 | 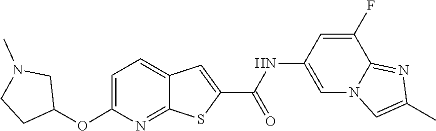 [SMILES: Cc1cn2cc(NC(=O)c3cc4ccc(OC5CCN(C)C5)nc4s3)cc(F)c2n1] | N-(8-fluoro-2- methylimidazo[1,2-a]pyridin- 6-yl)-6-((1-methylpyrrolidin- 3-yl)oxy)thieno[2,3- b]pyridine-2-carboxamide |
| 160 | 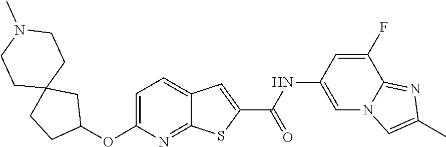 [SMILES: Cc1cn2cc(NC(=O)c3cc4ccc(OC5CCC6(CCN(C)CC6)C5)nc4s3)cc(F)c2n1] | N-(8-fluoro-2- methylimidazo[1,2-a]pyridin- 6-yl)-6-((8-methyl-8- azaspiro[4.5]decan-2- yl)oxy)thieno[2,3-b]pyridine- 2-carboxamide |
| 161 | 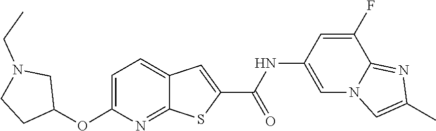 [SMILES: CCN1CCC(Oc2ccc3cc(C(=O)Nc4cc(F)c5nc(C)cn5c4)sc3n2)C1] | 6-((1-ethylpyrrolidin-3- yl)oxy)-N-(8-fluoro-2- methylimidazo[1,2-a]pyridin- 6-yl)thieno[2,3-b]pyridine-2- carboxamide |
| 162 | 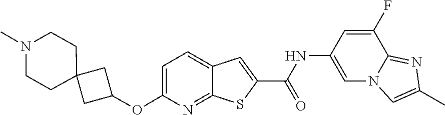 | N-(8-fluoro-2- methylimidazo[1,2-a]pyridin- 6-yl)-6-((7-methyl-7- azaspiro[3.5]nonan-2- yl)oxy)thieno[2,3-b]pyridine- 2-carboxamide |
| 163 | 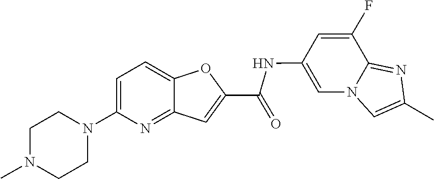 | N-(8-fluoro-2- methylimidazo[1,2-a]pyridin- 6-yl)-5-(1-methylpiperidin-4- yl)furo[3,2-b]pyridine-2- carboxamide |
| 164 | 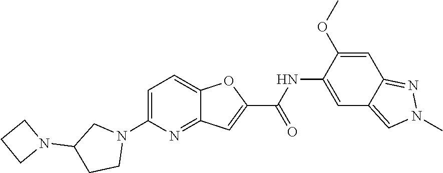 [SMILES: COc1cc2nn(C)cc2cc1NC(=O)c1cc2nc(N3CCC(N4CCC4)C3)ccc2o1] | 5-(3-(azetidin-1-yl)pyrrolidin- 1-yl)-N-(6-methoxy-2- methyl-2H-indazol-5- yl)furo[3,2-b]pyridine-2- carboxamide |
| 165 | 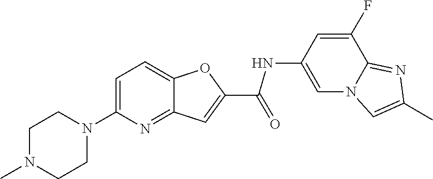 | N-(8-fluoro-2- methylimidazo[1,2-a]pyridin- 6-yl)-5-(4-methylpiperazin-1- yl)furo[3,2-b]pyridine-2- carboxamide |
| 166 | 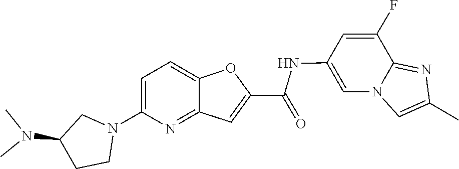 | (R)-5-(3- (dimethylamino)pyrrolidin-1- yl)-N-(8-fluoro-2- methylimidazo[1,2-a]pyridin- 6-yl)furo[3,2-b]pyridine-2- carboxamide |
| 167 | 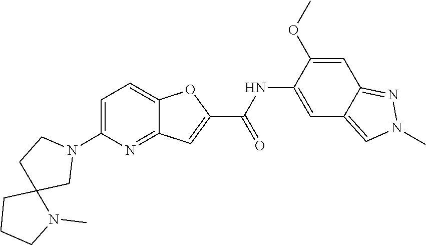 [SMILES: COc1cc2nn(C)cc2cc1NC(=O)c1cc2nc(N3CCC4(CCCN4C)C3)ccc2o1] | N-(6-methoxy-2-methyl-2H- indazol-5-yl)-5-(1-methyl- 1,7-diazaspiro[4.4]nonan-7- yl)furo[3,2-b]pyridine-2- carboxamide |
| 168 | 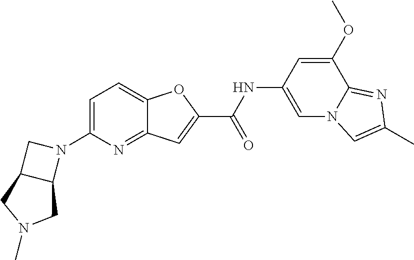 [SMILES: COc1cc(NC(=O)c2cc3nc(N4C[C@@H]5CN(C)C[C@@H]54)ccc3o2)cn2cc(C)nc12] | N-(8-methoxy-2- methylimidazo[1,2-a]pyridin- 6-yl)-5-((1S,5R)-3-methyl- 3,6- diazabicyclo[3.2.0]heptan-6- yl)furo[3,2-b]pyridine-2- carboxamide |
| 169 | 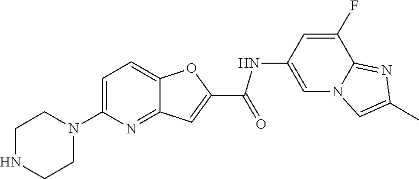 [SMILES: Cc1cn2cc(NC(=O)c3cc4nc(N5CCNCC5)ccc4o3)cc(F)c2n1] | N-(8-fluoro-2- methylimidazo[1,2-a]pyridin- 6-yl)-5-(piperazin-1- yl)furo[3,2-b]pyridine-2- carboxamide |
| 170 | 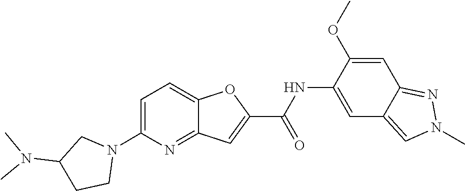 | 5-(3- (dimethylamino)pyrrolidin-1- yl)-N-(6-methoxy-2-methyl- 2H-indazol-5-yl)furo[3,2- b]pyridine-2-carboxamide |
| 171 | 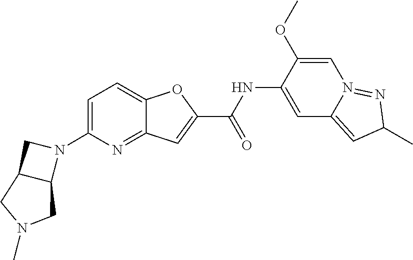 | N-(6-methoxy-2- methylpyrazolo[1,5-a]pyridin- 5-yl)-5-((1S,5R)-3-methyl- 3,6- diazabicyclo[3.2.0]heptan-6- yl)furo[3,2-b]pyridine-2- carboxamide |
| 172 | 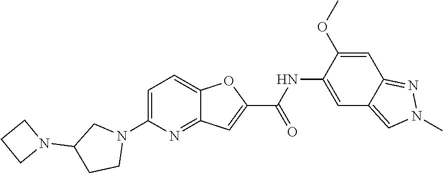 [SMILES: COc1cc2nn(C)cc2cc1NC(=O)c1cc2nc(N3CCC(N4CCC4)C3)ccc2o1] | (R)-5-(3-(azetidin-1- yl)pyrrolidin-1-yl)-N-(6- methoxy-2-methyl-2H- indazol-5-yl)furo[3,2- b]pyridine-2-carboxamide |
| 173 | 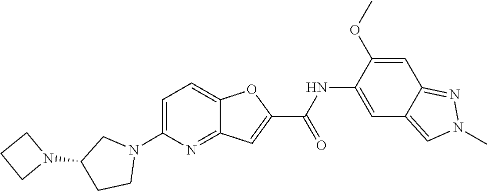 [SMILES: COc1cc2nn(C)cc2cc1NC(=O)c1cc2nc(N3CC[C@H](N4CCC4)C3)ccc2o1] | (S)-5-(3-(azetidin-1- yl)pyrrolidin-1-yl)-N-(6- methoxy-2-methyl-2H- indazol-5-yl)furo[3,2- b]pyridine-2-carboxamide |
| 174 | 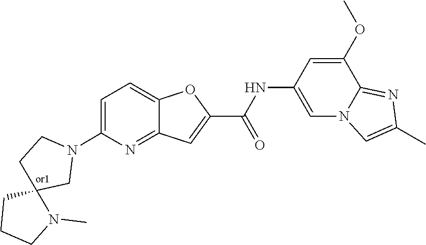 [SMILES: COc1cc(NC(=O)c2cc3nc(N4CC[C@@]5(CCCN5C)C4)ccc3o2)cn2cc(C)nc12] | rel-(R)-N-(8-methoxy-2- methylimidazo[1,2-a]pyridin- 6-yl)-5-(1-methyl-1,7- diazaspiro[4.4]nonan-7- yl)furo[3,2-b]pyridine-2- carboxamide |
| 175 | 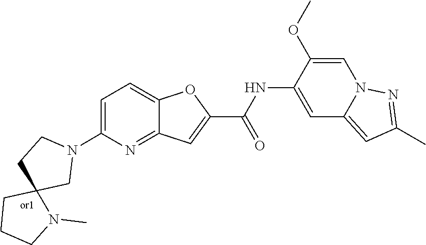 [SMILES: COc1cn2nc(C)cc2cc1NC(=O)c1cc2nc(N3CC[C@@]4(CCCN4C)C3)ccc2o1] | rel-(R)-N-(6-methoxy-2- methylpyrazolo[1,5-a]pyridin- 5-yl)-5-(1-methyl-1,7- diazaspiro[4.4]nonan-7- yl)furo[3,2-b]pyridine-2- carboxamide |
| 176 | 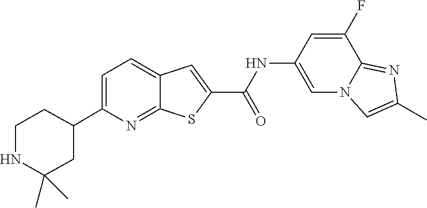 [SMILES: Cc1cn2cc(NC(=O)c3cc4ccc(C5CCNC(C)(C)C5)nc4s3)cc(F)c2n1] | 6-(2,2-dimethylpiperidin-4- yl)-N-(8-fluoro-2- methylimidazo[1,2-a]pyridin- 6-yl)thieno[2,3-b]pyridine-2- carboxamide |
| 177 | 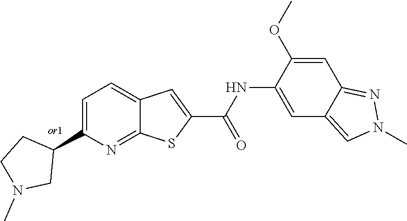 [SMILES: COc1cc2nn(C)cc2cc1NC(=O)c1cc2ccc([C@@H]3CCN(C)C3)nc2s1] | rel-(R)-N-(6-methoxy-2- methyl-2H-indazol-5-yl)-6-(1- methylpyrrolidin-3- yl)thieno[2,3-b]pyridine-2- carboxamide |
| 178 | 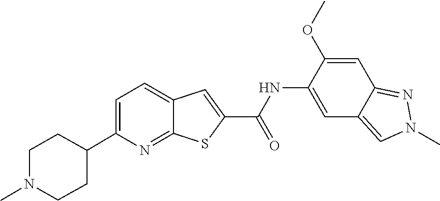 | N-(6-methoxy-2-methyl-2H- indazol-5-yl)-6-(1- methylpiperidin-4- yl)thieno[2,3-b]pyridine-2- carboxamide |
| 179 | 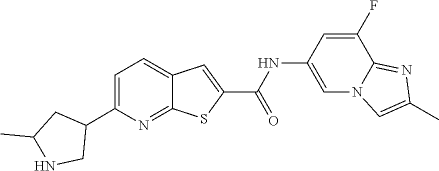 | N-(8-fluoro-2- methylimidazo[1,2-a]pyridin- 6-yl)-6-(5-methylpyrrolidin-3- yl)thieno[2,3-b]pyridine-2- carboxamide |
| 180 | 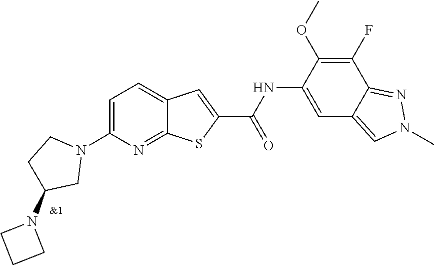 [SMILES: COc1c(NC(=O)c2cc3ccc(N4CC[C@H](N5CCC5)C4)nc3s2)cc2cn(C)nc2c1F] | rac-(R)-6-(3-(azetidin-1- yl)pyrrolidin-1-yl)-N-(7- fluoro-6-methoxy-2-methyl- 2H-indazol-5-yl)thieno[2,3- b]pyridine-2-carboxamide |
| 181 | 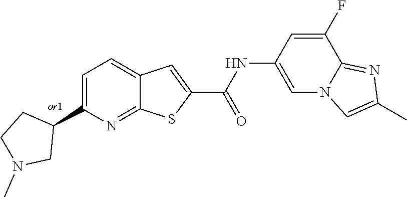 [SMILES: Cc1cn2cc(NC(=O)c3cc4ccc([C@@H]5CCN(C)C5)nc4s3)cc(F)c2n1] | rel-(R)-N-(8-fluoro-2- methylimidazo[1,2-a]pyridin- 6-yl)-6-(1-methylpyrrolidin-3- yl)thieno[2,3-b]pyridine-2- carboxamide |
| 182 | 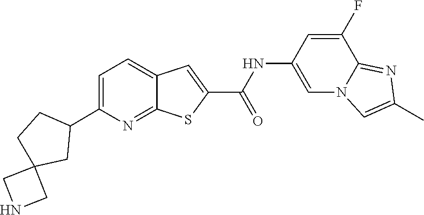 [SMILES: Cc1cn2cc(NC(=O)c3cc4ccc(C5CCC6(CNC6)C5)nc4s3)cc(F)c2n1] | N-(8-fluoro-2- methylimidazo[1,2-a]pyridin- 6-yl)-6-(2-azaspiro[3.4]octan- 6-yl)thieno[2,3-b]pyridine-2- carboxamide |
| 183 | 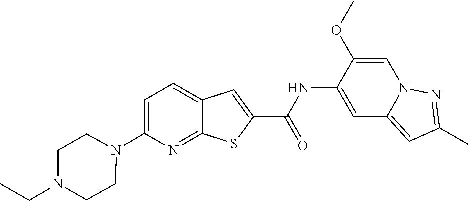 [SMILES: CCN1CCN(c2ccc3cc(C(=O)Nc4cc5cc(C)nn5cc4OC)sc3n2)CC1] | 6-(4-ethylpiperazin-1-yl)-N- (6-methoxy-2- methylpyrazolo[1,5-a]pyridin- 5-yl)thieno[2,3-b]pyridine-2- carboxamide |
| 184 | 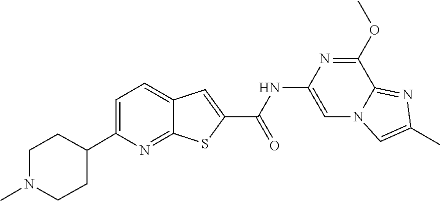 | N-(8-methoxy-2- methylimidazo[1,2-a]pyrazin- 6-yl)-6-(1-methylpiperidin-4- yl)thieno[2,3-b]pyridine-2- carboxamide |
| 185 | 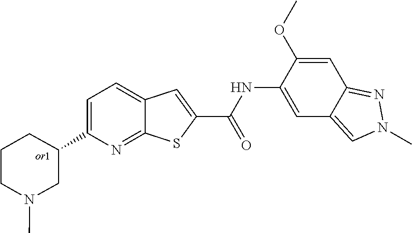 [SMILES: COc1cc2nn(C)cc2cc1NC(=O)c1cc2ccc([C@H]3CCCN(C)C3)nc2s1] | rel-(R)-N-(6-methoxy-2- methyl-2H-indazol-5-yl)-6-(1- methylpiperidin-3- yl)thieno[2,3-b]pyridine-2- carboxamide |
| 186 | 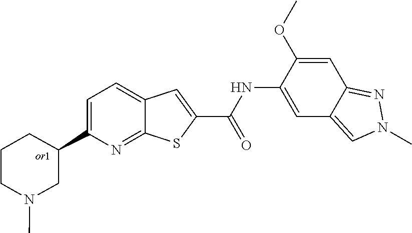 [SMILES: COc1cc2nn(C)cc2cc1NC(=O)c1cc2ccc([C@@H]3CCCN(C)C3)nc2s1] | rel-(R)-N-(6-methoxy-2- methyl-2H-indazol-5-yl)-6-(1- methylpiperidin-3- yl)thieno[2,3-b]pyridine-2- carboxamide |
| 187 | 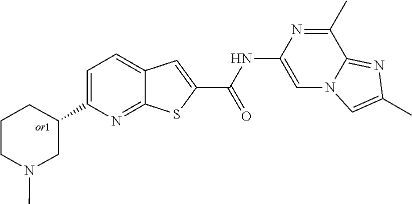 [SMILES: Cc1cn2cc(NC(=O)c3cc4ccc([C@H]5CCCN(C)C5)nc4s3)nc(C)c2n1] | rel-(R)-N-(2,8- dimethylimidazo[1,2- a]pyrazin-6-yl)-6-(1- methylpiperidin-3- yl)thieno[2,3-b]pyridine-2- carboxamide |
| 188 | 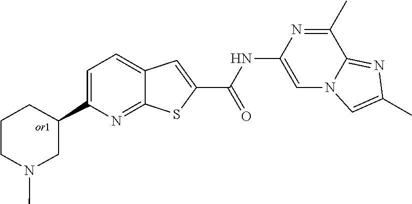 [SMILES: Cc1cn2cc(NC(=O)c3cc4ccc([C@@H]5CCCN(C)C5)nc4s3)nc(C)c2n1] | rel-(R)-N-(2,8- dimethylimidazo[1,2- a]pyrazin-6-yl)-6-(1- methylpiperidin-3- yl)thieno[2,3-b]pyridine-2- carboxamide |
| 189 | 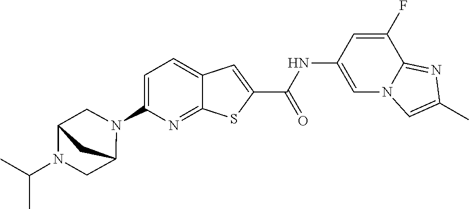 | N-(8-fluoro-2- methylimidazo[1,2-a]pyridin- 6-yl)-6-((1S,4S)-5-isopropyl- 2,5- diazabicyclo[2.2.1]heptan-2- yl)thieno[2,3-b]pyridine-2- carboxamide |
| 190 | 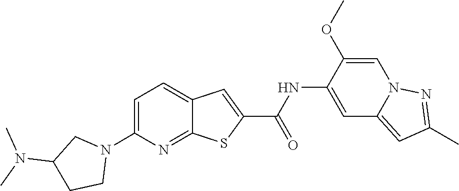 | 6-(3- (dimethylamino)pyrrolidin-1- yl)-N-(6-methoxy-2- methylpyrazolo[1,5-a]pyridin- 5-yl)thieno[2,3-b]pyridine-2- carboxamide |
| 191 | 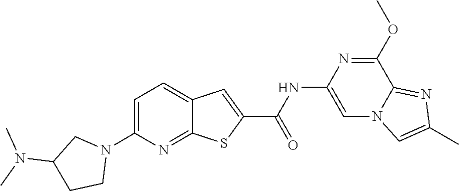 | 6-(3- (dimethylamino)pyrrolidin-1- yl)-N-(8-methoxy-2- methylimidazo[1,2-a]pyrazin- 6-yl)thieno[2,3-b]pyridine-2- carboxamide |
| 192 | 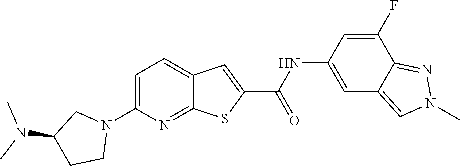 | (R)-6-(3- (dimethylamino)pyrrolidin-1- yl)-N-(7-fluoro-2-methyl-2H- indazol-5-yl)thieno[2,3- b]pyridine-2-carboxamide |
| 193 | 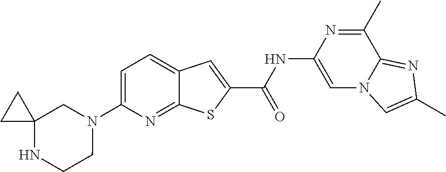 [SMILES: Cc1cn2cc(NC(=O)c3cc4ccc(N5CCNC6(CC6)C5)nc4s3)nc(C)c2n1] | N-(2,8-dimethylimidazo[1,2- a]pyrazin-6-yl)-6-(4,7- diazaspiro[2.5]octan-7- yl)thieno[2,3-b]pyridine-2- carboxamide |
| 194 | 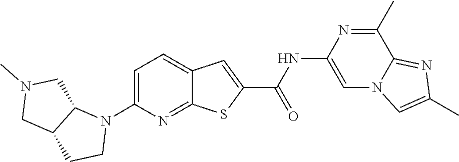 | N-(2,8-dimethylimidazo[1,2- a]pyrazin-6-yl)-6-((3aR,6aR)- 5- methylhexahydropyrrolo[3,4- b]pyrrol-1(2H)-yl)thieno[2,3- b]pyridine-2-carboxamide |
| 195 | 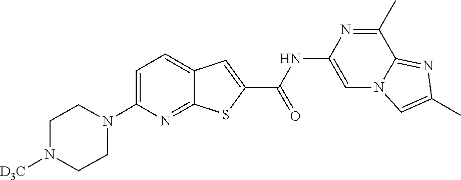 [SMILES: [2H]C([2H])([2H])N1CCN(c2ccc3cc(C(=O)Nc4cn5cc(C)nc5c(C)n4)sc3n2)CC1] | N-(2,8-dimethylimidazo[1,2- a]pyrazin-6-yl)-6-(4-(methyl- d3)piperazin-1-yl)thieno[2,3- b]pyridine-2-carboxamide |
| 196 | 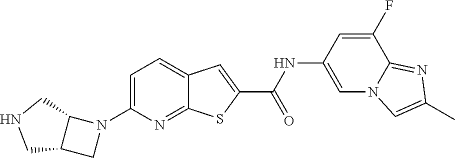 [SMILES: Cc1cn2cc(NC(=O)c3cc4ccc(N5C[C@@H]6CNC[C@@H]65)nc4s3)cc(F)c2n1] | 6-((1S,5R)-3,6- diazabicyclo[3.2.0]heptan-6- yl)-N-(8-fluoro-2- methylimidazo[1,2-a]pyridin- 6-yl)thieno[2,3-b]pyridine-2- carboxamide |
| 197 | 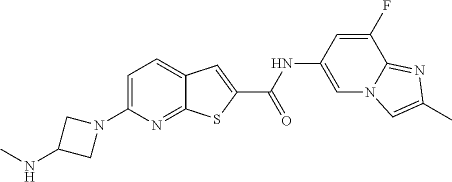 | N-(8-fluoro-2- methylimidazo[1,2-a]pyridin- 6-yl)-6-(3- (methylamino)azetidin-1- yl)thieno[2,3-b]pyridine-2- carboxamide |
| 198 | 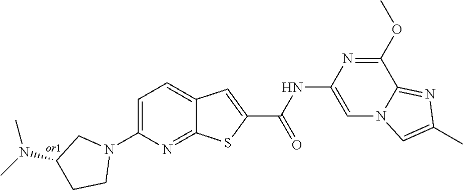 | rel-(R)-6-(3- (dimethylamino)pyrrolidin-1- yl)-N-(8-methoxy-2- methylimidazo[1,2-a]pyrazin- 6-yl)thieno[2,3-b]pyridine-2- carboxamide |
| 199 | 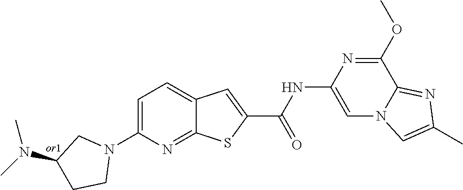 | rel-(R)-6-(3- (dimethylamino)pyrrolidin-1- yl)-N-(8-methoxy-2- methylimidazo[1,2-a]pyrazin- 6-yl)thieno[2,3-b]pyridine-2- carboxamide |
| 200 | 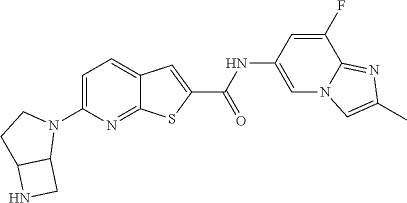 [SMILES: Cc1cn2cc(NC(=O)c3cc4ccc(N5CCC6NCC65)nc4s3)cc(F)c2n1] | 6-(2,6- diazabicyclo[3.2.0]heptan-2- yl)-N-(8-fluoro-2- methylimidazo[1,2-a]pyridin- 6-yl)thieno[2,3-b]pyridine-2- carboxamide |
| 201 | 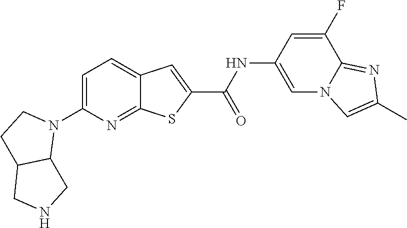 [SMILES: Cc1cn2cc(NC(=O)c3cc4ccc(N5CCC6CNCC65)nc4s3)cc(F)c2n1] | N-(8-fluoro-2- methylimidazo[1,2-a]pyridin- 6-yl)-6- (hexahydropyrrolo[3,4- b]pyrrol-1(2H)-yl)thieno[2,3- b]pyridine-2-carboxamide |
| 202 | 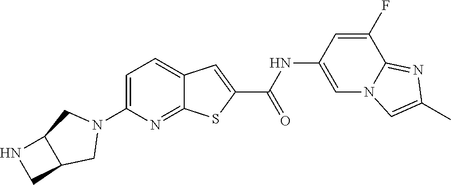 [SMILES: Cc1cn2cc(NC(=O)c3cc4ccc(N5C[C@H]6CN[C@H]6C5)nc4s3)cc(F)c2n1] | 6-((1R,5R)-3,6- diazabicyclo[3.2.0]heptan-3- yl)-N-(8-fluoro-2- methylimidazo[1,2-a]pyridin- 6-yl)thieno[2,3-b]pyridine-2- carboxamide |
| 203 | 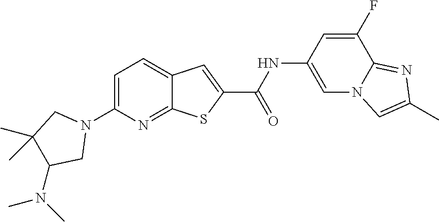 | 6-(4-(dimethylamino)-3,3- dimethylpyrrolidin-1-yl)-N- (8-fluoro-2- methylimidazo[1,2-a]pyridin- 6-yl)thieno[2,3-b]pyridine-2- carboxamide |
| 204 | 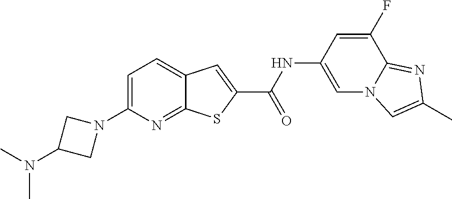 | 6-(3-(dimethylamino)azetidin- 1-yl)-N-(8-fluoro-2- methylimidazo[1,2-a]pyridin- 6-yl)thieno[2,3-b]pyridine-2- carboxamide |
| 205 | 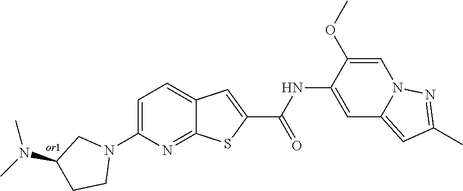 | rel-(R)-6-(3- (dimethylamino)pyrrolidin-1- yl)-N-(6-methoxy-2- methylpyrazolo[1,5-a]pyridin- 5-yl)thieno[2,3-b]pyridine-2- carboxamide |
| 206 | 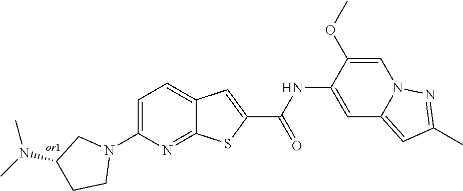 | rel-(R)-6-(3- (dimethylamino)pyrrolidin-1- yl)-N-(6-methoxy-2- methylpyrazolo[1,5-a]pyridin- 5-yl)thieno[2,3-b]pyridine-2- carboxamide |
| 207 | 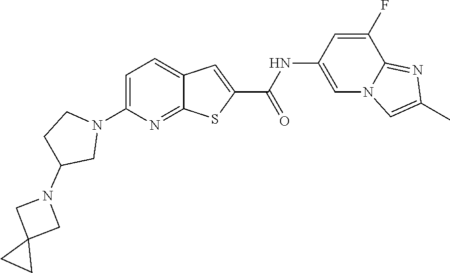 [SMILES: Cc1cn2cc(NC(=O)c3cc4ccc(N5CCC(N6CC7(CC7)C6)C5)nc4s3)cc(F)c2n1] | 6-(3-(5-azaspiro[2.3]hexan-5- yl)pyrrolidin-1-yl)-N-(8- fluoro-2-methylimidazo[1,2- a]pyridin-6-yl)thieno[2,3- b]pyridine-2-carboxamide |
| 208 | 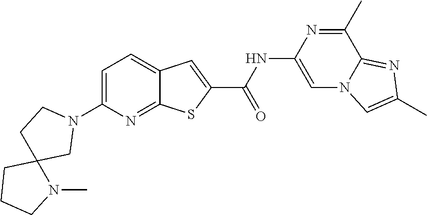 [SMILES: Cc1cn2cc(NC(=O)c3cc4ccc(N5CCC6(CCCN6C)C5)nc4s3)nc(C)c2n1] | N-(2,8-dimethylimidazo[1,2- a]pyrazin-6-yl)-6-(1-methyl- 1,7-diazaspiro[4.4]nonan-7- yl)thieno[2,3-b]pyridine-2- carboxamide |
| 209 | 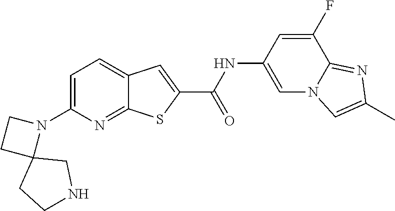 [SMILES: Cc1cn2cc(NC(=O)c3cc4ccc(N5CCC56CCNC6)nc4s3)cc(F)c2n1] | N-(8-fluoro-2- methylimidazo[1,2-a]pyridin- 6-yl)-6-(1,6- diazaspiro[3.4]octan-1- yl)thieno[2,3-b]pyridine-2- carboxamide |
| 210 | 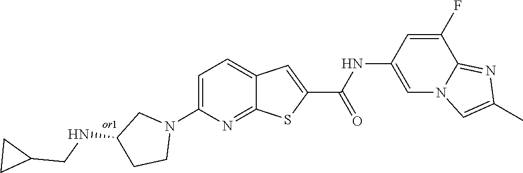 [SMILES: Cc1cn2cc(NC(=O)c3cc4ccc(N5CC[C@H](NCC6CC6)C5)nc4s3)cc(F)c2n1] | rel-(R)-6-(3- ((cyclopropylmethyl)amino) pyrrolidin-1-yl)-N-(8-fluoro-2- methylimidazo[1,2-a]pyridin- 6-yl)thieno[2,3-b]pyridine-2- carboxamide |
| 211 | 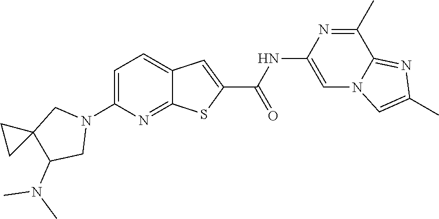 [SMILES: Cc1cn2cc(NC(=O)c3cc4ccc(N5CC(N(C)C)C6(CC6)C5)nc4s3)nc(C)c2n1] | 6-(7-(dimethylamino)-5- azaspiro[2.4]heptan-5-yl)-N- (2,8-dimethylimidazo[1,2- a]pyrazin-6-yl)thieno[2,3- b]pyridine-2-carboxamide |
| 212 | 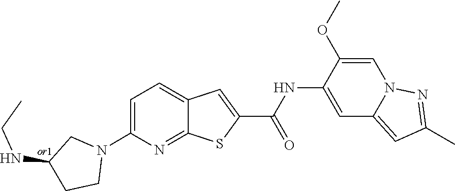 [SMILES: CCN[C@@H]1CCN(c2ccc3cc(C(=O)Nc4cc5cc(C)nn5cc4OC)sc3n2)C1] | rel-(R)-6-(3- (ethylamino)pyrrolidin-1-yl)- N-(6-methoxy-2- methylpyrazolo[1,5-a]pyridin- 5-yl)thieno[2,3-b]pyridine-2- carboxamide |
| 213 | 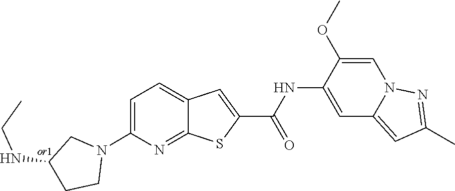 [SMILES: CCN[C@H]1CCN(c2ccc3cc(C(=O)Nc4cc5cc(C)nn5cc4OC)sc3n2)C1] | rel-(R)-6-(3- (ethylamino)pyrrolidin-1-yl)- N-(6-methoxy-2- methylpyrazolo[1,5-a]pyridin- 5-yl)thieno[2,3-b]pyridine-2- carboxamide |
| 214 | 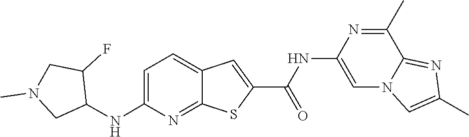 | N-(2,8-dimethylimidazo[1,2- a]pyrazin-6-yl)-6-((4-fluoro- 1-methylpyrrolidin-3- yl)amino)thieno[2,3- b]pyridine-2-carboxamide |
| 215 | 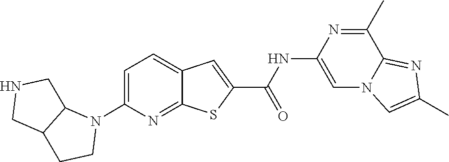 [SMILES: Cc1cn2cc(NC(=O)c3cc4ccc(N5CCC6CNCC65)nc4s3)nc(C)c2n1] | N-(2,8-dimethylimidazo[1,2- a]pyrazin-6-yl)-6- (hexahydropyrrolo[3,4- b]pyrrol-1(2H)-yl)thieno[2,3- b]pyridine-2-carboxamide |
| 216 | 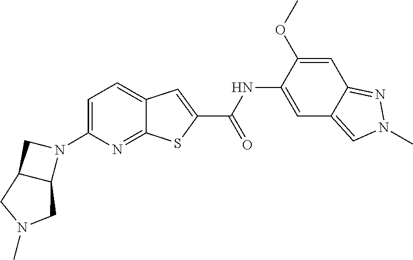 [SMILES: COc1cc2nn(C)cc2cc1NC(=O)c1cc2ccc(N3C[C@@H]4CN(C)C[C@@H]43)nc2s1] | N-(6-methoxy-2-methyl-2H- indazol-5-yl)-6-((1S,5R)-3- methyl-3,6- diazabicyclo[3.2.0]heptan-6- yl)thieno[2,3-b]pyridine-2- carboxamide |
| 217 | 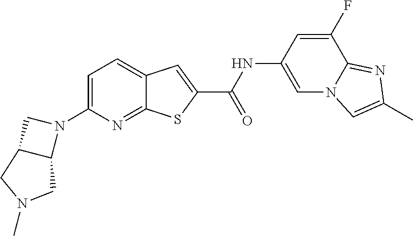 [SMILES: Cc1cn2cc(NC(=O)c3cc4ccc(N5C[C@H]6CN(C)C[C@H]65)nc4s3)cc(F)c2n1] | N-(8-fluoro-2- methylimidazo[1,2-a]pyridin- 6-yl)-6-((1R,5S)-3-methyl- 3,6- diazabicyclo[3.2.0]heptan-6- yl)thieno[2,3-b]pyridine-2- carboxamide |
| 218 | 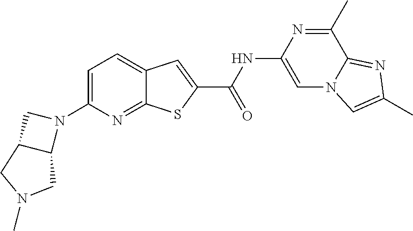 [SMILES: Cc1cn2cc(NC(=O)c3cc4ccc(N5C[C@H]6CN(C)C[C@H]65)nc4s3)nc(C)c2n1] | N-(2,8-dimethylimidazo[1,2- a]pyrazin-6-yl)-6-((1R,5S)-3- methyl-3,6- diazabicyclo[3.2.0]heptan-6- yl)thieno[2,3-b]pyridine-2- carboxamide |
| 219 | 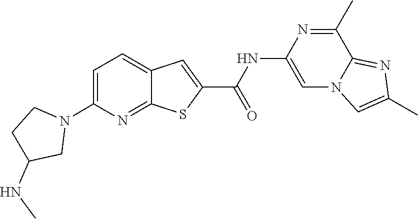 [SMILES: CNC1CCN(c2ccc3cc(C(=O)Nc4cn5cc(C)nc5c(C)n4)sc3n2)C1] | N-(2,8-dimethylimidazo[1,2- a]pyrazin-6-yl)-6-(3- (methylamino)pyrrolidin-1- yl)thieno[2,3-b]pyridine-2- carboxamide |
| 220 | 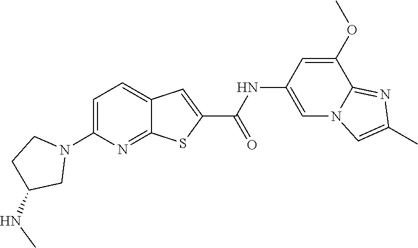 [SMILES: CN[C@@H]1CCN(c2ccc3cc(C(=O)Nc4cc(OC)c5nc(C)cn5c4)sc3n2)C1] | (R)-N-(8-methoxy-2- methylimidazo[1,2-a]pyridin- 6-yl)-6-(3- (methylamino)pyrrolidin-1- yl)thieno[2,3-b]pyridine-2- carboxamide |
| 221 | 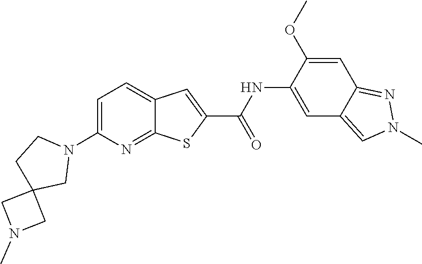 | N-(6-methoxy-2-methyl-2H- indazol-5-yl)-6-(2-methyl- 2,6-diazaspiro[3.4]octan-6- yl)thieno[2,3-b]pyridine-2- carboxamide |
| 222 | 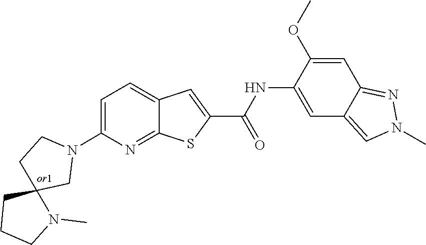 [SMILES: COc1cc2nn(C)cc2cc1NC(=O)c1cc2ccc(N3CC[C@]4(CCCN4C)C3)nc2s1] | rel-(R)-N-(6-methoxy-2- methyl-2H-indazol-5-yl)-6-(1- methyl-1,7- diazaspiro[4.4]nonan-7- yl)thieno[2,3-b]pyridine-2- carboxamide |
| 223 | 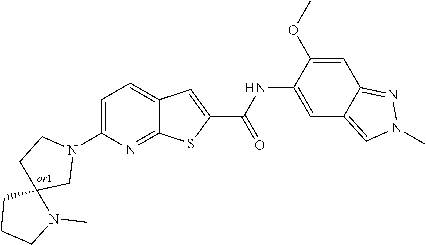 [SMILES: COc1cc2nn(C)cc2cc1NC(=O)c1cc2ccc(N3CC[C@@]4(CCCN4C)C3)nc2s1] | rel-(R)-N-(6-methoxy-2- methyl-2H-indazol-5-yl)-6-(1- methyl-1,7- diazaspiro[4.4]nonan-7- yl)thieno[2,3-b]pyridine-2- carboxamide |
| 224 | 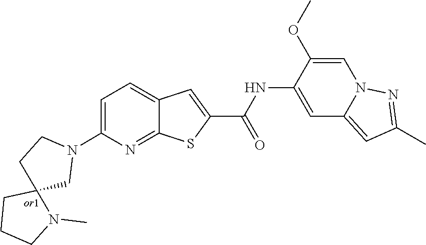 [SMILES: COc1cn2nc(C)cc2cc1NC(=O)c1cc2ccc(N3CC[C@@]4(CCCN4C)C3)nc2s1] | rel-(R)-N-(6-methoxy-2- methylpyrazolo[1,5-a]pyridin- 5-yl)-6-(1-methyl-1,7- diazaspiro[4.4]nonan-7- yl)thieno[2,3-b]pyridine-2- carboxamide |
| 225 | 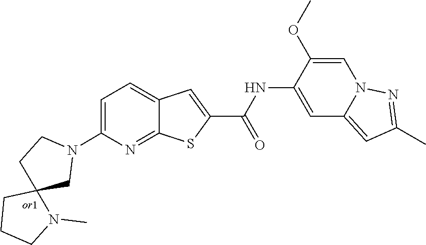 [SMILES: COc1cn2nc(C)cc2cc1NC(=O)c1cc2ccc(N3CC[C@]4(CCCN4C)C3)nc2s1] | rel-(R)-N-(6-methoxy-2- methylpyrazolo[1,5-a]pyridin- 5-yl)-6-(1-methyl-1,7- diazaspiro[4.4]nonan-7- yl)thieno[2,3-b]pyridine-2- carboxamide |
| 226 | 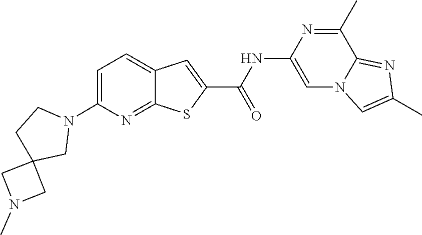 | N-(2,8-dimethylimidazo[1,2- a]pyrazin-6-yl)-6-(2-methyl- 2,6-diazaspiro[3.4]octan-6- yl)thieno[2,3-b]pyridine-2- carboxamide |
| 227 | 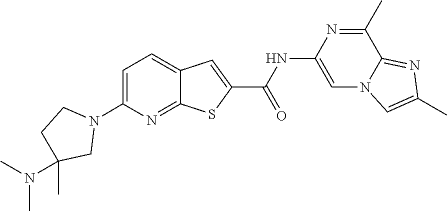 | 6-(3-(dimethylamino)-3- methylpyrrolidin-1-yl)-N- (2,8-dimethylimidazo[1,2- a]pyrazin-6-yl)thieno[2,3- b]pyridine-2-carboxamide |
| 228 | 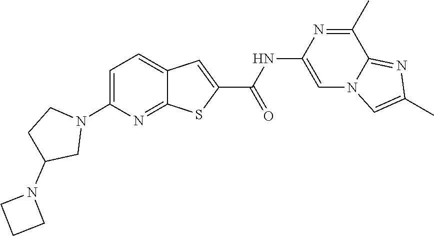 [SMILES: Cc1cn2cc(NC(=O)c3cc4ccc(N5CCC(N6CCC6)C5)nc4s3)nc(C)c2n1] | 6-(3-(azetidin-1-yl)pyrrolidin- 1-yl)-N-(2,8- dimethylimidazo[1,2- a]pyrazin-6-yl)thieno[2,3- b]pyridine-2-carboxamide |
| 229 | 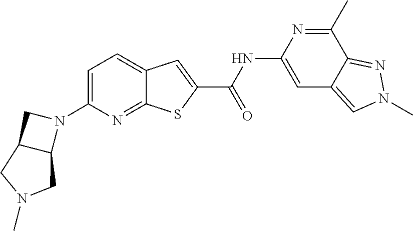 [SMILES: Cc1nc(NC(=O)c2cc3ccc(N4C[C@@H]5CN(C)C[C@@H]54)nc3s2)cc2cn(C)nc12] | N-(2,7-dimethyl-2H- pyrazolo[3,4-c]pyridin-5-yl)- 6-((1S,5R)-3-methyl-3,6- diazabicyclo[3.2.0]heptan-6- yl)thieno[2,3-b]pyridine-2- carboxamide |
| 230 | 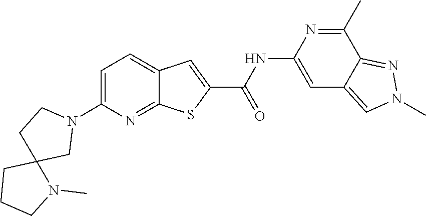 [SMILES: Cc1nc(NC(=O)c2cc3ccc(N4CCC5(CCCN5C)C4)nc3s2)cc2cn(C)nc12] | N-(2,7-dimethyl-2H- pyrazolo[3,4-c]pyridin-5-yl)- 6-(1-methyl-1,7- diazaspiro[4.4]nonan-7- yl)thieno[2,3-b]pyridine-2- carboxamide |
| 231 | 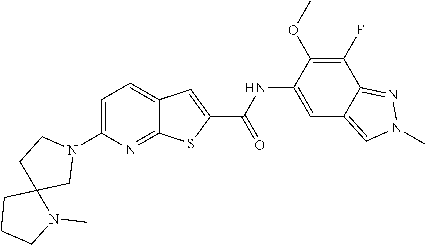 [SMILES: COc1c(NC(=O)c2cc3ccc(N4CCC5(CCCN5C)C4)nc3s2)cc2cn(C)nc2c1F] | N-(7-fluoro-6-methoxy-2- methyl-2H-indazol-5-yl)-6-(1- methyl-1,7- diazaspiro[4.4]nonan-7- yl)thieno[2,3-b]pyridine-2- carboxamide |
| 232 | 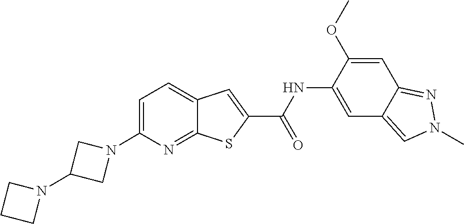 [SMILES: COc1cc2nn(C)cc2cc1NC(=O)c1cc2ccc(N3CC(N4CCC4)C3)nc2s1] | 6-([1,3′-biazetidin]-1′-yl)-N- (6-methoxy-2-methyl-2H- indazol-5-yl)thieno[2,3- b]pyridine-2-carboxamide |
| 233 | 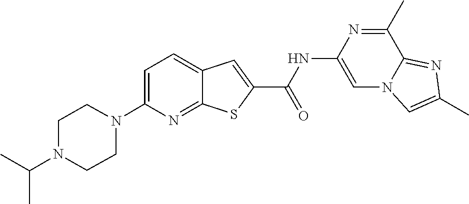 [SMILES: Cc1cn2cc(NC(=O)c3cc4ccc(N5CCN(C(C)C)CC5)nc4s3)nc(C)c2n1] | N-(2,8-dimethylimidazo[1,2- a]pyrazin-6-yl)-6-(4- isopropylpiperazin-1- yl)thieno[2,3-b]pyridine-2- carboxamide |
| 234 | 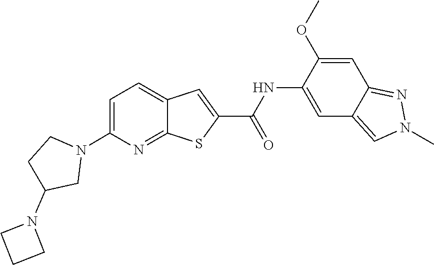 [SMILES: COc1cc2nn(C)cc2cc1NC(=O)c1cc2ccc(N3CCC(N4CCC4)C3)nc2s1] | 6-(3-(azetidin-1-yl)pyrrolidin- 1-yl)-N-(6-methoxy-2- methyl-2H-indazol-5- yl)thieno[2,3-b]pyridine-2- carboxamide |
| 235 | 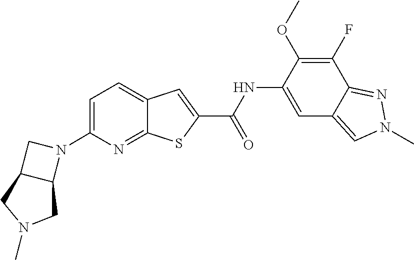 [SMILES: COc1c(NC(=O)c2cc3ccc(N4C[C@@H]5CN(C)C[C@@H]54)nc3s2)cc2cn(C)nc2c1F] | N-(7-fluoro-6-methoxy-2- methyl-2H-indazol-5-yl)-6- ((1S,5R)-3-methyl-3,6- diazabicyclo[3.2.0]heptan-6- yl)thieno[2,3-b]pyridine-2- carboxamide |
| 236 | 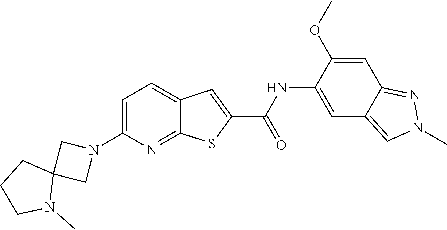 [SMILES: COc1cc2nn(C)cc2cc1NC(=O)c1cc2ccc(N3CC4(CCCN4C)C3)nc2s1] | N-(6-methoxy-2-methyl-2H- indazol-5-yl)-6-(5-methyl- 2,5-diazaspiro[3.4]octan-2- yl)thieno[2,3-b]pyridine-2- carboxamide |
| 237 | 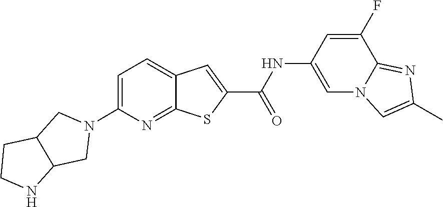 [SMILES: Cc1cn2cc(NC(=O)c3cc4ccc(N5CC6CCNC6C5)nc4s3)cc(F)c2n1] | N-(8-fluoro-2- methylimidazo[1,2-a]pyridin- 6-yl)-6- (hexahydropyrrolo[3,4- b]pyrrol-5(1H)-yl)thieno[2,3- b]pyridine-2-carboxamide |
| 238 | 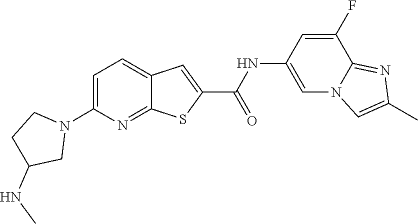 [SMILES: CNC1CCN(c2ccc3cc(C(=O)Nc4cc(F)c5nc(C)cn5c4)sc3n2)C1] | N-(8-fluoro-2- methylimidazo[1,2-a]pyridin- 6-yl)-6-(3- (methylamino)pyrrolidin-1- yl)thieno[2,3-b]pyridine-2- carboxamide |
| 239 | 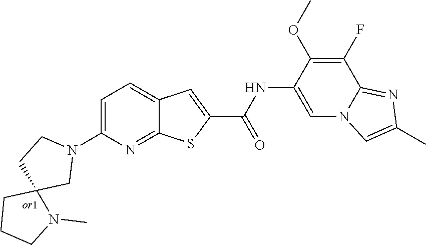 [SMILES: COc1c(NC(=O)c2cc3ccc(N4CC[C@]5(CCCN5C)C4)nc3s2)cn2cc(C)nc2c1F] | rel-(R)-N-(8-fluoro-7- methoxy-2- methylimidazo[1,2-a]pyridin- 6-yl)-6-(1-methyl-1,7- diazaspiro[4.4]nonan-7- yl)thieno[2,3-b]pyridine-2- carboxamide |
| 240 | 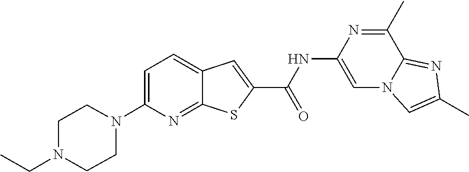 [SMILES: CCN1CCN(c2ccc3cc(C(=O)Nc4cn5cc(C)nc5c(C)n4)sc3n2)CC1] | N-(2,8-dimethylimidazo[1,2- a]pyrazin-6-yl)-6-(4- ethylpiperazin-1- yl)thieno[2,3-b]pyridine-2- carboxamide |
| 241 | 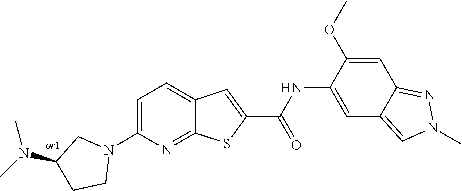 | rel-(R)-6-(3- (dimethylamino)pyrrolidin-1- yl)-N-(6-methoxy-2-methyl- 2H-indazol-5-yl)thieno[2,3- b]pyridine-2-carboxamide |
| 242 | 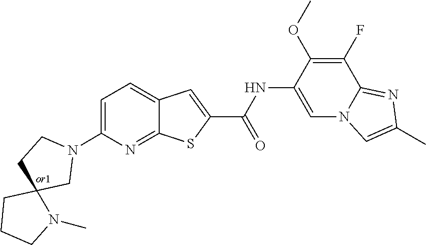 [SMILES: COc1c(NC(=O)c2cc3ccc(N4CC[C@@]5(CCCN5C)C4)nc3s2)cn2cc(C)nc2c1F] | rel-(R)-N-(8-fluoro-7- methoxy-2- methylimidazo[1,2-a]pyridin- 6-yl)-6-(1-methyl-1,7- diazaspiro[4.4]nonan-7- yl)thieno[2,3-b]pyridine-2- carboxamide |
| 243 | 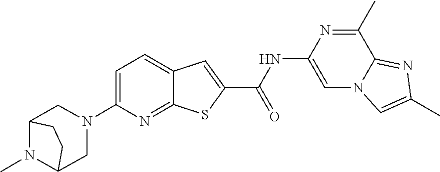 | N-(2,8-dimethylimidazo[1,2- a]pyrazin-6-yl)-6-(8-methyl- 3,8-diazabicyclo[3.2.1]octan- 3-yl)thieno[2,3-b]pyridine-2- carboxamide |
| 244 | 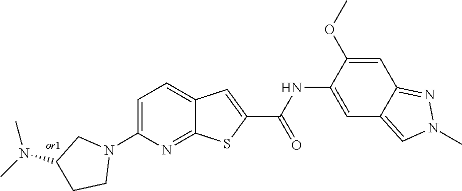 | rel-(R)-6-(3- (dimethylamino)pyrrolidin-1- yl)-N-(6-methoxy-2-methyl- 2H-indazol-5-yl)thieno[2,3- b]pyridine-2-carboxamide |
| 245 | 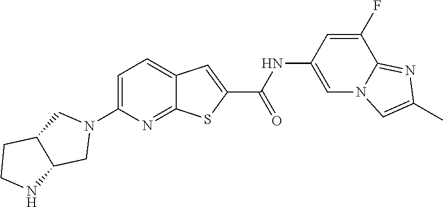 [SMILES: Cc1cn2cc(NC(=O)c3cc4ccc(N5C[C@H]6CCN[C@H]6C5)nc4s3)cc(F)c2n1] | N-(8-fluoro-2- methylimidazo[1,2-a]pyridin- 6-yl)-6-((3aR,6aR)- hexahydropyrrolo[3,4- b]pyrrol-5(1H)-yl)thieno[2,3- b]pyridine-2-carboxamide |
| 246 | 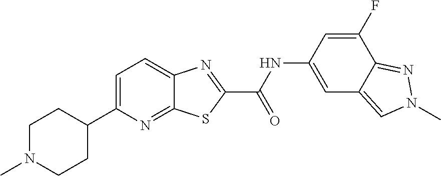 | N-(7-fluoro-2-methyl-2H- indazol-5-yl)-5-(1- methylpiperidin-4- yl)thiazolo[5,4-b]pyridine-2- carboxamide |
| 247 | 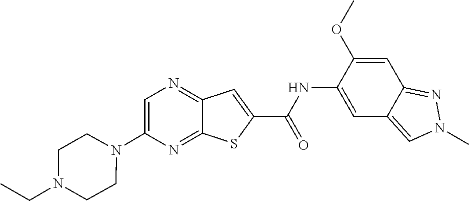 [SMILES: CCN1CCN(c2cnc3cc(C(=O)Nc4cc5cn(C)nc5cc4OC)sc3n2)CC1] | 3-(4-ethylpiperazin-1-yl)-N- (6-methoxy-2-methyl-2H- indazol-5-yl)thieno[2,3- b]pyrazine-6-carboxamide |
| 248 | 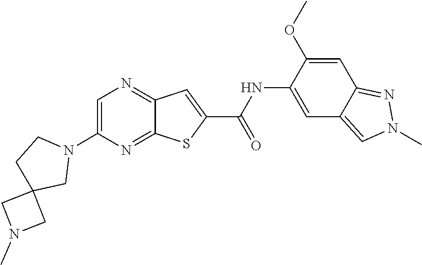 | N-(6-methoxy-2-methyl-2H- indazol-5-yl)-3-(2-methyl- 2,6-diazaspiro[3.4]octan-6- yl)thieno[2,3-b]pyrazine-6- carboxamide |
| 249 | 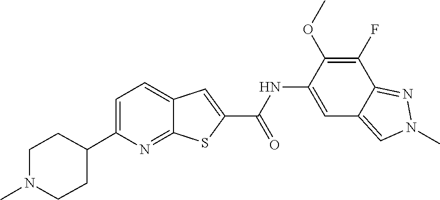 | N-(7-fluoro-6-methoxy-2- methyl-2H-indazol-5-yl)-6-(1- methylpiperidin-4- yl)thieno[2,3-b]pyridine-2- carboxamide |
| 250 | 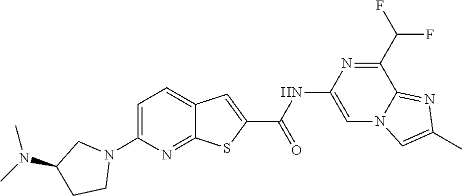 | (R)-N-(8-(difluoromethyl)-2- methylimidazo[1,2-a]pyrazin- 6-yl)-6-(3- (dimethylamino)pyrrolidin-1- yl)thieno[2,3-b]pyridine-2- carboxamide |
| 251 | 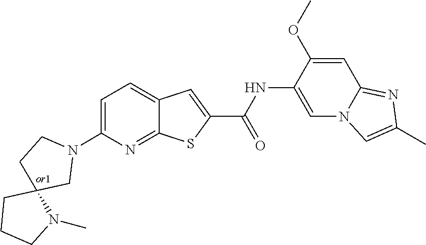 [SMILES: COc1cc2nc(C)cn2cc1NC(=O)c1cc2ccc(N3CC[C@]4(CCCN4C)C3)nc2s1] | rel-(R)-N-(7-methoxy-2- methylimidazo[1,2-a]pyridin- 6-yl)-6-(1-methyl-1,7- diazaspiro[4.4]nonan-7- yl)thieno[2,3-b]pyridine-2- carboxamide |
| 252 | 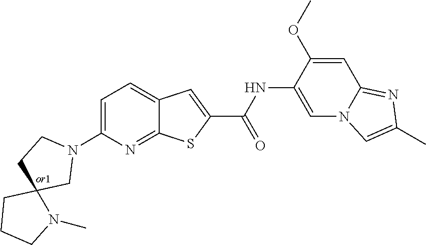 [SMILES: COc1cc2nc(C)cn2cc1NC(=O)c1cc2ccc(N3CC[C@@]4(CCCN4C)C3)nc2s1] | rel-(R)-N-(7-methoxy-2- methylimidazo[1,2-a]pyridin- 6-yl)-6-(1-methyl-1,7- diazaspiro[4.4]nonan-7- yl)thieno[2,3-b]pyridine-2- carboxamide |
| 253 | 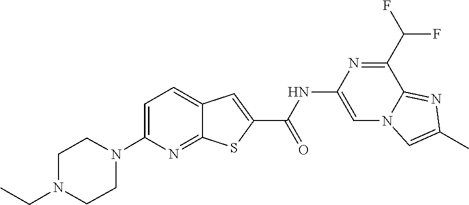 [SMILES: CCN1CCN(c2ccc3cc(C(=O)Nc4cn5cc(C)nc5c(C(F)F)n4)sc3n2)CC1] | N-(8-(difluoromethyl)-2- methylimidazo[1,2-a]pyrazin- 6-yl)-6-(4-ethylpiperazin-1- yl)thieno[2,3-b]pyridine-2- carboxamide |
| 254 | 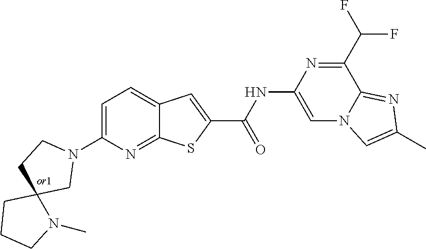 [SMILES: Cc1cn2cc(NC(=O)c3cc4ccc(N5CC[C@@]6(CCCN6C)C5)nc4s3)nc(C(F)F)c2n1] | rel-(R)-N-(8- (difluoromethyl)-2- methylimidazo[1,2-a]pyrazin- 6-yl)-6-(1-methyl-1,7- diazaspiro[4.4]nonan-7- yl)thieno[2,3-b]pyridine-2- carboxamide |
| 255 | 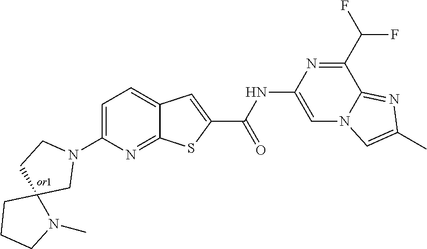 [SMILES: Cc1cn2cc(NC(=O)c3cc4ccc(N5CC[C@]6(CCCN6C)C5)nc4s3)nc(C(F)F)c2n1] | rel-(R)-N-(8- (difluoromethyl)-2- methylimidazo[1,2-a]pyrazin- 6-yl)-6-(1-methyl-1,7- diazaspiro[4.4]nonan-7- yl)thieno[2,3-b]pyridine-2- carboxamide |
| 256 | 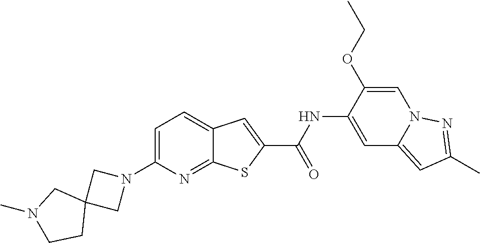 | N-(6-ethoxy-2- methylpyrazolo[1,5-a]pyridin- 5-yl)-6-(6-methyl-2,6- diazaspiro[3.4]octan-2- yl)thieno[2,3-b]pyridine-2- carboxamide |
| 257 | 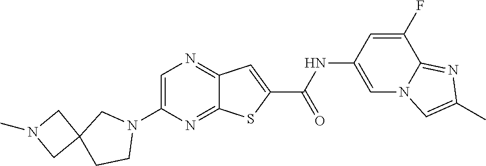 | N-(8-fluoro-2- methylimidazo[1,2-a]pyridin- 6-yl)-3-(2-methyl-2,6- diazaspiro[3.4]octan-6- yl)thieno[2,3-b]pyrazine-6- carboxamide |
| 258 | 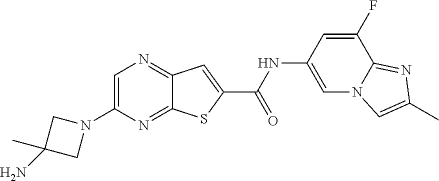 | 3-(3-amino-3-methylazetidin- 1-yl)-N-(8-fluoro-2- methylimidazo[1,2-a]pyridin- 6-yl)thieno[2,3-b]pyrazine-6- carboxamide |
| 259 | 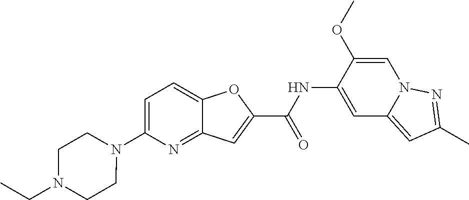 [SMILES: CCN1CCN(c2ccc3oc(C(=O)Nc4cc5cc(C)nn5cc4OC)cc3n2)CC1] | 5-(4-ethylpiperazin-1-yl)-N- (6-methoxy-2- methylpyrazolo[1,5-a]pyridin- 5-yl)furo[3,2-b]pyridine-2- carboxamide |
| 260 | 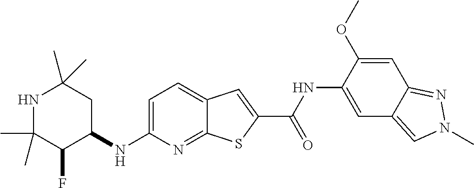 | 6-(((3R,4R)-3-fluoro-2,2,6,6- tetramethylpiperidin-4- yl)amino)-N-(6-methoxy-2- methyl-2H-indazol-5- yl)thieno[2,3-b]pyridine-2- carboxamide |
| 261 | 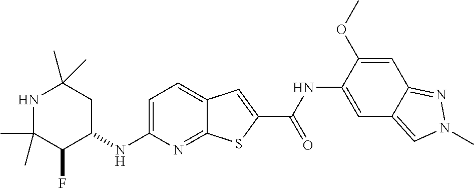 | 6-(((3R,4S)-3-fluoro-2,2,6,6- tetramethylpiperidin-4- yl)amino)-N-(6-methoxy-2- methyl-2H-indazol-5- yl)thieno[2,3-b]pyridine-2- carboxamide |
| 262 | 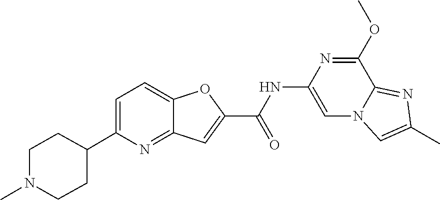 | N-(8-methoxy-2- methylimidazo[1,2-a]pyrazin- 6-yl)-5-(1-methylpiperidin-4- yl)furo[3,2-b]pyridine-2- carboxamide |
| 263 | 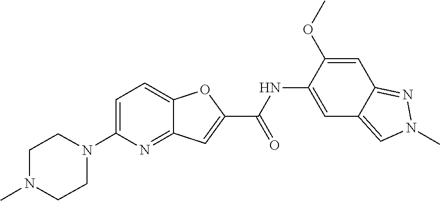 | N-(6-methoxy-2-methyl-2H- indazol-5-yl)-5-(4- methylpiperazin-1- yl)furo[3,2-b]pyridine-2- carboxamide |
| 264 | 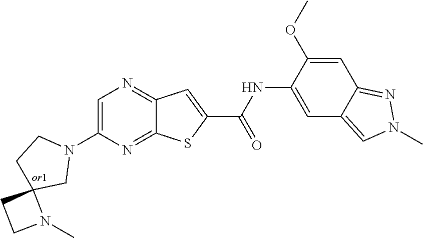 [SMILES: COc1cc2nn(C)cc2cc1NC(=O)c1cc2ncc(N3CC[C@]4(CCN4C)C3)nc2s1] | rel-(R)-N-(6-methoxy-2- methyl-2H-indazol-5-yl)-3-(1- methyl-1,6- diazaspiro[3.4]octan-6- yl)thieno[2,3-b]pyrazine-6- carboxamide |
| 265 | 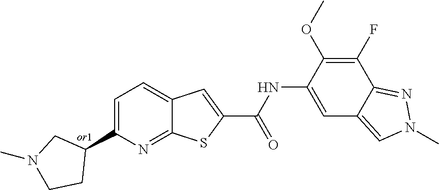 | rel-(R)-N-(7-fluoro-6- methoxy-2-methyl-2H- indazol-5-yl)-6-(1- methylpyrrolidin-3- yl)thieno[2,3-b]pyridine-2- carboxamide |
| 266 | 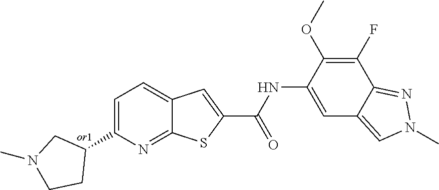 | rel-(R)-N-(7-fluoro-6- methoxy-2-methyl-2H- indazol-5-yl)-6-(1- methylpyrrolidin-3- yl)thieno[2,3-b]pyridine-2- carboxamide |
| 267 | 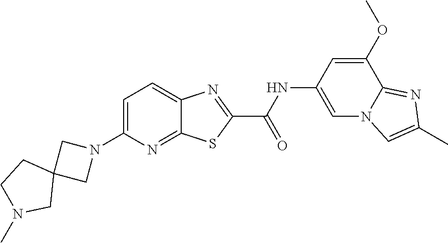 [SMILES: COc1cc(NC(=O)c2nc3ccc(N4CC5(CCN(C)C5)C4)nc3s2)cn2cc(C)nc12] | N-(8-methoxy-2- methylimidazo[1,2-a]pyridin- 6-yl)-5-(6-methyl-2,6- diazaspiro[3.4]octan-2- yl)thiazolo[5,4-b]pyridine-2- carboxamide |
| 268 | 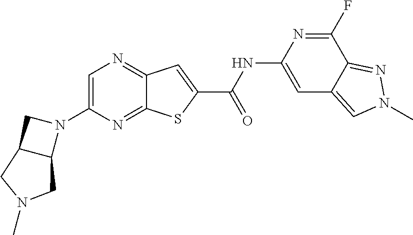 [SMILES: CN1C[C@H]2CN(c3cnc4cc(C(=O)Nc5cc6cn(C)nc6c(F)n5)sc4n3)[C@H]2C1] | N-(8-fluoro-2- methylimidazo[1,2-a]pyridin- 6-yl)-3-((1S,5R)-3-methyl- 3,6- diazabicyclo[3.2.0]heptan-6- yl)thieno[2,3-b]pyrazine-6- carboxamide |
| 269 | 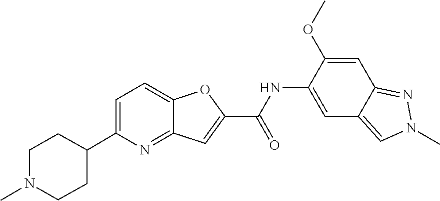 | N-(6-methoxy-2-methyl-2H- indazol-5-yl)-5-(1- methylpiperidin-4- yl)furo[3,2-b]pyridine-2- carboxamide |

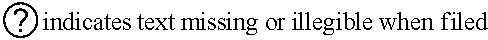

### 2. Definitions

The term “halo” or “halogen,” as used herein, refers to fluoride, chloride, bromide, or iodide.

The term “alkyl” used alone or as part of a larger moiety, such as “alkoxy” or “haloalkyl” and the like, means saturated aliphatic straight-chain or branched monovalent hydrocarbon radical of formula —CₙH₍₂ₙ₊₁₎. Unless otherwise specified, an alkyl group typically has 1-20, 1-10 or 1-6 carbon atoms. In some embodiments, an alkyl group has 1-6 carbon atoms, i.e. C₁₋₆alkyl. As used herein, a “C₁₋₆alkyl” group means a radical having from 1 to 6 carbon atoms in a linear or branched arrangement. Examples include methyl, ethyl, n-propyl, iso-propyl, n-butyl, iso-butyl, tert-butyl, n-pentyl, isopentyl, hexyl, and the like. In some embodiments, an alkyl group has 1-4 carbon atoms, i.e., C₁₋₄alkyl. In some embodiments, an alkyl group has 1-3 carbon atoms, i.e., C₁₋₃alkyl.

The term “alkoxy” or “alkoxyl,” as used herein, refers to O-alkyl groups wherein alkyl is as defined above.

The term “haloalkyl” means alkyl, as the case may be, substituted with one or more halogen atoms. In one embodiment, the alkyl can be substituted by one to three halogens. Examples of haloalkyl, include, but are not limited to, trifluoromethyl, trichloromethyl, pentafluoroethyl and the like.

The term “alkylene” as used herein, means a straight or branched chain divalent hydrocarbon group of formula —CₙH₂ₙ—. Non-limiting examples include ethylene, and propylene.

The term “cycloalkyl” refers to a monocyclic, bicyclic, tricyclic, or polycyclic saturated hydrocarbon groups having 3 to 12 ring carbons. In one embodiment, cycloalkyl may have 3 to 7 or 3 to 6 ring carbons. Any substitutable ring atom can be substituted (e.g., by one or more substituents). Examples of cycloalkyl groups include, without limitation, cyclopropyl, cyclobutyl, cyclopentyl, cyclohexyl, cycloheptyl, and cyclooctyl. Cycloalkyl may include multiple fused and/or bridged rings. Non-limiting examples of fused/bridged cycloalkyl include: bicyclo[1.1.0]butane, bicyclo[2.1.0]pentane, bicyclo[1.1.0]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like.

The term “heterocyclyl” or “heterocyclic” refers to a radical of a 3- to 12-membered non-aromatic ring system having ring carbon atoms and 1 to 4 ring heteroatoms, wherein each heteroatom is independently selected from nitrogen, quaternary nitrogen, oxidized nitrogen (e.g., NO), oxygen, and sulfur, including sulfoxide and sulfone (“3-12 membered heterocyclyl”). In some embodiments, a heterocyclyl group is a 3-7 membered non-aromatic ring system having ring carbon atoms and 1-4 ring heteroatoms, wherein each heteroatom is independently selected from nitrogen, oxygen, and sulfur (“3-7 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a bicyclic system (“bicyclic heterocyclyl”) or tricyclic system (“tricyclic heterocyclyl”); polycyclic ring systems include fused, bridged, or spiro ring systems). Exemplary monocyclic heterocyclyl groups include azetidinyl, oxetanyl, thietanyl, tetrahydrofuranyl, pyrrolidinyl, piperidinyl, tetrahydropyranyl, piperazinyl, morpholinyl, azepanyl, oxepanyl, thiepanyl, tetrahydropyridinyl, and the like. Heterocyclyl polycyclic ring systems can include heteroatoms in one or more rings in the polycyclic ring system. Substituents may be present on one or more rings in the polycyclic ring system. In some embodiments, a heterocyclyl group is a saturated heterocyclyl group. In some embodiments, a heterocyclyl group is a partially saturaturated heterocyclyl group. A partially saturaturated heterocyclyl group can contain one or more (e.g., 2 or 3) double bonds. A partially satuturated polycyclic heterocyclyl group can have one or more ring in the polycyclic ring system that are aromatic and at least one ring in the polyclyclic ring system is non-aromatic (e.g., fully saturated or practically saturated). For example, a practically saturated bicyclic heterocyclyl group can have a phenyl or a heteroaryl ring fused to a partially saturated heterocyclic ring.

Spiro heterocyclyl refers to 5 to 12 membered polycyclic heterocyclyl with rings connected through one common carbon atom (called as spiro atom), wherein said rings have one or more heteroatoms selected from the group consisting of nitrogen, quaternary nitrogen, oxidized nitrogen (e.g., NO), oxygen, and sulfur, including sulfoxide and sulfone, the remaining ring atoms being C, wherein one or more rings may contain one or more double bonds, but none of the rings has a completely conjugated π-electron system. Representative examples of spiro heterocyclyl include, but are not limited to the following groups:

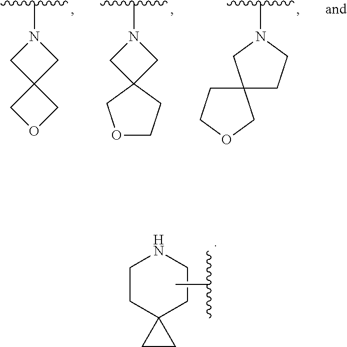

Fused heterocyclyl refers to a 5 to 12 membered polycyclic heterocyclyl group, wherein each ring in the group shares an adjacent pair of carbon atoms with another ring in the group, wherein one or more rings can contain one or more double bonds, but none of the rings has a completely conjugated π-electron system, and wherein said rings have one or more heteroatoms selected from the group consisting of nitrogen, quaternary nitrogen, oxidized nitrogen (e.g., NO), oxygen, and sulfur, including sulfoxide and sulfone, the remaining ring atoms being C. Representative examples of fused heterocyclyl include, but are not limited to the following groups:

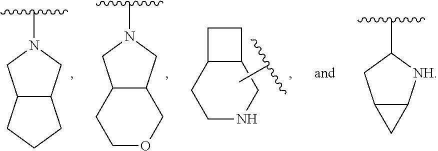

Bridged heterocyclyl refers to a 5 to 12 membered polycyclic heterocyclyl group, wherein any two rings in the group share two disconnected atoms, the rings can have one or more double bonds but have no completely conjugated π-electron system, and the rings have one or more heteroatoms selected from the group consisting of nitrogen, quaternary nitrogen, oxidized nitrogen (e.g., NO), oxygen, and sulfur, including sulfoxide and sulfone as ring atoms, the remaining ring atoms being C. Representative examples of bridged heterocyclyl include, but are not limited to the following groups:

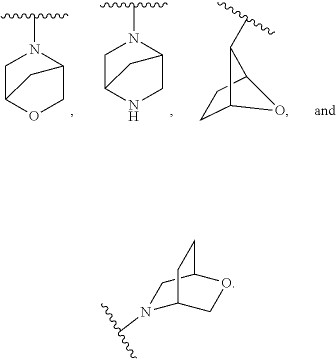

Generally, the cycloalkyl, or the heterocyclyl may be unsubstituted, or be substituted with one or more substituents as valency allows, wherein the substituents can be independently selected from a number of groups. Exemplary substituents include but are not limited to, oxo, —CN, halogen, alkyl and alkoxyl, optionally, the alkyl substitution may be further substituted.

The term “aryl” refers to a 6 to 10 membered all-carbon monocyclic ring or a polycyclic fused ring (a “fused” ring system means that each ring in the system shares an adjacent pair of carbon atoms with other ring in the system) group, and has a completely conjugated π-electron system. The term “aryl” may be used interchangeably with the terms “aryl ring” “carbocyclic aromatic ring”, “aryl group” and “carbocyclic aromatic group”. Representative examples of aryl are phenyl and naphthyl.

The term “heteroaryl,” as used herein, refers to a monocyclic or multicyclic (e.g., bicyclic) aromatic hydrocarbon in which at least one of the ring carbon atoms has been replaced with a heteroatom independently selected from oxygen, nitrogen and sulfur. Preferably, the heteroaryl is based on a C₅₋₁₀ aryl with one or more of its ring carbon atoms replaced by the heteroatom. A heteroaryl group may be attached through a ring carbon atom or, where valency permits, through a ring nitrogen atom. Generally, the heteroaryl may be unsubstituted, or be substituted with one or more substituents as valency allows. Exemplary substituents include, but are not limited to, halogen, OH, alkyl, alkoxyl, and amino (e.g., NH₂, NHalkyl, N(alkyl)₂), optionally, the alkyl may be further substituted. A heteroaryl group can either be monocyclic (“monocyclic heteroaryl”) or polycyclic (e.g., a bicyclic system (“bicyclic heteroaryl”) or tricyclic system (“tricyclic heteroaryl”); polycyclic ring systems include fused, bridged, or spiro ring systems).

Examples of monocyclic 5-6 membered heteroaryl groups include furanyl (e.g., 2-furanyl, 3-furanyl), imidazolyl (e.g., N-imidazolyl, 2-imidazolyl, 4-imidazolyl, 5-imidazolyl), isoxazolyl (e.g., 3-isoxazolyl, 4-isoxazolyl, 5-isoxazolyl), oxadiazolyl (e.g., 2-oxadiazolyl, 5-oxadiazolyl), oxazolyl (e.g., 2-oxazolyl, 4-oxazolyl, 5-oxazolyl), pyrazolyl (e.g., 3-pyrazolyl, 4-pyrazolyl), pyrrolyl (e.g., 1-pyrrolyl, 2-pyrrolyl, 3-pyrrolyl), pyridyl (e.g., 2-pyridyl, 3-pyridyl, 4-pyridyl), pyrimidinyl (e.g., 2-pyrimidinyl, 4-pyrimidinyl, 5-pyrimidinyl), pyridazinyl (e.g., 3-pyridazinyl), thiazolyl (e.g., 2-thiazolyl, 4-thiazolyl, 5-thiazolyl), triazolyl (e.g., 2-triazolyl, 5-triazolyl), tetrazolyl (e.g., tetrazolyl), thienyl (e.g., 2-thienyl, 3-thienyl), pyrimidinyl, pyridinyl and pyridazinyl. Examples of polycyclic aromatic heteroaryl groups include carbazolyl, benzimidazolyl, benzothienyl, benzofuranyl, indolyl, quinolinyl, benzotriazolyl, benzothiazolyl, benzoxazolyl, benzimidazolyl, isoquinolinyl, indolyl, isoindolyl, acridinyl, or benzisoxazolyl. A “substituted heteroaryl group” is substituted at any one or more substitutable ring atom, which is a ring carbon or ring nitrogen atom bonded to a hydrogen.

As used herein, many moieties (e.g., alkyl, alkylene, cycloalkyl, aryl, heteroaryl, or heterocyclyl) are referred to as being either “substituted” or “optionally substituted”. When a moiety is modified by one of these terms, unless otherwise noted, it denotes that any portion of the moiety that is known to one skilled in the art as being available for substitution can be substituted, which includes one or more substituents. Where if more than one substituent is present, then each substituent may be independently selected. Such means for substitution are well-known in the art and/or taught by the instant disclosure. The optional substituents can be any substituents that are suitable to attach to the moiety.

Where suitable substituents are not specifically enumerated, exemplary substituents include, but are not limited to: C₁₋₅alkyl, C₁₋₅hydroxyalkyl, C₁₋₅haloalkyl, C₁₋₅alkoxy, C₁₋₅ haloalkoxy, halogen, hydroxyl, cyano, amino, —CN, —NO₂, —OR^{c1}, —NR^{a1}R^{b1}, —S(O)ᵢR^{a1}, —NR^{a1}S(O)ᵢR^{b1}, —S(O)ᵢNR^{a1}R^{b1}, —C(═O)OR^{a1}, —OC(═O)OR^{a1}, —C(═S)OR^{a1}, —O(C═S)R^{a1}, —C(═O)NR^{a1}R^{b1}, NR^{a1}C(═O)R^{b1}, —C(═S)NR^{a1}R^{b1}, —C(═O)Ral, —C(═S)Ral, NR^{a1}C(═S)R^{b1}, —O(C═O)NR^{a1}R^{b1}, —NR^{a1}(C═S)OR^{b1}, —O(C═S)NR^{a1}R^{b1}, —NR^{a1}(C═O)NR^{a1}R^{b1}, —NR^{a1}(C═S)NR^{a1}R^{b1} phenyl, or 5-6 membered heteroaryl. Each R^{a1} and each R^{b1} are independently selected from —H and C₁₋₅alkyl, optionally substituted with hydroxyl or C₁₋₃alkoxy; R^{c1} is —H, C₁₋₅haloalkyl or C₁₋₅alkyl, wherein the C₁₋₅alkyl is optionally substituted with hydroxyl or C₁-C₃alkoxy.

The symbol “

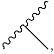

” as used herein, refers to the point where the moiety attaches.

### Pharmaceutically Acceptable Salts

The term “pharmaceutically-acceptable salt” refers to a pharmaceutical salt that is, within the scope of sound medical judgment, suitable for use in contact with the tissues of humans and lower animals without undue toxicity, irritation, and allergic response, and is commensurate with a reasonable benefit/risk ratio. Pharmaceutically-acceptable salts are well known in the art. For example, S. M. Berge et al. describes pharmacologically acceptable salts in J. Pharm. Sci., 1977, 66, 1-19.

Pharmaceutically acceptable salts of the compounds of any one of the formulae described above include acid addition and base salts.

Included in the present teachings are pharmaceutically acceptable salts of the compounds disclosed herein. Compounds having basic groups can form pharmaceutically acceptable salts with pharmaceutically acceptable acid(s). Suitable pharmaceutically acceptable acid addition salts of the compounds described herein include salts of inorganic acids (such as hydrochloric, hydrobromic, phosphoric, metaphosphoric, nitric, and sulfuric acids) and of organic acids (such as acetic, benzenesulfonic, benzoic, ethanesulfonic, methanesulfonic, and succinic acids). Compounds of the present teachings with acidic groups such as carboxylic acids can form pharmaceutically acceptable salts with pharmaceutically acceptable base(s). Suitable pharmaceutically acceptable basic salts include ammonium salts, alkali metal salts (such as sodium and potassium salts) and alkaline earth metal salts (such as magnesium and calcium salts).

Pharmaceutically acceptable salts of compounds of any one of the formulae described above may be prepared by one or more of three methods:
(i) by reacting the compound of any one of the formulae described above with the desired acid or base;
(ii) by removing an acid- or base-labile protecting group from a suitable precursor of the compound of any one of the formulae described above or by ring-opening a suitable cyclic precursor, for example, a lactone or lactam, using the desired acid or base; or
(iii) by converting one salt of the compound of any one of the formulae described above to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of any one of the formulae described above, and pharmaceutically acceptable salts thereof, may exist in unsolvated and solvated forms.

### Stereoisomers and Other Variations

The compounds of any one of the formulae described above may exhibit one or more kinds of isomerism (e.g. optical, geometric or tautomeric isomerism). Such variation is implicit to the compounds of any one of the formulae described above defined as they are by reference to their structural features and therefore within the scope of the present disclosure.

Compounds having one or more chiral centers can exist in various stereoisomeric forms, i.e., each chiral center can have an R or S configuration, or can be a mixture of both. Stereoisomers are compounds that differ only in their spatial arrangement. Stereoisomers include all diastereomeric and enantiomeric forms of a compound. Enantiomers are stereoisomers that are mirror images of each other. Diastereomers are stereoisomers having two or more chiral centers that are not identical and are not mirror images of each other.

When a compound is designated by its chemical name (e.g., where the configuration is indicated in the chemical name by “R” or “S”) or its structure (e.g., the configuration is indicated by “wedge” bonds) that indicates a single enantiomer, unless indicated otherwise, the compound is at least 60%, 70%, 80%, 90%, 99% or 99.9% optically pure (also referred to as “enantiomerically pure”). Optical purity is the weight in the mixture of the named or depicted enantiomer divided by the total weight in the mixture of both enantiomers.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers is included. It is to be further understood that the stereoisomeric purity of the named or depicted stereoisomers at least 60%, 70%, 80%, 90%, 99% or 99.9% by weight. The stereoisomeric purity in this case is determined by dividing the total weight in the mixture of the stereoisomers encompassed by the name or structure by the total weight in the mixture of all of the stereoisomers.

When two stereoisomers are depicted by their chemical names or structures, and the chemical names or structures are connected by an “and”, a mixture of the two stereoisomers is intended.

When two stereoisomers are depicted by their chemical names or structures, and the names or structures are connected by an “or”, one or the other of the two stereoisomers is intended, but not both.

When a disclosed compound having a chiral center is depicted by a structure without showing a configuration at that chiral center, the structure is meant to encompass the compound with the S configuration at that chiral center, the compound with the R configuration at that chiral center, or the compound with a mixture of the R and S configuration at that chiral center. When a disclosed compound having a chiral center is depicted by its chemical name without indicating a configuration at that chiral center with “S” or “R”, the name is meant to encompass the compound with the S configuration at that chiral center, the compound with the R configuration at that chiral center or the compound with a mixture of the R and S configuration at that chiral center.

Racemic mixture means 50% of one enantiomer and 50% of the corresponding enantiomer. When a compound with one chiral center is named or depicted without indicating the stereochemistry of the chiral center, it is understood that the name or structure encompasses both possible enantiomeric forms (e.g., both enantiomerically-pure, enantiomerically-enriched or racemic) of the compound. When a compound with two or more chiral centers is named or depicted without indicating the stereochemistry of the chiral centers, it is understood that the name or structure encompasses all possible diasteriomeric forms (e.g., diastereomerically pure, diastereomerically enriched and equimolar mixtures of one or more diastereomers (e.g., racemic mixtures) of the compound.

The term “geometric isomer” means isomers that differ in the orientation of substituent atoms in relationship to a carbon-carbon double bond, to a carbocyclic ring, or to a bridged bicyclic system. Substituent atoms (other than hydrogen) on each side of a carbon-carbon double bond may be in an E or Z configuration according to the Cahn-Ingold-Prelog priority rules. In the “E” configuration, the substituents having the highest priorities are on opposite sides in relationship to the carbon-carbon double bond. In the “Z” configuration, the substituents having the highest priorities are oriented on the same side in relationship to the carbon-carbon double bond.

Substituents around a carbon-carbon double bond can also be referred to as “cis” or “trans,” where “cis” represents substituents on the same side of the double bond and “trans” represents substituents on opposite sides of the double bond. The arrangement of substituents around a carbocyclic ring can also be designated as “cis” or “trans.” The term “cis” represents substituents on the same side of the plane of the ring, and the term “trans” represents substituents on opposite sides of the plane of the ring. Mixtures of compounds wherein the substituents are disposed on both the same and opposite sides of plane of the ring are designated “cis/trans.”

Where structural isomers are interconvertible via a low energy barrier, tautomeric isomerism (“tautomerism”) can occur. This can take the form of proton tautomerism in compounds of any one of the formulae described above containing, for example, an imino, keto, or oxime group, or so-called valence tautomerism in compounds which contain an aromatic moiety. It follows that a single compound may exhibit more than one type of isomerism.

In certain instances tautomeric forms of the disclosed compounds exist, such as the tautomeric structures shown below:

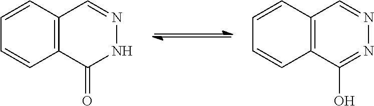 [SMILES: O=c1[nH]ncc2ccccc12 ; Oc1nncc2ccccc12]

When a geometric isomer is depicted by name or structure, it is to be understood that the named or depicted isomer exists to a greater degree than another isomer, that is that the geometric isomeric purity of the named or depicted geometric isomer is greater than 50%, such as at least 60%, 70%, 80%, 90%, 99%, or 99.9% pure by weight. Geometric isomeric purity is determined by dividing the weight of the named or depicted geometric isomer in the mixture by the total weight of all of the geometric isomers in the mixture.

Cis trans isomers may be separated by conventional techniques well known to those skilled in the art, for example, chromatography and fractional crystallisation.

Conventional techniques for the preparation/isolation of individual enantiomers/diastereomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where the compound of any one of the formulae described above contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person. Chiral compounds of any one of the formulae described above (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture. Chiral chromatography using sub- and supercritical fluids may be employed. Methods for chiral chromatography useful in some embodiments of the present disclosure are known in the art (see, for example, Smith, Roger M., Loughborough University, Loughborough, UK; Chromatographic Science Series (1998), 75 (Supercritical Fluid Chromatography with Packed Columns), pp. 223-249 and references cited therein). Columns can be obtained from Chiral Technologies, Inc, West Chester, Pa., USA, a subsidiary of Daicel® Chemical Industries, Ltd., Tokyo, Japan.

It must be emphasized that the compounds of any one of the formulae described above have been drawn herein in a single tautomeric form, all possible tautomeric forms are included within the scope of the present disclosure.

### 3. Administration and Dosing

Typically, a compound of the present disclosure is administered in an amount effective to treat a condition as described herein. The compounds of the present disclosure can be administered as compound per se, or alternatively, as a pharmaceutically acceptable salt. For administration and dosing purposes, the compound per se or pharmaceutically acceptable salt thereof will simply be referred to as the compounds of the present disclosure.

The compounds of the present disclosure are administered by any suitable route in the form of a pharmaceutical composition adapted to such a route, and in a dose effective for the treatment intended. The compounds of the present disclosure may be administered orally, rectally, vaginally, parenterally, or topically.

The compounds of the present disclosure may be administered orally. Oral administration may involve swallowing, so that the compound enters the gastrointestinal tract, or buccal or sublingual administration may be employed by which the compound enters the bloodstream directly from the mouth.

In another embodiment, the compounds of the present disclosure may also be administered directly into the bloodstream, into muscle, or into an internal organ. Suitable means for parenteral administration include intravenous, intra-arterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

In another embodiment, the compounds of the present disclosure may also be administered topically to the skin or mucosa, that is, dermally or transdermally. In another embodiment, the compounds of the present disclosure can also be administered intranasally or by inhalation. In another embodiment, the compounds of the present disclosure may be administered rectally or vaginally. In another embodiment, the compounds of the present disclosure may also be administered directly to the eye or ear.

The dosage regimen for the compounds of the present disclosure and/or compositions containing said compounds is based on a variety of factors, including the type, age, weight, sex and medical condition of the patient; the severity of the condition; the route of administration; and the activity of the particular compound employed. Thus the dosage regimen may vary widely. In one embodiment, the total daily dose of a compound of the present disclosure is typically from about 0.001 to about 100 mg/kg (i.e., mg compound of the present disclosure per kg body weight) for the treatment of the indicated conditions discussed herein.

For oral administration, the compositions may be provided in the form of tablets containing 0.1-500 milligrams of the active ingredient for the symptomatic adjustment of the dosage to the patient. A medicament typically contains from about 0.01 mg to about 500 mg of the active ingredient. Intravenously, doses may range from about 0.01 to about 10 mg/kg/minute during a constant rate infusion.

Suitable subjects according to the present disclosure include mammalian subjects, including non-human mammal such as primates, rodents (mice, rats, hamsters, rabbits etc). In one embodiment, humans are suitable subjects. Human subjects may be of either gender and at any stage of development.

### 4. Pharmaceutical Compositions

In another embodiment, the present disclosure comprises pharmaceutical compositions. Such pharmaceutical compositions comprise a compound of the present disclosure presented, a pharmaceutically acceptable salt, or a stereoisomer thereof with a pharmaceutically acceptable carrier or excipient. Other pharmacologically active substances can also be present.

As used herein, “pharmaceutically acceptable carrier or excipient” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition. Pharmaceutically acceptable substances such as wetting agents or minor amounts of auxiliary substances such as wetting or emulsifying agents, preservatives or buffers, which enhance the shelf life or effectiveness of the antibody or antibody portion.

The compositions of present disclosure may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form depends on the intended mode of administration and therapeutic application.

Typical compositions are in the form of injectable or infusible solutions, such as compositions similar to those used for passive immunization of humans with antibodies in general. One mode of administration is parenteral (e.g. intravenous, subcutaneous, intraperitoneal, intramuscular). In another embodiment, the antibody is administered by intravenous infusion or injection. In yet another embodiment, the antibody is administered by intramuscular or subcutaneous injection.

Oral administration of a solid dose form may be, for example, presented in discrete units, such as hard or soft capsules, pills, cachets, lozenges, or tablets, each containing a predetermined amount of at least one compound of the present disclosure. In another embodiment, the oral administration may be in a powder or granule form. In another embodiment, the oral dose form is sub-lingual, such as, for example, a lozenge. In such solid dosage forms, the compounds of any one of the formulae described above are ordinarily combined with one or more adjuvants. Such capsules or tablets may contain a controlled release formulation. In the case of capsules, tablets, and pills, the dosage forms also may comprise buffering agents or may be prepared with enteric coatings.

In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

In another embodiment, the present disclosure comprises a parenteral dose form.

“Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using suitable dispersing, wetting agents, and/or suspending agents.

In another embodiment, the present disclosure comprises a topical dose form.

“Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of present disclosure are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, Finnin and Morgan, J. Pharm. Sci., 88:955-958, 1999.

Formulations suitable for topical administration to the eye include, for example, eye drops wherein the compound of present disclosure is dissolved or suspended in a suitable carrier. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration or administration by inhalation, the compounds of the present disclosure are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the present disclosure comprises a rectal dose form. Such rectal dose form may be in the form of, for example, a suppository. Cocoa butter is a traditional suppository base, but various alternatives may be used as appropriate.

Other carrier materials and modes of administration known in the pharmaceutical art may also be used. Pharmaceutical compositions of the present disclosure may be prepared by any of the well-known techniques of pharmacy, such as effective formulation and administration procedures.

The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman et al., Eds., Pharmaceutical Dosage Forms, Marcel Decker, New York, N.Y., 1980; and Kibbe et al., Eds., Handbook of Pharmaceutical Excipients (3^{rd} Ed.), American Pharmaceutical Association, Washington, 1999.

### 5. Method of Treatment

The terms “subject,” “individual,” or “patient,” used interchangeably, refer to any animal, including mammals, preferably mice, rats, other rodents, rabbits, dogs, cats, swine, cattle, sheep, horses, or primates, and most preferably humans.

The terms “treatment,” “treat,” and “treating” refer to reversing, alleviating, or inhibiting the progress of a disease described herein. In some embodiments, treatment may be administered after one or more signs or symptoms of the disease have developed or have been observed (i.e., therapeutic treatment). In other embodiments, treatment may be administered in the absence of signs or symptoms of the disease. For example, treatment may be administered to a susceptible subject prior to the onset of symptoms (i.e., prophylactic treatment) (e.g., in light of a history of symptoms and/or in light of exposure to a pathogen). Treatment may also be continued after symptoms have resolved, for example, to delay or prevent recurrence.

The term “prevention” (or “prevent” or “preventing”), as used herein, refers to precluding, averting, obviating, forestalling, reducing the incidence of, stopping, or hindering the symptoms of a disease, disorder and/or condition. Prevention includes administration to a subject who does not exhibit symptoms of a disease, disorder, and/or condition at the time of administration.

The terms “condition,” “disease,” and “disorder” are used interchangeably.

The term “administer,” “administering,” or “administration” refers to methods introducing a compound disclosed herein, or a composition thereof, in or on a patient. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa.

Generally, an effective amount of a compound taught herein varies depending upon various factors, such as the given drug or compound, the pharmaceutical formulation, the route of administration, the type of disease or disorder, the identity of the subject or host being treated, and the like, but can nevertheless be routinely determined by one skilled in the art. An effective amount of a compound of the present teachings may be readily determined by one of ordinary skill by routine methods known in the art.

The term “therapeutically effective amount” means an amount when administered to the subject which results in beneficial or desired results, including clinical results, e.g., inhibits, suppresses or reduces the symptoms of the condition being treated in the subject as compared to a control. The exact amount required will vary from subject to subject, depending on the species, age, and general condition of the subject, the severity of the disease, the particular anticancer agent, its mode of administration, combination treatment with other therapies, and the like.

The present disclosure is directed to compounds of formula (I) (including all its embodiments), which are useful in the treatment and/or prevention of a disease and/or condition associated with or modulated by HTT, especially wherein lowering mHTT in a subject is of therapeutic benefit, including but not limited to the treatment and/or prevention of HD.

In one embodiment, the present disclosure relates to a compound of formula (I) or a pharmaceutically acceptable salt thereof for use as a medicament.

In one embodiment, the present disclosure relates to a compound of (I) or a pharmaceutically acceptable salt thereof for use in a method of treatment of the human or animal body.

The present disclosure further provides a method of treating HD in a subject in need thereof, comprising administering to the subject an effective amount of (1) a compound of Formula (I) or a pharmaceutically acceptable salt thereof; or (2) a pharmaceutically acceptable composition comprising a compound of Formula (I) or a pharmaceutically acceptable salt thereof, and a pharmaceutically acceptable carrier.

In one embodiment, the present disclosure provides a use for a compound of Formula (I) or a pharmaceutically acceptable salt thereof for treating HD in a subject in need thereof comprising, administering to the subject an effective amount of the compound of Formula (I) or a pharmaceutically acceptable salt thereof.

In one embodiment, the present disclosure provides a use for a compound of Formula (I) or a pharmaceutically acceptable salt thereof in the manufacture of a medicament for treating HD in a subject in need thereof comprising, administering to the subject an effective amount of the medicament.

### 6. Treatment Kits

One aspect of the present invention relates to a kit for conveniently and effectively carrying out the methods or uses in accordance with the present invention. In general, the pharmaceutical pack or kit comprises one or more containers filled with one or more of the ingredients of the pharmaceutical compositions of the invention. Such kits are especially suited for the delivery of solid oral forms such as tablets or capsules. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Optionally associated with such container(s) can be a notice in the form prescribed by a governmental agency regulating the manufacture, use or sale of pharmaceutical products, which notice reflects approval by the agency of manufacture, use or sale for human administration.

The following representative examples contain important additional information, exemplification and guidance which can be adapted to the practice of this invention in its various embodiments and the equivalents thereof. These examples are intended to help illustrate the invention, and are not intended to, nor should they be construed to, limit its scope. Indeed, various modifications of the invention, and many further embodiments thereof, in addition to those shown and described herein, will become apparent to those skilled in the art upon review of this document, including the examples which follow and the references to the scientific and patent literature cited herein.

The contents of the cited references are incorporated herein by reference to help illustrate the state of the art.

In addition, for purposes of this invention, the chemical elements are identified in accordance with the Periodic Table of the Elements, CAS version, Handbook of Chemistry and Physics, 75^{th} Ed., inside cover. Additionally, general principles of organic chemistry, as well as specific functional moieties and reactivity, are described in “Organic Chemistry,” Thomas Sorrell, University Science Books, Sausalito: 1999, and “Organic Chemistry,” Morrison & Boyd (3d Ed), the entire contents of both of which are incorporated herein by reference.

### 7. Preparation

The compounds of any one of the formulae described above, may be prepared by the general and specific methods described below, using the common general knowledge of one skilled in the art of synthetic organic chemistry. Such common general knowledge can be found in standard reference books such as Comprehensive Organic Chemistry, Ed. Barton and Ollis, Elsevier; Comprehensive Organic Transformations: A Guide to Functional Group Preparations, Larock, John Wiley and Sons; and Compendium of Organic Synthetic Methods, Vol. I-XII (published by Wiley-Interscience). The starting materials used herein are commercially available or may be prepared by routine methods known in the art.

In the preparation of the compounds of any one of the formulae described above, it is noted that some of the preparation methods described herein may require protection of remote functionality (e.g., primary amine, secondary amine, carboxyl in any one of the formulae described above precursors). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see Greene, Protective Groups in Organic Synthesis, John Wiley & Sons, New York, 1991.

For example, certain compounds contain primary amines or carboxylic acid functionalities which may interfere with reactions at other sites of the molecule if left unprotected. Accordingly, such functionalities may be protected by an appropriate protecting group which may be removed in a subsequent step. Suitable protecting groups for amine and carboxylic acid protection include those protecting groups commonly used in peptide synthesis (such as N-t-butoxycarbonyl (Boc), benzyloxycarbonyl (Cbz), and 9-fluorenylmethylenoxycarbonyl (Fmoc) for amines, and lower alkyl or benzyl esters for carboxylic acids) which are generally not chemically reactive under the reaction conditions described and can typically be removed without chemically altering other functionality in the any one of the formulae described above compounds.

The Schemes described below are intended to provide a general description of the methodology employed in the preparation of the compounds of the present disclosure. Some of the compounds of the present disclosure may contain single or multiple chiral centers with the stereochemical designation (R) or (S). It will be apparent to one skilled in the art that all of the synthetic transformations can be conducted in a similar manner whether the materials are enantio-enriched or racemic. Moreover, the resolution to the desired optically active material may take place at any desired point in the sequence using well known methods such as described herein and in the chemistry literature.

### EXAMPLES

| Abbreviation | Meaning |
|---|---|
| ACN or CH₃CN or MeCN | acetonitrile |
| ° C. | Degrees centigrade |
| Cl | Chloride |
| Cs₂CO₃ | Cesium carbonate |
| h/hr/hrs/min/s | hour/minute/second |
| d | doublet |
| dd | Doublet of doublets |
| DCM or CH₂Cl₂ | dichloromethane |
| DIPEA | N,N-diisopropylamine |
| DMSO | dimethylsulfoxide |
| EtOAc | Ethyl acetate |
| EtOH | ethanol |
| Eq or equiv. | equivalent |
| FA | Formic acid |
| H₂ | Hydrogen |
| HCl | Hydrochloric acid |
| HPLC | High performance liquid |
| | chromatography |
| J | Coupling constant |
| LC/MS | Liquid chromatographic |
| or LCMS | mass spectroscopy |
| m | multiplet |
| MeOH | methanol |
| m/z | Mass to charge ratio |
| NEt₃ or Et₃N | Triethylamine |
| or TEA | |
| NH₄Cl | Ammonium chloride |
| NMR | Nuclear magnetic resonance |
| PE | Petroleum ether |
| Pd | palladium |
| Pd/C | Palladium on carbon |
| Pd(dppf)Cl₂—CH₂Cl₂ | [1,1′-bis (diphenylphosphino)ferrocene] |
| | dichloropalladium(II), |
| | complex with dichloromethane |
| ppm | Parts per million |
| POM | polyformaldehyde |
| rt | Room temperature |
| RT | Retention time |
| s | singlet |
| t | triplet |
| TFA | Trifluoroacetic acid |
| THF | tetrahydrofuran |
| TLC | Thin layer chromatography |
| t-Bu | Tert-butyl |

### Section 1. General Methods and Analytical Methods

a. General Methods

The compounds of the Examples were analyzed or purified according to one of the Purification Methods referred to below unless otherwise described. Where preparative TLC/HPLC or silica gel chromatography have been used, one skilled in the art may choose any combination of solvents to purify the desired compound. Silica gel column chromatography was performed using 20-40 mM (particle size), 250-400 mesh, or 400-632 mesh silica gel using either a Teledyne ISCO Combiflash RE or a Grace Reveleris X2 with ELSD purification systems or using pressurized nitrogen (˜10-15 psi) to drive solvent through the column (“flash chromatography”). Wherein an SCX column has been used, the eluant conditions are MeOH followed by methanolic ammonia. Where indicated, solutions and reaction mixtures were concentrated by rotary evaporation under vacuum.

b. Analytical Methods

### Analytical LCMS Instrumentation Specifications:

Waters Acquity iClass UPLC with QDa mass spectrometer and PDA (photodiode array detector)

### RxnQC/FrxQC/PurityQC Analysis LC/MS Method Conditions:

### Ammonium Hydroxide (Basic pH) Conditions

### Method 1

MS mode: MS:ESI+ scan range 165-650 daltons
PDA: 200-400 nm scan range
Column: Waters ACQUITY UPLC BEH C18 2.1×50 mm, 1.7 um; Part No. 186002350
Modifier: Ammonium hydroxide 0.2% (v/v) conc.
Method: 95% H₂0/5% MeCN (initial conditions) hold 0.1 min, linear gradient to 5% H₂0/95% MeCN at 3.25 min, hold 5% H₂0/95% MeCN to 3.5 min. Flow rate, 0.8 mL/min.

### Method 2

MS mode: MS:ESI+ scan range 165-650 daltons
PDA: 200-400 nm scan range
Column: Waters ACQUITY UPLC BEH C18 2.1×30 mm, 1.7 um; Part No. 186002349
Modifier: Ammonium hydroxide 0.2% (v/v) conc.
Method: 95% H20/5% MeCN (initial conditions), linear gradient to 5% H20/95% MeCN at 1.0 min, hold 5% H20/95% MeCN to 1.3 min. Flow rate, 0.7 mL/min.

### Trifluoroacetic Acid (Acidic pH) Conditions

### Method 3

MS mode: MS:ESI+ scan range 165-650 daltons
PDA: 200-400 nm scan range
Column: Waters ACQUITY UPLC BEH C18 2.1×50 mm, 1.7 um; Part No. 186002350
Modifier: Trifluoroacetic acid 0.1% (v/v) conc.
Method: 95% H20/5% MeCN (initial conditions) hold 0.1 min, linear gradient to 5% H20/95% MeCN at 3.25 min, hold 5% H20/95% MeCN to 3.5 min. Flow rate, 0.8 mL/min.

### Method 4

MS mode: MS:ESI+ scan range 165-650 daltons
PDA: 200-400 nm scan range
Column: Waters ACQUITY UPLC BEH C18 2.1×50 mm, 1.7 um; Part No. 186002349
Modifier: Trifluoroacetic acid 0.1% (v/v) conc.
Method: 95% H20/5% MeCN (initial conditions), linear gradient to 5% H20/95% MeCN at 1.0 min, hold 5% H20/95% MeCN to 1.3 min. Flow rate, 0.7 mL/min.

### Analytical LCMS Instrumentation Specifications:

Agilent 1200 Series LC/MSD system with DAD\LSD Alltech 3300 and Agilent LC\MSD G6130A, G6120B mass-spectrometer; Agilent Technologies 1260 Infinity LC/MSD system with DAD\LSD Alltech 3300 and Agilent LC\MSD G6120B mass-spectrometer; Agilent Technologies 1260 Infinity II LC/MSD system with DAD\LSD G7102A 1290 Infinity II and Agilent LC\MSD G6120B mass-spectrometer; Agilent 1260 Series LC/MSD system with DAD\LSD and Agilent LC\MSD (G6120B) mass-spectrometer; UHPLC Agilent 1290 Series LC/MSD system with DAD\LSD and Agilent LC\MSD (G6125B) mass-spectrometer, Shimadzu LCMS-2020.

### RxnQC/FrxQC/PurityQC Analysis LC/MS Method Conditions:

### Formic Acid (Acidic pH) Conditions

### Method 5

Inject volume: 0.5 μl, Column Temperature: 60° C., UV scan: 207-223 nM, 246-262 nM, 272-288 nM, Agilent Poroshell 120 SB-C18 4.6×30 mm 2.7 μm with UHPLC Guard Infinity Lab Poroshell 120 SB-C18 4.6×5 mm 2.7 μm, Mobile phase A: 0.1% FA in Water, Mobile phase B: 0.1% FA in Acetonitrile.

### Details of Elution

| Time (min) | Flow (mL/min) | % A | % B |
|---|---|---|---|
| 0.00 | 3.00 | 99 | 1 |
| 0.01 | 3.00 | 99 | 1 |
| 1.5 | 3.00 | 0 | 100 |
| 1.73 | 3.00 | 0 | 100 |
| 1.74 | 3.00 | 99 | 1 |

### Method 6

Inject volume: 0.5 μl; Column Temperature: 60° C.; UV scan: 207-223 nM, 246-262 nM, 272-288 nM
Agilent Poroshell 120 SB-C18 4.6×30 mm 2.7 μm with UHPLC Guard Infinity Lab Poroshell 120 SB-C18 4.6×5 mm 2.7 μm, Mobile phase A: 0.1% FA in Water, Mobile phase B: 0.1% FA in Acetonitrile

### Details of Elution

| Time (min) | Flow (mL/min) | % A | % B |
|---|---|---|---|
| 0.00 | 1.5 | 99 | 1 |
| 0.01 | 1.5 | 99 | 1 |
| 5.00 | 1.5 | 0 | 100 |
| 5.99 | 1.5 | 0 | 100 |
| 6.00 | 1.5 | 99 | 1 |

### Method 7

MS mode: MS ESI+ scan range 100-1000 daltons
PDA: 190-370 nm scan range
Column: Xtimate C18 2.1*30 mm, 3 μm
Modifier: A Phase: water (4 L)+TFA (1.5 mL), B Phase: acetonitrile (4 L)+TFA (0.75 mL)
Method: using the elution gradient 10%-80% (solvent B) over 1.35 or 3.35 minutes and holding at 80% for 0.9 minutes at a flow rate of 0.8 ml/min.

### Method 8

5-95AB_4 min.M

| Instrument | | Agilent 1260\G6125B | | |
|---|---|---|---|---|
| Software | | Agilent ChemStation Rev. C. 01.10[201] | | |
| HPLC | Column | Shim-pack Velox SP-C18 2.7 μm 3.0*30 mm | | |
| | Mobile Phase | A: 0.0375% TFA in water (v/v) | | |
| | | B: 0.01875% TFA in ACN (v/v) | | |
| | | Time(min) | B(%) | Flow(mL/min) |
| | Gradient | 0.0 | 5 | 1.0 |
| | | 3.00 | 95 | 1.0 |
| | | 3.60 | 95 | 1.5 |
| | | 3.61 | 5 | 1.5 |
| | | 4.00 | 5 | 1.5 |

| | | |
|---|---|---|
| | Column Temp | 50° C. |
| | Detector | DAD&ELSD |
| MS | Ionization source | ESI |
| | Drying Gas | N2 |
| | Drying Gas Flow | 10(L/min) |
| | Nebulizer Pressure | 40(psig) |
| | Drying Gas Temp | 350(° C.) |
| | Capillary Voltage | 4000(V) |
| | MS Polarity | Positive |
| | MS Mode | Scan |

### Description

Mobile phase: Ramp from 5% ACN (0.0185% TFA) in water (0.037 FA) to 95 ACN in 3.0 min, Flow rate is set at 1.0 mL/min; then hold at 95% ACN for 0.60 minutes Flow rate is 5 set from 1.0 mL/min to 1.5 mL/min; return back to 5%˜ACN in water and hold for 0.40 min.

Flow rate is set at 1.5 mL/min.

Column temperature at 50° C.

The column is of Shim-pack Velox SP-C18 2.7 μm 3.0*30 mm.

### Method 9

5-95AB_0.8 min

| Instrument | | SHIMADZU LCMS-2020; | | |
|---|---|---|---|---|
| Software | | LabSolution Version 5.97SP1 | | |
| HPLC | Column | Kinetex ® EVO C18 2.1 × 30 mm 5 um | | |
| | Mobile Phase | A: 0.0375% TFA in water (v/v) | | |
| | | B: 0.01875% TFA in Acetonitrile (v/v) | | |
| | | Time(min) | B(%) | Flow(mL/min) |
| | Gradient | 0.0 | 5 | 2.0 |
| | | 0.60 | 95 | 2.0 |
| | | 0.78 | 95 | 2.0 |
| | | 0.79 | 5 | 2.0 |
| | | 0.80 | 5 | 2.0 |

| | | |
|---|---|---|
| | Column Temp | 50° C. |
| | Detector | PDA |
| MS | Ionization source | ESI |
| | Drying Gas | N2 |
| | Drying Gas Flow | 15(L/min) |
| | DL Voltage | 120(v) |
| | Qarray DC Voltage | 20(V) |
| | MS Polarity | Positive |
| | MS Mode | Scan |

### Description

Mobile phase: Ramp from 500 ACN (0.01875% TFA) in water (0.0375% TFA) to 95% ACN in water in 0.60 min, Flow rate is set at 2.0 mL/min; then hold at 95% ACN for 0.18 minutes
Flow rate is set at 2.0 mL/min; return back to 500 ACN in water and hold for 0.02 min. Flow rate is set at 2.0 mL/min.
Column temperature at 50° C.
The column is of Kinetex® EVO C18 2.1×30 mm 5 um.

### Method 10

### Description

Mobile phase: Ramp from 500 ACN (0.01875% TFA) in water (0.0375% TFA) to 95% ACN in 3.20 min, Flow rate is set at 1.5 mL/min; then hold at 95% ACN for 0.30 minutes Flow rate is set at 1.5 mL/min; return back to 5% ACN in water and hold for 0.30 min. Flow rate is set at 2.0 mL/min. Column temperature at 50° C. The column is of Kinetex® EVO C18 4.6×50 mm 5 um.

### Preparative HPLC-MS Conditions:

### HPLC-MS Instrumentation Specifications

Waters Autopurification with QDa mass spectrometer and PDA (photodiode array detector).

### Ammonium Hydroxide (Basic pH) Conditions

Flow rate: 30 mL/min
MS mode: MS:ESI+ scan range 165-650 daltons
PDA: 200-400 nm scan range
Column: Waters XSELECT CSH C18 PREP 19×100 mm, 5 um; Part No. 186005421
Modifier: 0.2% Ammonium hydroxide (v/v) conc.

Method: A % H20/B % MeCN (initial conditions) hold 0.5 min, linear gradient to A % H20/B % MeCN at 8 min, ramp to 5% H20/95% MeCN at 8.5 min, HOLD 5% H20/95% MeCN to 10 min.
Flow rate: 50 mL/min
MS mode: MS:ESI+ scan range 165-650 daltons
PDA: 200-400 nm scan range
Column: Waters XSELECT CSH C18 PREP 30×100 mm, 5 um; Part No. 186005425
Modifier: 0.2% Ammonium hydroxide (v/v) conc.
Method: A % H20/B % MeCN (initial conditions) hold 0.5 min, linear gradient to A % H20/B % MeCN at 8 min, ramp to 5% H20/95% MeCN at 8.5 min, HOLD 5% H20/95% MeCN to 10 min.
Flow rate, 60 mL/min
MS mode: MS:ESI+ scan range 165-650 daltons
PDA: 200-400 nm scan range
Column: Waters XSELECT CSH C18 PREP 30×mm, 5 um; Part No. 186005423
Modifier: 0.2% Ammonium hydroxide (v/v) conc.
Method: A % H20/B % MeCN (initial conditions) hold 0.5 min, linear gradient to A % H20/B % MeCN at 8 min, ramp to 5% H20/95% MeCN at 8.5 min, HOLD 5% H20/95% MeCN to 10 min.
Column: Boston Prime C18 150×30 mm×5 um; Condition: water (NH3H2O+NH4HCO3)- ACN; Gradient (% organic): 0-100% optimized for each example; Flow Rate (mL/min) 25.
Column: YMC Actus Trial C18 20*100 5 mkm column; gradient mixture H2O—MeOH— Ammonia 0.1% as a mobile phase optimized for each example

### Trifluoroacetic Acid (Acidic pH) Conditions

Flow rate, 30 mL/min
MS mode: MS:ESI+ scan range 165-650 daltons
PDA: 200-400 nm scan range
Column: Waters Sunfire OBD C18 PREP 19×100 mm, 5 um; Part No. 186002567
Modifier: 0.1% Trifluoroacetic acid (v/v) conc.
Method: A % H20/B % MeCN (initial conditions) hold 0.5 min, linear gradient to A % H20/B % MeCN at 8 min, ramp to 5% H20/95% MeCN at 8.5 min, HOLD 5% H20/95% MeCN to 10 min.
Flow rate, 50 mL/min
MS mode: MS:ESI+ scan range 165-650 daltons
PDA: 200-400 nm scan range
Column: Waters Sunfire OBD C18 PREP 30×100 mm, 5 um; Part No. 186002572
Modifier: 0.1% Trifluoroacetic acid (v/v) conc.
Method: A % H20/B % MeCN (initial conditions) hold 0.5 min, linear gradient to A % H20/B % MeCN at 8 min, ramp to 5% H20/95% MeCN at 8.5 min, HOLD 5% H20/95% MeCN to 10 min.
Flow rate, 60 mL/min
MS mode: MS:ESI+ scan range 165-650 daltons
PDA: 200-400 nm scan range
Column: Waters Sunfire OBD C18 PREP 30×mm, 5 um; Part No. 186002570
Modifier: 0.1% Trifluoroacetic acid (v/v) conc.
Method: A % H20/B % MeCN (initial conditions) hold 0.5 min, linear gradient to A % H20/B % MeCN at 8 min, ramp to 5% H20/95% MeCN at 8.5 min, HOLD 5% H20/95% MeCN to 10 min.

### Formic Acid (FA, Acidic pH) Conditions

Column: Welch Xtimate C18 150×30 mm×5 μm or Phenomenex luna C18 150×25 mm×10 um; Condition: water (FA)-ACN; Gradient (% organic): optimized for each example; Flow Rate (mL/min) 25.

### Hydrochloric Acid (HCl, Acidic pH) Conditions

Column: Boston Green ODS 150×30 mm×5 um; Condition: water (HCl)-ACN; Gradient (% organic): 0-100% optimized for each example; Flow Rate (mL/min) 25.

### Analytical SFC Instrumentation Specifications

Waters Acquity UPC² SFC with QDa mass spectrometer and PDA (photodiode array detector).

### Analytical Screening Conditions

MS mode: MS:ESI+ scan range 100-650 daltons
PDA: 200-400 nm scan range
Columns: See below
Solvent: Airgas Bone Dry CO₂
Cosolvents: Methanol, Ethanol, or Isopropanol with either 0.1% Diethylamine, 0.1% Dimethylethanolamine, or neutral
Method: Isocratic conditions; typically 60% CO₂: 40% cosolvent or 70% CO₂: 30% cosolvent,
Flow rate, 3.0 mL/min.

### Preparative SFC Instrumentation Specifications

Waters Prep100 SFC with QDa mass spectrometer, PDA (photodiode array detector,) and 2767 Collection bed.

### Preparative Conditions

Method: X % Cosolvent w/Y % modifier in CO₂, isocratic conditions.
Flow rate: 100 mL/min
Automated back pressure regulator: 120 bar
Manual back pressure regulator: 40 psi for MeOH or EtOH, 60 psi for iPrOH
Column oven temperate: 40° C.
MS mode: MS:ESI+ scan range 150-650 daltons
PDA: 200-400 nm scan range.

### SFC Columns, Analytical:

AD-H: Daicel Chiralpak AD-H, 4.6 mm×250 mm, 5 um, Part No 19325
AS-H: Daicel Chiralpak AS-H, 4.6 mm×250 mm, 5 um, Part No 20325
OD-H: Daicel Chiralpak OD-H, 4.6 mm×250 mm, 5 um, Part No 14325
OX-H: Daicel Chiralpak OX-H, 4.6 mm×250 mm, 5 um, Part No 63325
IA: Daicel Chiralpak IA, 4.6 mm×250 mm, 5 um, Part No 80325
IB: Daicel Chiralpak IB, 4.6 mm×250 mm, 5 um, Part No 81325
IC: Daicel Chiralpak IC, 4.6 mm×250 mm, 5 um, Part No 83325
IG: Daicel Chiralpak IG, 4.6 mm×250 mm, 5 um, Part No 87325
Cell-2: Phenomenex Lux Cellulose-2, 4.6 mm×150 mm, 3 um, Part No. 00F-4456-E0
Cell-4: Phenomenex Lux Cellulose-4, 4.6 mm×150 mm, 3 um, Part No. 00F-4490-E0

### SFC Columns, Preparative:

AD-H: Daicel Chiralpak AD-H, 30 mm×250 mm, 5 um, Part No 19475
AS-H: Daicel Chiralpak AS-H, 30 mm×250 mm, 5 um, Part No 20475
OD-H: Daicel Chiralpak OD-H, 30 mm×250 mm, 5 um, Part No 14475
OX-H: Daicel Chiralpak OX-H, 30 mm×250 mm, 5 um, Part No 63475
IA: Daicel Chiralpak IA, 30 mm×250 mm, 5 um, Part No 80475
IB: Daicel Chiralpak IB, 30 mm×250 mm, 5 um, Part No 81475
IC: Daicel Chiralpak IC, 30 mm×250 mm, 5 um, Part No 83475
IG: Daicel Chiralpak IG, 30 mm×250 mm, 5 um, Part No 87475
Cell-2: Phenomenex Lux Cellulose-2, 30 mm×250 mm, 5 um, Part No. 00G-4457-UO-AX
Cell-4: Phenomenex Lux Cellulose-4, 30 mm×250 mm, 5 um, Part No. 00G-4491-UO-AX

### ¹H-NMR

¹H nuclear magnetic resonance (NMR) spectra were in all cases consistent with the proposed structures. The ¹H NMR spectra were recorded on a Bruker Avance III HD 500 MHz, Bruker Avance III 500 MHz, Bruker Avance DRX 500, Bruker Avance III 400 MHz, Varian-400 VNMRS, Varian Unityplus 400, or Varian-400 MR. Characteristic chemical shifts (d) are given in parts-per-million downfield from tetramethylsilane (for ¹H-NMR) using conventional abbreviations for designation of major peaks: e.g. s, singlet; d, doublet; t, triplet; q, quartet; dd, double doublet; dt, double triplet; m, multiplet; br, broad. The following abbreviations have been used for common solvents: CDCl₃, deuterochloroform; DMSO-d₆, hexadeuterodimethyl sulfoxide; and MeOH-d₄, deuteromethanol. Where appropriate, tautomers may be recorded within the NMR data; and some exchangeable protons may not be visible.

### Section 2. Preparation of Intermediates

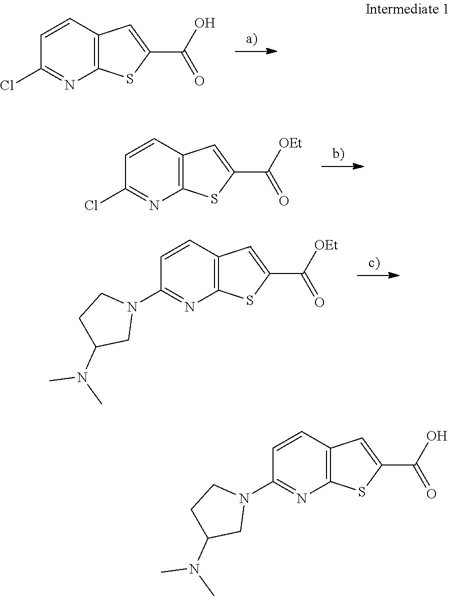 [SMILES: O=C(O)c1cc2ccc(Cl)nc2s1 ; CCOC(=O)c1cc2ccc(Cl)nc2s1 ; CCOC(=O)c1cc2ccc(N3CCC(N(C)C)C3)nc2s1 ; CN(C)C1CCN(c2ccc3cc(C(=O)O)sc3n2)C1]

Step a: 6-Chlorothieno[2,3-b]pyridine-2-carboxylic acid (500 mg, 2.34 mmol, 1.0 eq.) was dissolved in ethanol (11.70 mL, 0.2 M) before HCl in dioxane (4 M, 1.76 mL, 3 eq.) was added. The solution was then stirred at 80° C. for 16 hours before being concentrated to obtain ethyl 6-chlorothieno[2,3-b]pyridine-2-carboxylate (503.4 mg, 80% yield) as an off-white powder that was carried forward crude. MS: m/z 242.0 [M+H]⁺.

Step b: N,N-Dimethylpyrrolidin-3-amine (190.98 mg, 1.67 mmol, 1.1 eq.), ethyl 6-chlorothieno[2,3-b]pyridine-2-carboxylate (408.31 mg, 1.52 mmol (1.0 eq.), and DIPEA (1.38 g, 10.64 mmol, 1.85 mL, 7 eq.) were dissolved in dioxane (7.60 mL, 0.2 M) which was heated to 80° C. for 72 hours before it was dry loaded onto normal phase silica column and purified via 0-25% MeOH:DCM to obtain ethyl 6-[3-(dimethylamino)pyrrolidin-1-yl]thieno[2,3-b]pyridine-2-carboxylate (468.0 mg, 93% yield). MS: m/z 320.0 [M+H]⁺.

Step c: Ethyl 6-[3-(dimethylamino)pyrrolidin-1-yl]thieno[2,3-b]pyridine-2-carboxylate (482.47 mg, 1.47 mmol, 1.0 eq.) was dissolved in dioxane (515 μL, 1.42 M) and water (515 L, 1.42 M) before lithium hydroxide (35.09 mg, 1.47 mmol, 1.0 eq.) was added. The solution was then heated at 80° C. for 16 hours before it was concentrated to obtain 6-[3-(dimethylamino)pyrrolidin-1-yl]thieno[2,3-b]pyridine-2-carboxylic acid (535.7 mg, 99% yield) as an off-white powder. MS: RT m/z 292.0 [M+H]⁺.

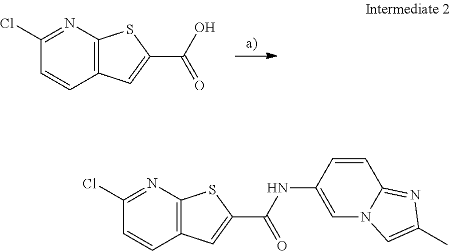 [SMILES: O=C(O)c1cc2ccc(Cl)nc2s1 ; Cc1cn2cc(NC(=O)c3cc4ccc(Cl)nc4s3)ccc2n1]

6-Chlorothieno[2,3-b]pyridine-2-carboxylic acid (500 mg, 2.34 mmol, 1.0 eq) was dissolved in dichloromethane (11.70 mL, 0.2 M) before 2-methylimidazo[1,2-a]pyridin-6-amine (344.45 mg, 2.34 mmol, 1.0 eq.), HATU (978.87 mg, 2.57 mmol, 1.0 eq.), and DIPEA (665.43 mg, 5.15 mmol, 896 μL) were added. The solution was then stirred at rt for 1 hour before it was concentrated and injected directly onto normal phase silica column and purified via 0-25% MeOH:DCM over 3.5 min to obtain 6-chloro-N-(2-methylimidazo[1,2-a]pyridin-6-yl)thieno[2,3-b]pyridine-2-carboxamide (420.9 mg, 44% yield) as a brown solid that was carried forward as is. MS: m/z 343.0 [M+H]⁺.

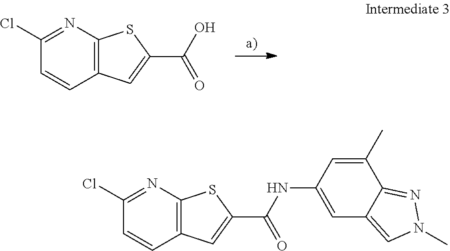 [SMILES: O=C(O)c1cc2ccc(Cl)nc2s1 ; Cc1cc(NC(=O)c2cc3ccc(Cl)nc3s2)cc2cn(C)nc12]

6-Chlorothieno[2,3-b]pyridine-2-carboxylic acid (25 mg, 117.02 μmol, 1.0 eq.), 2,7-dimethylindazol-5-amine (22.64 mg, 140.42 μmol, 1.2 eq.), HATU (48.94 mg, 128.72 μmol, 1.1 eq.), and DIPEA (33.27 mg, 257.44 μmol, 44 μL, 2.2 eq.) were dissolved in dichloromethane (585.09 μL, 0.2 M) before being stirred at rt for 2 hours. The solution was then purified via silica column chromatography (0-25% MeOH:DCM over 3.5 min) to obtain 6-chloro-N-(2,7-dimethylindazol-5-yl)thieno[2,3-b]pyridine-2-carboxamide (49.5 mg, 100% yield) as a brown solid. MS: m/z 357.0 [M+H]⁺.

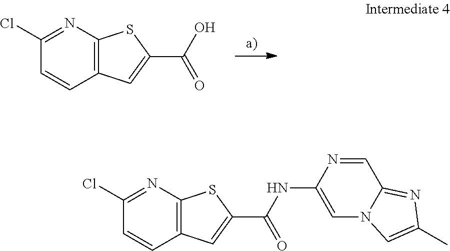 [SMILES: O=C(O)c1cc2ccc(Cl)nc2s1 ; Cc1cn2cc(NC(=O)c3cc4ccc(Cl)nc4s3)ncc2n1]

6-Chlorothieno[2,3-b]pyridine-2-carboxylic acid (25 mg, 117.02 μmol, 1.0 eq.), 2-methylimidazo[1,2-a]pyrazin-6-amine (20.81 mg, 140.42 μmol, 1.2 eq.), HATU (48.94 mg, 128.72 μmol, 1.1 eq.), and triethylamine (26.05 mg, 257.4 μmol, 36 μL, 2.2 eq.) were dissolved in dichloromethane (585.09 μL, 0.2 M) and then stirred at rt for 2 hours before it was injected directly onto normal phase silica column and purified via 0-25% MeOH:DCM over 3.5 minutes to obtain 6-chloro-N-(2-methylimidazo[1,2-a]pyrazin-6-yl)thieno[2,3-b]pyridine-2-carboxamide (25.8 mg, 61% yield) as a brown solid that was carried forward as is. MS: m/z 344.0 [M+H]⁺.

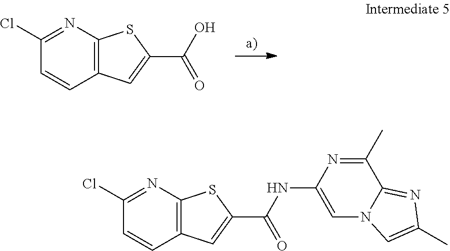 [SMILES: O=C(O)c1cc2ccc(Cl)nc2s1 ; Cc1cn2cc(NC(=O)c3cc4ccc(Cl)nc4s3)nc(C)c2n1]

6-Chlorothieno[2,3-b]pyridine-2-carboxylic acid (25 mg, 117.02 μmol, 1.0 eq.), 2,8-dimethylimidazo[1,2-a]pyrazin-6-amine (22.78 mg, 140.42 μmol, 1.2 eq.), HATU (48.94 mg, 128.72 μmol, 1.1 eq.), and Triethylamine (26.05 mg, 257.44 μmol, 35.88 μL, 2.2 eq.) were dissolved in Dichloromethane (585.09 μL, 0.2 M) then stirred at RT for 2 hours then injected crude onto normal phase silica column and purified via 0-25% MeOH:DCM over 3.5 minutes. Product elutes at 20%. Identified fractions were collected and concentrated to obtain 6-chloro-N-(2,8-dimethylimidazo[1,2-a]pyrazin-6-yl)thieno[2,3-b]pyridine-2-carboxamide (42.9 mg, 91% yield) as brown solid that was carried forward as is. MS: m/z 358.1 [M+H]⁺.

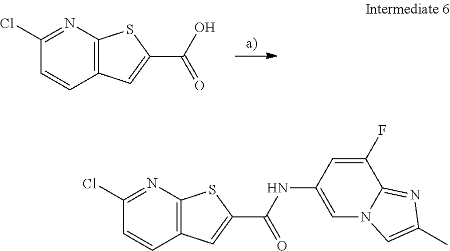 [SMILES: O=C(O)c1cc2ccc(Cl)nc2s1 ; Cc1cn2cc(NC(=O)c3cc4ccc(Cl)nc4s3)cc(F)c2n1]

To a solution of 6-chlorothieno[2,3-b]pyridine-2-carboxylic acid (150 mg, 702.1 mol) in DMF (20 mL) was added N-ethyl-N-isopropyl-propan-2-amine (2.11 mmol, 367 L), [dimethylamino(triazolo[4,5-b]pyridin-3-yloxy)methylene]-dimethyl-ammonium;hexafluorophosphate (320.36 mg, 842.54 μmol) and 8-fluoro-2-methyl-imidazo[1,2-a]pyridin-6-amine (115.97 mg, 702.11 μmol) at 20° C. The reaction mixture was stirred at 20° C. for 1 h. The reaction mixture was quenched with water (50 ml) and it was extracted with EtOAC (40 mL×3). The combined organic layers were dried over Na₂SO₄, filtered and concentrated under reduced pressure to give a residue, which was purified by flash silica gel chromatography (from PE/EtOAc=3/1 to 0/1, TLC: PE/EtOAc=0/1) to give the 6-chloro-N-(8-fluoro-2-methyl-imidazo[1,2-a]pyridin-6-yl)thieno[2,3-b]pyridine-2-carboxamide (180 mg, 64% yield) as a brown solid. MS: m/z 361.2 [M+H]⁺.

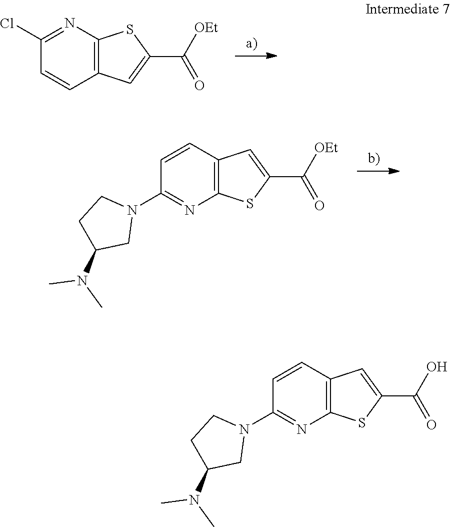 [SMILES: CCOC(=O)c1cc2ccc(Cl)nc2s1 ; CCOC(=O)c1cc2ccc(N3CC[C@H](N(C)C)C3)nc2s1 ; CN(C)[C@H]1CCN(c2ccc3cc(C(=O)O)sc3n2)C1]

Step a: Ethyl 6-chlorothieno[2,3-b]pyridine-2-carboxylate (265 mg, 1.02 mmol, 1.0 eq.) was dissolved in dioxane (5 mL, 0.2 M) before TEA (309.54 mg, 3.06 mmol, 426.36 μL, 3.0 eq.) and (3S)—N,N-dimethylpyrrolidin-3-amine (465.89 mg, 4.08 mmol, 518.23 mL, 4 eq.) were added. The solution was then heated to 80° C. for 16 hours before it was concentrated and dry loaded onto normal phase silica column and purified via 0-25% MeOH:DCM over 12 minutes. Product elutes at 11% MeOH. Identified fractions were collected and concentrated to obtain ethyl 6-[(3S)-3-(dimethylamino)pyrrolidin-1-yl]thieno[2,3-b]pyridine-2-carboxylate (258.2 mg, 808.34 μmol, 79% yield) as a white powder. MS: m/z 320.0 [M+H]⁺.

Step b: Ethyl 6-[(3S)-3-(dimethylamino)pyrrolidin-1-yl]thieno[2,3-b]pyridine-2-carboxylate (258.2 mg, 842.10 μmol, 1.0 eq.) was dissolved in THF (1 mL, 0.4 M) and water (1 mL, 0.4 M) before lithium hydroxide (23.23 mg, 970.01 μmol, 1.2 eq.) was added. The solution was then heated at 60° C. for 96 hours before it was then concentrated to a white powder to obtain 6-[(3S)-3-(dimethylamino)pyrrolidin-1-yl]thieno[2,3-b]pyridine-2-carboxylic acid (241.1 mg) as an off yellow white powder that was carried forward as is. MS: m/z 292.0 [M+H]⁺.

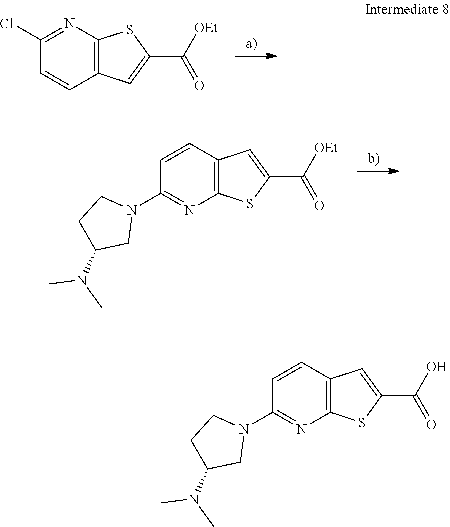 [SMILES: CCOC(=O)c1cc2ccc(Cl)nc2s1 ; CCOC(=O)c1cc2ccc(N3CC[C@@H](N(C)C)C3)nc2s1 ; CN(C)[C@@H]1CCN(c2ccc3cc(C(=O)O)sc3n2)C1]

Step a: Ethyl 6-chlorothieno[2,3-b]pyridine-2-carboxylate (150 mg, 552.35 μmol, 1.0 eq.) was dissolved in dioxane (1.84 mL, 0.3 M) before TEA (167.68 mg, 1.66 mmol, 230.96 μL, 3.0 eq.) and (3R)—N,N-dimethylpyrrolidin-3-amine (828.5 μmol, 105 μL, 1.5 eq.) were added. The solution was then heated to 80 C for 16 hours before it was concentrated and dry loaded onto normal phase silica and purified via 0-25% MeOH:DCM over 12 minutes. Product elutes at 11% MeOH. Identified fractions were collected and concentrated to obtain ethyl 6-[(3R)-3-(dimethylamino)pyrrolidin-1-yl]thieno[2,3-b]pyridine-2-carboxylate (162.9 mg, 89% yield) as a white powder. MS: m/z 320.0 [M+H]⁺.

Step b: Ethyl 6-[(3R)-3-(dimethylamino)pyrrolidin-1-yl]thieno[2,3-b]pyridine-2-carboxylate (162.9 mg, 494.69 μmol, 1.0 eq.) was dissolved in dioxane (1.24 mL, 0.2 M) and water (1.24 mL, 0.2 M) before lithium hydroxide (13.03 mg, 544.15 μmol, 1.1 eq.) was added. The solution was then heated at 50° C. for 16 hours before it was concentrated to a white powder to obtain 6-[(3R)-3-(dimethylamino)pyrrolidin-1-yl]thieno[2,3-b]pyridine-2-carboxylic acid (157.9 mg) as an off-white powder that was carried forward as is. MS: m/z 292.0 [M+H]⁺.

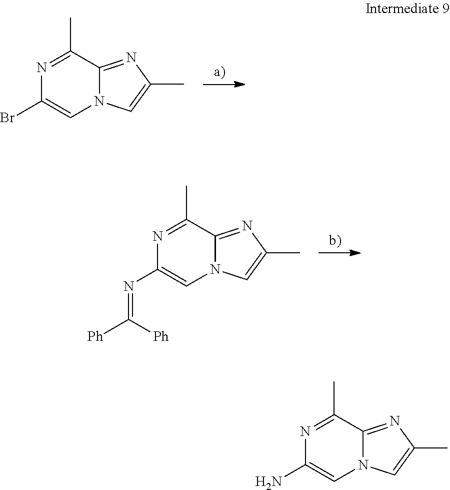 [SMILES: Cc1cn2cc(Br)nc(C)c2n1 ; Cc1cn2cc(N=C(c3ccccc3)c3ccccc3)nc(C)c2n1 ; Cc1cn2cc(N)nc(C)c2n1]

Step a: To a solution of 6-bromo-2,8-dimethyl-imidazo[1,2-a]pyrazine (22.2 g, 98.20 mmol) in toluene (250 mL) was added sodium tert-butoxide (16.99 g, 176.76 mmol) and BINAP (18.34 g, 29.46 mmol), then diphenylmethanimine (35.59 g, 196.40 mmol, 32.96 mL) was added under N₂. The mixture was stirred at 90° C. for 16 h. The mixture was then diluted with EtOAc (50 mL) and filtered. The filtrate was evaporated in vacuo and the crude product was purified by flash column chromatography (silica; EtOAc in petroleum 0% to 100%). The desired product was collected, and the solvents were evaporated in vacuo to yield N-(diphenylmethylene)-2,8-dimethylimidazo[1,2-a]pyrazin-6-amine (25.2 g) as a yellow oil. MS: m/z 327.1 [M+H]⁺.

Step b: To a mixture of N-(2,8-dimethylimidazo[1,2-a]pyrazin-6-yl)-1,1-diphenyl-methanimine (25.2 g, 77.21 mmol) in THE (150 mL) was added HCl (2 M, 120 mL) at 20° C. The mixture was stirred at 20° C. for 1 h. The solution was concentrated and then dissolved in water (150 mL). The mixture was extracted with DCM (200 mL×3). The aqueous phase was neutralized with 2 N NaOH (to pH=13) and extracted with DCM (200 mL×3). The combined organic layers were washed with brine (200 mL), dried over Na₂SO₄, filtered and concentrated to give 2,8-dimethylimidazo[1,2-a]pyrazin-6-amine (8.2 g, 50.56 mmol, 65% yield) as a brown solid. MS: m/z 163.1 [M+H]⁺.

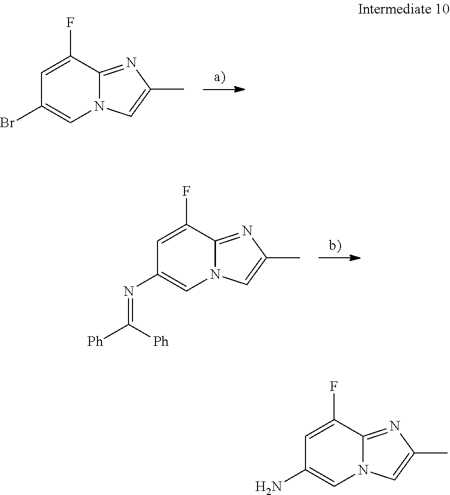 [SMILES: Cc1cn2cc(Br)cc(F)c2n1 ; Cc1cn2cc(N=C(c3ccccc3)c3ccccc3)cc(F)c2n1 ; Cc1cn2cc(N)cc(F)c2n1]

Step a: To a solution of 6-bromo-8-fluoro-2-methyl-imidazo[1,2-a]pyridine (1 g, 4.37 mmol), diphenylmethanimine (1.19 g, 6.55 mmol, 1.10 mL) and (5-diphenylphosphanyl-9,9-dimethyl-xanthen-4-yl)-diphenyl-phosphane (505.24 mg, 873.18 mol) in dioxane (30 mL) was added cesium carbonate (4.27 g, 13.10 mmol) and Pd₂(dba)₃ (399.79 mg, 436.59 μmol) at 20° C. under N₂. The reaction was stirred at 100° C. for 14 hours. The mixture was filtered, and the filtrate was evaporated under vacuum. The residue was purified by column chromatography (PE:EtOAc from 5:1 to 1:1) to give N-(8-fluoro-2-methyl-imidazo[1,2-a]pyridin-6-yl)-1,1-diphenyl-methanimine (1.3 g, 3.95 mmol, 90% yield) as an off-white solid. MS: m/z 330.2 [M+H]⁺.

Step b: To a solution of N-(8-fluoro-2-methyl-imidazo[1,2-a]pyridin-6-yl)-1,1-diphenyl-methanimine (1.3 g, 3.95 mmol) in HCl (4 M, 8 mL) was added HCl (143.91 mg, 3.95 mmol, 0.5 mL) at 20° C. The reaction was stirred at 20° C. for 14 hours. The solution was evaporated under vacuum, and the residue was adjusted to pH=7 with saturated aq. NaHCO₃ (100 mL), extracted with DCM (50 mL×3). The combined organic layers were dried over Na₂SO₄ and filtered. The filtrate was evaporated under vacuum. The residue was purified by column chromatography (PE:EtOAc from 3:1 to 0:1) to give 8-fluoro-2-methyl-imidazo[1,2-a]pyridin-6-amine (470 mg, 2.85 mmol, 72% yield) as a brown solid. MS: m/z 166.1 [M+H]⁺.

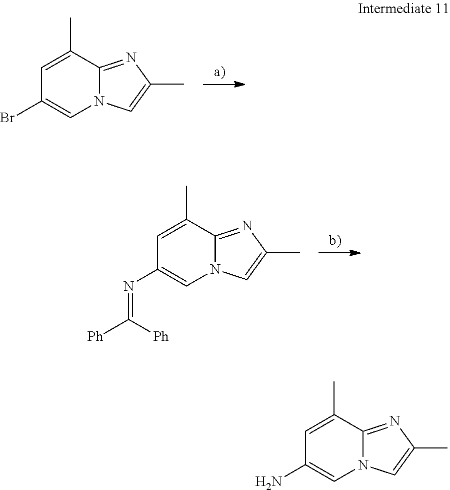 [SMILES: Cc1cn2cc(Br)cc(C)c2n1 ; Cc1cn2cc(N=C(c3ccccc3)c3ccccc3)cc(C)c2n1 ; Cc1cn2cc(N)cc(C)c2n1]

Step a: A mixture of Pd₂(dba)₃ (122.05 mg, 133.28 μmol), 6-bromo-2,8-dimethyl-imidazo[1,2-a]pyridine (300 mg, 1.33 mmol), sodium tert-butoxide (256.18 mg, 2.67 mmol) and [1-(2-diphenylphosphanyl-1-naphthyl)-2-naphthyl]-diphenyl-phosphane (165.98 mg, 266.57 μmol) was added toluene (6 mL) and diphenylmethanimine (483.11 mg, 2.67 mmol, 447.32 μL) under N₂. The reaction mixture was stirred at 130° C. for 12 h. The mixture was concentrated, and the residue was purified by column chromatography (0% to 70% EtOAc in heptane) to give N-(2,8-dimethylimidazo[1,2-a]pyridin-6-yl)-1,1-diphenyl-methanimine (455.1 mg) as pale-yellow solid. MS: m/z 326.1 [M+H]⁺.

Step b: To a mixture of N-(diphenylmethylene)-2,8-dimethylimidazo[1,2-a]pyridin-6-amine (95.4 mg, 293.17 μmol) in THE (2 mL) was added hydrochloric acid (4 M in dioxane, 219.88 μL) at 20° C. The mixture was stirred at 20° C. for 1 h. The mixture was concentrated under vacuum and then DCM (1 mL) was added. The mixture was filtered. The precipitate was collected to give 2,8-dimethylimidazo[1,2-a]pyridin-6-amine (52.4 mg, 265.10 μmol, 90% yield, Hydrochloride) as a pale-yellow solid. MS: m/z 162.1 [M+H]⁺.

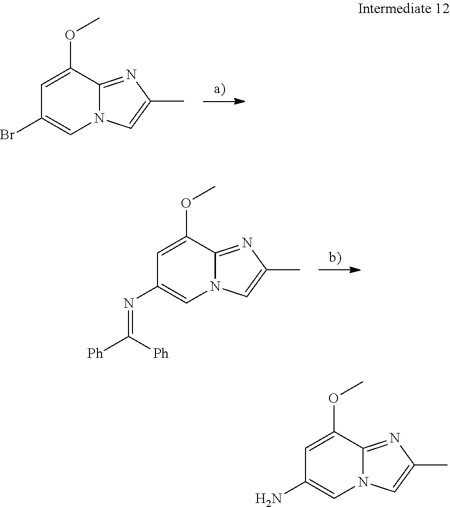 [SMILES: COc1cc(Br)cn2cc(C)nc12 ; COc1cc(N=C(c2ccccc2)c2ccccc2)cn2cc(C)nc12 ; COc1cc(N)cn2cc(C)nc12]

Step a: To a mixture of 6-bromo-8-methoxy-2-methylimidazo[1,2-a]pyridine (800 mg, 3.32 mmol) in toluene (30 mL) was added diphenylmethanimine (902.09 mg, 4.98 mmol, 835.27 μL), sodium tert-butoxide (574.03 mg, 5.97 mmol), Pd₂(dba)₃ (303.87 mg, 331.83 mol) and [1-(2-diphenylphosphanyl-1-naphthyl)-2-naphthyl]-diphenyl-phosphane (413.25 mg, 663.67 mol) at 20° C. The mixture was stirred at 130° C. for 16 h under N₂ atmosphere. The mixture was filtered, and the filtrate was concentrated. Then water (50 mL) was added, and the mixture was extracted with EtOAc (50 mL). The organic layer was washed with brine (50 mL), dried over Na₂SO₄, filtered and concentrated. The crude material was purified by chromatography (petroleum ether/EtOAc=5/1 to 0/1) to yield N-(diphenylmethylene)-8-methoxy-2-methylimidazo[1,2-a]pyridin-6-amine (1 g, 2.93 mmol, 88% yield) as brown oil. MS: m/z 342.3 [M+H]⁺.

Step b: To a mixture of N-(diphenylmethylene)-8-methoxy-2-methylimidazo[1,2-a]pyridin-6-amine (1 g, 2.93 mmol) in THF (10 mL) was added HCl (2 M, 2.71 mL) at 20° C. The mixture was stirred at 20° C. for 2 h under N₂ atmosphere. The mixture was concentrated under reduced pressure. The water phase was adjusted to pH 3-4 with HCl (2 N) and extracted with DCM (50 mL). The water phase was then added sat. NaOH (1 N) to adjust pH to 14 and extracted with DCM (100 mL). The organic layer was washed with brine (80 mL), dried over Na₂SO₄, filtered and concentrated under reduced pressure to yield 8-methoxy-2-methylimidazo [1,2-a]pyridin-6-amine (200 mg, 1.13 mmol, 39% yield) as a yellowish-brown solid. MS: m/z 178.2 [M+H]⁺.

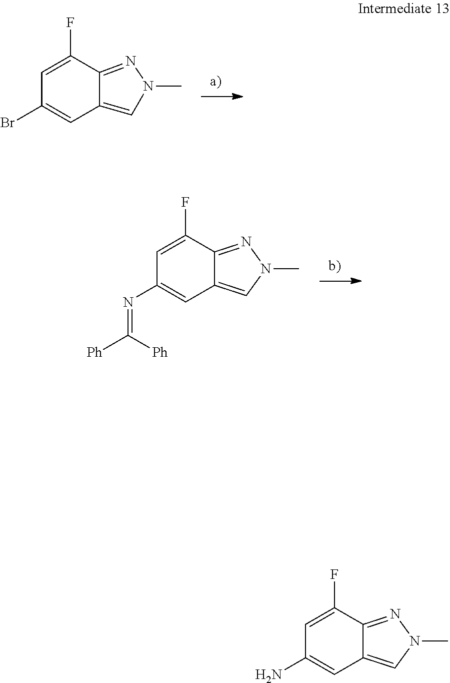

Step a: To a mixture of 5-bromo-7-fluoro-2-methyl-indazole (100 mg, 436.59 μmol), rac-BINAP-Pd-G3 (43.31 mg, 43.66 μmol) and sodium tert-butoxide (83.92 mg, 873.18 μmol) was added toluene (1 mL) and diphenylmethanimine (94.95 mg, 523.91 μmol, 87.92 μL) under N₂ atmosphere. The reaction mixture was stirred at 110° C. for 12 h. The reaction mixture was concentrated, and the residue was purified by column flash chromatography (0% to 60% EtOAc in heptane) to give N-(7-fluoro-2-methyl-indazol-5-yl)-1,1-diphenyl-methanimine (107.3 mg, 325.77 μmol, 74% yield) as a pale-yellow solid. MS: m/z 330.1 [M+H]⁺.

Step b: To a mixture of N-(7-fluoro-2-methyl-indazol-5-yl)-1,1-diphenyl-methanimine (107.3 mg, 325.77 μmol) in THF (2 mL) was added hydrochloric acid (4 M in dioxane, 244.33 μL) at 20° C. The mixture was stirred at 20° C. for 1 h. The mixture was concentrated and then water was added. The mixture was extracted with DCM (5 mL×3). Aqueous phase neutralized with 2 N NaOH to pH=11 and the residue was extracted with DCM (5 mL×3). The combined organic layers were washed with brine, dried over MgSO₄, filtered and concentrated to give 7-fluoro-2-methyl-indazol-5-amine (36.1 mg, 218.57 μmol, 67% yield) as a pale-yellow solid.

MS: m/z 166.0 [M+H]⁺.

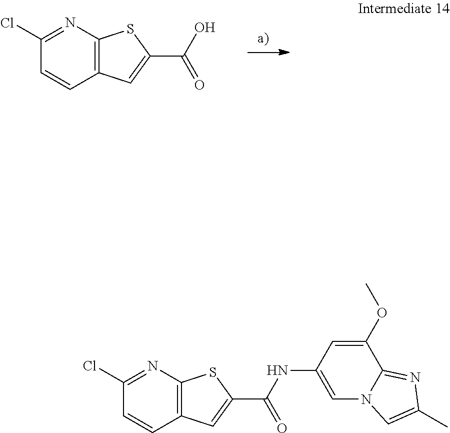 [SMILES: O=C(O)c1cc2ccc(Cl)nc2s1 ; COc1cc(NC(=O)c2cc3ccc(Cl)nc3s2)cn2cc(C)nc12]

8-methoxy-2-methyl-imidazo[1,2-a]pyridin-6-amine (91.24 mg, 514.88 mol) and 6-chlorothieno[2,3-b]pyridine-2-carboxylic acid (100 mg, 468.07 mol) were dissolved in dioxane (2 mL) before 2,4,6-tripropyl-1,3,5,2,4,6trioxatriphosphinane 2,4,6-trioxide (446.80 mg, 702.11 μmol, 417.96 μL, 50% purity) was added. The reaction mixture was stirred at 60° C. for 16 h and then concentrated under vacuum. The residue was purified by column flash chromatography (0% to 10% MeOH in DCM) to give 6-chloro-N-(8-methoxy-2-methyl-imidazo[1,2-a]pyridin-6-yl)thieno[2,3-b]pyridine-2-carboxamide (145.3 mg, 389.72 μmol, 83.26% yield). MS: m/z 373.1 [M+H]⁺.

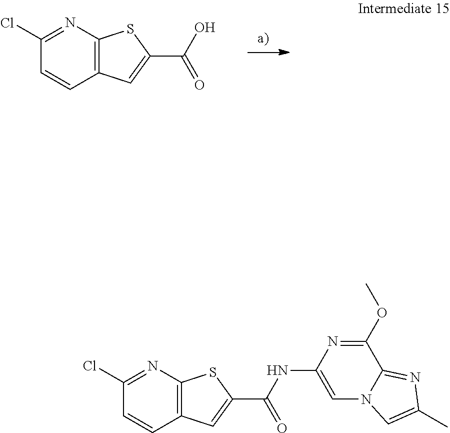 [SMILES: O=C(O)c1cc2ccc(Cl)nc2s1 ; COc1nc(NC(=O)c2cc3ccc(Cl)nc3s2)cn2cc(C)nc12]

6-chlorothieno[2,3-b]pyridine-2-carboxylic acid (422.11 mg, 1.98 mmol) was dissolved in dioxane (10.87 mL) before 2,8-dimethylimidazo[1,2-a]pyrazin-6-amine (352.5 mg, 2.17 mmol), T₃P (3.77 g, 5.93 mmol, 2.66 mL, 50% purity), and TEA (599.79 mg, 5.93 mmol, 826.15 μL) were added. The solution was heated to 60° C. for 16 hours and then concentrated before being purified by column flash chromatography (0-15% MeOH:DCM over 12 minutes). Identified fractions were collected and concentrated to yield 6-chloro-N-(2,8-dimethylimidazo[1,2-a]pyrazin-6-yl)thieno[2,3-b]pyridine-2-carboxamide (661.1 mg, 1.81 mmol, 91.64% yield, 98% purity) as a tannish white plastic solid. MS: m/z 358.1 [M+H]⁺. 1H NMR (400 MHz, DMSO-d₆) δ ppm 2.38-2.41 (m, 3H) 2.73-2.75 (m, 3H) 7.58-7.65 (m, 1H) 7.96-8.01 (m, 1H) 8.43-8.49 (m, 1H) 8.56-8.61 (m, 1H) 9.12-9.17 (m, 1H) 11.31-11.36 (m, 1H).

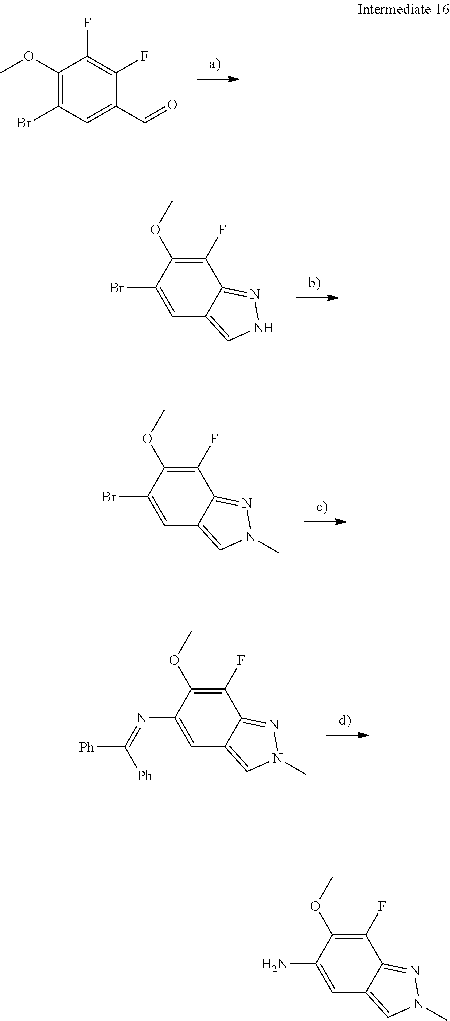 [SMILES: COc1c(Br)cc(C=O)c(F)c1F ; COc1c(Br)cc2c[nH]nc2c1F ; COc1c(Br)cc2cn(C)nc2c1F ; COc1c(N=C(c2ccccc2)c2ccccc2)cc2cn(C)nc2c1F ; COc1c(N)cc2cn(C)nc2c1F]

Step a: A mixture of 5-bromo-2,3-difluoro-4-methoxy-benzaldehyde (20.4 g, 81.27 mmol), O-methylhydroxylamine hydrochloride (8.82 g, 105.65 mmol) and K₂CO₃ (24.71 g, 178.79 mmol) in DME (200 mL) was degassed and purged with N₂ for 3 times, and then the mixture was stirred at 50° C. for 16 hr under N₂ atmosphere. The reaction mixture was filtered and concentrated to give (E)-1-(5-bromo-2,3-difluoro-4-methoxy-phenyl)-N-methoxy-methanimine (22.1 g, 78.91 mmol, 97.10% yield). To a solution of (E)-1-(5-bromo-2,3-difluoro-4-methoxy-phenyl)-N-methoxy-methanimine (2.6 g, 9.28 mmol) in THE (30 mL) was added NH₂NH₂·H₂O (9.89 g, 197.56 mmol, 8.2 mL) at 25° C. under N₂ atmosphere. The mixture was stirred at 80° C. under N₂ for 90 hours. The mixture was diluted with water (100 mL) and extracted with EtOAc (100 mL×3). The pH of aqueous phase was adjusted to neutral. The combined organic layers were washed with brine (150 mL), dried over Na₂SO₄, filtered and concentrated. The residue was purified by column chromatography (SiO₂, Petroleum ether/Ethyl acetate=100/1 to 9/2) to give 5-bromo-7-fluoro-6-methoxy-1H-indazole (2 g, 8.16 mmol, 87.92% yield) as a yellowish solid. MS: m/z 246.9 [M+H]⁺.

Step b: A solution of 5-bromo-7-fluoro-6-methoxy-1H-indazole (6.5 g, 26.53 mmol) in EtOAc (100 mL) was added trimethyloxonium tetrafluoroborate (5.88 g, 39.79 mmol). The reaction was stirred at 25° C. for 3 hr. The mixture was filtered and concentrated to give 5-bromo-7-fluoro-6-methoxy-2-methyl-indazole (5.9 g, 22.77 mmol, 85.85% yield). MS: m/z 259.0 [M+H]⁺.

Step c: A mixture of 5-bromo-7-fluoro-6-methoxy-2-methyl-indazole (5.9 g, 22.77 mmol), diphenylmethanimine (6.19 g, 34.16 mmol, 5.73 mL), sodium 2-methylpropan-2-olate (6.57 g, 68.32 mmol), [2-(2-aminophenyl)phenyl]-methylsulfonyloxy-palladium ditert-butyl-[2-(2,4,6-triisopropylphenyl)phenyl]phosphane (1.81 g, 2.28 mmol) in dioxane (100 mL) was degassed and purged with N₂ for 3 times. The mixture was stirred at 90° C. for 3 hr under N₂ atmosphere. The mixture was concentrated and purified with column chromatography (SiO₂, Petroleum ether/Ethyl acetate=100/1 to 9/2) to yield N-(7-fluoro-6-methoxy-2-methyl-indazol-5-yl)-1,1-diphenyl-methanimine (5.7 g, 15.86 mmol, 69.64% yield). MS: m/z 360.1 [M+H]⁺.

Step d: A mixture of N-(7-fluoro-6-methoxy-2-methyl-indazol-5-yl)-1,1-diphenyl-methanimine (5.7 g, 15.86 mmol) and HCl/EtOAc (2 M, 200.00 mmol, 100 mL) in EtOAc (1000 mL) was stirred at 25° C. for 1 hr. The mixture was filtered, and the precipitates were washed with EtOAc to give 7-fluoro-6-methoxy-2-methyl-indazol-5-amine (3.91 g, 14.58 mmol, 91.95% yield, Hydrochloride). MS: m/z 268.1 [M+H]⁺. ¹H NMR (400 MHz, MeOD) δ ppm: 9.34-9.31 (m, 1H), 7.92 (s, 1H), 7.50-7.44 (m, 2H), 7.34-7.27 (m, 3H), 2.88-2.86 (m, 3H), 2.53 (s, 3H).

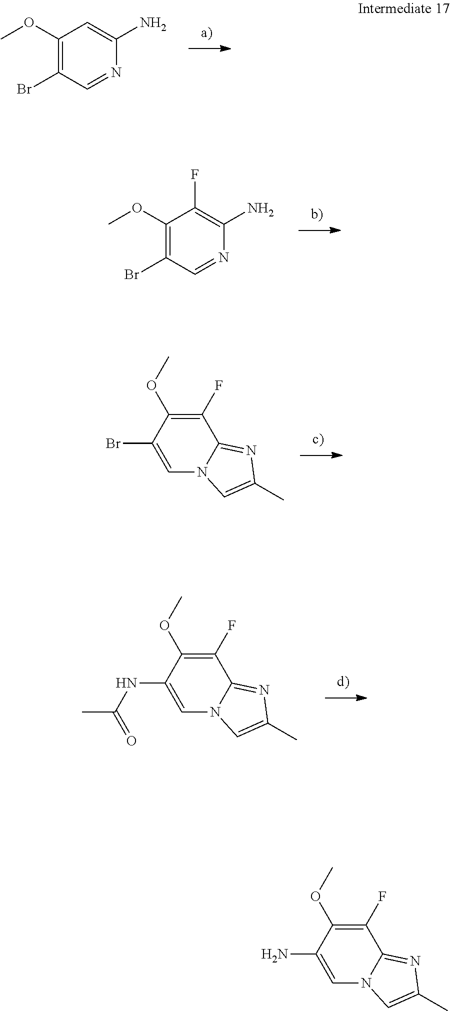

Step a: To a solution of 5-bromo-4-methoxy-pyridin-2-amine (57 g, 280.74 mmol) in DCM (300 mL) and H₂O (300 mL) was added 1-(chloromethyl)-4-fluoro-1,4-diazoniabicyclo[2.2.2]octane ditetrafluoroborate (198.91 g, 561.48 mmol), the mixture was stirred at 25° C. for 3 hours.

The aqueous phase was adjusted to pH 8 with NaHCO₃ and extracted with DCM (200 mL×3). The organic phase was combined and dried over Na₂SO₄, filtered and concentrated under reduced pressure. The residue was purified by column chromatography (SiO₂, Petroleum ether/Ethyl acetate=100/1 to 4/1) to give 5-bromo-3-fluoro-4-methoxy-pyridin-2-amine (5.6 g, 25.34 mmol, 9.02% yield) as a orange red solid. MS: m/z 222.8 [M+H]⁺.

Step b: To a solution of 5-bromo-3-fluoro-4-methoxy-pyridin-2-amine (4.5 g, 20.36 mmol) in EtOH (50 mL) was added 1-chloropropan-2-one (18.78 g, 202.97 mmol, 16.16 mL). The mixture was stirred at 100° C. for 8 hours. The mixture was concentrated in vacuo and redissolved in H₂O (100 mL), and adjusted to pH 8 with NaHCO₃. Then the mixture was re-extracted with ethyl acetate (3×60 mL). The combined organic phase was washed with brine (30 mL), dried over Na₂SO₄ and concentrated to give 6-bromo-8-fluoro-7-methoxy-2-methyl-imidazo[1,2-a]pyridine (4 g, crude) as a brown oil. MS: m/z 258.9 [M+H]⁺.

Step c: A mixture of 6-bromo-8-fluoro-7-methoxy-2-methyl-imidazo[1,2-a]pyridine (6.2 g, 23.93 mmol), acetamide (14.14 g, 239.31 mmol), Cs₂CO₃ (15.59 g, 47.86 mmol) in dioxane (100 mL) was degassed and purged with N₂ for 3 times, and then BrettPhos Pd G3 (6.51 g, 7.18 mmol) was added. The mixture was stirred at 100° C. for 2 hours under N₂. The mixture was diluted with H₂O (200 ml), and extracted with ethyl acetate (100 ml×4). The organic phase were combined and dried over Na₂SO₄, filtered and concentrated under reduced pressure. The residue was purified by column chromatography (SiO2, Petroleum ether/Ethyl acetate=100/1 to 3/2) to give N-(8-fluoro-7-methoxy-2-methyl-imidazo[1,2-a]pyridin-6-yl)acetamide (4.3 g, 18.13 mmol, 75.74% yield) as a yellow solid. MS: m/z 237.9 [M+H]⁺.

Step d: To a solution of N-(8-fluoro-7-methoxy-2-methyl-imidazo[1,2-a]pyridin-6-yl)acetamide (4.1 g, 17.28 mmol) in MeOH (60 mL) was added HCl (12 M, 129.62 mmol, 10.80 mL). The mixture was stirred at 60° C. for 3 hours. The reaction was concentrated, diluted with H₂O (100 ml) and adjusted to pH 7 with NaHCO₃. The mixture was extracted with ethyl acetate (100 ml×3). The organic phases were combined, dried over Na₂SO₄, filtered and concentrated under reduced pressure. The residue was purified by column chromatography (SiO2, Petroleum ether/Ethyl acetate=100/1 to 0/1) to give 8-fluoro-7-methoxy-2-methyl-imidazo[1,2-a]pyridin-6-amine (1.8 g, 9.22 mmol, 53.36% yield) as a gray solid. MS: m/z 195.9 [M+H]⁺.

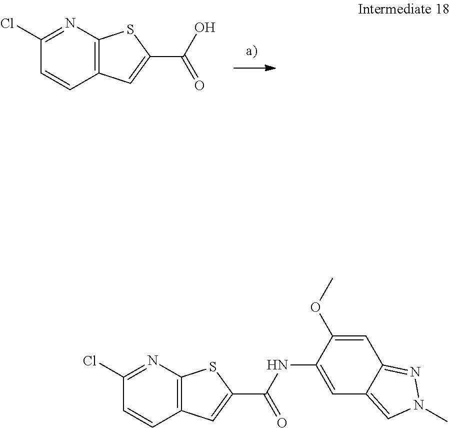 [SMILES: O=C(O)c1cc2ccc(Cl)nc2s1 ; COc1cc2nn(C)cc2cc1NC(=O)c1cc2ccc(Cl)nc2s1]

6-methoxy-2-methyl-indazol-5-amine (99.53 mg, 561.69 μmol) and 6-chlorothieno[2,3-b]pyridine-2-carboxylic acid (100 mg, 468.07 μmol) were dissolved in dioxane (2 mL) before 2,4,6-tripropyl-1,3,5,2,4,6-trioxatriphosphinane-2,4,6-trioxide (446.80 mg, 702.11 μmol, 417.96 μL, 50% purity) was added. The reaction mixture was stirred at 40° C. for 16 h and then concentrated under vacuum. The residue was purified by column flash chromatography (0% to 10% MeOH in DCM) to give 6-chloro-N-(6-methoxy-2-methyl-indazol-5-yl)thieno[2,3-b]pyridine-2-carboxamide (138.6 mg, 371.75 μmol, 79.42% yield). MS: m/z 373.1 [M+H]⁺.

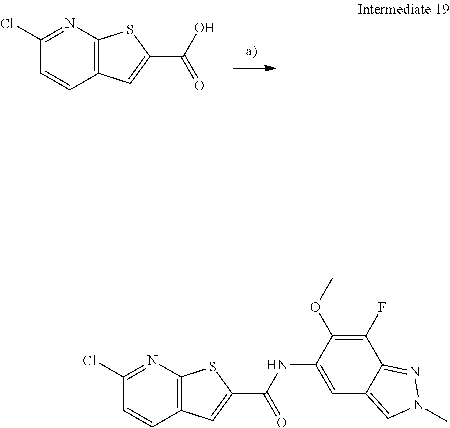 [SMILES: O=C(O)c1cc2ccc(Cl)nc2s1 ; COc1c(NC(=O)c2cc3ccc(Cl)nc3s2)cc2cn(C)nc2c1F]

To a mixture of 6-chlorothieno[2,3-b]pyridine-2-carboxylic acid (140 mg, 655.31 mol) and 7-fluoro-6-methoxy-2-methyl-indazol-5-amine (127.91 mg, 655.31 μmol) in DMF (3 mL) was added HATU (373.75 mg, 982.96 mol) and DIPEA (127.04 mg, 982.96 μmol, 171.21 L) in one portion at 25° C. The mixture was stirred at 25° C. for 90 mins and then concentrated in reduced pressure. The suspension was filtered and solid was collected, washed with EtOAc (50 mL×5). The residue was evacuated under vacuum to yield 6-chloro-N-(7-fluoro-6-methoxy-2-methyl-indazol-5-yl)thieno[2,3-b]pyridine-2-carboxamide (148.5 mg, 379.97 μmol, 57.98% yield) as a yellow solid. MS: m/z 391.0 [M+H]⁺.

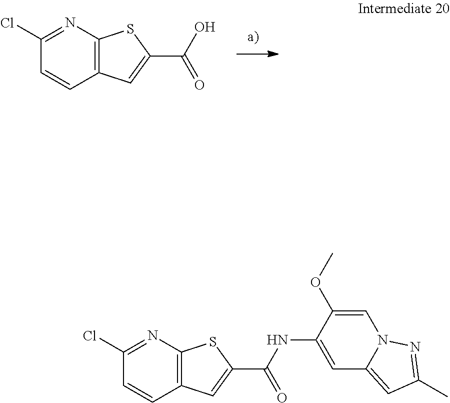 [SMILES: O=C(O)c1cc2ccc(Cl)nc2s1 ; COc1cn2nc(C)cc2cc1NC(=O)c1cc2ccc(Cl)nc2s1]

To a solution of 6-chlorothieno[2,3-b]pyridine-2-carboxylic acid (72.34 mg, 338.60 μmol) and 6-methoxy-2-methyl-pyrazolo[1,5-a]pyridin-5-amine (60 mg, 338.60 μmol) in pyridine (3 mL) was added T₄P (3 mL). The reaction was stirred at 20° C. for 2 h and then was quenched, filtered and concentrated under reduced pressure to give 6-chloro-N-(6-methoxy-2-methyl-pyrazolo[1,5-a]pyridin-5-yl)thieno[2,3-b]pyridine-2-carboxamide (80 mg, 132.93 μmol, 39.26% yield, 61.95% purity) as a yellow solid. MS: m/z 373.1 [M+H]⁺.

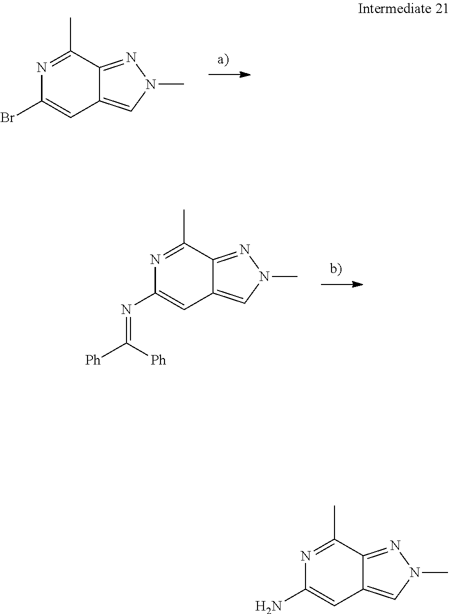 [SMILES: Cc1nc(Br)cc2cn(C)nc12 ; Cc1nc(N=C(c2ccccc2)c2ccccc2)cc2cn(C)nc12 ; Cc1nc(N)cc2cn(C)nc12]

Step a: 5-bromo-2,7-dimethyl-pyrazolo[3,4-c]pyridine (400 mg, 1.77 mmol), sodium;2-methylpropan-2-olate (340.08 mg, 3.54 mmol) and Pd-binap-G3 (175.52 mg, 176.93 μmol) was added in an microwave vial, evacuated under vacuum and refilled with N₂ three times. Diphenylmethanimine (384.79 mg, 2.12 mmol, 356.29 μL) and toluene (10 mL) were then added under N₂. The reaction mixture was stirred at 110° C. for 12 h. Then the reaction mixture was concentrated, and the residue was purified by column chromatography (0% to 100% EA/heptane) to give N-(2,7-dimethylpyrazolo[3,4-c]pyridin-5-yl)-1,1-diphenyl-methanimine (454.9 mg, 1.39 mmol, 78.77% yield). MS: m/z 327.1 [M+H]⁺.

Step b: To a mixture of N-(2,7-dimethylpyrazolo[3,4-c]pyridin-5-yl)-1,1-diphenyl-methanimine (454.9 mg mg, 1.39 mmol) in THF (4 mL) was added hydrochloric acid (4 M, 1.05 mL) at 20° C. The reaction was stirred at 20° C. for 1 h. The mixture was concentrated, then dioxane and toluene were added. The mixture was filtered and the precipitates were collected to give 2,7-dimethylpyrazolo[3,4-c]pyridin-5-amine (245.6 mg, 1.24 mmol, 88.71% yield, hydrochloride) as a pale yellow solid. MS: m/z 163.1 [M+H]⁺. ¹H NMR (400 MHz, DMSO-d₆) δ ppm 2.90 (s, 3H), 4.28 (s, 3H), 6.94 (s, 1H), 8.42 (s, 1H).

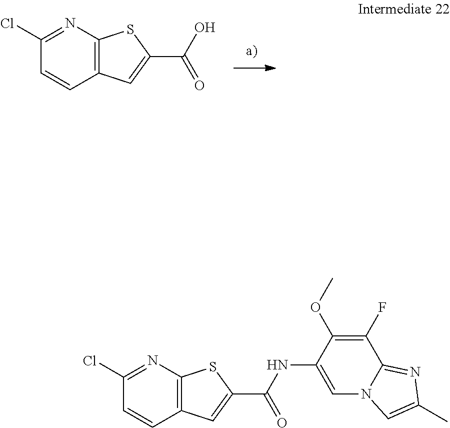 [SMILES: O=C(O)c1cc2ccc(Cl)nc2s1 ; COc1c(NC(=O)c2cc3ccc(Cl)nc3s2)cn2cc(C)nc2c1F]

To a solution of 6-chlorothieno[2,3-b]pyridine-2-carboxylic acid (200 mg, 936.15 mol) in DMF (10 mL) was added 8-fluoro-7-methoxy-2-methyl-imidazo[1,2-a]pyridin-6-amine (182.73 mg, 936.15 mol), DIPEA (362.97 mg, 2.81 mmol, 489.18 L) and HATU (427.14 mg, 1.12 mmol). The reaction mixture was stirred at 25° C. for 2 hours. The reaction mixture was diluted with H₂O (5 mL) and extracted with DCM (10 mL×3). The combined organic layers were washed with brine (10 mL), dried over Na₂SO₄, filtered and concentrated under reduced pressure. The residue was purified by flash silica gel chromatography (from PE/EtOAc=10/1 to 0/1) to yield 6-chloro-N-(8-fluoro-7-methoxy-2-methyl-imidazo[1,2-a]pyridin-6-yl)thieno[2,3-b]pyridine-2-carboxamide (350 mg, 888.39 μmol, 94.90% yield, 99.2% purity) as a yellow solid. MS: m/z 391.0 [M+H]⁺.

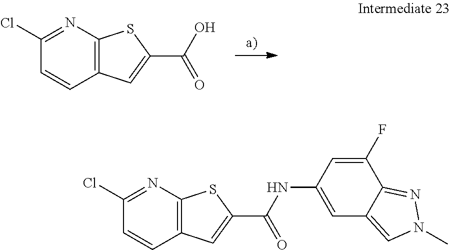 [SMILES: O=C(O)c1cc2ccc(Cl)nc2s1 ; Cn1cc2cc(NC(=O)c3cc4ccc(Cl)nc4s3)cc(F)c2n1]

7-fluoro-2-methyl-indazol-5-amine (83.34 mg, 504.59 μmol, Hydrochloride) and 6-chlorothieno[2,3-b]pyridine-2-carboxylic acid (100 mg, 458.71 μmol) were dissolved in dioxane (2 mL) before 2,4,6-tripropyl-1,3,5,2,4,6trioxatriphosphinane 2,4,6-trioxide (437.86 mg, 688.07 μmol, 409.60 μL, 50% purity) was added. The reaction mixture was stirred at 40° C. for 16 h and then concentrated under vacuum. The residue was purified by column flash chromatography (0% to 10% MeOH in DCM) to give 6-chloro-N-(7-fluoro-2-methyl-indazol-5-yl)thieno[2,3-b]pyridine-2-carboxamide (105.6 mg, 292.69 μmol, 63.81% yield) as a white solid. MS: m/z 361.1 [M+H]⁺.

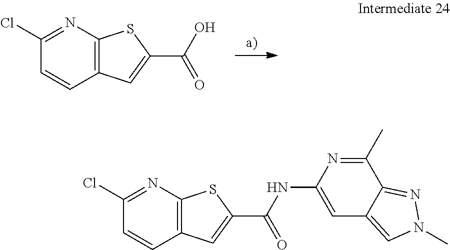 [SMILES: O=C(O)c1cc2ccc(Cl)nc2s1 ; Cc1nc(NC(=O)c2cc3ccc(Cl)nc3s2)cc2cn(C)nc12]

2,7-dimethylpyrazolo[3,4-c]pyridin-5-amine (111.58 mg, 561.69 μmol, Hydrochloride) and 6-chlorothieno[2,3-b]pyridine-2-carboxylic acid (100 mg, 468.08 μmol) were dissolved in dioxane (2 mL) before 2,4,6-tripropyl-1,3,5,2,4,6trioxatriphosphinane 2,4,6-trioxide (446.80 mg, 702.11 μmol, 417.96 μL, 50% purity) was added. The reaction mixture was stirred at 40° C. for 16 h and then concentrated under vacuum. The residue was purified by column flash chromatography (0% to 10% MeOH in DCM) to give 6-chloro-N-(2,7-dimethylpyrazolo[3,4-c]pyridin-5-yl)thieno[2,3-b]pyridine-2-carboxamide (138.7 mg, 387.63 μmol, 82.81% yield) as a white solid. MS: m/z 358.1 [M+H]+

### Section 3. Synthetic Processes to Prepare Compounds of the Disclosure

### Example 1—Compound 2

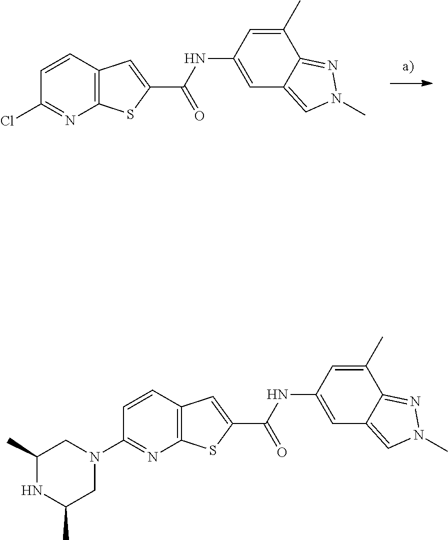

6-Chloro-N-(2,7-dimethylindazol-5-yl)thieno[2,3-b]pyridine-2-carboxamide (25.78 mg, 69.36 μmol, 1.0 eq.) was dissolved in dioxane (346.80 μL) and potassium tert-butoxide (31.13 mg, 277.44 mol) was added. (2R,6S)-2,6-dimethylpiperazine (11.9 mg, 104.04 μmol, 1.5 eq.) was then added to the solution, which was then heated at 120° C. for 6 hours before being concentrated then taken back up in DMSO, filtered, and purified via preparative HPLC (Column: Sunfire C18 100×19 mm, 5 mm; Mobile phase A: MeCN; Mobile phase B: H₂O; Modifier: 0.1% TFA) to obtain N-(2,7-dimethylindazol-5-yl)-6-[(3S,5R)-3,5-dimethylpiperazin-1-yl]thieno[2,3-b]pyridine-2-carboxamide (9.7 mg, 25% yield) as an orange oil. MS: m/z 435.3 [M+H]⁺; RT: 1.17 min (Method 3). ¹HNMR (600 MHz, DMSO-d₆) δ ppm 1.19 (br dd, J=11.25, 6.68 Hz, 2H) 1.29-1.34 (m, 6H) 2.52-2.55 (m, 3H) 2.83-2.89 (m, 2H) 4.11-4.19 (m, 3H) 4.58-4.65 (m, 2H) 7.24-7.27 (m, 1H) 7.96-8.00 (m, 1H) 8.13-8.19 (m, 2H) 8.25-8.29 (m, 1H) 8.52-8.59 (m, 1H) 9.11-9.17 (m, 1H) 10.22-10.25 (m, 1H).

Using the procedure described for Example 1 above, additional compounds described herein were prepared by substituting the appropriate amine starting material in step a, suitable reagents and reaction conditions, obtaining compounds such as those selected from:

| Cpd ID | Data |
|---|---|
| 4 | Starting material: N,N-dimethylpyrrolidin-3-amine |
| | MS: m/z 435.3 [M + H]⁺; RT 1.08 min (Method 3) |
| | ¹H NMR (400 MHz, DMSO-d₆) δ ppm 2.17-2.27 (m, 2 H) 2.48-2.49 (m, |
| | 3 H) 2.84-2.96 (m, 6 H) 3.46-3.53 (m, 2 H) 3.64-3.66 (m, 1 H) 3.73-3.78 |
| | (m, 2 H) 4.14-4.16 (m, 3 H) 6.73-6.77 (m, 1 H) 7.96-7.98 (m, 1 H) 8.10- |
| | 8.13 (m, 1 H) 8.13-8.15 (m, 1 H) 8.24-8.28 (m, 1 H) 9.97-10.05 (m, 1 |
| | H) 10.16-10.21 (m, 1 H) |

### Example 2—Compound 1

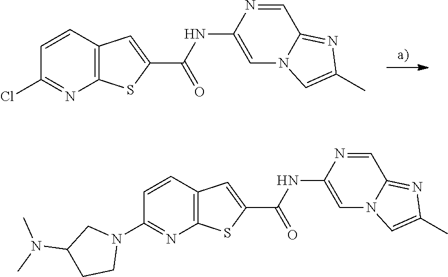

N,N-Dimethylpyrrolidin-3-amine (42.64 mg, 373.39 μmol, 2.0 eq.), 6-chloro-N-(2-methylimidazo[1,2-a]pyridin-6-yl)thieno[2,3-b]pyridine-2-carboxamide (100 mg, 186.70 μmol, 1.0 eq.), and DIPEA (560.09 μmol, 97 μL, 3.0 eq.) were dissolved in dioxane (933 μL, 0.2 M) and heated to 80° C. for 16 hours before it was concentrated then taken back up in a minimal amount of DMSO, filtered, and submitted to reverse phase HPLC purification (Column: Sunfire C18 100×19 mm, 5 mm; Mobile phase A: MeCN; Mobile phase B: H₂O; Modifier: 0.1% TFA) to obtain 6-[3-(dimethylamino)pyrrolidin-1-yl]-N-(2-methylimidazo[1,2-a]pyridin-6-yl)thieno[2,3-b]pyridine-2-carboxamide (29.5 mg, 30% yield) as a brown oil. MS: m/z 421.1 [M+H]⁺; RT 0.87 min (Method 3). 1H NMR (600 MHz, DMSO-d6) δ ppm 2.20-2.27 (m, 1H) 2.44-2.49 (m, 3H) 2.83-2.97 (m, 6H) 3.47-3.53 (m, 2H) 3.63-3.69 (m, 1H) 3.75-3.81 (m, 1H) 3.97-4.06 (m, 2H) 6.75-6.84 (m, 1H) 7.89-8.02 (m, 1H) 8.14-8.24 (m, 2H) 9.50-9.57 (m, 1H) 9.94-10.11 (m, 1H) 10.77-10.86 (m, 1H).

Using the procedure described for Example 2 above, additional compounds described herein were prepared by substituting the appropriate amine starting material in step a, suitable reagents and reaction conditions, obtaining compounds such as those selected from:

| Cpd ID | Data |
|---|---|
| 11 | Starting material: N,N-dimethylpiperidin-4-amine |
| | MS: m/z 433.0 [M − H]⁻; RT 0.80 min (Method 5) |
| | ¹H NMR (400 MHz, DMSO-d6) δ ppm 1.30-1.45 (m, 2 H) 1.86 (d, J = 11.01 |
| | Hz, 2 H) 2.20 (s, 6 H) 2.32 (s, 3 H) 2.35-2.42 (m, 1 H) 2.90-3.01 (m, 2 H) |
| | 4.43 (d, J = 13.01 Hz, 2 H) 7.07 (d, J = 9.51 Hz, 1 H) 7.30-7.40 (m, 1 H) 7.42- |
| | 7.51 (m, 1 H) 7.75 (s, 1 H) 8.06 (d, J = 9.01 Hz, 1 H) 8.10 (s, 1 H) 9.13 (m, 1 |
| | H) 10.36 (s, 1 H) |
| 12 | Starting material: hexahydro-1H-pyrrolizin-2-amine |
| | MS: m/z 433.2 [M + H]⁺; RT 0.58 min (Method 5) |
| | ¹H NMR (400 MHz, METHANOL-d4) δ ppm 1.78-1.89 (m, 2 H) 1.94-2.05 |
| | (m, 1 H) 2.19-2.30 (m, 1 H) 2.37 (m, 1 H) 2.46-2.59 (m, 5 H) 3.11-3.22 (m, |
| | 1 H) 3.33-3.37 (m, 1 H) 3.83-3.98 (m, 2 H) 4.36 (m, 1 H) 4.61-4.70 (m, 1 |
| | H) 6.77 (d, J = 9.01 Hz, 1 H) 7.82 (d, J = 9.51 Hz, 1 H) 7.98 (s, 1 H) 8.00-8.07 |
| | (m, 3 H) 9.54 (s, 1 H) |
| 13 | Starting material: (3aS,6aR)-2-methyloctahydrocyclopenta[c]pyrrol-4-amine |
| | MS: m/z 445.2 [M − H]⁻; RT 1.64 min (Method 5) |
| | ¹H NMR (400 MHz, METHANOL-d4) δ ppm 1.44-1.59 (m, 1 H) 1.62-1.75 |
| | (m, 1 H) 1.86-2.00 (m, 1 H) 2.11-2.33 (m, 3 H) 2.54 (d, J = 1.00 Hz, 3 H) |
| | 2.74-2.85 (m, 2 H) 2.97 (s, 3 H) 3.47-3.57 (m, 1 H) 3.74-3.86 (m, 1 H) 3.97- |
| | 4.13 (m, 1 H) 4.17-4.30 (m, 1 H) 6.63 (d, J = 9.01 Hz, 1 H) 7.80-7.86 (m, 2 |
| | H) 7.97 (s, 1 H) 7.98 (s, 1 H) 8.01-8.04 (m, 1 H) 9.53 (s, 1 H) |
| 14 | Starting material: octahydroindolizin-7-amine |
| | MS: m/z 447.2 [M + H]⁺; RT 0.80 min (Method 5) |
| | ¹H NMR (400 MHz, METHANOL-d₄) δ ppm 1.51-1.64 (m, 1 H) 1.69-1.83 |
| | (m, 2 H) 2.03-2.37 (m, 7 H) 2.44 (d, J = 13.51 Hz, 1 H) 2.54 (s, 3 H) 3.09 (d, |
| | J = 9.51 Hz, 1 H) 3.15-3.21 (m, 1 H) 3.58-3.69 (m, 2 H) 3.71-3.79 (m, 1 H) |
| | 6.64 (d, J = 9.01 Hz, 1 H) 7.82 (d, J = 9.51 Hz, 1 H) 7.87 (d, J = 9.01 Hz, 1 H) |
| | 7.97 (s, 1 H) 7.98 (s, 1 H) 8.02 (m, 1 H) 9.53 (s, 1 H) |
| 15 | Starting material: 4-methyloctahydro-2H-pyrido[4,3-b][1,4]oxazine |
| | MS: m/z 461.0 [M − H]⁻; RT 0.79 min (Method 5) |
| | ¹H NMR (600 MHz, DMSO-d₆) δ ppm 1.68-1.83 (m, 1 H) 2.46 (s, 3 H) 2.84- |
| | 3.09 (m, 4 H) 3.10-3.22 (m, 2 H) 3.50 (s, 3 H) 3.68-3.81 (m, 1 H) 3.87- |
| | 4.35 (m, 4 H) 7.18 (d, J = 10.49 Hz, 1 H) 7.91 (d, J = 9.71 Hz, 1 H) 7.96-8.01 |
| | (m, 1 H) 8.15-8.18 (m, 2 H) 8.19 (s, 1 H) 9.54 (s, 1 H) 10.84 (s, 1 H) |
| 16 | Starting material: 6-methyloctahydro-1H-pyrrolo[2,3-c]pyridine |
| | MS: m/z 445.0 [M − H]⁻; RT 0.69 min (Method 5) |
| | ¹H NMR (600 MHz, DMSO-d₆) δ ppm 1.93-2.03 (m, 2 H) 2.06-2.16 (m, 1 |
| | H) 2.22-2.31 (m, 1 H) 2.46 (s, 3 H) 2.76-2.87 (m, 5 H) 3.05-3.17 (m, 2 H) |
| | 3.59 (m, 2 H) 3.69-3.77 (m, 1 H) 6.70 (d, J = 9.02 Hz, 1 H) 7.92 (d, J = 9.62 |
| | Hz, 1 H) 7.95-8.07 (m, 2 H) 8.13 (d, J = 8.93 Hz, 1 H) 8.18 (s, 2 H) 9.35-9.49 |
| | (m, 1 H) 9.53 (s, 1 H) 10.82 (s, 1 H) |
| 17 | Starting material: 4-(3,3-dimethylazetidin-1-yl)piperidine |
| | MS: m/z 475.2 [M + H]⁺; RT 0.84 min (Method 5) |
| | ¹H NMR (600 MHz, DMSO-d₆) δ ppm 1.21 (s, 3 H) 1.30 (s, 5 H) 1.99 (d, J = |
| | 10.84 Hz, 2 H) 2.46 (s, 3 H) 2.91 (m, 2 H) 3.76-3.82 (m, 2 H) 3.93 (m, 2 H) |
| | 4.55 (d, J = 13.09 Hz, 2 H) 7.00 (s, 1 H) 7.09 (s, 1 H) 7.14 (d, J = 9.19 Hz, 1 H) |
| | 7.17 (s, 1 H) 7.91 (d, J = 9.62 Hz, 1 H) 7.98 (d, J = 9.45 Hz, 1 H) 8.13 (d, J = |
| | 9.19 Hz, 1 H) 8.15-8.19 (m, 2 H) 9.53 (s, 1 H) 9.81 (s, 1 H) 10.82 (s, 1 H) |
| 18 | Starting material: 1,2,6-trimethylpiperazine |
| | MS: m/z 435.2 [M + H]⁺; RT 0.59 min (Method 5) |
| | ¹H NMR (600 MHz, DMSO-d₆) δ ppm 1.39 (d, J = 6.24 Hz, 6 H) 2.45 (s, 3 H) |
| | 2.68 (s, 1 H) 2.85 (s, 3 H) 2.98-3.09 (m, 2 H) 3.13-3.21 (m, 1 H) 4.62 (m, 2 |
| | H) 7.23 (m, 1 H) 7.85-7.92 (m, 1 H) 7.94-8.03 (m, 1 H) 8.15 (s, 1 H) 8.19- |
| | 8.23 (m, 2 H) 9.47 (m, 1 H) 9.51 (s, 1 H) 10.86 (s, 1 H) |
| 19 | Starting material: 1,3′-bipyrrolidine |
| | MS: m/z 407.2 [M + H]⁺; RT 0.54 min (Method 5) |
| 20 | Starting material: 3-methyl-3,9-diazaspiro[5.5]undecane |
| | MS: m/z 473.2 [M − H]⁻; RT 0.75 min (Method 5) |
| | ¹H NMR (600 MHz, DMSO-d₆) δ ppm 1.43 (d, J = 4.77 Hz, 2 H) 1.47-1.54 |
| | (m, 2 H) 1.67 (d, J = 5.46 Hz, 2 H) 1.89 (d, J = 14.22 Hz, 2 H) 2.46 (s, 3 H) |
| | 2.78 (d, J = 4.33 Hz, 3 H) 3.04-3.13 (m, 2 H) 3.26 (s, 2 H) 3.66 (s, 4 H) 7.08 |
| | (d, J = 9.19 Hz, 1 H) 7.91 (d, J = 9.62 Hz, 1 H) 8.00 (d, J = 9.62 Hz, 1 H) 8.07 |
| | (J = 9.19 Hz, 1 H) 8.15 (s, 1 H) 8.17 (s, 1 H) 9.21 (s, 1 H) 9.54 (s, 1 H) 10.79 |
| | (s, 1 H) |
| 21 | Starting material: cis-2-methyloctahydropyrrolo[3,4-c]pyrrole |
| | MS: m/z 431.0 [M − H]⁻; RT 0.75 min (Method 5) |
| | ¹H NMR (600 MHz, DMSO-d₆) δ ppm 2.46 (s, 3 H) 2.82 (s, 1 H) 2.86 (d, J = |
| | 2.95 Hz, 1 H) 2.92 (d, J = 7.02 Hz, 1 H) 3.12 (s, 1 H) 3.32 (s, 3 H) 3.59 (d, |
| | J = 11.70 Hz, 3 H) 3.68-3.71 (m, 1 H) 3.74 (d, J = 11.01 Hz, 1 H) 3.84 (s, 1 H) |
| | 6.71-6.83 (m, 1 H) 7.93 (d, J = 9.54 Hz, 1 H) 8.01 (m, 1 H) 8.12 (d, J = 9.02 |
| | Hz, 1 H) 8.18 (s, 2 H) 9.54 (s, 1 H) 9.73-9.98 (m, 1 H) 10.81 (s, 1 H) |
| 22 | Starting material: N,N-dimethylazetidin-3-amine |
| | MS: m/z 407.2 [M + H]⁺; RT 0.54 min (Method 5) |
| | ¹H NMR (400 MHz, METHANOL-d₄) δ ppm 2.54 (s, 3 H) 2.96 (s, 6 H) 4.21- |
| | 4.35 (m, 3 H) 4.43-4.55 (m, 2 H) 6.65 (d, J = 9.01 Hz, 1 H) 7.83 (d, J = 9.51 |
| | Hz, 1 H) 7.98 (s, 1 H) 8.01 (d, J = 2.00 Hz, 1 H) 8.03-8.05 (m, 1 H) 8.08 (d, J = |
| | 9.01 Hz, 1 H) 9.54 (s, 1 H) |
| 23 | Starting material: N1,N1,N2-trimethylethane-1,2-diamine |
| | MS: m/z 409.2 [M + H]⁺; RT 0.79 min (Method 5) |
| | ¹H NMR (600 MHz, DMSO-d6) δ ppm 2.46 (s, 3 H) 2.88 (s, 6 H) 3.10 (s, 3 H) |
| | 3.98 (t, J = 6.59 Hz, 2 H) 6.92 (d, J = 9.10 Hz, 1 H) 7.90 (d, J = 9.54 Hz, 1 H) |
| | 7.94-8.04 (m, 1 H) 8.15 (d, J = 9.02 Hz, 1 H) 8.16 (s, 1 H) 8.19 (s, 1 H) 9.25- |
| | 9.40 (m, 1 H) 9.54 (s, 1 H) 10.81 (s, 1 H) |
| 24 | Starting material: N,N-dimethyl-3-azabicyclo[3.1.0]hexan-6-amine |
| | MS: m/z 431.2 [M]⁻; RT 0.84 min (Method 5) |
| | 1H NMR (600 MHz, DMSO-d₆) δ ppm 2.32-2.36 (m, 2 H) 2.45-2.48 (m, 3 |
| | H) 2.85-2.91 (m, 6 H) 3.53-3.58 (m, 2 H) 3.86-3.93 (m, 2 H) 6.71-6.77 (m, |
| | 1 H) 7.89-7.95 (m, 1 H) 7.95-8.01 (m, 1 H) 8.09-8.14 (m, 1 H) 8.17-8.22 |
| | (m, 1 H) 9.52-9.58 (m, 1 H) 10.78-10.83 (m, 1 H) |
| 27 | Starting material: 8-methyl-3,8-diazabicyclo[3.2.1]octane |
| | MS: m/z 431.2 [M − H]⁻; RT 0.78 min (Method 5) |
| 28 | Starting material: cis-5-methyloctahydro-1H-pyrrolo[3,2-c]pyridine |
| | MS: m/z 445.2 [M − H]⁻; RT 0.78 min (Method 5) |
| | ¹H NMR (400 MHz, METHANOL-d₄) δ ppm 2.21-2.39 (m, 2 H) 2.54 (m, 3 |
| | H) 2.61-2.68 (m, 1 H) 2.70-2.79 (m, 1 H) 2.93 (s, 3 H) 3.11-3.20 (m, 1 H) |
| | 3.38-3.61 (m, 4 H) 3.66-3.79 (m, 2 H) 4.41-4.53 (m, 1 H) 6.69 (d, J = 8.51 |
| | Hz, 1 H) 7.82 (d, J = 10.01 Hz, 1 H) 7.97-8.04 (m, 4 H) 9.53 (s, 1 H) |
| 29 | Starting material: 1-((3aR,6aS)-hexahydropyrrolo[3,4-c]pyrrol-2(1H)- |
| | yl)ethan-1-one |
| | MS: m/z 461.2 [M + H]⁺; RT 0.92 min (Method 5) |
| 30 | Starting material: cis-1-methyloctahydro-1H-pyrrolo[2,3-c]pyridine |
| | MS: m/z 445.2 [M − H]⁻; RT 0.79 min (Method 5) |
| 31 | Starting material: 1-methyl-2,3,4,5-tetrahydro-1H-pyrido[2,3-e][1,4]diazepine |
| | MS: m/z 470.2 [M + H]⁺; RT 0.63 min (Method 5) |
| 32 | Starting material: N,N-dimethyloctahydro-1H-isoindol-5-amine |
| | MS: m/z 473.2 [M − H]⁻; RT 0.85 min (Method 5) |
| | ¹H NMR (400 MHz, METHANOL-d4) δ ppm 1.40-1.49 (m, 1 H) 1.54-1.67 |
| | (m, 1 H) 1.90-2.00 (m, 2 H) 2.04-2.15 (m, 2 H) 2.23 (d, J = 11.51 Hz, 1 H) |
| | 2.34 (m, 1 H) 2.54 (m, 3 H) 2.84 (s, 3 H) 2.89 (s, 3 H) 2.97 (d, J = 5.00 Hz, 1 |
| | H) 3.47-3.56 (m, 2 H) 3.61-3.76 (m, 2 H) 6.67 (d, J = 9.01 Hz, 1 H) 7.82 (d, |
| | J = 9.51 Hz, 1 H) 7.95-8.04 (m, 4 H) 9.53 (s, 1 H) |
| 33 | Starting material: cis-2,6-dimethyl-1-(2,2,2-trifluoroethyl)piperazine |
| | MS: m/z 503.0 [M + H]⁺; RT 1.27 min (Method 5) |
| 34 | Starting material: (1R,4R)-2-isopropyl-2,5-diazabicyclo[2.2.1]heptane |
| | MS: m/z 448.2 [M + H]⁺; RT 0.78 min (Method 5) |
| 35 | Starting material: 3-(3,3-dimethylazetidin-1-yl)pyrrolidine |
| | MS: m/z 462.2 [M + H]⁺; RT 0.78 min (Method 5) |
| 36 | Starting material: 1-methyldecahydro-1,6-naphthyridine |
| | MS: m/z 462.2 [M + H]⁺; RT 0.79 min (Method 5) |
| 37 | Starting material: 4-methyl-1-oxa-4,9-diazaspiro[5.5]undecane |
| | MS: m/z 478.2 [M + H]⁺; RT 0.81 min (Method 5) |
| 38 | Starting material: N-methyl-1-azabicyclo[2.2.1]heptan-3-amine |
| | MS: m/z 448.2 [M + H]⁺; RT 0.78 min (Method 5) |
| 39 | Starting material: 4-(pyrrolidin-1-ylmethyl)aniline |
| | MS: m/z 483.0 [M + H]⁺; RT 0.65 min (Method 5) |
| 40 | Starting material: 1-methyl-1,4-diazepane |
| | MS: m/z 422.2 [M + H]⁺; RT 0.75 min (Method 5) |
| 41 | Starting material: N1,N1,2,2-tetramethylpropane-1,3-diamine |
| | MS: m/z 438.2 [M + H]⁺; RT 0.70 min (Method 5) |
| 42 | Starting material: 3-(3,3-dimethylazetidin-1-yl)aniline |
| | MS: m/z 483.2 [M + H]⁺; RT 1.21 min (Method 5) |
| 43 | Starting material: cis-1-methyloctahydropyrrolo[3,4-b]pyrrole |
| | MS: m/z 434.2 [M + H]⁺; RT 0.67 min (Method 5) |
| 44 | Starting material: 4-(3,3-dimethylazetidin-1-yl)aniline |
| | MS: m/z 483.2 [M + H]⁺; RT 1.01 min (Method 5) |
| 45 | Starting material: 3-methyloctahydropyrrolo[1,2-a]pyrazine |
| | MS: m/z 447.2 [M + H]⁺; RT 0.88 min (Method 5) |
| 46 | Starting material: 2-ethylisoindolin-4-amine |
| | MS: m/z 469.0 [M + H]⁺; RT 0.65 min (Method 5) |
| 47 | Starting material: 1,4-dimethyl-2,3,4,5-tetrahydro-1H-benzo[e][1,4]diazepin- |
| | 8-amine |
| | MS: m/z 498.2 [M + H]⁺; RT 0.87 min (Method 5) |
| 48 | Starting material: 1-(azetidin-2-yl)-N,N-dimethylmethanamine |
| | MS: m/z 421.2 [M + H]⁺; RT 0.58 min (Method 5) |

### Example 3—Compound 3

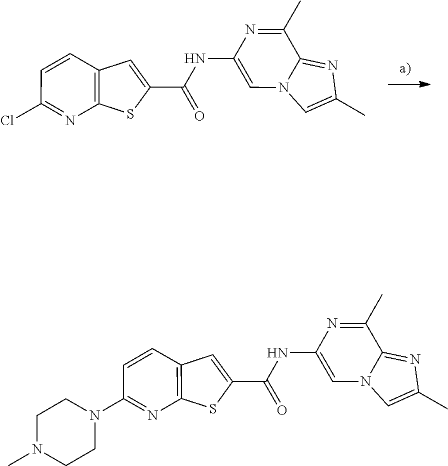

6-Chloro-N-(2,8-dimethylimidazo[1,2-a]pyrazin-6-yl)thieno[2,3-b]pyridine-2-carboxamide (22.90 mg, 56.95 μmol, 1.0 eq.) was dissolved in dioxane (1.17 mL, 0.05 M) before 1-methylpiperazine (14.26 mg, 142.3 μmol, 2.5 eq.), potassium tert-butoxide (25.56 mg, 227.8 μmol), and DIPEA (85.4 μmol, 15 L) were added. The solution was at 120° C. for 72 hours 10 before it was concentrated then taken back up in DMSO, filtered, and purified via reversed phase HPLC purification (Column: Sunfire C18 100×19 mm, 5 mm; Mobile phase A: MeCN; Mobile phase B: H₂O; Modifier: 0.1% TFA) to obtain N-(2,8-dimethylimidazo[1,2-a]pyrazin-6-yl)-6-(4-methylpiperazin-1-yl)thieno[2,3-b]pyridine-2-carboxamide (10.4 mg, 34% yield) as an orange oil. MS: m/z 422.2 [M+H]⁺; RT 0.88 min (Method 3). ¹H NMR (400 MHz, DMSO-d₆) δ ppm 2.36-2.42 (m, 3H) 2.42-2.46 (m, 3H) 2.52-2.57 (m, 2H) 2.71-2.78 (m, 3H) 2.83-2.88 (m, 2H) 3.13-3.20 (m, 3H) 3.53-3.58 (m, 2H) 4.53-4.60 (m, 2H) 7.14-7.18 (m, 1H) 7.95-8.00 (m, 1H) 8.12-8.16 (m, 1H) 8.38-8.41 (m, 1H) 9.10-9.13 (m, 1H) 9.82-9.90 (m, 1H) 10.99-11.04 (m, 1H).

Using the procedure described for Example 3 above, additional compounds described herein were prepared by substituting the appropriate amine starting material in step a, suitable reagents and reaction conditions, obtaining compounds such as those selected from:

| Cpd ID | Data |
|---|---|
| 5 | Starting material: N,N-dimethylpyrrolidin-3-amine |
| | MS: m/z 436.2 [M + H]⁺; RT 0.86 min (Method 3) |
| | 1H NMR (600 MHz, DMSO-d6) δ ppm 2.22-2.27 (m, 1 H) 2.38-2.42 (m, |
| | 3 H) 2.54-2.54 (m, 3 H) 2.87-2.92 (m, 6 H) 3.50-3.52 (m, 2 H) 3.66 (br |
| | d, J = 4.58 Hz, 1 H) 3.76-3.79 (m, 1 H) 3.97-4.03 (m, 2 H) 6.73-6.77 (m, |
| | 1 H) 7.97-8.00 (m, 1 H) 8.06-8.09 (m, 1 H) 8.35-8.41 (m, 1 H) 9.10-9.16 |
| | (m, 1 H) 10.04-10.12 (m, 1 H) 10.94-10.99 (m, 1 H) |
| 57 | Starting material: pyrrolidine |
| | MS: m/z 393.2 [M + H]⁺; RT 0.52 min (Method 3) |
| 240 | Starting materials: 1-ethylpiperazine |
| | MS: m/z. 436.3 [M + H]⁺; RT 0.43 min (Method 4) |
| 233 | Starting materials: 1-isopropylpiperazine |
| | MS: m/z. 449.2 [M + H]⁺; RT 2.17 min (Method 1) |
| | 1H NMR (600 MHz, DMSO-d6) δ ppm 1.28-1.30 (m, 6 H) 2.40-2.42 (m, |
| | 3 H) 2.73-2.75 (m, 3 H) 3.09-3.16 (m, 2 H) 3.23-3.30 (m, 2 H) 3.52-3.57 |
| | (m, 3 H) 4.60-4.64 (m, 2 H) 7.16 (d, J = 9.16 Hz, 1 H) 8.00-8.03 (m, 1 H) |
| | 8.12-8.16 (m, 1 H) 8.38-8.40 (m, 1 H) 9.14-9.18 (m, 1 H) |
| 208 | Starting materials: (5R)-1-methyl-1,7-diazaspiro[4.4]nonane |
| | MS: m/z. 462.2 [M + H]⁺; RT 0.45 min (Method 4) |
| 243 | Starting materials: 8-methyl-3,8-diazabicyclo[3.2.1]octane |
| | MS: m/z. 447.0 [M + H]⁺; RT 0.9 min (Method 1) |
| | 1H NMR (400 MHz, METHANOL-d4) δ ppm 2.05-2.13 (m, 2 H) 2.32- |
| | 2.41 (m, 2 H) 2.58-2.62 (m, 3 H) 2.85-2.89 (m, 3 H) 2.90-2.96 (m, 3 H) |
| | 3.40-3.46 (m, 2 H) 4.10-4.18 (m, 2 H) 4.45-4.54 (m, 2 H) 7.00-7.06 (m, |
| | 1 H) 8.07-8.14 (m, 3 H) 9.39-9.42 (m, 1 H) |
| 227 | Starting materials: (3R)-N,N,3-trimethylpyrrolidin-3-amine |
| | MS: m/z. 449.2 [M + H]⁺; RT 1.0 min (Method 1) |
| 226 | Starting materials: 2-methyl-2,7-diazaspiro[3.4]octane |
| | MS: m/z 447.2 [M + H]⁺; RT 1.05 min (Method 3) |
| 228 | Starting materials: (3S)-3-(azetidin-1-yl)pyrrolidine |
| | MS: m/z. 448.2 [M + H]⁺; RT 0.42 min (Method 4) |
| 218 | Starting materials: (1S,5S)-3-methyl-3,6-diazabicyclo[3.2.0]heptane |
| | MS: m/z. 434.3 [M + H]⁺; RT 0.41 min (Method 4) |
| 214 | Starting materials: (3R,4S)-4-fluoro-1-methyl-pyrrolidin-3-amine |
| | MS: m/z 440.1 [M + H]⁺; RT 0.88 min (Method 3) |
| 194 | Starting materials: rac-(3aR,6aR)-5-methyl-2,3,3a,4,6,6a-hexahydro-1H- |
| | pyrrolo[3,4-b]pyrrole |
| | MS: m/z. 448.2 [M + H]⁺; RT 0.44 min (Method 3) |
| 211 | Starting materials: (7S)-N,N-dimethyl-5-azaspiro[2.4]heptan-7-amine |
| | MS: m/z 462.1 [M + H]⁺; RT 0.98 min (Method 3) |
| | ¹H NMR (600 MHz, DMSO-d6) δ ppm 0.82-0.91 (m, 2 H) 1.03-1.09 (m, |
| | 1 H) 1.36-1.42 (m, 1 H) 2.38-2.43 (m, 3 H) 2.72-2.76 (m, 3 H) 2.77-2.81 |
| | (m, 3 H) 2.84-2.89 (m, 3 H) 3.31-3.35 (m, 1 H) 3.72-3.75 (m, 1 H) 3.82- |
| | 3.85 (m, 1 H) 3.90-3.95 (m, 1 H) 4.26-4.31 (m, 1 H) 6.68-6.75 (m, 1 H) |
| | 7.97-8.02 (m, 1 H) 8.07-8.11 (m, 1 H) 8.37-8.41 (m, 1 H) 9.11-9.17 (m, |
| | 1 H) 9.34-9.44 (m, 1 H) 10.95-11.02 (m, 1 H). |
| 219 | Starting materials: tert-butyl (R)-methyl(pyrrolidin-3-yl)carbamate |
| | MS: m/z 422.1. [M + H]⁺; RT 1.52 min (Method 1) |
| 193 | Starting materials: 4,7-diazaspiro[2.5]octane |
| | MS: m/z 434.1 [M + H]⁺; RT 0.96 min (Method 3) |
| 195 | Starting materials: 1-(trideuteriomethyl)piperazine |
| | MS: m/z 425.2 [M + H]⁺; RT 0.90 min (Method 3) |

### Example 4—Compound 6

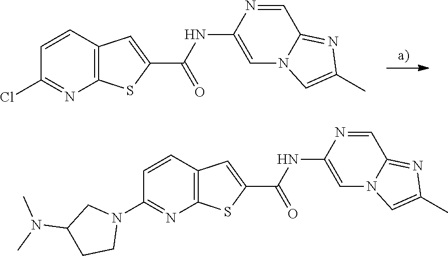

N,N-Dimethylpyrrolidin-3-amine (26.68 mg, 142.59 μmol, 2Hydrochloride, 2.0 eq.) and 6-chloro-N-(2-methylimidazo[1,2-a]pyrazin-6-yl)thieno[2,3-b]pyridine-2-carboxamide (25.8 mg, 71.29 μmol, 1.0 eq.) were dissolved in dioxane (356 μL, 0.2 M) before DIPEA (178.2 μmol, 31 μL, 2.5 eq.) was added. The solution was then raised to 120° C. for 72 hours before it was concentrated, taken back up in DMSO, filtered, and purified via reversed phase HPLC purification (Column: Sunfire C18 100×19 mm, 5 mm; Mobile phase A: MeCN; Mobile phase B: H₂O; Modifier: 0.1% IFA) to obtain 6-[(3R)-3-(dimethylamino)pyrrolidin-1-yl]-N-(2-methylimidazo[1,2-a]pyrazin-6-yl)thieno[2,3-b]pyridine-2-carboxamide (10.2 mg, 26% yield) as an orange oil. MS: m/z 422.2 [M+H]⁺; RT 0.82 min (Method 3); ¹H NMR (400 MHz, DMSO-d₆) δ ppm 2.38-2.44 (m, 3H) 2.84-2.96 (m, 6H) 3.66 (br d, J=4.50 Hz, 2H) 3.73-3.82 (m, 3H) 3.98-4.07 (m, 2H) 6.74-6.79 (m, 1H) 8.01-8.05 (m, 1H) 8.07-8.12 (m, 1H) 8.34-8.38 (m, 1H) 8.86-8.89 (m, 1H) 9.25-9.28 (m, 1H) 10.01-10.11 (m, 1H) 11.01-11.07 (m, 1H).

### Example 5—Compound 66

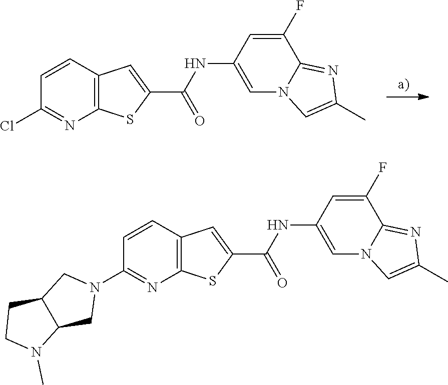 [SMILES: Cc1cn2cc(NC(=O)c3cc4ccc(Cl)nc4s3)cc(F)c2n1 ; Cc1cn2cc(NC(=O)c3cc4ccc(N5C[C@@H]6CCN(C)[C@@H]6C5)nc4s3)cc(F)c2n1]

To a solution 6-chloro-N-(8-fluoro-2-methyl-imidazo[1,2-a]pyridin-6-yl)thieno[2,3-b]pyridine-2-carboxamide (15 mg, 41.58 μmol) and (3aS,6aS)-1-methyl-3,3a,4,5,6,6a-hexahydro-2H-pyrrolo[2,3-c]pyrrole (10.49 mg, 83.15 mol) in Dioxane (2 mL) was added TEA (124.73 μmol, 17 μL). The reaction mixture was stirred at 90° C. for 12 h. It was filtered and concentrated under reduced pressure to give a residue, which was purified by prep-HPLC (HCl condition) to give 6-[(3aS,6aS)-1-methyl-2,3,3a,4,6,6a-hexahydropyrrolo[3,4-b]pyrrol-5-yl]-N-(8-fluoro-2-methyl-imidazo[1,2-a]pyridin-6-yl)thieno[2,3-b]pyridine-2-carboxamide (5.3 mg, 28% yield) as a yellow solid. MS: m/z 451.0 [M+H]⁺; RT 1.53 min (Method 7).

Using the procedure described for Example 5 above, additional compounds described herein were prepared by substituting the appropriate amine and amide starting materials in step a, suitable reagents and reaction conditions, obtaining compounds such as those selected from:

| Cpd ID | Data |
|---|---|
| 70 | Starting material: (3aR,6aR)-1-methyl-3,3a,4,5,6,6a-hexahydro-2H- |
| | pyrrolo[2,3-c]pyrrole |
| | MS: m/z 451.2 [M + H]⁺; RT 0.25 min (Method 7) |
| 69 | Starting material: (2R,6S)-1,2,6-trimethylpiperazine |
| | MS: m/z 453.0 [M + H]⁺; RT 1.62 min (Method 7) |
| 79 | Starting material: 1-cyclopropylpiperazine |
| | MS: m/z 451.0 [M + H]⁺; RT 0.26 min (Method 7) |
| | ¹H NMR (400 MHz, METHANOL-d₄) δ = 9.43 (d, J = 1.2 Hz, 1H), 8.15- |
| | 8.13 (m, 2H), 8.01-7.96 (m, 1H), 7.99 (dd, J = 1.6, 11.6 Hz, 1H), 7.14 (d, |
| | J = 8.8 Hz, 1H), 4.83-4.68 (m, 2H), 3.82-3.48 (m, 6H), 2.96-2.92 (m, 1H), |
| | 2.58 (d, J = 0.8 Hz, 3H), 1.11-1.03 (m, 4H) |
| 77 | Starting material: 1-methylpiperazine |
| | MS: m/z 425.0 [M + H]⁺; RT 0.25 min (Method 7) |
| | ¹H NMR: (400 MHz, METHANOL-d₄) δ ppm = 9.43 (d, J = 1.2 Hz, 1H), |
| | 8.17-8.08 (m, 3H), 8.04-8.02 (m, 1H), 7.14 (d, J = 9.2 Hz, 1H), 4.71 (d, |
| | J = 14.9 Hz, 2H), 3.64 (d, J = 12.4 Hz, 2H), 3.44-3.35 (m, 2H), 3.27-3.17 |
| | (m, 2H), 2.98 (s, 3H), 2.58 (d, J = 0.8 Hz, 3H) |
| 76 | Starting material: 6-methyl-2,6-diazaspiro[3.5]nonane |
| | MS: m/z 465.0 [M + H]⁺; RT 0.27 min (Method 7) |
| | ¹H NMR: (400 MHz, METHANOL-d₄) δ ppm = 9.42 (d, J = 1.2 Hz, 1H), |
| | 8.18-8.11 (m, 3H), 8.05-8.01 (m, 1H), 6.69 (d, J = 9.2 Hz, 1H), 4.34 (d, |
| | J = 8.8 Hz, 1H), 4.08 (d, J = 8.8 Hz, 1H), 4.03 (s, 2H), 3.91-3.89 (m, 1H), |
| | 3.53-3.47 (m, 1H), 3.19 (d, J = 12.4 Hz, 1H), 3.06-2.97 (m, 1H), 2.93 (s, |
| | 3H), 2.58 (s, 3H), 2.28-2.18 (m, 1H), 1.93-1.76 (m, 2H), 1.32-1.28 (m, |
| | 1H) |
| 75 | Starting material: piperazine |
| | MS: m/z 410.9 [M + H]⁺; RT 0.24 min (Method 7) |
| | ¹H NMR: (400 MHz, METHANOL-d₄) δ ppm = 9.43 (d, J = 1.2 Hz, 1H), |
| | 8.15-8.11 (m, 2H), 8.14 (s, 1H), 8.04-8.01 (m, 1H), 4.07-3.97 (m, 4H), |
| | 3.39-3.35 (m, 4H), 2.58 (d, J = 0.8 Hz, 3H) |
| 239 | Starting materials: Intermediate 22 and |
| | rel-(5R)-1-methyl-1,7-diazaspiro[4.4]nonane |
| | MS: m/z. 495.2 [M + H]⁺; RT 2.007 min (Method 10) |
| 242 | Starting materials: Intermediate 22 and rel-(5R)-1-methyl-1,7- |
| | diazaspiro[4.4]nonane |
| | MS: m/z. 495.2 [M + H]⁺; RT 0.275 min (Method 9) |
| 207 | Starting materials: 5-azaspiro[2.3]hexane |
| | MS: m/z. 477.2 [M + H]⁺; RT 1.843 min (Method 10) |
| 203 | Starting materials: (3S)-N,N,4,4-tetramethylpyrrolidin-3-amine |
| | MS: m/z. 467.3 [M + H]⁺; RT 0.45 min (Method 4) |
| 204 | Starting materials: N,N-dimethylazetidin-3-amine |
| | MS: m/z. 425.2 [M + H]⁺; RT 0.40 min (Method 4) |
| 217 | Starting materials: (1R,5R)-3-methyl-3,6-diazabicyclo[3.2.0]heptane |
| | MS: m/z 437.2. [M + H]⁺; RT 0.41 min (Method 4) |
| 189 | Starting materials: 2-iodopropane |
| | MS: m/z. 465.0 [M + H]⁺; RT 1.095 min (Method 8) |
| 192 | Starting materials 7-fluoro-2-methyl-indazol-5-amine |
| | MS: m/z. 439.2 [M + H]⁺; RT 1.12 min (Method 3) |
| 235 | Starting materials: 6-[(1S,5R)-3,6-diazabicyclo[3.2.0]heptan-6-yl]-N-(7- |
| | fluoro-6-methoxy-2-methyl-indazol-5-yl)thieno[2,3-b]pyridine-2- |
| | carboxamide |
| | MS: m/z 467.2. [M + H]⁺; RT 1.710 min (Method 10) |
| 241 | Starting materials: Intermediate 18 and (3R)-N,N-dimethylpyrrolidin-3- |
| | amine |
| | MS: m/z 451.1 [M + H]⁺; RT 1.433 min (Method 10) |
| 216 | Starting materials: Intermediate 18 and (1S,5S)-3-methyl-3,6- |
| | diazabicyclo[3.2.0]heptane |
| | MS: m/z 449.2 [M + H]⁺; RT 0.49 min (Method 4) |
| 234 | Starting materials (3S)-3-(azetidin-1-yl)pyrrolidine |
| | MS: m/z 463.2 [M + H]⁺; RT 0.49 min (Method 4) |
| 221 | Starting materials: 6-(2,6-diazaspiro[3.4]octan-6-yl)-N-(7-methoxy-2- |
| | methyl-imidazo[1,2-a]pyridin-6-yl)thieno[2,3-b]pyridine-2-carboxamide |
| | MS: m/z. 463.3 [M + H]⁺; RT 1.093 min (Method 10) |
| 231 | Starting materials: Intermediate 19 and rac-(5S)-1-methyl-1,7- |
| | diazaspiro[4.4]nonane |
| | MS: m/z. 492.2 [M + H]⁺; RT 1.883 min (Method 10) |
| 244 | Starting materials: Intermediate 18 and (3R)-N,N-dimethylpyrrolidin-3- |
| | amine |
| | MS: m/z. 451.0 [M + H]⁺; RT 2.006 min (Method 8) |
| 222 | Starting materials: Intermediate 18 and (5R)-1-methyl-1,7- |
| | diazaspiro[4.4]nonane |
| | MS: m/z. 477.2 [M + H]⁺; RT 2.083 min (Method 8) |
| 223 | Starting materials: Intermediate 18 and (5R)-1-methyl-1,7- |
| | diazaspiro[4.4]nonane |
| | MS: m/z. 477.2 [M + H]⁺; RT 1.348 min (Method 8) |
| 232 | Starting materials: Intermediate 18 and 1-(azetidin-3-yl)azetidine |
| | MS: m/z 449.1 [M + H]⁺; RT 1.831 min (Method 8) |
| 236 | Starting materials: Intermediate 18 and 5-methyl-2,5-diazaspiro[3.4]octane |
| | MS: m/z 463.2 [M + H]⁺; RT 0.5 min (Method 4) |
| 229 | Starting materials: Intermediate 24 and (1S,5S)-3-methyl-3,6- |
| | diazabicyclo[3.2.0]heptane |
| | MS: m/z 434.2 [M + H]⁺; RT 0.42 min (Method 4) |
| 230 | Starting materials: Intermediate 24 and (5R)-1-methyl-1,7- |
| | diazaspiro[4.4]nonane |
| | MS: m/z 462.2 [M + H]⁺; RT 0.45 min (Method 4) |
| 205 | Starting materials: Intermediate 20 and rac-(R)-N,N-dimethylpyrrolidin-3- |
| | amine |
| | MS: m/z 451.3. [M + H]⁺; RT 1.638. min (Method 8) |
| 206 | Starting materials: Intermediate 20 and Intermediate 20 and rac-(R)-N,N- |
| | dimethylpyrrolidin-3-amine |
| | MS: m/z 451.3 [M + H]⁺; RT 1.333 min (Method 10) |
| 190 | Starting materials: Intermediate 20 and N,N-dimethylpyrrolidin-3-amine |
| | MS: m/z 451.1 [M + H]⁺; RT 1.561 min (Method 8) |
| 224 | Starting materials: Intermediate 20 and rac-(R)-1-methyl-1,7- |
| | diazaspiro[4.4]nonane |
| | MS: m/z 477.2 [M + H]⁺; RT 1.647 min (Method 10) |
| 225 | Starting materials: Intermediate 20 and rac-(R)-1-methyl-1,7- |
| | diazaspiro[4.4]nonane |
| | MS: m/z 477.2 [M + H]⁺; RT 1.640 min (Method 8) |
| 198 | Starting materials: Intermediate 15 and N,N-dimethylpyrrolidin-3-amine |
| | MS: m/z 452.2 [M + H]⁺; RT 1.700 min (Method 8) |
| 199 | Starting materials: Intermediate 15 and N,N-dimethylpyrrolidin-3-amine |
| | MS: m/z 452.1. [M + H]⁺; RT 1.697 min (Method 8) |
| 191 | Starting materials: Intermediate 15 and (3R)-N,N-dimethylpyrrolidin-3- |
| | amine |
| | MS: m/z 452.2 [M + H]⁺; RT 2.137 min (Method 8) |
| 260 | Starting materials: Intermediate 18 and rac-(3S,4S)-3-fluoro-2,2,6,6- |
| | tetramethylpiperidin-4-amine |
| | MS: m/z 511.2 [M + H]⁺; RT 1.33 min (Method 3) |
| 261 | Starting materials: Intermediate 18 and rac-(3R,4S)-3-fluoro-2,2,6,6- |
| | tetramethylpiperidin-4-amine |
| | MS: m/z 511.2 [M + H]⁺; RT 1.34 min (Method 3) |
| 180 | Starting materials: Intermediate 19 and 3-(azetidin-1-yl)pyrrolidine |
| | MS: m/z 481.2 [M + H]⁺; RT 1.883 min (Method 10) |
| 183 | Starting materials: Intermediate 20 and 1-ethylpiperazine |
| | MS: m/z 451.2 [M + H]⁺; RT 1.633 min (Method 10) |

### Example 6—Compound 67 and 68

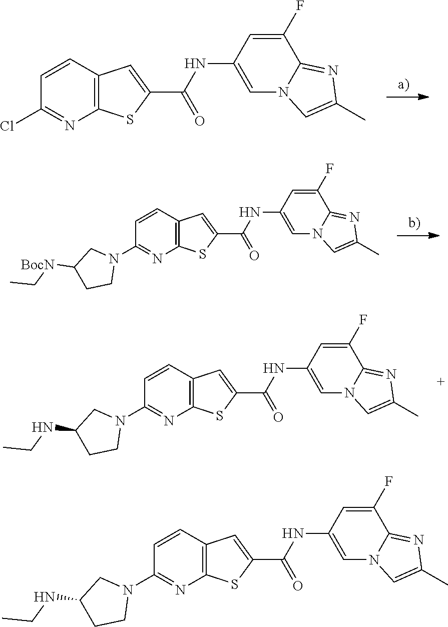

Step a: To a stirred solution of 6-chloro-N-(8-fluoro-2-methyl-imidazo[1,2-a]pyridin-6-5 yl)thieno[2,3-b]pyridine-2-carboxamide (15 mg, 41.58 μmol) in Dioxane (1 mL) was added TEA (207.88 umol, 29 μL) and tert-butyl N-ethyl-N-[(3S)-pyrrolidin-3-yl]carbamate (8.91 mg, 41.58 μmol). The reaction mixture was stirred at 90° C. for 12 h. The mixture was filtered and concentrated to give a residue which was purified by prep-HPLC (neutral condition) to give tert-butyl N-ethyl-N-[(3S)-1-[2-[(8-fluoro-2-methyl-imidazo[1,2-a]pyridin-6-10 yl)carbamoyl]thieno[2,3-b]pyridin-6-yl]pyrrolidin-3-yl]carbamate (15 mg, 54% yield) as a yellow solid. MS: m/z 539.2 [M+H]⁺; RT 0.71 min (Method 7) Step b: To a solution of tert-butyl N-ethyl-N-[(3S)-1-[2-[(8-fluoro-2-methyl-imidazo[1,2-a]pyridin-6-yl)carbamoyl]thieno[2,3-b]pyridin-6-yl]pyrrolidin-3-yl]carbamate (15 mg, 27.85 μmol) in DCM (1 mL) was added 2 M HCl in EtOAc (1 mL). The reaction mixture was stirred at 20° C. for 2 h. The mixture was filtered and concentrated to give a residue which was purified by prep-HPLC (neutral condition) to give 6-[(3S)-3-(ethylamino)pyrrolidin-1-yl]-N-(8-fluoro-2-methyl-imidazo[1,2-a]pyridin-6-yl)thieno[2,3-b]pyridine-2-carboxamide (8 mg, 63% yield) MS: m/z 439.0 [M+H]⁺; RT 1.53 min (Method 7) as a white solid which was separated by prep-SFC (Column: DAICEL CHIRALCEL OD (250 mm*30 mm, 10 um); Mobile Phase: from 40% to 40% of 0.1% NH₃H₂O MEOH; Flow Rate (ml/min): 150; Column temp: 35° C.) to give 6-[(3S)-3-(ethylamino)pyrrolidin-1-yl]-N-(8-fluoro-2-methyl-imidazo[1,2-a]pyridin-6-yl)thieno[2,3-b]pyridine-2-carboxamide (3.4 mg, 42% yield) MS: m/z 439.1 [M+H]⁺; RT 1.67 min (Method 7) and 6-[(3R)-3-(ethylamino)pyrrolidin-1-yl]-N-(8-fluoro-2-methyl-imidazo[1,2-a]pyridin-6-yl)thieno[2,3-b]pyridine-2-carboxamide (3.9 mg, 470 yield) both as a white solid. MS: m/z 439.1 [M+H]⁺; RT 1.50 min (Method 7).

Using the procedure described for Example 5 above, additional compounds described herein were prepared by substituting the appropriate amine starting material in step a, suitable reagents and reaction conditions, obtaining compounds such as those selected from:

| Cpd ID | Data |
|---|---|
| 80 | Starting material: tert-butyl (3aR,6aS)-hexahydropyrrolo[3,4-c]pyrrole- |
| | 2(1H)-carboxylate |
| | MS: m/z 436.9 [M + H]⁺; RT 0.26 min (Method 7) |
| | ¹H NMR (400 MHz, METHANOL-d₄) δ = 8.96 (s, 1H), 8.48 (s, 1H), 8.00 |
| | (d, J = 8.0 Hz, 1H), 7.94 (s, 1H), 7.72 (d, J = 4.0 Hz, 1H), 7.26-7.23 (m, |
| | 1H), 6.72 (d, J = 8.0 Hz, 1H), 3.77-3.61 (m, 8H), 3.26-3.25 (m, 2H), 2.43 |
| | (s, 3H) |
| 78 | Starting material: 1-(pyrrolidin-3-yl)cyclopropan-1-amine |
| | MS: m/z 451.1 [M + H]⁺; RT 0.28 min (Method 7) |
| | ¹H NMR: (400 MHz, METHANOL-d₄) δ ppm = 9.43 (d, J = 1.2 Hz, 1H), |
| | 8.30 (d, J = 9.2 Hz, 1H), 8.22 (s, 1H), 8.12 (d, J = 1.2 Hz, 1H), 8.07-8.04(m, |
| | 1H), 7.01 (d, J = 9.2 Hz, 1H), 4.02-3.90 (m, 2H), 3.70-3.68 (m, 1H), 3.41- |
| | 3.37 (m, 1H), 2.97-2.84 (m, 1H), 2.57 (d, J = 0.8 Hz, 3H), 2.37-2.34 (m, |
| | 1H), 1.99-1.85 (m, 1H), 1.07-1.01 (m, 4H) |
| 237 | Starting materials: tert-butyl-3,3a,4,5,6,6a-hexahydro-2H-pyrrolo[3,4- |
| | b]pyrrole-1-carboxylate |
| | MS: m/z 437.2. [M + H]⁺; RT 0. min (Method 4) |
| 238 | Starting materials: tert-butyl N-methyl-N-[-pyrrolidin-3-yl]carbamate |
| | MS: m/z 425.2. [M + H]⁺; RT 0.41 min (Method 4) |
| 200 | Starting materials: tert-butyl-2,6-diazabicyclo[3.2.0]heptane-6-carboxylate |
| | MS: m/z 423.3. [M + H]⁺; RT 0.41 min (Method 4) |
| 196 | Starting materials: tert-butyl-3,6-diazabicyclo[3.2.0]heptane-3-carboxylate |
| | MS: m/z 423.2. [M + H]⁺; RT 1.41 min (Method 1) |
| 201 | Starting materials: tert-butyl-2,3,3a,4,6,6a-hexahydro-1H-pyrrolo[3,4- |
| | b]pyrrole-5-carboxylate |
| | MS: m/z 437.2. [M + H]⁺; RT 0.43 min (Method 4) |
| 202 | Starting materials: tert-butyl (1R,5S)-3,6-diazabicyclo[3.2.0]heptane-6- |
| | carboxylate |
| | MS: m/z 423.3. [M + H]⁺; RT 0.40 min (Method 4) |
| 197 | Starting materials: tert-butyl N-(azetidin-3-yl)-N-methyl-carbamate |
| | MS: m/z 411.2. [M + H]⁺; RT 0.41 min (Method 4) |
| 245 | Starting materials: |
| | tert-butyl (3aS,6aS)-3,3a,4,5,6,6a-hexahydro-2H-pyrrolo[2,3-c]pyrrole-1- |
| | carboxylate |
| | MS: m/z 437.2. [M + H]⁺; RT 0.42 min (Method 4) |
| 209 | Starting materials: tert-butyl-1,7-diazaspiro[3.4]octane-7-carboxylate |
| | MS: m/z 437.2. [M + H]⁺; RT 0.48 min (Method 4) |
| 212 | Starting materials: Intermediate 20 and tert-butyl ethyl(pyrrolidin-3- |
| | yl)carbamate |
| | MS: m/z 451.2 [M + H]⁺; RT 1.693 min (Method 10) |
| 213 | Starting materials: Intermediate 18 and tert-butyl ethyl(pyrrolidin-3- |
| | yl)carbamate |
| | MS: m/z. 451.2 [M + H]⁺; RT 1.693 min (Method 10) |
| 220 | Starting materials: Intermediate 14 and tert-butyl (S)-methyl(pyrrolidin-3- |
| | yl)carbamate |
| | MS: m/z 437.1 [M + H]⁺; RT 1.085 min (Method 8) |
| 215 | Starting materials: Intermediate 5 and rac-tert-butyl (3aS,6aS)- |
| | hexahydropyrrolo[3,4-b]pyrrole-5(1H)-carboxylate |
| | MS: m/z 434.1 [M + H]⁺; RT 0.95 min (Method 3) |

### Example 7—Compound 10

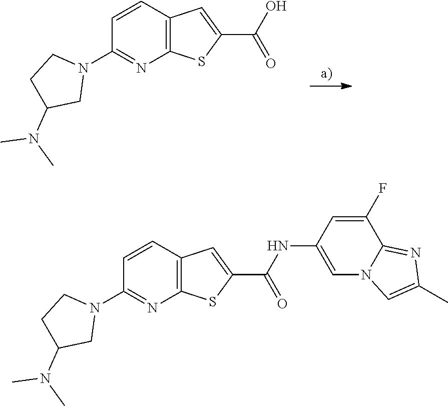

8-Fluoro-2-methyl-imidazo[1,2-a]pyridin-6-amine (14.81 mg, 89.66 μmol, 1.1 eq.) was dissolved in pyridine (407 μL, 0.2 M) before 6-[3-(dimethylamino)pyrrolidin-1-yl]thieno[2,3-b]pyridine-2-carboxylic acid (25 mg, 81.51 μmol, 1.0 eq.) and T3P (155.61 mg, 244.54 μmol, 145.57 μL, 50% purity in ethyl acetate, 3.0 eq.) were added. The solution then stirred at rt for 16 hours before it was concentrated, taken back up in a minimal amount of DMSO, filtered, and purified via reversed phase HPLC purification (column: XSelect CSH Prep C18 5 μm OBD 19×100 mm; Mobile phase A: MeCN; Mobile phase B: H20, Modifier: 0.1% NH₄OH) to obtain 6-[(3R)-3-(dimethylamino)pyrrolidin-1-yl]-N-(8-fluoro-2-methyl-imidazo[1,2-a]pyridin-6-yl)thieno[2,3-b]pyridine-2-carboxamide (26.3 mg, 74% yield) as a brown oil. MS: m/z 439.1 [M+H]⁺; RT 1.07 min (Method 3). 1H NMR (600 MHz, DMSO-d₆) δ ppm 2.20-2.27 (m, 1H) 2.36-2.42 (m, 3H) 2.86-2.91 (m, 6H) 3.48 (br s, 2H) 3.66 (br d, J=4.58 Hz, 1H) 3.75-3.78 (m, 1H) 3.99-4.05 (m, 2H) 6.76-6.80 (m, 1H) 7.47-7.55 (m, 1H) 8.01-8.15 (m, 2H) 9.07-9.12 (m, 1H) 9.96-10.04 (m, 1H) 10.53-10.59 (m, 1H).

Using the procedure described for Example 7 above, additional compounds described herein were prepared by substituting the appropriate amine starting material in step a, suitable reagents and reaction conditions, obtaining compounds such as those selected from:

| Cpd ID | Data |
|---|---|
| 49 | Starting material: 6-amino-1-methyl-1,3-dihydro-2H-benzo[d]imidazol-2- |
| | one |
| | MS: m/z 437.2 [M + H]⁺; RT 0.68 min (Method 5) |
| 65 | Starting material: 7-fluoro-2-methyl-indazol-5-amine |
| | MS: m/z 439.3 [M + H]⁺; RT 1.32 min (Method 3) |
| 71 | Starting material: 4-fluoro-2-methyl-1,3-benzoxazol-6-amine |
| | MS: m/z 440.1 [M + H]⁺; RT 1.35 min (Method 3) |
| | ¹H NMR (600 MHz, DMSO-d₆) δ ppm 1.82-1.92 (m, 1 H) 2.16-2.22 (m, |
| | 1 H) 2.26 (s, 6 H) 2.53-2.57 (m, 1 H) 2.63 (s, 3 H) 3.20-3.27 (m, 1 H) 3.44 |
| | (m, 1 H) 3.67-3.73 (m, 1 H) 3.80 (s, 1 H) 6.70 (d, J = 9.16 Hz, 1 H) 7.59 |
| | (m, 1 H) 8.00 (d, J = 1.53 Hz, 1 H) 8.06 (d, J = 8.77 Hz, 1 H) 8.12 (s, 1 H) |
| | 10.56 (s, 1 H) |
| 72 | Starting material: 8-methoxy-2-methyl-imidazo[1,2-a]pyridin-6-amine |
| | MS: m/z 451.2 [M + H]⁺; RT 1.00 min (Method 3) |
| | ¹H NMR (600 MHz, DMSO-d₆) δ ppm 1.83-1.95 (m, 1 H) 2.17-2.25 (m, |
| | 2 H) 2.27-2.36 (m, 9 H) 3.22-3.28 (m, 1 H) 3.44 (m, 1 H) 3.65-3.74 (m, |
| | 1 H) 3.77-3.87 (m, 1 H) 3.94 (s, 3 H) 6.70 (d, J = 9.16 Hz, 1 H) 6.85 (d, J = |
| | 1.14 Hz, 1 H) 7.71 (s, 1 H) 8.05 (d, J = 9.16 Hz, 1 H) 8.09 (s, 1 H) 8.78 (d, |
| | J = 1.14 Hz, 1 H) 10.25 (s, 1 H) |
| 73 | Starting material: 2,8-dimethylimidazo[1,2-a]pyridin-6-amine |
| | MS: m/z 435.2 [M + H]⁺; RT 0.94 min (Method 3) |
| 58 | Starting material: 5-amino-1,3-dimethyl-1,3-dihydro-2H-benzo[d]imidazol- |
| | 2-one |
| | MS: m/z 451.2 [M + H]⁺; RT 0.89 min (Method 5) |
| 59 | Starting material: 5-amino-3-methylbenzo[d]thiazol-2(3H)-one |
| | MS: m/z 454.0 [M + H]⁺; RT 1.02 min (Method 5) |
| 60 | Starting material: 7-(trifluoromethyl)pyrazolo[1,5-a]pyrimidin-3-amine |
| | MS: m/z 476.0 [M + H]⁺; RT 0.80 min (Method 5) |
| 61 | Starting material: 1-isopropyl-1H-indazol-6-amine |
| | MS: m/z 449.1 [M + H]⁺; RT 1.18 min (Method 5) |
| 62 | Starting material: imidazo[1,5-a]pyridin-6-amine |
| | MS: m/z 407.0 [M + H]⁺; RT 0.64 min (Method 5) |
| 63 | Starting material: benzo[c][1,2,5]thiadiazol-5-amine |
| | MS: m/z 425.1 [M + H]⁺; RT 1.07 min (Method 5) |
| 64 | Starting material: [1,2,4]triazolo[1,5-a]pyrazin-2-amine |
| | MS: m/z 409.1 [M + H]⁺; RT 0.85 min (Method 3) |
| 74 | Starting material: 6-methoxy-2-methyl-2H-indazol-5-amine |
| | MS: m/z 451.6 [M + H]⁺; RT 0.48 min (Method 4) |

### Example 8—Compound 54

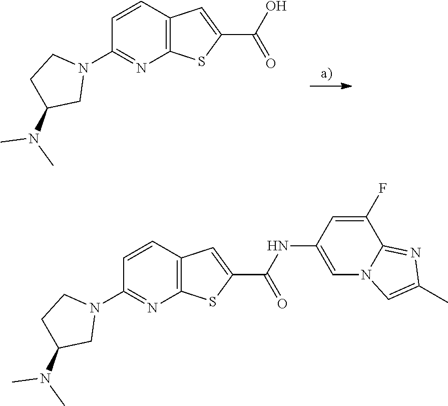

6-[(3S)-3-(Dimethylamino)pyrrolidin-1-yl]thieno[2,3-b]pyridine-2-carboxylic acid (25 mg, 83.81 μmol, 1.0 eq.) was dissolved in pyridine (1 mL, 0.083 M) before 8-fluoro-2-methyl-imidazo[1,2-a]pyridin-6-amine (13.84 mg, 83.81 μmol, 1.0 eq.) and T3P (148 μL, 251.42 μmol, 50% purity in ethyl acetate, 3.0 eq.) was added. The solution was then stirred at 50° C. for 4 hours before being concentrated, taken up in a minimal amount of DMSO, filtered, and purified via HPLC purification (column: XSelect CSH Prep C18 5 um OBD 19×100 mm; Mobile phase A: MeCN; Mobile phase B: H₂O, Modifier: 0.1% NH₄OH) to obtain 6-[(3S)-3-(dimethylamino)pyrrolidin-1-yl]-N-(8-fluoro-2-methyl-imidazo[1,2-a]pyridin-6-yl)thieno[2,3-b]pyridine-2-carboxamide (11.1 mg, 28% yield) as a yellow solid. MS: m/z 439.2 [M+H]⁺; RT 0.89 min (Method 3).

Using the procedure described for Example 8 above, additional compounds described herein were prepared by substituting the appropriate amine starting material in step a, suitable reagents and reaction conditions, obtaining compounds such as those selected from:

| Cpd ID | Data |
|---|---|
| 50 | Starting material: 2,8-dimethylimidazo[1,2-a]pyrazin-6-amine |
| | MS: m/z 436.3 [M + H]⁺; RT 0.89 min (Method 3) |
| | ¹H NMR (600 MHz, DMSO-d₆) δ ppm 1.82-1.90 (m, 1 H) 2.16-2.20 (m, |
| | 1 H) 2.23-2.31 (m, 6 H) 2.37-2.41 (m, 3 H) 2.70-2.75 (m, 3 H) 2.81-2.93 |
| | (m, 1 H) 3.20-3.26 (m, 1 H) 3.39-3.46 (m, 1 H) 3.66-3.74 (m, 1 H) 3.76- |
| | 3.85 (m, 1 H) 6.64-6.73 (m, 1 H) 7.89-8.02 (m, 2 H) 8.29-8.36 (m, 1 H) |
| | 9.08-9.13 (m, 1 H) 10.84-10.90 (m, 1 H) |
| 52 | Starting material: 2-methylimidazo[1,2-b]pyridazin-6-amine |
| | MS: m/z 422.3 [M + H]⁺; RT 0.85 min (Method 3) |
| 26 | Starting material: 2-methylimidazo[1,2-a]pyridin-6-amine |
| | MS: m/z 421.3 [M + H]⁺; RT 0.93 min (Method 3) |
| | ¹H NMR (400 MHz, METHANOL-d₄) δ ppm 2.30-2.40 (m, 1 H) 2.54- |
| | 2.57 (m, 3 H) 2.60-2.71 (m, 1 H) 3.00-3.02 (m, 6 H) 3.60-3.67 (m, 1 H) |
| | 3.77-3.82 (m, 1 H) 3.86-3.92 (m, 1 H) 4.05-4.18 (m, 2 H) 6.75-6.80 (m, |
| | 1 H) 7.81-7.85 (m, 1 H) 7.98-8.08 (m, 4 H) 9.54-9.56 (m, 1 H) |

### Example 9—Compound 55

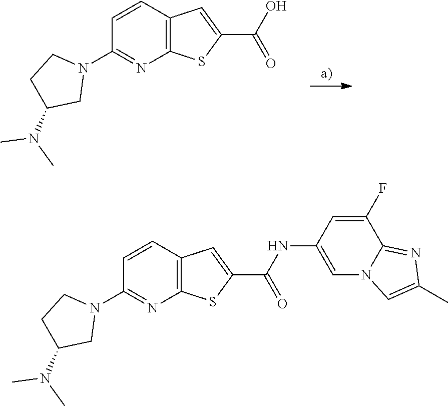

6-[(3R)-3-(Dimethylamino)pyrrolidin-1-yl]thieno[2,3-b]pyridine-2-carboxylic acid (25 mg, 85.80 μmol, 1.0 eq.) was dissolved in pyridine (1 mL, 0.09 M) before 8-fluoro-2-methyl-imidazo[1,2-a]pyridin-6-amine (14.17 mg, 85.80 μmol, 1.0 eq.) and T3P (163.80 mg, 257.4 μmol, 3.0 eq., 50% purity in ethyl acetate) were added. The solution was stirred at 50° C. for 4 hours before being concentrated, taken up in DMSO, filtered, and purified via HPLC purification (column: XSelect CSH Prep C18 5 um OBD 19×100 mm; Mobile phase A: MeCN; Mobile phase B: H₂O, Modifier: 0.1% NH₄OH) to obtain 6-[(3R)-3-(dimethylamino)pyrrolidin-1-yl]-N-(8-fluoro-2-methyl-imidazo[1,2-a]pyridin-6-yl)thieno[2,3-b]pyridine-2-carboxamide (7.3 mg, 19% yield). MS: m/z 439.2 [M+H]⁺; RT 0.90 min (Method 3). ¹H NMR (600 MHz, DMSO-d₆) δ ppm 1.81-1.90 (m, 1H) 2.17-2.21 (m, 1H) 2.23-2.30 (m, 6H) 2.33-2.38 (m, 3H) 2.82-2.91 (m, 1H) 3.19-3.26 (m, 1H) 3.40-3.47 (m, 1H) 3.65-3.73 (m, 1H) 3.75-3.82 (m, 1H) 6.67-6.72 (m, 1H) 7.27-7.34 (m, 1H) 7.88-7.93 (m, 1H) 8.03-8.10 (m, 2H) 8.98-9.02 (m, 1H) 10.35-10.40 (m, 1H).

Using the procedure described for Example 9 above, additional compounds described herein were prepared by substituting the appropriate amine starting material in step a, suitable reagents and reaction conditions, obtaining compounds such as those selected from:

| Cpd ID | Data |
|---|---|
| 51 | Starting material: 2,8-dimethylimidazo[1,2-a]pyrazin-6-amine |
| | MS: m/z 436.2 [M + H]⁺; RT 0.88 min (Method 3) |
| | ¹H NMR (400 MHz, METHANOL-d₄) δ ppm 2.36-2.45 (m, 1 H) 2.62- |
| | 2.64 (m, 3 H) 2.65-2.72 (m, 1 H) 2.89-2.91 (m, 3 H) 3.01-3.05 (m, 6 H) |
| | 3.67-3.74 (m, 1 H) 3.85-3.90 (m, 1 H) 3.92-3.99 (m, 1 H) 4.12-4.21 (m, |
| | 2 H) 6.87-6.93 (m, 1 H) 8.14-8.21 (m, 3 H) 9.46-9.49 (m, 1 H) |
| 53 | Starting material: 2-methylimidazo[1,2-b]pyridazin-6-amine |
| | MS: m/z 422.2 [M + H]⁺; RT 0.85 min (Method 3) |
| | ¹H NMR (600 MHz, DMSO-d₆) δ ppm 1.80-1.87 (m, 1 H) 2.14-2.19 (m, |
| | 1 H) 2.21-2.25 (m, 6 H) 2.36-2.41 (m, 3 H) 2.76-2.84 (m, 1 H) 3.18- |
| | 3.24 (m, 1 H) 3.40-3.47 (m, 1 H) 3.65-3.73 (m, 1 H) 3.75-3.83 (m, 1 H) |
| | 6.65-6.72 (m, 1 H) 7.77-7.84 (m, 1 H) 7.93-8.04 (m, 2 H) 8.26-8.32 (m, |
| | 1 H) 11.19-11.24 (m, 1 H) |
| 56 | Starting material: 2,8-dimethylimidazo[1,2-b]pyridazin-6-amine |
| | MS: m/z 436.2 [M + H]⁺; RT 0.90 min (Method 3) |
| | ¹H NMR (600 MHz, DMSO-d₆) δ ppm 1.81-1.89 (m, 1 H) 2.17-2.22 (m, |
| | 1 H) 2.24-2.34 (m, 6 H) 2.53-2.57 (m, 3 H) 2.83-2.93 (m, 1 H) 3.20- |
| | 3.25 (m, 1 H) 3.40-3.47 (m, 1 H) 3.66-3.73 (m, 1 H) 3.74-3.81 (m, 1 H) |
| | 6.66-6.73 (m, 1 H) 7.65-7.71 (m, 1 H) 7.87-7.96 (m, 1 H) 7.99-8.05 (m, |
| | 1 H) 8.26-8.31 (m, 1 H) 11.10-11.17 (m, 1 H) |
| 25 | Starting material: 2-methylimidazo[1,2-a]pyridin-6-amine |
| | MS: m/z 421.3 [M + H]⁺; RT 0.93 min (Method 3) |
| | ¹H NMR (400 MHz, METHANOL-d₄) δ ppm 2.28-2.39 (m, 1 H) 2.54- |
| | 2.57 (m, 3 H) 2.61-2.70 (m, 1 H) 3.01-3.03 (m, 6 H) 3.60-3.68 (m, 1 H) |
| | 3.77-3.82 (m, 1 H) 3.86-3.89 (m, 1 H) 4.04-4.05 (m, 1 H) 4.04-4.15 (m, |
| | 1 H) 6.76-6.81 (m, 1 H) 7.81-7.87 (m, 1 H) 7.99-8.08 (m, 4 H) 9.54- |
| | 9.57 (m, 1 H) |
| 252 | Starting materials: 6-chlorothieno[2,3-b]pyridine-2-carboxylic acid, 7- |
| | methoxy-2-methylimidazo[1,2-a]pyridin-6-amine and (S)-1-methyl-1,7- |
| | diazaspiro[4.4]nonane |
| | MS: m/z 477.2 [M + H]⁺; RT 1.798 min (Method 8) |
| 251 | Starting materials: 6-chlorothieno[2,3-b]pyridine-2-carboxylic acid, 7- |
| | methoxy-2-methylimidazo[1,2-a]pyridin-6-amine and (R)-1-methyl-1,7- |
| | diazaspiro[4.4]nonane |
| | MS: m/z 477.2 [M + H]⁺; RT 1.792 min (Method 8) |
| 250 | Starting materials: 6-chlorothieno[2,3-b]pyridine-2-carboxylic acid, 8- |
| | (difluoromethyl)-2-methylimidazo[1,2-a]pyrazin-6-amine and (S)-N,N- |
| | dimethylpyrrolidin-3-amine |
| | MS: m/z 472.2 [M + H]⁺; RT 1.918 min (Method 8) |
| 253 | Starting materials: 6-chlorothieno[2,3-b]pyridine-2-carboxylic acid, 8- |
| | (difluoromethyl)-2-methylimidazo[1,2-a]pyrazin-6-amine and 1- |
| | ethylpiperazine |
| | MS: m/z 472.2 [M + H]⁺; RT 1.087 min (Method 10) |
| 254 | Starting materials: 6-chlorothieno[2,3-b]pyridine-2-carboxylic acid, 8- |
| | (difluoromethyl)-2-methylimidazo[1,2-a]pyrazin-6-amine and (S)-1- |
| | methyl-1,7-diazaspiro[4.4]nonane |
| | MS: m/z 498.2 [M + H]⁺; RT 2.038 min (Method 8) |
| 255 | Starting materials: 6-chlorothieno[2,3-b]pyridine-2-carboxylic acid, 8- |
| | (difluoromethyl)-2-methylimidazo[1,2-a]pyrazin-6-amine and (R)-1- |
| | methyl-1,7-diazaspiro[4.4]nonane |
| | MS: m/z 498.2 [M + H]⁺; RT 2.032 min (Method 8) |
| 256 | Starting materials: 6-chlorothieno[2,3-b]pyridine-2-carboxylic acid, 6- |
| | methoxy-2-methylpyrazolo[1,5-a]pyridin-5-amine and tert-butyl 2,6- |
| | diazaspiro[3.4]octane-6-carboxylate |
| | MS: m/z 477.2 [M + H]⁺; RT 1.908 min (Method 8) |

### Example 10—Compound 8

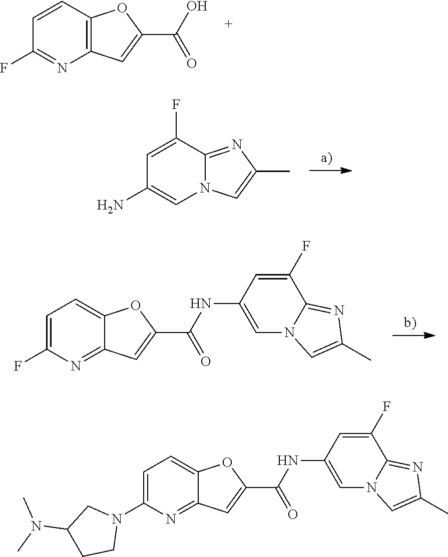

Step a: To a vial with 8-fluoro-2-methyl-imidazo[1,2-a]pyridin-6-amine (50.16 mg, 303.68 μmol) and 5-chlorofuro[3,2-b]pyridine-2-carboxylic acid (50 mg, 253.07 μmol) was added 2,4,6-tripropyl-1,3,5,2,4,6trioxatriphosphinane 2,4,6-trioxide (483.13 mg, 759.20 μmol, 451.94 μL, 50% purity), N-ethyl-N-isopropyl-propan-2-amine (98.12 mg, 759.20 μmol, 132.24 μL) and dioxane (2 mL). The reaction mixture was stirred at 60° C. overnight. The mixture was concentrated under vacuum. The residue was purified by column chromatography (0% to 20% MeOH/DCM) to give 5-chloro-N-(8-fluoro-2-methyl-imidazo[1,2-a]pyridin-6-yl)furo[3,2-b]pyridine-2-carboxamide (58.6 mg, 67% yield) as a pale-yellow solid. MS: m/z 345.0 [M+H]⁺; RT 0.52 min (Method 4)

Step b: A microwave vial with 5-chloro-N-(8-fluoro-2-methyl-imidazo[1,2-a]pyridin-6-yl)furo[3,2-b]pyridine-2-carboxamide (25 mg, 64.54 μmol), diacetoxypalladium (1.45 mg, 6.45 μmol), [1-(2-diphenylphosphanyl-1-naphthyl)-2-naphthyl]-diphenyl-phosphane (8.04 mg, 12.91 μmol), sodium tert-butoxide (18.61 mg, 193.63 μmol) was evacuated under vacuum and refilled with N₂ three times. Tetrahydrofuran (1 mL) and (3S)—N,N-dimethylpyrrolidin-3-amine (44.22 mg, 387.26 μmol) was then added under N₂ atmosphere. The reaction mixture was stirred at 90° C. for 12 h. The mixture was concentrated under vacuum and purified by HPLC (Column: Sunfire C18 100×19 mm, 5 mm; Mobile phase A: MeCN; Mobile phase B: H₂O; Modifier: 0.1% TFA) to obtain 5-[(3S)-3-(dimethylamino)pyrrolidin-1-yl]-N-(8-fluoro-2-methyl-imidazo[1,2-a]pyridin-6-yl)furo[3,2-b]pyridine-2-carboxamide (19.5 mg, 71% yield). MS: m/z 423.1 [M+H]⁺; RT 0.73 min (Method 3).

Using the procedure described for Example 10 above, additional compounds described herein were prepared by substituting the appropriate amine starting material in step a, suitable reagents and reaction conditions, obtaining compounds such as those selected from:

| Cpd Id | Data |
|---|---|
| 7 | Starting material: 2,7-dimethylindazol-5-amine |
| | MS: m/z 421.3 [M + H]⁺; RT 0.93 min (Method 3) ¹H NMR (600 MHz, |
| | DMSO-d₆) δ ppm 2.20-2.27 (m, 1 H) 2.53-2.56 (m, 3 H) 2.83-2.95 (m, |
| | 6 H) 3.45-3.47 (m, 2 H) 3.60-3.66 (m, 1 H) 3.69-3.76 (m, 1 H) 3.91-3.96 |
| | (m, 1 H) 3.98-4.05 (m, 1 H) 4.11-4.21 (m, 3 H) 6.70-6.75 (m, 1 H) 7.28- |
| | 7.32 (m, 1 H) 7.55-7.60 (m, 1 H) 7.96-8.06 (m, 1 H) 8.26-8.32 (m, 1 H) |
| | 9.97-10.05 (m, 1 H) 10.29-10.35 (m, 1 H). |

### Example 11—Compound 9

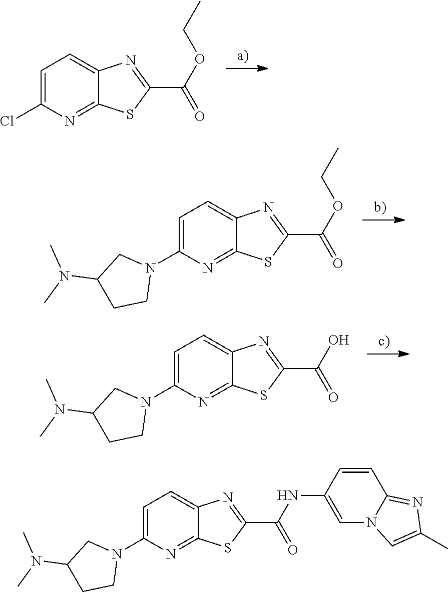

Step a: N,N-Dimethylpyrrolidin-3-amine (57.83 mg, 309.05 μmol, 2Hydrochloride, 3.0 eq.), ethyl 5-chlorothiazolo[5,4-b]pyridine-2-carboxylate (25 mg, 103.02 μmol, 1.0 eq.), and DIPEA (13.31 mg, 103.02 μmol, 1.0 eq.) were dissolved in Dioxane (515.08 μL, 0.2 M) which was heated to 80° C. for 16 hours before being injected crude onto normal phase and purified via 0-25% MeOH:DCM over 3.5 minutes. Product elutes at 22% MeOH. Identified fractions were collected, combined, and concentrated to yield ethyl 5-[3-(dimethylamino)pyrrolidin-1-yl]thiazolo[5,4-b]pyridine-2-carboxylate as a wet beige solid that was carried forward as is (assumed 100% yield). MS: m/z 321.1 [M+H]⁺; RT 0.49 min (Method 4) Step b: Ethyl 5-[3-(dimethylamino)pyrrolidin-1-yl]thiazolo[5,4-b]pyridine-2-carboxylate (121.31 mg, 318.03 μmol, 1.0 eq.) was dissolved in Dioxane (795.08 μL, 0.2 M) and water (795.08 μL, 0.2 M) before lithium hydroxide (7.62 mg, 318.03 μmol, 1.0 eq.) was added. The solution was then heated to 80° C. and stirred for 16 hours before it was concentrated and carried forward crude as 5-[3-(dimethylamino)pyrrolidin-1-yl]thiazolo[5,4-b]pyridine-2-carboxylic acid (50.8 mg, 52% yield) as a yellow white solid. MS: m/z 293.0 [M+H]⁺; RT 0.35 min (Method 4)

Step c: 5-[3-(Dimethylamino)pyrrolidin-1-yl]thiazolo[5,4-b]pyridine-2-carboxylic acid (50.80 mg, 165.07 μmol, 1.0 eq.) was dissolved in acetonitrile (695.04 μL, 0.24 M) before 2-methylimidazo[1,2-a]pyridin-6-amine (24.29 mg, 165.07 μmol, 1.0 eq.), HATU (69.04 mg, 181.58 μmol, 1.1 eq.), and DIPEA (46.93 mg, 363.16 μmol, 2.2 eq.) were added. The solution was then stirred at rt for 3 hours before it was concentrated and taken up in a minimal amount of DMSO, filtered, and purified via reversed phase HPLC purification (Column: Sunfire C18 100×19 mm, 5 mm; Mobile phase A: MeCN; Mobile phase B: H₂O; Modifier: 0.1% TFA) to obtain 5-[3-(dimethylamino)pyrrolidin-1-yl]-N-(2-methylimidazo[1,2-a]pyridin-6-yl)thiazolo[5,4-b]pyridine-2-carboxamide (11.7 mg, 13% yield) as a brown solid. MS: m/z 422.2 [M+H]⁺; RT 0.87 min (Method 3)

### Example 12—Compound 84

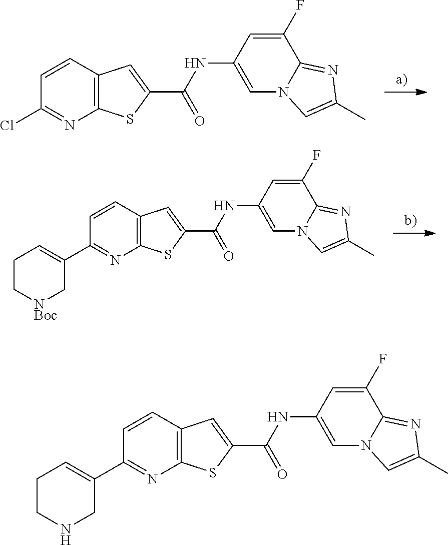

Step a: To a solution of 6-chloro-N-(8-fluoro-2-methyl-imidazo[1,2-a]pyridin-6-yl)thieno[2,3-b]pyridine-2-carboxamide (41 mg, 113.64 μmol) in Dioxane (5 mL) and water (1 mL) was added Pd(dppf)Cl₂ (8.32 mg, 11.36 μmol), K₂CO₃ (31.41 mg, 227.28 μmol) and tert-butyl 5-(4,4,5,5-tetramethyl-1,3,2-dioxaborolan-2-yl)-3,6-dihydro-2H-pyridine-1-carboxylate (35.14 mg, 113.64 μmol). The reaction mixture was stirred at 90° C. for 12 h under N₂. The mixture was filtered and concentrated to give a residue. The residue was purified by flash silica gel chromatography (from PE/EtOAc=10/1 to 5/1) to yield tert-butyl 5-[2-[(8-fluoro-2-methyl-imidazo[1,2-a]pyridin-6-yl)carbamoyl]thieno[2,3-b]pyridin-6-yl]-3,6-dihydro-2H-pyridine-1-carboxylate (30 mg, 50% yield). MS: m/z 508.0 [M+H]⁺; RT 0.43 min (Method 7)

Step b: To a stirred solution of tert-butyl 5-[2-[(8-fluoro-2-methyl-imidazo[1,2-a]pyridin-6-yl)carbamoyl]thieno[2,3-b]pyridin-6-yl]-3,6-dihydro-2H-pyridine-1-carboxylate (10 mg, 19.70 μmol) in DCM (1 mL) was added 4 M HCl in EtOAc (2 mL). The reaction mixture was stirred at 20° C. for 2 h. The mixture was filtered and concentrated to give a residue which was purified by prep-HPLC (neutral condition) to afford N-(8-fluoro-2-methyl-imidazo[1,2-a]pyridin-6-yl)-6-(1,2,3,6-tetrahydropyridin-5-yl)thieno[2,3-b]pyridine-2-carboxamide (2.3 mg, 27% yield) as a yellow solid. MS: m/z 407.8 [M+H]⁺; RT 0.25 min (Method 7); 1H NMR (400 MHz, METHANOL-d4) δ=9.44 (s, 1H), 8.35 (d, J=8.4 Hz, 1H), 8.24 (s, 1H), 8.12 (s, 1H), 8.01 (d, J=12.8 Hz, 1H), 7.89 (d, J=8.4 Hz, 1H), 7.08-7.06 (m, 1H), 4.35 (d, J=1.6 Hz, 2H), 3.45-3.42 (m, 2H), 2.72-3.71 (m, 2H), 2.58 (s, 3H).

Using the procedure described for Example 12 above, additional compounds described herein were prepared by substituting the appropriate boronic ester starting material in step a, suitable reagents and reaction conditions, obtaining compounds such as those selected from:

| Cpd ID | Data |
|---|---|
| 83 | Starting material: tert-butyl 3-(4,4,5,5-tetramethyl-1,3,2-dioxaborolan-2-yl)- |
| | 8-azabicyclo[3.2.1]oct-2-ene-8-carboxylate |
| | MS: m/z 433.7 [M + H]⁺; RT 0.26 min (Method 7) |
| | ¹H NMR (400 MHz, METHANOL-d₄) δ = 9.39 (d, J = 0.8 Hz, 1H), 8.35 (d, |
| | J = 8.4 Hz, 1H), 8.23 (s, 1H), 8.08 (s, 1H), 7.92 (d, J = 10.8 Hz, 1H), 7.79 (d, |
| | J = 8.4 Hz, 1H), 7.07 (d, J = 5.6 Hz, 1H), 4.51-4.48 (m, 1H), 4.42 (d, J = 5.2 |
| | Hz, 1H), 2.56 (s, 3H), 2.42-2.23 (m, 4H), 2.07-1.99 (m, 2H) |
| 249 | Starting materials: 1-methyl-4-(4,4,5,5-tetramethyl-1,3,2-dioxaborolan-2- |
| | yl)-1,2,3,6-tetrahydropyridine |
| | MS: m/z 453.54 [M + H]⁺; RT 1.968 min (Method 8) |

### Example 13—Compound 81 and 82

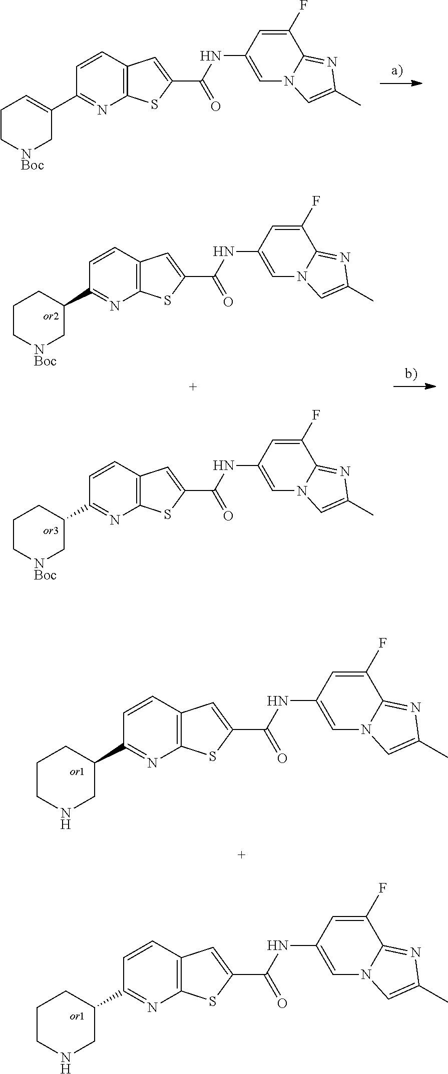 [SMILES: Cc1cn2cc(NC(=O)c3cc4ccc(C5=CCCN(C(=O)OC(C)(C)C)C5)nc4s3)cc(F)c2n1 ; Cc1cn2cc(NC(=O)c3cc4ccc([C@@H]5CCCN(C(=O)OC(C)(C)C)C5)nc4s3)cc(F)c2n1 ; Cc1cn2cc(NC(=O)c3cc4ccc([C@H]5CCCN(C(=O)OC(C)(C)C)C5)nc4s3)cc(F)c2n1 ; Cc1cn2cc(NC(=O)c3cc4ccc([C@@H]5CCCNC5)nc4s3)cc(F)c2n1 ; Cc1cn2cc(NC(=O)c3cc4ccc([C@H]5CCCNC5)nc4s3)cc(F)c2n1]

Step a: To a solution of tert-butyl 5-[2-[(8-fluoro-2-methyl-imidazo[1,2-a]pyridin-6-yl)carbamoyl]thieno[2,3-b]pyridin-6-yl]-3,4-dihydro-2H-pyridine-1-carboxylate (32 mg, 63.04 umol) in MeOH (10 mL) was added Pd/C (6.71 mg, 63.04 umol) under N₂. The mixture was stirred at 25° C. under 50 psi of H2 for 12 hours. The reaction mixture was filtered and concentrated under reduced pressure to give the tert-butyl 3-[2-[(8-fluoro-2-methyl-imidazo[1,2-a]pyridin-6-yl)carbamoyl]thieno[2,3-b]pyridin-6-yl]piperidine-1-carboxylate (9 mg, 27% yield) as a white solid MS: m/z 510.2 [M+H]⁺; RT 0.42 min (Method 7) which was purified further by prep-SFC (Column: Chiralpak AD-3 50×4.6 mm I.D., 3 um, Mobile phase: Phase A for CO2, and Phase B for IPA (0.05% DEA); Isocratic elution: 40% B in A Flow rate: 3 mL/min; Detector: PDA; Column Temp: 35° C.; Back Pressure: 100 Bar) to obtain tert-butyl (3R)-3-[2-[(8-fluoro-2-methyl-imidazo[1,2-a]pyridin-6-yl)carbamoyl]thieno[2,3-b]pyridin-6-yl]piperidine-1-carboxylate and tert-butyl (3S)-3-[2-[(8-fluoro-2-methyl-imidazo[1,2-a]pyridin-6-yl)carbamoyl]thieno[2,3-b]pyridin-6-yl]piperidine-1-carboxylate.

Step b: In separate vials, tert-butyl (3R)-3-[2-[(8-fluoro-2-methyl-imidazo[1,2-a]pyridin-6-yl)carbamoyl]thieno[2,3-b]pyridin-6-yl]piperidine-1-carboxylate (10.00 mg, 19.62 umol) and tert-butyl (3S)-3-[2-[(8-fluoro-2-methyl-imidazo[1,2-a]pyridin-6-yl)carbamoyl]thieno[2,3-b]pyridin-6-yl]piperidine-1-carboxylate (10.00 mg, 19.62 umol) were dissolved in DCM (1 mL) and then treated with 2 M HCl in EtOAc. After stirring for 2 h at rt, the mixture was filtered and concentrated to give a residue which was purified by prep-HPLC (neutral condition) to give N-(8-fluoro-2-methyl-imidazo[1,2-a]pyridin-6-yl)-6-[(3R)-3-piperidyl]thieno[2,3-b]pyridine-2-carboxamide (1.6 mg, 19% yield) MS: m/z 410.0 [M+H]⁺; RT 0.26 min (Method 7); ¹H NMR (400 MHz, METHANOL-d₄) δ=9.41 (s, 1H), 8.37 (d, J=8.4 Hz, 1H), 8.24 (s, 1H), 8.09 (s, 1H), 7.94 (d, J=12.0 Hz, 1H), 7.52 (d, J=8.4 Hz, 1H), 3.62-3.48 (m, 4H), 3.21-3.14 (m, 1H), 2.57 (s, 3H), 2.26-2.22 (m, 1H), 2.03-1.99 (m, 1H), 1.98-1.92 (m, 2H) and N-(8-fluoro-2-methyl-imidazo[1,2-a]pyridin-6-yl)-6-[(3S)-3-piperidyl]thieno[2,3-b]pyridine-2-carboxamide (3.8 mg, 45% yield) both as a yellow solid. MS: m/z 410.0 [M+H]⁺; RT 0.25 min (Method 7); 1H NMR (400 MHz, METHANOL-d₄) δ=9.38 (s, 1H), 8.37 (d, J=8.0 Hz, 1H), 8.23 (s, 1H), 8.07 (s, 1H), 7.90 (d, J=11.6 Hz, 1H), 7.52 (d, J=8.4 Hz, 1H), 3.61-3.19, (m, 4H), 3.19-3.13 (m, 1H), 2.56 (s, 3H), 2.26-2.20 (m, 1H), 2.00-1.92 (m, 3H).

Using the procedure described for Example 5 above, additional compounds described herein were prepared by substituting the appropriate amine starting material in step a, suitable reagents and reaction conditions, obtaining compounds such as those selected from:

| Cpd ID | Data |
|---|---|
| 85 | Starting material: tert-butyl 4-(4,4,5,5-tetramethyl-1,3,2-dioxaborolan-2- |
| | yl)-3,6-dihydropyridine-1(2H)-carboxylate |
| | MS: m/z 409.9 [M + H]⁺; RT 0.25 min (Method 7) |
| | ¹H NMR (400 MHz, METHANOL-d4) δ = 9.46 (s, 1H), 8.35 (d, J = 8.4 Hz, |
| | 1H), 8.25 (s, 1H), 8.14 (s, 1H), 8.05 (d, J = 11.2 Hz, 1H), 7.49 (d, J = 8.4 Hz, |
| | 1H), 3.56 (br d, J = 12.8 Hz, 2H), 3.25-3.18 (m, 3H), 2.59 (s, 3H), 2.25- |
| | 2.15 (m, 4H) |

### Example 14—Compound 86

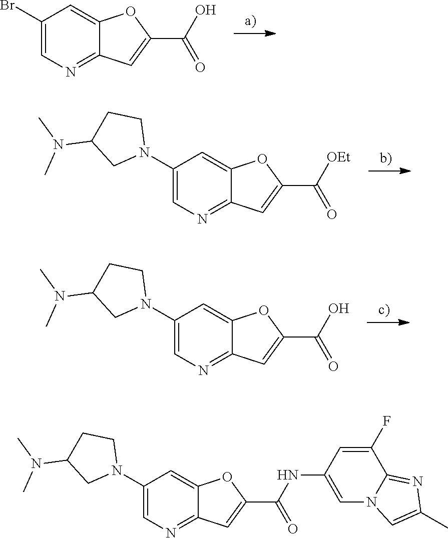

Step a: 6-Bromofuro[3,2-b]pyridine-2-carboxylic acid (200 mg, 826.36 μmol) was dissolved in ethanol (10 mL) before HCl in dioxane (4 M, 619.77 μL) was added. The solution then stirred at 80° C. for 16 hours, before being concentrated to obtain ethyl 6-chlorothieno[2,3-b]pyridine-2-carboxylate (ethyl 6-bromofuro[3,2-b]pyridine-2-carboxylate (244 mg) as an off-white powder that was carried forward crude. MS: m/z 271.9 [M+H]+.

A microwave vial with ethyl 6-bromofuro[3,2-b]pyridine-2-carboxylate (55.80 mg, 183.86 μmol), cesium carbonate (179.72 mg, 551.59 μmol), (5-diphenylphosphanyl-9,9-dimethyl-xanthen-4-yl)-diphenyl-phosphane (21.28 mg, 36.77 μmol) and tris(dibenzylideneacetone)dipalladium (16.84 mg, 18.39 μmol) was evacuated under vacuum and refilled with N₂ three times. Dioxane (1 mL) and N,N-dimethylpyrrolidin-3-amine (41.99 mg, 367.73 μmol) were then added under N₂ atmosphere. The reaction mixture was stirred at 90° C. for 12 h. The mixture was concentrated under vacuum and purified by flash silica gel chromatography (MeOH/CH₂Cl₂/=0 to 10/1) to obtain ethyl 6-[3-(dimethylamino)pyrrolidin-1-yl]furo[3,2-b]pyridine-2-carboxylate (28.4 mg, 51% yield). MS: m/z 304.1 [M+H]+.

Step b: Ethyl 6-[3-(dimethylamino)pyrrolidin-1-yl]furo[3,2-b]pyridine-2-carboxylate (28.42 mg, 83.37 μmol) was dissolved in dioxane (0.5 mL) and water (0.5 mL) before lithium hydroxide (2.99 mg, 125.06 μmol, 1.5 eq.) was added. The solution was then heated at 50° C. for 2 hours before it was concentrated to obtain 6-[3-(dimethylamino)pyrrolidin-1-yl]furo[3,2-b]pyridine-2-carboxylic acid (22.9 mg) as an off-white powder. MS: RT m/z 276.0 [M+H]+.

Step c: 8-Fluoro-2-methyl-imidazo[1,2-a]pyridin-6-amine (27.52 mg, 166.60 μmol), 6-[3-(dimethylamino)pyrrolidin-1-yl]furo[3,2-b]pyridine-2-carboxylic acid (22.93 mg, 83.3 μmol), 3-(ethyliminomethyleneamino)-N, N-dimethyl-propan-1-amine hydrochloride salt (31.94 mg, 166.60 μmol) and 1-hydroxybenzotriazole hydrate (25.51 mg, 166.60 μmol) were added in a vial. DMF (1 mL) and N-ethyl-N-isopropyl-propan-2-amine (43.06 mg, 333.20 μmol, 58.04 μL) were then added. The reaction mixture was stirred at room temperature overnight at 40° C. overnight before being concentrated, taken up in DMSO, filtered, and purified via HPLC purification (Column: Sunfire C18 100×19 mm, 5 mm; Mobile phase A: MeCN; Mobile phase B: H20; Modifier: 0.1% TFA) to obtain 6-[3-(dimethylamino)pyrrolidin-1-yl]-N-(8-fluoro-2-methyl-imidazo[1,2-a]pyridin-6-yl)furo[3,2-b]pyridine-2-carboxamide (2.6 mg, 6.15 μmol, 7.39% yield). MS: m/z 423.1 [M+H]⁺; RT 0.79 min (Method 3). ¹H NMR (600 MHz, DMSO-d₆) δ ppm 1.87 (m, 1H) 2.17-2.22 (m, 1H) 2.24 (s, 7H) 2.35 (s, 3H) 2.84-2.89 (m, 1H) 3.18 (t, J=8.77 Hz, 1H) 3.52 (t, J=8.39 Hz, 1H) 3.59 (m, 1H) 7.09 (d, J=1.53 Hz, 1H) 7.37 (d, J=12.59 Hz, 1H) 7.76 (s, 1H) 7.93 (d, J=2.67 Hz, 1H) 8.17 (d, J=2.29 Hz, 1H) 9.08 (d, J=0.76 Hz, 1H) 10.50 (s, 1H).

### Example 15—Compound 89

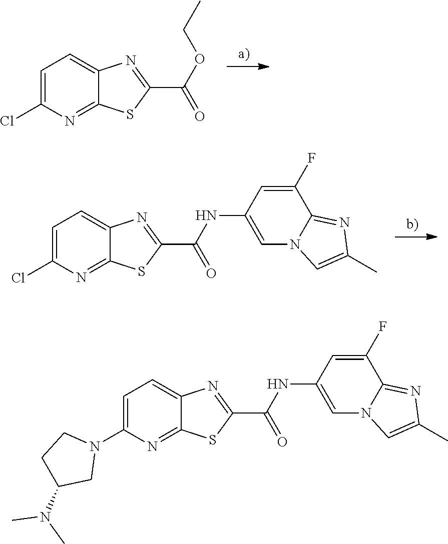

Step a: (3R)—N,N-dimethylpyrrolidin-3-amine (35.29 mg, 0.309 mmol, 0.75 eq.), ethyl 5-chlorothiazolo[5,4-b]pyridine-2-carboxylate (100 mg, 0.412 mmol, 1.0 eq.), and DIPEA (106.51 mg, 0.824 mmol, 2.0 eq.) were dissolved in Dioxane (1 mL, 0.4 M) before being heated to 80° C. for 16 hours. The solution was then concentrated via biotage V10 before being taken back up in a minimal amount of methanol and purified via 0-20% MeOH:DCM over 7 minutes. Product elutes around 10% MeOH. Obtained ethyl 5-[(3R)-3-(dimethylamino)pyrrolidin-1-yl]thiazolo[5,4-b]pyridine-2-carboxylate (47.3 mg, 0.148 mmol, 35.9% yield). MS: m/z 321.0. [M+H]⁺; RT 0.50 min (Method 4).

Step b: 8-fluoro-2-methyl-imidazo[1,2-a]pyridin-6-amine (17.73 mg, 107.32 umol, 1.2 eq.) and methyl-5-[(3R)-3-(dimethylamino)pyrrolidin-1-yl]thiazolo[5,4-b]pyridine-2-carboxylate (27.4 mg, 89.43 umol, 1.0 eq.) were dissolved in Toluene (447.15 uL, 0.2 M) before LiHMDS (1 M, 178.86 umol, 178.86 uL, 2.0 eq.) was added. The solution then stirred at RT for 16 hours before it was concentrated then taken back up in a minimal amount of DMSO, water, and methanol then filtered and injected directly onto reversed phase under acidic conditions. Identified fractions were collected, combined, and concentrated to yield an orange yellow solid that was registered as is. Obtained 5-[(3R)-3-(dimethylamino)pyrrolidin-1-yl]-N-(8-fluoro-2-methyl-imidazo[1,2-a]pyridin-6-yl)thiazolo[5,4-b]pyridine-2-carboxamide (19.8 mg, 0.035 mmol, 39.2% yield). MS: m/z 440.2. [M+H]⁺; RT 0.42 min (Method 3). ¹H NMR (400 MHz, METHANOL-d4) δ ppm 2.32-2.43 (m, 1H) 2.55-2.59 (m, 3H) 2.62-2.67 (m, 1H) 2.99-3.05 (m, 6H) 3.60-3.69 (m, 1H) 3.79-3.86 (m, 1H) 3.87-3.94 (m, 1H) 4.06-4.19 (m, 2H) 6.87-6.93 (m, 1H) 8.09-8.14 (m, 2H) 8.21-8.27 (m, 1H) 9.44-9.48 (m, 1H).

Using the procedure described for Example 15 above, additional compounds described herein were prepared by substituting the appropriate amine starting materials in steps a and b, suitable reagents and reaction conditions, obtaining compounds such as those selected from:

| Cpd ID | Data |
|---|---|
| 87 | Starting materials: 1-methyl-1,7-diazaspiro[4.4]nonane |
| | MS: m/z 478.2. [M + H]⁺; RT 2.19 min (Method 8) |
| 88 | Starting materials: 2-methyl-2,7-diazaspiro[3.4]octane |
| | MS: m/z 464.3. [M + H]⁺; RT 1.33 min (Method 3) |
| 90 | Starting materials: (1S,5S)-3-methyl-3,6-diazabicyclo[3.2.0]heptane |
| | MS: m/z 450.2. [M + H]⁺; RT 0.50 min (Method 4) |
| 91 | Starting materials: (3S)-N,N-dimethylpyrrolidin-3-amine |
| | MS: m/z 470.0. [M + H]⁺; RT 0.70 min (Method 7) |
| 92 | Starting materials: (3S)-3-(azetidin-1-yl)pyrrolidine |
| | MS: m/z 452.2. [M + H]⁺; RT 0.42 min (Method 7) |
| 93 | Starting materials: 8-fluoro-2-methyl-imidazo[1,2-a]pyridin-6-amine |
| | MS: m/z 440.2. [M + H]⁺; RT 0.43 min (Method 4) |
| | ¹H NMR (400 MHz, METHANOL-d4) δ ppm 2.32-2.42 (m, 1 H) 2.56- |
| | 2.59 (m, 3 H) 2.61-2.69 (m, 1 H) 2.99-3.04 (m, 6 H) 3.61-3.69 (m, 1 H) |
| | 3.79-3.85 (m, 1 H) 3.87-3.95 (m, 1 H) 4.06-4.20 (m, 2 H) 6.89-6.93 (m, |
| | 1 H) 8.09-8.15 (m, 2 H) 8.23-8.29 (m, 1 H) 9.45-9.51 (m, 1 H). |
| 94 | Starting materials: rac-(3S)-3-(azetidin-1-yl)pyrrolidine |
| | MS: m/z 464.1. [M + H]⁺; RT 0.52 min (Method 4) |
| 95 | Starting materials: 1-methyl-1,7-diazaspiro[4.4]nonane |
| | MS: m/z 478.2 [M + H]⁺; RT 1.45 min (Method 1) |
| 96 | Starting materials: 8-methoxy-2-methyl-imidazo[1,2-a]pyrazin-6-amine |
| | MS: m/z 453.2 [M + H]⁺; RT 0.66 min (Method 7) |
| 97 | Starting materials: tert-butyl-1,7-diazaspiro[3.4]octane-1-carboxylate |
| | MS: m/z 438.2. [M + H]⁺; RT 0.44 min (Method 4) |
| 98 | Starting materials: 1-methyl-1,7-diazaspiro[4.4]nonane |
| | MS: m/z 466.2. [M + H]⁺; RT 0.46 min (Method 4) |
| 99 | Starting materials: 3-(azetidin-1-yl)pyrrolidine |
| | MS: m/z 452.2. [M + H]⁺; RT 0.42 min (Method 4) |
| 100 | Starting materials: 8-methoxy-2-methyl-imidazo[1,2-a]pyrazin-6-amine |
| | MS: m/z 453.2 [M + H]⁺; RT 1.29 min (Method 2) |
| 101 | Starting materials: 3-(azetidin-1-yl)pyrrolidine |
| | MS: m/z 452.2. [M + H]⁺; RT 0.42 min (Method 4) |
| 102 | Starting materials: Piperizine |
| | MS: m/z 412.2. [M + H]⁺; RT 0.41 min (Method 4) |
| 103 | Starting materials: 2,2,6,6-tetramethylpiperidin-4-amine |
| | MS: m/z 482.2. [M + H]⁺; RT 0.43 min (Method 4) |
| 104 | Starting materials: (1S,5S)-3-methyl-3,6-diazabicyclo[3.2.0]heptane |
| | MS: m/z 438.2. [M + H]⁺; RT 0.41 min (Method 4) |
| 105 | Starting materials: tert-butyl (3aS,6aS)-3,3a,4,5,6,6a-hexahydro-2H- |
| | pyrrolo[3,4-b]pyrrole-1-carboxylate |
| | MS: m/z 438.2. [M + H]⁺; RT 0.42 min (Method 4) |
| 106 | Starting materials: (1R,5R)-3-methyl-3,6-diazabicyclo[3.2.0]heptane |
| | MS: m/z 438.2. [M + H]⁺; RT 0.41 min (Method 4) |
| 107 | Starting materials: (1S,5S)-3-methyl-3,6-diazabicyclo[3.2.0]heptane |
| | MS: m/z 426.2. [M + H]⁺; RT 0.42 min (Method 4) |
| 108 | Starting materials: tert-butyl (3aR,6aR)-3,3a,4,5,6,6a-hexahydro-2H- |
| | pyrrolo[3,4-b]pyrrole-1-carboxylate |
| | MS: m/z 438.2. [M + H]⁺; RT 0.42 min (Method 4) |
| 109 | Starting materials: 1-methyl-1,7-diazaspiro[4.4]nonane |
| | MS: m/z 479.1. [M + H]⁺; RT 0.70 min (Method 7) |
| 110 | Starting materials: tert-butyl (3aS,6aS)-3,3a,4,5,6,6a-hexahydro-2H- |
| | pyrrolo[3,4-b]pyrrole-1-carboxylate |
| | MS: m/z 438.2. [M + H]⁺; RT 0.42 min (Method 4) |
| 111 | Starting materials: tert-butyl-1,7-diazaspiro[3.4]octane-1-carboxylate |
| | MS: m/z 438.2. [M + H]⁺; RT 0.44 min (Method 4) |
| 112 | Starting materials: tert-butyl-1,7-diazaspiro[3.4]octane-1-carboxylate |
| | MS: m/z 438.2. [M + H]⁺; RT 0.43 min (Method 4) |
| 113 | Starting materials: N,2,2,6,6-pentamethylpiperidin-4-amine |
| | MS: m/z 496.3. [M + H]⁺; RT 1.20 min (Method 3) |
| | ¹H NMR (600 MHz, DMSO-d6) δ ppm 1.43 (s, 6 H), 1.54 (s, 6 H), 1.78- |
| | 1.82 (m, 2 H), 1.87-1.93 (m, 2 H), 2.34-2.41 (m, 1 H), 2.37 (s, 1 H), 2.55 |
| | (s, 2 H), 3.00 (s, 3 H), 5.03-5.19 (m, 1 H), 7.11 (s, 1 H), 7.63 (br d, J = 12.59 |
| | Hz, 1 H), 7.83-7.91 (m, 1 H), 7.98 (d, J = 1.91 Hz, 1 H), 8.26 (d, J = 9.16 Hz, |
| | 1 H), 8.73-8.82 (m, 1 H), 9.21 (d, J = 1.14 Hz, 1 H), 11.18 (s, 1 H) |
| 127 | Starting materials: rel-(5R)-1-methyl-1,7-diazaspiro[4.4]nonane |
| | MS: m/z 496.1. [M + H]⁺; RT 0.71 min (Method 7) |
| 128 | Starting materials: 8-fluoro-7-methoxy-2-methylimidazo[1,2-a]pyridin-6- |
| | amine and rel-(5S)-1-methyl-1,7-diazaspiro[4.4]nonane |
| | MS: m/z 496.2 [M + H]⁺; RT 2.03 min (Method 10) |
| | ¹H NMR (400 MHz, METHANOL-d₄) δ ppm = 9.09 (s, 1H), 8.47 (s, 0.5H), |
| | 8.13 (d, J = 9.2 Hz, 1H), 7.55 (d, J = 2.4 Hz, 1H), 6.75 (d, J = 9.2 Hz, 1H), |
| | 4.31 (d, J = 3.2 Hz, 3H), 3.75-3.67 (m, 1H), 3.65 (d, J = 10.4 Hz, 1H), 3.54- |
| | 3.47 (m, 1H), 3.41-3.36 (m, 1H), 2.94 (d, J = 4.2 Hz, 2H), 2.44 (s, 3H), |
| | 2.37 (s, 3H), 2.33-2.25 (m, 1H), 2.00-1.89 (m, 5H) |
| 123 | Starting materials: 7-fluoro-6-methoxy-2-methyl-2H-indazol-5-amine and |
| | rel-(5S)-1-methyl-1,7-diazaspiro[4.4]nonane |
| | MS: m/z 496.2 [M + H]⁺; RT 1.822 min (Method 8) |
| | ¹H NMR (400 MHz, METHANOL-d₄) δ ppm = 8.44 (s, 1 H), 8.23 (d, J = |
| | 2.8 Hz, 1 H), 8.17 (d, J = 9.2 Hz, 1 H), 6.81 (d, J = 9.2 Hz, 1 H), 4.21 (s, 3 |
| | H), 4.19 (s, 3 H), 3.80 (s, 1 H), 3.66-3.63 (m, 1 H), 3.60-3.53 (m, 1 H), 3.43- |
| | 3.38 (m, 1 H), 2.91-2.84 (m, 2 H), 2.39 (s, 3 H), 2.03-1.86 (m, 6 H). |
| 124 | Starting materials: 7-fluoro-6-methoxy-2-methyl-2H-indazol-5-amine and |
| | rel-(5R)-1-methyl-1,7-diazaspiro[4.4]nonane |
| | MS: m/z 496.2 [M + H]⁺; RT 2.207 min (Method 10) |
| | ¹H NMR (500 MHz, METHANOL-d₄) δ ppm = 8.44 (s, 1 H), 8.23 (d, J = |
| | 2.8 Hz, 1 H), 8.17 (d, J = 9.2 Hz, 1 H), 6.81 (d, J = 9.2 Hz, 1 H), 4.21 (s, 3 |
| | H), 4.19 (s, 3 H), 3.80 (s, 1 H), 3.66-3.63 (m, 1 H), 3.60-3.53 (m, 1 H), 3.43- |
| | 3.38 (m, 1 H), 2.91-2.84 (m, 2 H), 2.39 (s, 3 H), 2.03-1.86 (m, 6 H). |
| 125 | Starting materials: 2,8-dimethylimidazo[1,2-a]pyrazin-6-amine and 1- |
| | methyl-1,7-diazaspiro[4.4]nonane |
| | MS: m/z 463.2 [M + H]⁺; RT 1.91 min (Method 10) |
| | ¹H NMR (500 MHz, METHANOL-d₄) δ ppm = 9.05 (s, 1 H), 8.08 (d, J = |
| | 9.0 Hz, 1 H), 7.72 (s, 1 H), 6.72 (d, J = 9.5 Hz, 1 H), 3.75-3.62 (m, 1 H), |
| | 3.55 (d, J = 11.0 Hz, 1 H), 3.50-3.43 (m, 1 H), 3.35-3.30 (m, 1 H), 2.80- |
| | 2.74 (m, 2 H), 2.69 (s, 3 H), 2.38 (s, 3 H), 2.29 (s, 3 H), 2.20-2.18 (m, 1 |
| | H), 1.86-1.78 (m, 5 H) |
| 126 | Starting materials: 8-fluoro-7-methoxy-2-methylimidazo[1,2-a]pyridin-6- |
| | amine and rac-3-(azetidin-1-yl)pyrrolidine |
| | MS: m/z 482.2 [M + H]⁺; RT 1.86 min (Method 8) |
| | ¹H NMR (400 MHz, CHLOROFORM-d) δ ppm = 9.56 (s, 1H), 9.22 (s, 1H), |
| | 8.06 (d, J = 9.2 Hz, 1H), 7.30 (d, J = 2.8 Hz, 1H), 6.63 (d, J = 9.2 Hz, 1H), |
| | 4.31 (d, J = 3.6 Hz, 3H), 3.68-3.60 (m, 1H), 3.57-3.54 (m, 2H), 3.53-3.49 |
| | (m, 1H), 3.33-3.13 (m, 4H), 3.13 (s, 1H), 2.45 (s, 3H), 2.15-2.10 (m, 2H), |
| | 2.07-2.03 (m, 1H), 1.98-1.96 (m, 1H). |
| 117 | Starting materials: 8-methoxy-2-methylimidazo[1,2-a]pyrazin-6-amine |
| | MS: m/z 453.1 [M + H]⁺; RT 1.677 min (Method 8) |
| | ¹H NMR (400 MHz, CHLOROFORM-d) δ ppm = 9.23 (s, 1H), 8.84 (s, 1H), |
| | 8.07 (d, J = 9.2 Hz, 1H), 7.42 (s, 1H), 6.63 (d, J = 9.2 Hz, 1H), 4.16 (s, 3H), |
| | 3.94-3.79 (m, 2H), 3.54-3.37 (m, 2H), 2.93-2.87 (m, 1H), 2.47 (s, 3H), 2.37 |
| | (s, 6H), 2.32-2.29 (m, 1H), 2.02-1.99 (m, 1H) |
| 118 | Starting materials: 6-methoxy-2-methyl-2H-indazol-5-amine and 1- |
| | ethylpiperazine |
| | MS: m/z 452.1 [M + H]⁺; RT 2.018 min (Method 8x |
| | ¹H NMR 1H NMR (400 MHz, DMSO-d₆) δ ppm = 9.79 (s, 1H), 8.53 (s, |
| | 1H), 8.30-8.20 (m, 2H), 7.20-7.10 (m, 1H), 7.15 (s, 1H), 4.10 (s, 3H), 3.99 |
| | (s, 3H), 3.80-3.70 (m, 4H), 2.80-2.70 (m, 4H), 2.50-2.40 (m, 2H), 2.40-2.30 |
| | (m, 3H), 1.05 (t, J = 7.6 Hz, 3H). |
| 119 | Starting materials: 2,8-dimethylimidazo[1,2-a]pyrazin-6-amine and 1- |
| | ethylpiperazin |
| | MS: m/z 437.1 [M + H]⁺; RT 1.732 min (Method 8) |
| | ¹H NMR (400 MHz, DMSO-d₆) δ ppm = 10.09 (s, 1H), 9.07 (s, 1H), 8.25 |
| | (d, J = 8.8 Hz, 1H), 8.02 (s, 1H), 7.24 (d, J = 8.8 Hz, 1H), 3.70-3.60 (m, 4H), |
| | 2.80-2.90 (m, 3H), 2.60-2.50 (m, 4H), 2.40-2.30 (m, 5H), 1.10-1.00 (m, 3H). |
| 120 | Starting materials: 6-methoxy-2-methyl-2H-indazol-5-amine and (R)-3- |
| | (azetidin-1-yl)pyrrolidine |
| | MS: m/z 464.2 [M + H]⁺; RT 1.29 min (Method 3) |
| 121 | Starting materials: 6-methoxy-2-methyl-2H-indazol-5-amine and (S)-3- |
| | (azetidin-1-yl)pyrrolidine |
| | MS: m/z 464.2 [M + H]⁺; RT 1.26 min (Method 3) |
| 122 | Starting materials: 2,8-dimethylimidazo[1,2-a]pyrazin-6-amine and (S)-3- |
| | (azetidin-1-yl)pyrrolidine |
| | MS: m/z 449.1 [M + H]⁺; RT 1.844 min (Method 10) |
| | ¹H NMR (500 MHz, METHANOL-d₄) δ ppm = 9.13 (s, 1H), 9.07 (s, 1H), |
| | 8.15 (d, J = 9.0 Hz, 1H), 7.81 (s, 1H), 6.79 (d, J = 9.0 Hz, 1H), 3.70-3.60 |
| | (m, 3H), 3.50-3.40 (m, 1H), 3.40-3.30 (m, 4H), 3.25-3.15 (m, 1H), 2.78 (s, |
| | 3H), 2.47 (s, 3H), 2.20-2.05 (m, 3H), 2.00-1.90 (m, 1H). |
| 115 | Starting materials: 6-methoxy-2-methyl-2H-indazol-5-amine and N,N- |
| | dimethylpyrrolidin-3-amine |
| | MS: m/z 452.2 [M + H]⁺; RT 1.543 min (Method 10) |
| | ¹H NMR (400 MHz, METHANOL-d₄) δ ppm 8.66 (s, 1 H), 8.20 (d, J = 9.6 |
| | Hz, 1 H), 8.11 (s, 1 H), 7.08 (s, 1 H), 6.85 (d, J = 9.6 Hz, 1 H), 4.16 (s, 3 H), |
| | 4.08 (s, 3 H), 3.50-3.45 (m, 1 H), 3.40-3.30 (m, 4 H), 2.56 (s, 6 H), 1.28- |
| | 1.31 (m, 2 H). |
| 116 | Starting materials: 6-methoxy-2-methylpyrazolo[1,5-a]pyridin-5-amine and |
| | 1-methyl-1,7-diazaspiro[4.4]nonane |
| | MS: m/z 478.2 [M + H]⁺; RT 2.278 min (Method 8) |
| | ¹H NMR (400 MHz, METHANOL-d₄) δ ppm = 8.31 (s, 1H), 8.06-8.01 (m, |
| | 2H), 6.66 (d, J = 10.2 Hz, 1H), 6.20 (s, 1H), 4.02 (s, 3H), 3.71-3.61 (m, |
| | 2H), 3.49-3.37 (m, 2H), 3.11-3.04 (m, 2H), 2.52 (s, 3H), 2.36 (s, 3H), 2.32- |
| | 2.27 (m, 1H), 2.03-1.96 (m, 5H) |
| 267 | Starting materials: 8-methoxy-2-methylimidazo[1,2-a]pyridin-6-amine and |
| | 6-methyl-2,6-diazaspiro[3.4]octane |
| | MS: m/z 464.0 [M + H]⁺; RT 1.407 min (Method 10) |

### Example 16—Compound 140 and 139

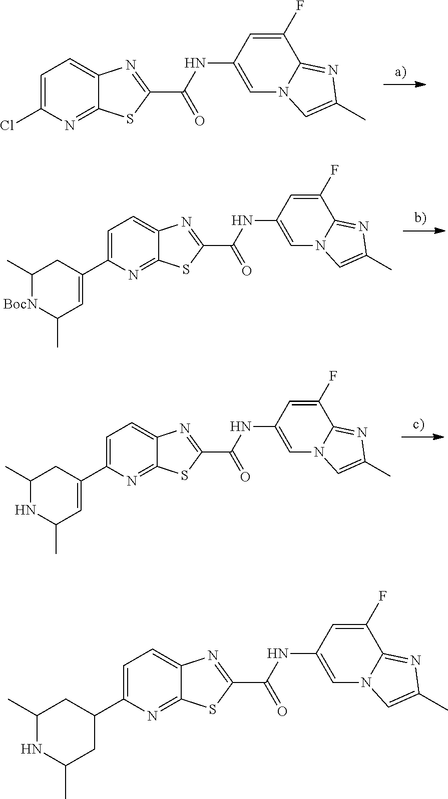

Step a: To a mixture of 5-chloro-N-(8-fluoro-2-methyl-imidazo[1,2-a]pyridin-6-yl)thiazolo[5,4-b]pyridine-2-carboxamide (115.85 mg, 320.23 umol, 1.2 eq.), tert-butyl 2,6-dimethyl-4-(4,4,5,5-tetramethyl-1,3,2-dioxaborolan-2-yl)-3,6-dihydro-2H-pyridine-1-carboxylate (90 mg, 266.86 umol, 1.0 eq.) and K2CO3 (110.64 mg, 800.57 umol, 3.0 eq.) in Dioxane (2 mL, 133 mM) and water (0.4 mL, 133 mM) was added ditert-butyl(cyclopentyl)phosphane;dichloropalladium;iron (17.39 mg, 26.69 umol, 0.1 eq.) at 20° C. The mixture was stirred at 90° C. for 1 h. The reaction mixture was concentrated to give a crude, which was purified by prep-TLC (DCM:MeOH=10/1) to give tert-butyl 4-[2-[(8-fluoro-2-methyl-imidazo[1,2-a]pyridin-6-yl)carbamoyl]thiazolo[5,4-b]pyridin-5-yl]-2,6-dimethyl-3,6-dihydro-2H-pyridine-1-carboxylate (70 mg, 130.45 umol, 48.88% yield) as a yellow solid. MS: m/z 537.3. [M+H]⁺; RT 0.95 min (Method 7).

Step b: To a mixture of tert-butyl-4-[2-[(8-fluoro-2-methyl-imidazo[1,2-a]pyridin-6-yl)carbamoyl]thiazolo[5,4-b]pyridin-5-yl]-2,6-dimethyl-3,6-dihydro-2H-pyridine-1-carboxylate (60 mg, 111.81 umol, 1.0 eq.) in DCM (2 mL, 56 mM) was added HCl/Dioxane (2 mL, 70 eq.) at 20° C. The mixture was stirred at 20° C. for 1 h. The reaction mixture was concentrated to give a crude, which was purified by prep-HPLC (Column: Boston Green ODS 150×30 mm×5 um; Condition: water (FA)-ACN, Begin B 3, End B 18, Gradient Time (min) 12, 100% B Hold Time (min) 2, Flow Rate (mL/min) 25, Injections 12.) to give 5-[(2S,6R)-2,6-dimethyl-1,2,3,6-tetrahydropyridin-4-yl]-N-(8-fluoro-2-methyl-imidazo[1,2-a]pyridin-6-yl)thiazolo[5,4-b]pyridine-2-carboxamide (25.5 mg, 58.42 umol, 52.25% yield) as a white solid. MS: m/z 437.1. [M+H]⁺; RT 1.65 min (Method 8).

Step c: To a mixture of 5-[(2S,6R)-2,6-dimethyl-1,2,3,6-tetrahydropyridin-4-yl]-N-(8-fluoro-2-methyl-imidazo[1,2-a]-pyridin-6-yl)thiazolo[5,4-b]pyridine-2-carboxamide (25 mg, 57.27 umol, 1.0 eq.) in MeOH (20 mL, 2.86 mM) was added Pd/C (60.95 mg, 57.27 umol, 8)% purity, 1.0 eq.) at 20° C. The mixture was stirred at 20° C. under H2 (15 psi) for 16 hrs. The reaction mixture was filtered and concentrated to give a crude, which was purified by prep-HPLC (Column: Boston Green ODS 150×30 mm×5 um; Condition: water (HCl)-ACN, Begin B 10, End B 40, Gradient Time (min) 10, 100% B Hold Time (min) 2, Flow Rate (mL/min) 25, Injections 1.) to give 5-[(2S,6R)-2,6-dimethyl-4-piperidyl]-N-(8-fluoro-2-methyl-imidazo[1,2-a]pyridin-6-yl)thiazolo[5,4-b]pyridine-2-carboxamide (2.2 mg, 5.02 umol, 8.76 yield) as a yellow solid. MS: m/z 439.1. [M+H]⁺; RT 1.63 min (Method 8)

Using the procedure described for Example 15 above, additional compounds described herein were prepared by substituting the appropriate boronic acid/ester and amide starting materials in step a, suitable reagents and reaction conditions, obtaining compounds such as those selected from:

| Cpd ID | Data |
|---|---|
| 129 | Starting materials: tert-butyl 4-(4,4,5,5-tetramethyl-1,3,2-dioxaborolan-2- |
| | yl)-3,6-dihydro-2H-pyridine-1-carboxylate |
| | MS: m/z 411.1. [M + H]⁺; RT 1.29 min (Method 8) |
| 130 | Starting materials: tert-butyl 3-(4,4,5,5-tetramethyl-1,3,2-dioxaborolan-2- |
| | yl)-8-azabicyclo[3.2.1]oct-2-ene-8-carboxylate |
| | MS: m/z 435.1. [M + H]⁺; RT 1.71 min (Method 8) |
| 131 | Starting materials: 1-methyl-3-(4,4,5,5-tetramethyl-1,3,2-dioxaborolan-2- |
| | yl)-2,5-dihydropyrrole |
| | MS: m/z 411.3. [M + H]⁺; RT 0.70 min (Method 7) |
| 132 | Starting materials: 2,2,6,6-tetramethyl-4-(4,4,5,5-tetramethyl-1,3,2- |
| | dioxaborolan-2-y1)-1,3-dihydropyridine |
| | MS: m/z 465.2. [M + H]⁺; RT 2.20 min (Method 8) |
| 133 | Starting materials: 1-methyl-5-(4,4,5,5-tetramethyl-1,3,2-dioxaborolan-2- |
| | yl)-3,6-dihydro-2H-pyridine |
| | MS: m/z 423.3. [M + H]⁺; RT 2.307 min (Method 8) |
| 134 | Starting materials: 1-methyl-5-(4,4,5,5-tetramethyl-1,3,2-dioxaborolan-2- |
| | y1)-3,6-dihydro-2H-pyridine |
| | MS: m/z 425.0 [M + H]⁺; RT 1.67 min (Method 8) |
| 135 | Starting materials: 1-methyl-4-(4,4,5,5-tetramethyl-1,3,2-dioxaborolan-2- |
| | yl)-3,6-dihydro-2H-pyridine |
| | MS: m/z 437.2. [M + H]⁺; RT 0.78 min (Method 7) |
| 136 | Starting materials: 2,8-dimethylimidazo[1,2-a]pyrazin-6-amine, 1-methyl- |
| | 4-(4,4,5,5-tetramethyl-1,3,2-dioxaborolan-2-yl)-3,6-dihydro-2H-pyridine |
| | MS: m/z 422.1. [M + H]⁺; RT 0.92 min (Method 7) |
| 137 | Starting materials: 6-methoxy-2-methyl-indazol-5-amine, 1-methyl-3- |
| | (4,4,5,5-tetramethyl-1,3,2-dioxaborolan-2-yl)-2,5-dihydropyrrole |
| | MS: m/z 423.2. [M + H]⁺; RT 0.92 min (Method 7) |
| 138 | Starting materials: 2,8-dimethylimidazo[1,2-a]pyrazin-6-amine, 1-methyl-3- |
| | (4,4,5,5-tetramethyl-1,3,2-dioxaborolan-2-yl)-2,5-dihydropyrrole |
| | MS: m/z 408.1. [M + H]⁺; RT 1.11 min (Method 7) |
| 246 | Starting materials: Intermediate 25 and 1-methyl-4-(4,4,5,5-tetramethyl- |
| | 1,3,2-dioxaborolan-2-y1)-1,2,3,6-tetrahydropyridine |
| | MS: m/z 425.1 [M + H]⁺; RT 2.014 min (Method 8) |

### Example 17 Compound 268

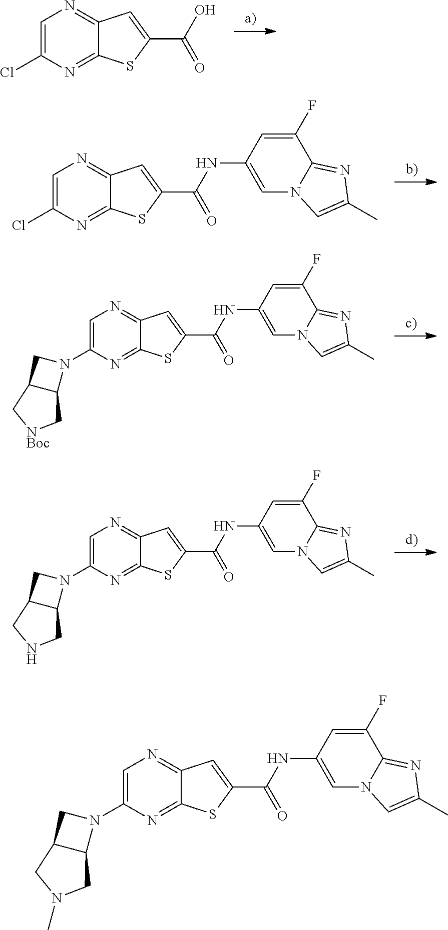

Step a: To a solution of 3-chlorothieno[2,3-b]pyrazine-6-carboxylic acid (50 mg, 233 μmol) in Pyridine (5 mL) was added EDCl (45 mg, 233 μmol) and 8-fluoro-2-methyl-imidazo[1,2-a]pyridin-6-amine (39 mg, 233 mol). The mixture was stirred at 80° C. for 2 h. The mixture was concentrated. The crude product was triturated with Ethyl Acetate (3 mL) and water (10 mL) at 25° C. for 1 h. The crude compound was used into the next step without further purification. Compound 3-chloro-N-(8-fluoro-2-methyl-imidazo[1,2-a]pyridin-6-yl)thieno[2,3-b]pyrazine-6-carboxamide (50 mg, 138 μmol) was obtained as black solid. MS: m/z 362.0 [M+H]⁺; RT 0.423 min (Method 9)

Step b: To a solution of tert-butyl (1R,5R)-3,6-diazabicyclo[3.2.0]heptane-3-carboxylate (17 mg, 83 μmol) in Dioxane (2 mL) was added TEA (25 mg, 249 μmol, 35 μL) and 3-chloro-N-(8-fluoro-2-methyl-imidazo[1,2-a]pyridin-6-yl)thieno[2,3-b]pyrazine-6-carboxamide (30 mg, 83 μmol). The mixture was stirred at 90° C. for 2 hr. The mixture was quenched with water (30 mL) and extracted with Ethyl Acetate (20 mL×3). The combined organic layers were washed with brine (20 mL), dried over Na₂SO₄, filtered, and concentrated under vacuum (low temperature) to give the crude. The crude was purified by Prep-HPLC (Column: Boston Prime C18 150*30 mm*5 um; Condition: water(FA)-ACN, Begin B 2, End B 32; Gradient Time (min): 14; 100% B Hold Time (min): 2; Flow Rate (ml/min): 25) to give tert-butyl (1R,5R)-6-[6-[(8-fluoro-2-methyl-imidazo[1,2-a]pyridin-6-yl)carbamoyl]thieno[2,3-b]pyrazin-3-yl]-3,6-diazabicyclo[3.2.0]heptane-3-carboxylate (30 mg, 57 μmol, 69% yield) as yellow solid. MS: m/z 524.2 [M+H]⁺; RT 1.135 min (Method 10)

Step c: To a solution of tert-butyl (1R,5R)-6-[6-[(8-fluoro-2-methyl-imidazo[1,2-a]pyridin-6-yl)carbamoyl]thieno[2,3-b]pyrazin-3-yl]-3,6-diazabicyclo[3.2.0]heptane-3-carboxylate (30 mg, 57 mol) in DCM (2 mL) was added TFA (7 mg, 57 μmol, 5 μL). The mixture was stirred at 25° C. for 0.5 hr. The mixture was quenched with water (30.0 mL) and extracted with Ethyl Acetate (20.0 mL×3). The combined organic layers were washed with brine (20.0 mL), dried over Na₂SO₄, filtered and concentrated under vacuum (low temperature) to give 3-[(1S,5R)-3,6-diazabicyclo[3.2.0]heptan-6-yl]-N-(8-fluoro-2-methyl-imidazo[1,2-a]pyridin-6-yl)thieno[2,3-b]pyrazine-6-carboxamide (20 mg, crude) as yellow solid. MS: m/z 424.1 [M+H]⁺; RT 1.300 min (Method 8)

Step d: To a solution of 3-[(1S,5R)-3,6-diazabicyclo[3.2.0]heptan-6-yl]-N-(8-fluoro-2-methyl-imidazo[1,2-a]pyridin-6-yl)thieno[2,3-b]pyrazine-6-carboxamide (20 mg, 47 μmol) in MeOH (20 mL) was added TEA (14 mg, 142 μmol, 20 μL) and paraformaldehyde (57 mg, 47 μmol, 64 μL). The mixture was stirred at 25° C. for 0.5 hr. Then the mixture was added NaCNBH₃ (8 mg, 118 μmol). The mixture was stirred at 25° C. for 16 hr. The mixture was quenched with water (30.0 mL) and extracted with Ethyl Acetate (20 mL×3). The combined organic layers were washed with brine (20 mL), dried over Na₂SO₄, filtered and concentrated under vacuum (low temperature) to give the crude. The crude was purified by Prep-HPLC (Column: Boston Prime C18 150*30 mm*5 um; Condition: water(FA)-ACN, Begin B 2, End B 32; Gradient Time (min): 14; 100% B Hold Time (min): 2; Flow Rate (ml/min): 25) to give N-(8-fluoro-2-methyl-imidazo[1,2-a]pyridin-6-yl)-3-[(1S,5R)-3-methyl-3,6-diazabicyclo[3.2.0]heptan-6-yl]thieno[2,3-b]pyrazine-6-carboxamide (5 mg, 12 μmol) as yellow solid. MS: m/z 438.2 [M+H]⁺; RT 1.17 min (Method 10)

Using the procedure described for Example 15 above, additional compounds described herein were prepared by substituting the appropriate amine starting materials in steps a and b, suitable reagents and reaction conditions, obtaining compounds such as those selected from:

| Cpd ID | Data |
|---|---|
| 247 | Starting materials: 6-methoxy-2-methyl-2H-indazol-5-amine and 1- |
| | ethylpiperazine |
| | MS: m/z 452.2 [M + H]⁺; RT 1.442 min (Method 10) |
| | ¹H NMR (400 MHz, METHANOL-d₄) δ ppm = 8.46 (s, 1H), 8.27 (s, 1H), |
| | 8.09 (s, 1H), 8.01 (s, 1H), 7.04 (s, 1H), 4.15 (s, 3H), 4.01 (s, 3H), 3.84-3.80 |
| | (m, 4H), 2.66-2.63 (m, 4H), 2.56-2.50 (m, 2H), 1.19-1.15 (m, 3H). |
| 248 | Starting materials: 6-methoxy-2-methyl-2H-indazol-5-amine and 2-methyl- |
| | 2,6-diazaspiro[3.4]octane |
| | MS: m/z 464.2 [M + H]⁺; RT 1.191 min (Method 10) |
| | ¹H NMR (400 MHz, METHANOL-d₄) δ = 8.28 (s, 1H), 8.13 (s, 1H), 8.09 |
| | (s, 1H), 7.99 (s, 1H), 7.04 (s, 1H), 4.15 (s, 3H), 4.02 (s, 3H), 3.75 (s, 2H), |
| | 3.67-3.63 (m, 2H), 3.36 (d, J = 2.0 Hz, 4H), 2.41 (s, 3H), 2.30-2.25 (m, 2H). |
| 257 | Starting materials: 2-methyl-2,6-diazaspiro[3.4]octane |
| | MS: m/z 452.1 [M + H]⁺; RT 1.617 min (Method 10) |
| | ¹HNMR (400 MHz, METHANOL-d₄) δ ppm = 8.99 (s, 1H), 8.28 (d, J = 1.2 |
| | Hz, 1H), 8.17 (s, 1H), 8.11 (s, 1H), 7.73 (d, J = 2.0 Hz, 1H), 7.30-7.27 (m, |
| | 1H), 4.27-4.19 (m, 4H), 3.93 (s, 2H), 3.74-3.71 (m, 2H), 2.98 (s, 3H), 2.43 |
| | (s, 3H), 1.33-1.30 (m, 2H) |
| 258 | Starting materials: 3-methylazetidin-3-amine |
| | MS: m/z 412.1 [M + H]⁺; RT 1.590 min (Method 10) |
| | ¹HNMR (400 MHz, METHANOL-d₄) δ ppm = 8.99 (d, J = 1.6 Hz, 1H), 8.09 |
| | (s, 1H), 8.01 (s, 1H), 7.72 (d, J = 2.4 Hz, 1H), 7.29-7.26 (m, 1H), 4.11-4.09 |
| | (m, 2H), 4.03-4.01 (m, 2H), 2.43 (s, 3H), 1.54 (s, 3H) |
| 264 | Starting materials: 6-methoxy-2-methyl-2H-indazol-5-amine and tert-butyl |
| | 1,6-diazaspiro[3.4]octane-1-carboxylate |
| | MS: m/z 464.2 [M + H]⁺; RT 1.293 min (Method 10) |

### Example 18—Compound 147 and 148

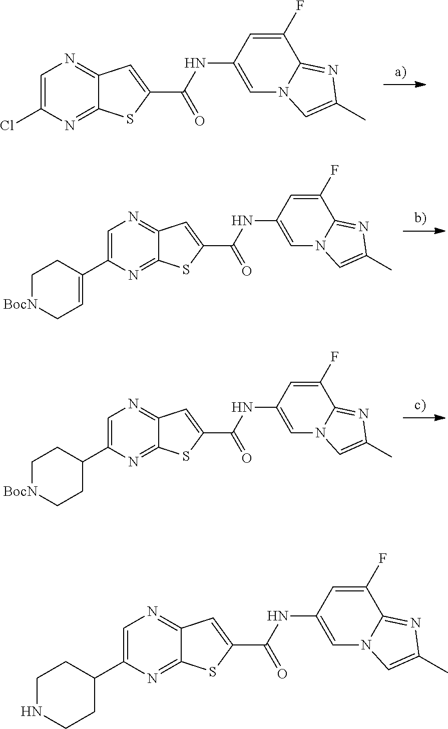 [SMILES: Cc1cn2cc(NC(=O)c3cc4ncc(Cl)nc4s3)cc(F)c2n1 ; Cc1cn2cc(NC(=O)c3cc4ncc(C5=CCN(C(=O)OC(C)(C)C)CC5)nc4s3)cc(F)c2n1 ; Cc1cn2cc(NC(=O)c3cc4ncc(C5CCN(C(=O)OC(C)(C)C)CC5)nc4s3)cc(F)c2n1 ; Cc1cn2cc(NC(=O)c3cc4ncc(C5CCNCC5)nc4s3)cc(F)c2n1]

Step a: To a solution of 3-chloro-N-(8-fluoro-2-methyl-imidazo[1,2-a]pyridin-6-yl)thieno[2,3-b]pyrazine-6-carboxamide (120 mg, 332 mol) in Dioxane (2.5 mL) and water (0.5 mL) was added K₂CO₃ (138 mg, 995 mol), tert-butyl 4-(4,4,5,5-tetramethyl-1,3,2-dioxaborolan-2-yl)-3,6-dihydro-2H-pyridine-1-carboxylate (103 mg, 332 μmol) and PdCl₂(dppf) (24 mg, 33 μmol). The mixture was stirred at 90° C. for 2 h under N₂. The mixture was filtered and concentrated to give a residue. The residue was purified by column chromatography (SiO₂, DCM:MeOH=20:1 to 10:1). Compound tert-butyl 4-[6-[(8-fluoro-2-methyl-imidazo[1,2-a]pyridin-6-yl)carbamoyl]thieno[2,3-b]pyrazin-3-yl]-3,6-dihydro-2H-pyridine-1-carboxylate (100 mg, 197 mol) was obtained as yellow solid. MS: m/z 509.4 [M+H]⁺; RT 0.392 min (Method 9)

Step b: tert-butyl 4-[6-[(8-fluoro-2-methyl-imidazo[1,2-a]pyridin-6-yl)carbamoyl]thieno[2,3-b]pyrazin-3-yl]-3,6-dihydro-2H-pyridine-1-carboxylate (20 mg, 39 μmol) was added into MeOH (10 mL) under Argon. The mixture was added Pd/C (20 mg, 188 μmol) under Argon. Then the mixture was stirred at 25° C. and 35 Psi for 16 hr under H₂. The mixture was quenched with water (30.0 mL) and extracted with Ethyl Acetate (20 mL×3). The combined organic layers were washed with brine (20 mL), dried over Na₂SO₄, filtered and concentrated under vacuum (low temperature) to give the crude. The crude was purified by chromatography column on silica gel (DCM/MeOH=10/0 to 10/1) to give tert-butyl 4-[6-[(8-fluoro-2-methyl-imidazo[1,2-a]pyridin-6-yl)carbamoyl]thieno[2,3-b]pyrazin-3-yl]piperidine-1-carboxylate (15 mg, 29 μmol) as yellow solid. MS: m/z 511.3 [M+H]⁺; RT 0.953 min (Method 10).

Step c: Tert-butyl 4-[6-[(8-fluoro-2-methyl-imidazo[1,2-a]pyridin-6-yl)carbamoyl]thieno[2,3-b]pyrazin-3-yl]piperidine-1-carboxylate (15 mg, 29 mol) was added into HCl/EA (2 mL). The mixture was stirred at 25° C. for 1 hr. The mixture was quenched with water (30 mL) and extracted with Ethyl Acetate (20 mL×3). The combined organic layers were washed with brine (20 mL), dried over Na₂SO₄, filtered and concentrated under vacuum (low temperature) to give the crude. The crude was purified by Prep-HPLC (Column: Boston Prime C18 150*30 mm*5 um; Condition: water(FA)-ACN, Begin B 2, End B 32; Gradient Time (min): 14; 100% B Hold Time (min): 2; Flow Rate (ml/min): 25) to give N-(8-fluoro-2-methyl-imidazo[1,2-a]pyridin-6-yl)-3-(4-piperidyl)thieno[2,3-b]pyrazine-6-carboxamide (5.4 mg, 13 μmol) as yellow solid. MS: m/z 411.1 [M+H]⁺; RT 1.570 min (Method 10).

Using the procedure described for Example 15 above, additional compounds described herein were prepared by substituting the appropriate boronic acid/ester and amide starting materials in step a, suitable reagents and reaction conditions, obtaining compounds such as those selected from:

| Cpd ID | Data |
|---|---|
| 141 | Starting materials: tert-butyl N-[rac-(1S)-4-(4,4,5,5-tetramethyl-1,3,2- |
| | dioxaborolan-2-yl)cyclohex-3-en-1-yl]carbamate |
| | MS: m/z 425.1 [M + H]⁺; RT 0.363 min (Method 9) |
| 142 | Starting materials: tert-butyl 3-(4,4,5,5-tetramethyl-1,3,2-dioxaborolan-2- |
| | yl)-8-azabicyclo[3.2.1]oct-2-ene-8-carboxylate |
| | MS: m/z 437.1 [M + H]⁺; RT 1.387 min (Method 8) |
| 143 | Starting materials: rac-tert-butyl (R)-(3-(4,4,5,5-tetramethyl-1,3,2- |
| | dioxaborolan-2-yl)cyclohex-3-en-1-yl)carbamate |
| | MS: m/z 423.3 [M + H]⁺; RT 0.244 min (Method 9) |
| 144 | Starting materials: tert-butyl 6-(4,4,5,5-tetramethyl-1,3,2-dioxaborolan-2- |
| | yl)-3,4-dihydro-2H-pyridine-1-carboxylate |
| | MS: m/z. 411.1 [M + H]⁺; RT 1.302 min (Method 8) |
| 145 | Starting materials: rac-tert-butyl (R)-(3-(4,4,5,5-tetramethyl-1,3,2- |
| | dioxaborolan-2-y1)cyclohex-3-en-1-yl)carbamate |
| | MS: m/z 423.3 [M + H]⁺; RT 0.254 min (Method 9) |
| 146 | Starting materials: tert-butyl 3-(4,4,5,5-tetramethyl-1,3,2-dioxaborolan-2- |
| | yl)-2,5-dihydropyrrole-1-carboxylate |
| | MS: m/z 397.0 [M + H]⁺; RT 1.247 min (Method 8) |

### Example 19—Compound 152

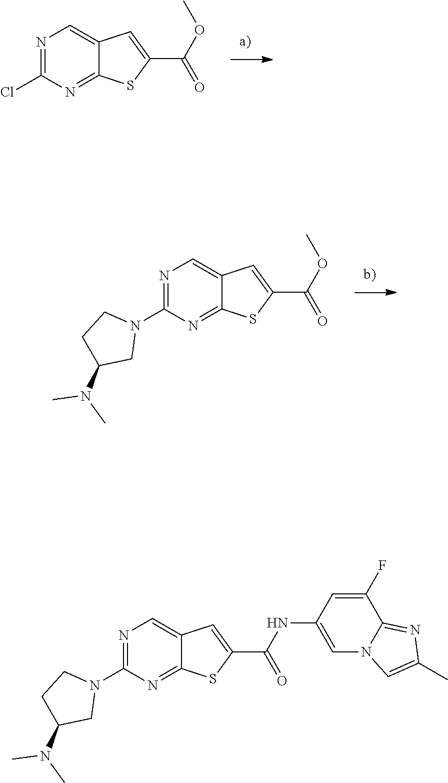

Step a: (3S)—N,N-dimethylpyrrolidin-3-amine (49.94 mg, 437.3 mmol, 1.0 eq.), methyl 2-chlorothieno[2,3-d]pyrimidine-6-carboxylate (100 mg, 437.3 mmol, 1.0 eq.), and DIPEA (113 mg, 875 mmol, 2.0 eq.) were dissolved in Dioxane (1 mL, 0.44 M) before being heated to 80° C. for 16 hours. The solution was then concentrated via biotage V10 before being taken back up in a minimal amount of methanol and purified via 0-20% MeOH:DCM over 7 minutes. Product elutes at 10% MeOH. Identified fractions were collected, combined, and concentrated to yield methyl 2-[(3S)-3-(dimethylamino)pyrrolidin-1-yl]thieno[2,3-d]pyrimidine-6-carboxylate (111.3 mg, 363 mmol, 83% yield). MS: m/z 307.0. [M+H]⁺; RT 0.45 min (Method 4)

Step b: 8-fluoro-2-methyl-imidazo[1,2-a]pyridin-6-amine (16.17 mg, 97.92 umol, 1.2 eq.) and methyl 2-[(3S)-3-(dimethylamino)pyrrolidin-1-yl]thieno[2,3-d]pyrimidine-6-carboxylate (25.00 mg, 81.60 umol, 1.0 eq.) were dissolved in Toluene (407.99 uL, 0.2 M) before LiHMDS (1 M, 163.19 umol, 2.0 eq.) was added. The solution then stirred at RT for 16 hours before it was concentrated then taken back up in DMSO, methanol, and water then filtered and injected directly onto reversed phase column under acidic conditions. Identified fractions were collected and concentrated then registered as is. Obtained 2-[(3S)-3-(dimethylamino)pyrrolidin-1-yl]-N-(8-fluoro-2-methyl-imidazo[1,2-a]pyridin-6-yl)thieno[2,3-d]pyrimidine-6-carboxamide (22.8 mg, 41.19 umol, 50.48% yield, Trifluoroacetic acid) as an orange yellow solid. ¹H NMR (400 MHz, MVIETHANOL-d4) δ ppm 2.30-2.40 (m, 1H) 2.54-2.57 (m, 3H) 2.58-2.65 (m, 1H) 2.99-3.02 (m, 6H) 3.66-3.74 (m, 1H) 3.82-3.88 (m, 1H) 3.96-4.04 (m, 1H) 4.05-4.13 (m, 1H) 4.18-4.25 (m, 1H) 7.94-7.99 (m, 1H) 8.06-8.08 (m, 1H) 8.08-8.10 (m, 1H) 8.91-8.94 (m, 1H) 9.37-9.40 (m, 1H). MS: m/z 440.2. [M+H]⁺; RT 0.43 min (Method 4).

Using the procedure described for Example 15 above, additional compounds described herein were prepared by substituting the appropriate amine starting materials in steps a and b, suitable reagents and reaction conditions, obtaining compounds such as those selected from:

| Cpd ID | Data |
|---|---|
| 149 | Starting materials: (3R)-N,N-dimethylpyrrolidin-3-amine |
| | MS: m/z 440.2. [M + H]⁺; RT 0.44 min (Method 4) |
| 150 | Starting materials: (3S)-N,N-dimethylpyrrolidin-3-amine, 2,8- |
| | dimethylimidazo[1,2-a]pyrazin-6-amine |
| | MS: m/z 437.2. [M + H]⁺; RT 0.46 min (Method 4) |
| 151 | Starting materials: (3R)-N,N-dimethylpyrrolidin-3-amine, 2,8- |
| | dimethylimidazo[1,2-a]pyrazin-6-amine |
| | MS: m/z 437.2. [M + H]⁺; RT 0.48 min (Method 4) |

### Example 20—Compound 153

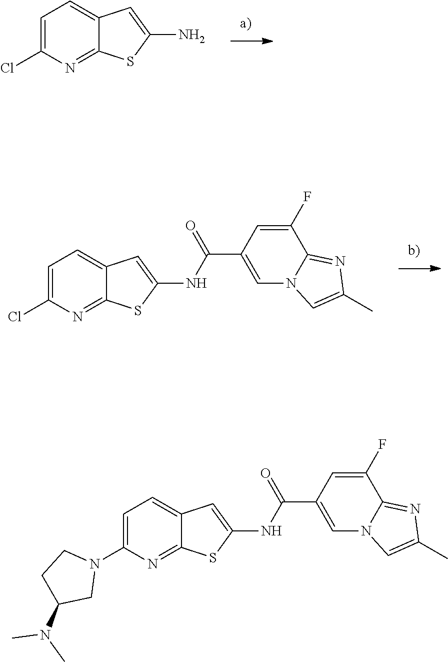

Step a: To a DCM (2 mL) solution of 8-fluoro-2-methyl-imidazo[1,2-a]pyridine-6-carboxylic acid (80 mg, 412.03 μmol) and 6-chlorothieno[2,3-b]pyridin-2-amine (91.29 mg, 494.43 μmol) were added oxalyl dichloride (2 M, 824.05 μL) and N,N-diethylethanamine (166.77 mg, 1.65 mmol, 229.71 μL). The reaction mixture was stirred at 60° C. overnight and then concentrated. The residue was purified by column chromatography (0 to 10% MeOH/DCM) to yield N-(6-chlorothieno[2,3-b]pyridin-2-yl)-8-fluoro-2-methyl-imidazo[1,2-a]pyridine-6-carboxamide (66.0 mg, 182.93 μmol, 44.40% yield) as pale solid. MS: m/z 361.1 [M+H]⁺.

Step b: N-(6-chlorothieno[2,3-b]pyridin-2-yl)-8-fluoro-2-methyl-imidazo[1,2-a]pyridine-6-carboxamide (25 mg, 69.29 μmol), sodium;2-methylpropan-2-olate (19.98 mg, 207.88 μmol) and [2-(2-aminophenyl)phenyl]-methylsulfonyloxy-palladium;dicyclohexyl-[3,6-dimethoxy-2-(2,4,6-triisopropylphenyl)phenyl]phosphane (6.28 mg, 6.93 μmol) were added in a microwave vial. The mixture was evacuated under vacuum and refill with N₂ three times. 2-Me-THF (1 mL) and (3S)—N,N-dimethylpyrrolidin-3-amine (23.74 mg, 207.88 μmol, 26.67 μL) were then added. The reaction mixture was stirred at 90° C. for 12 h and then concentrated under vacuum. The reside was purified by HPLC (Column: Sunfire C18 100×19 mm, 5 mm; Mobile phase A: MeCN; Mobile phase B: H20; Modifier: 0.1% TFA) to obtain N-[6-[(3S)-3-(dimethylamino)pyrrolidin-1-yl]thieno[2,3-b]pyridin-2-yl]-8-fluoro-2-methyl-imidazo[1,2-a]pyridine-6-carboxamide (3.7 mg, 8.44 μmol, 12.18% yield) as a yellow solid. MS: m/z 361.1 [M+H]⁺. ¹H NMR (600 MHz, DMSO-d₆) δ ppm 2.09-2.20 (m, 1H), 2.35 (s, 3H), 2.39 (br s, 1H), 2.55 (s, 1H), 2.59-2.85 (m, 6H), 3.45-3.57 (m, 2H), 3.76 (br t, J=8.39 Hz, 1H), 3.94 (br s, 1H), 6.75 (d, J=8.77 Hz, 1H), 7.31 (d, J=12.59 Hz, 1H), 7.91 (d, J=1.91 Hz, 1H), 8.10-8.14 (m, 2H), 9.00 (s, 1H), 10.42 (s, 1H).

Using the procedure described for Example 15 above, additional compounds described herein were prepared by substituting the appropriate acid starting material in step a, suitable reagents and reaction conditions, obtaining compounds such as those selected from:

| Cpd ID | Data |
|---|---|
| 154 | Starting materials: 7-methoxy-2-methyl-2H-indazole-5-carboxylic acid and |
| | (1S,5S)-3-methyl-3,6-diazabicyclo[3.2.0]heptane |
| | MS: m/z 449.2 [M + H]⁺; RT 1.08 min (Method 3) |

### Example 21—Compound 156

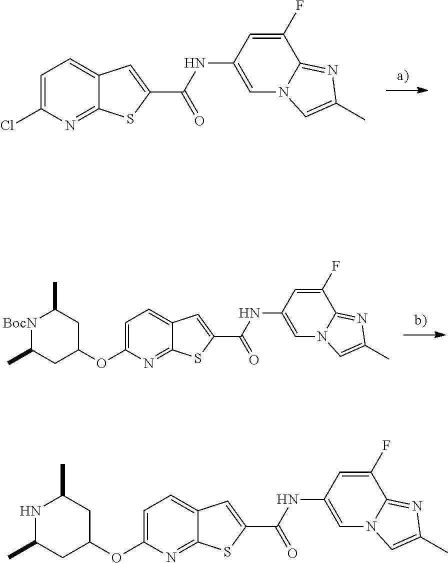

Step a: tert-Butyl rac-(2 S, 6R)-4-hydroxy-2,6-dimethyl-piperidine-1l-carboxylate (23.83 mg, 103.94 μmol) and 6-chloro-N-(8-fluoro-2-methyl-imidazo[1,2-a]pyridin-6-yl)thieno[2,3-b]pyridine-2-carboxamide (25 mg, 69.29 μmol) were dissolved in DMF (1 mL), sodium hydride (9.98 mg, 415.75 μmol) was then added. The solution was then heated to 40° C. for 16 hours. The reaction was concentrated under vacuum. The residue was used directly for next step.

Step b: To a DCM solution (1 mL) of tert-butyl (2S,6R)-4-((2-((8-fluoro-2-methylimidazo[1,2-a]pyridin-6-yl)carbamoyl)thieno[2,3-b]pyridin-6-yl)oxy)-2,6-dimethylpiperidine-1-carboxylate was added HCl (4 M, 1.11 mmol, 277.17 μL). The mixture was stirred for 2 h and then concentrated. The residue was purified by HPLC (Column: Sunfire C18 100×19 mm, 5 mm; Mobile phase A: MeCN; Mobile phase B: H20; Modifier: 0.1% TFA) to obtain 6-[[(2SR,6RS)-2,6-dimethyl-4-piperidyl]oxy]-N-(8-fluoro-2-methyl-imidazo[1,2-a]pyridin-6-yl)thieno[2,3-b]pyridine-2-carboxamide (4.8 mg, 10.58 μmol, 15.27% yield) as a yellow solid. MS: m/z 454.2 [M+H]⁺; RT 1.10 min (Method 3).

Using the procedure described for Example 15 above, additional compounds described herein were prepared by substituting the appropriate alcohol and amide starting materials in step a, suitable reagents and reaction conditions, obtaining compounds such as those selected from:

| Cpd ID | Data |
|---|---|
| 155 | Starting materials: rel-(1S,3R)-3-(Dimethylamino)cyclopentan-1-ol |
| | MS: m/z 453.2 [M + H]⁺; RT 1.08 min (Method 3) |
| 157 | Starting materials: tert-butyl rac-(2R, 6R)-4-hydroxy-2,6-dimethyl- |
| | piperidine-1-carboxylate |
| | MS: m/z 454.2 [M + H]⁺; RT 1.11 min (Method 3) |
| 158 | Starting materials: tert-butyl rac-(4S)-4-hydroxy-2,2-dimethyl-piperidine-1- |
| | carboxylate |
| | MS: m/z 454.2 [M + H]⁺; RT 1.11 min (Method 3) |
| 159 | Starting materials: rac-(3S)-1-methylpyrrolidin-3-ol |
| | MS: m/z 426.1 [M + H]⁺; RT 1.00 min (Method 3) |
| 160 | Starting materials: tert-butyl (S)-2-hydroxy-8-azaspiro[4.5]decane-8- |
| | carboxylate |
| | MS: m/z 494.2 [M + H^{]+}; RT 1.24 min (Method 3) |
| 161 | Starting materials: rac-(3S)-1-ethylpyrrolidin-3-ol |
| | MS: m/z 440.1 [M + H]⁺; RT 1.01 min (Method 3) |
| | ¹H NMR (600 MHz, DMSO-d₆) δ ppm 1.24 (t, J = 7.25 Hz, 3 H), 2.14-2.21 |
| | (m, 1 H), 2.32-2.36 (m, 1 H), 2.38 (s, 3 H), 2.63-2.70 (m, 1 H), 3.19-3.28 |
| | (m, 2 H), 3.53 (ddd, J = 13.26, 7.92, 5.91 Hz, 1 H), 3.72-3.78 (m, 1 H), 3.83 |
| | (br dd, J = 12.59, 4.20 Hz, 1 H), 5.74 (br d, J = 1.91 Hz, 1 H), 7.42 (br d, |
| | J = 12.21 Hz, 1 H), 7.98 (d, J = 1.91 Hz, 1 H), 8.29 (s, 1 H), 8.41 (d, J = 8.77 Hz, |
| | 1 H), 9.09 (s, 1 H), 10.02-10.14 (m, 1 H), 10.71 (s, 1 H). |
| 162 | Starting materials: tert-butyl 2-hydroxy-7-azaspiro[3.5]nonane-7- |
| | carboxylate |
| | MS: m/z 480.2 [M + H]⁺; RT 1.16 min (Method 3) |

### Example 22—Compound 163

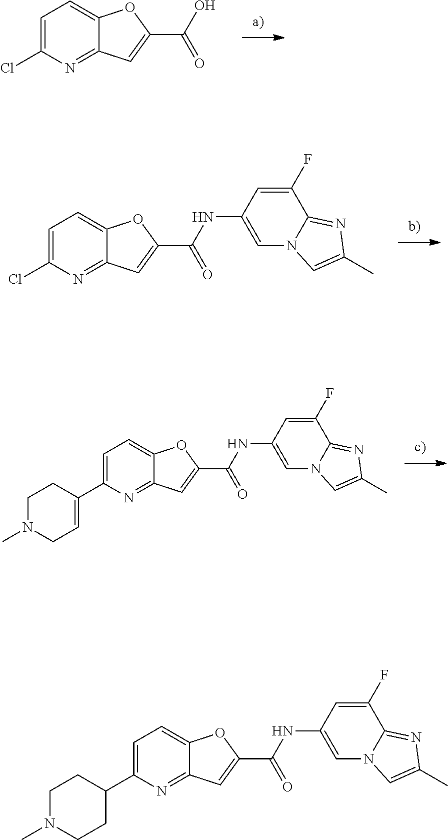

Step a: To a mixture of 5-chlorofuro[3,2-b]pyridine-2-carboxylic acid (20 mg, 101.23 mol) and 8-fluoro-2-methyl-imidazo[1,2-a]pyridin-6-amine (16.72 mg, 101.23 μmol) in DMF (2 mL) was added HATU (57.73 mg, 151.84 μmol) and DIPEA (19.62 mg, 151.84 μmol, 26.45 L) in one portion at 25° C. The mixture was stirred at 90° C. for 80 min. Then the mixture was cooled to 25° C. and concentrated in reduced pressure. The residue was concentrated in vacuum. The residue was purified by silica gel chromatography (DCM/MeOH=50/1 to 20/1) to afford 5-chloro-N-(8-fluoro-2-methyl-imidazo[1,2-a]pyridin-6-yl)furo[3,2-b]pyridine-2-carboxamide (21 mg, 60.92 μmol, 60.18% yield) as a brown solid. MS: m/z 345.1 [M+H]⁺.

Step b: A mixture of 5-chloro-N-(8-fluoro-2-methyl-imidazo[1,2-a]pyridin-6-yl)furo[3,2-b]pyridine-2-carboxamide (20 mg, 58.02 mol), 1-methyl-4-(4,4,5,5-tetramethyl-1,3,2-dioxaborolan-2-yl)-3,6-dihydro-2H-pyridine (12.94 mg, 58.02 μmol) in dioxane (3 mL) was added K₂CO₃ (24.05 mg, 174.05 μmol), Pd(dppf)Cl₂ (42.45 mg, 58.02 μmol) and stirred at 90° C. for 6 h. The reaction mixture was filtered and concentrated to afford crude product. The mixture was further purified by silica gel column chromatography (DCM/MeOH=50/1 to 10/1) to give N-(8-fluoro-2-methyl-imidazo[1,2-a]pyridin-6-yl)-5-(1-methyl-3,6-dihydro-2H-pyridin-4-yl)furo[3,2-b]pyridine-2-carboxamide (15.6 mg, 38.48 μmol, 66.32% yield) as a brown solid. MS: m/z 406.2 [M+H]⁺.

Step c: To a solution of N-(8-fluoro-2-methyl-imidazo[1,2-a]pyridin-6-yl)-5-(1-methyl-3,6-dihydro-2H-pyridin-4-yl)furo[3,2-b]pyridine-2-carboxamide (25 mg, 61.66 mol) in MeOH (5 mL) and THE (5 mL) was added Pd/C (19.69 mg, 18.50 μmol, 10% purity). The suspension was degassed under vacuum and purged with H₂ several times. The mixture was heated to 35° C. (45 psi) and stirred for 5 hours. The mixture was filtered and concentrated in vacuum. The residue was purified by preparative HPLC (Column Boston Prime C18 150×30 mm×5 um; Condition water(NH3H2+NH4HCO3)-ACN; Begin B 44; End B 74; Gradient Time (min) 10; 100% B Hold Time (min) 2; FlowRate (ml/min) 25) to afford N-(8-fluoro-2-methyl-imidazo[1,2-a]pyridin-6-yl)-5-(1-methyl-4-piperidyl)furo[3,2-b]pyridine-2-carboxamide (4.42 mg, 10.85 μmol, 17.59% yield, 100% purity) as a white solid. MS: m/z 408.2 [M+H]⁺; RT 2.572 min (Method 8). ¹H NMR (400 MHz, METHANOL-d₄) δ ppm=9.10 (d, J=1.6 Hz, 1H), 8.07 (d, J=9.2 Hz, 1H), 7.79-7.74 (m, 2H), 7.50 (d, J=8.8 Hz, 1H), 7.39-7.36 (m, 1H), 3.14 (d, J=10.8 Hz, 2H), 2.99-2.90 (m, 1H), 2.46-2.42 (m, 6H), 2.40-2.31 (m, 2H), 2.04-2.02 (m, 4H).

Using the procedure described for Example 15 above, additional compounds described herein were prepared by substituting the appropriate amine starting material in step a and boronic acid/ester starting material in step b, suitable reagents and reaction conditions, obtaining compounds such as those selected from:

| Cpd ID | Data |
|---|---|
| 262 | Starting materials: 8-methoxy-2-methylimidazo[1,2-a]pyrazin-6-amine |
| | MS: m/z 421.2 [M + H]⁺; RT 1.722 min (Method 8) |
| 269 | Starting materials: 6-methoxy-2-methyl-2H-indazol-5-amine |
| | MS: m/z 420.2 [M + H]⁺; RT 1.556 min (Method 8) |

### Example 23—Compound 168

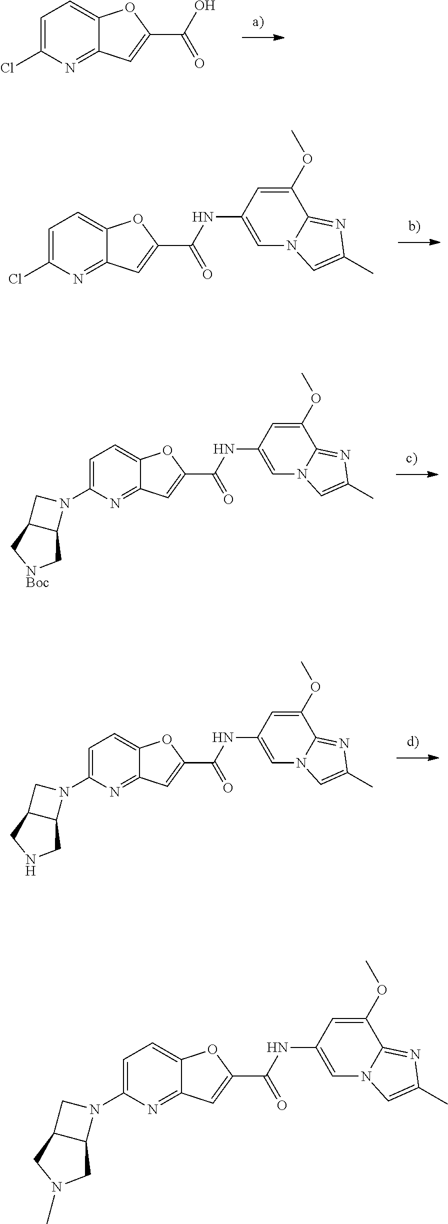 [SMILES: O=C(O)c1cc2nc(Cl)ccc2o1 ; COc1cc(NC(=O)c2cc3nc(Cl)ccc3o2)cn2cc(C)nc12 ; COc1cc(NC(=O)c2cc3nc(N4C[C@@H]5CN(C(=O)OC(C)(C)C)C[C@@H]54)ccc3o2)cn2cc(C)nc12 ; COc1cc(NC(=O)c2cc3nc(N4C[C@@H]5CNC[C@@H]54)ccc3o2)cn2cc(C)nc12 ; COc1cc(NC(=O)c2cc3nc(N4C[C@@H]5CN(C)C[C@@H]54)ccc3o2)cn2cc(C)nc12]

Step a: To a solution of 5-chlorofuro[3,2-b]pyridine-2-carboxylic acid (100 mg, 506.14 gmol) and HATU (384.90 mg, 1.01 mmol) in DMF (2 mL) was added N-ethyl-N-isopropyl-propan-2-amine (196.24 mg, 1.52 mmol, 264.48 μL) and 8-methoxy-2-methyl-imidazo[1,2-a]pyridin-6-amine (89.69 mg, 506.14 μmol). The mixture was stirred at 25° C. for 16 hr. The mixture then was quenched with water (30.0 mL) and extracted with EA (20.0 mL×3). The combined organic layers were washed with brine (20.0 mL), dried over Na₂SO₄, filtered and concentrated under vacuum (low temperature) to give 5-chloro-N-(8-methoxy-2-methyl-imidazo[1,2-a]pyridin-6-yl)furo[3,2-b]pyridine-2-carboxamide (70 mg, 196.21 μmol, 38.77% yield) as yellow solid. MS: m/z 356.8 [M+H]⁺.

Step b: To a solution of 5-chloro-N-(8-methoxy-2-methyl-imidazo[1,2-a]pyridin-6-yl)furo[3,2-b]pyridine-2-carboxamide (60 mg, 168.18 μmol) in THE (15 mL) was added sodium tert-butoxide (48.49 mg, 504.54 μmol), 5-chloro-N-(8-methoxy-2-methyl-imidazo[1,2-a]pyridin-6-yl)furo[3,2-b]pyridine-2-carboxamide (60 mg, 168.18 mol) and tert-butyl (1S,5S)-3,6-diazabicyclo[3.2.0]heptane-3-carboxylate (50.02 mg, 252.27 mol). The mixture was stirred at 80° C. for 2 h under N₂. The mixture was filtered and concentrated. The residue was purified by column chromatography (DCM:MeOH=10:1) to yield tert-butyl (1S,5S)-6-[2-[(8-methoxy-2-methyl-imidazo[1,2-a]pyridin-6-yl)carbamoyl]furo[3,2-b]pyridin-5-yl]-3,6-diazabicyclo[3.2.0]heptane-3-carboxylate (79 mg, 152.34 μmol, 90.58% yield) as yellow oil. MS: m/z 519.3 [M+H]⁺.

Step c: To a solution of tert-butyl (1S,5S)-6-[2-[(8-methoxy-2-methyl-imidazo[1,2-a]pyridin-6-yl)carbamoyl]furo[3,2-b]pyridin-5-yl]-3,6-diazabicyclo[3.2.0]heptane-3-carboxylate (79 mg, 152.34 mol) in HFIP (3 mL) was added TFA (34.74 mg, 304.69 μmol, 23.33 μL). The mixture was stirred at 25° C. for 1 h. The mixture was filtered and concentrated. The crude compound was used into the next step without further purification. MS: m/z 419.3 [M+H]⁺.

Step d: To a solution of 5-[(1R,5S)-3,6-diazabicyclo[3.2.0]heptan-6-yl]-N-(8-methoxy-2-methyl-imidazo[1,2-a]pyridin-6-yl)furo[3,2-b]pyridine-2-carboxamide (50 mg, 119.49 μmol) in DCE/EtOH (4 mL) was added TEA (36.27 mg, 358.47 μmol, 49.96 μL) and paraformaldehyde (143.33 mg, 119.49 μmol, 162.88 μL). The mixture was stirred at 25° C. for 10 min. Then added sodium triacetoxyboranuide (75.97 mg, 358.47 mol). The mixture was stirred at 25° C. for 2 h. The mixture was filtered and concentrated. The residue was purified by HPLC purification (Column Boston Prime C18 150×30 mm×5 um; Condition water (NH3H2O+NH4HCO3)-ACN; Begin B 44; End B 74; Gradient Time (min) 10; 100% B Hold Time (min) 2; FlowRate (ml/min) 25) to yield N-(8-methoxy-2-methyl-imidazo[1,2-a]pyridin-6-yl)-5-[(1R,5S)-3-methyl-3,6-diazabicyclo[3.2.0]heptan-6-yl]furo[3,2-b]pyridine-2-carboxamide (6.27 mg, 14.50 μmol, 12.130 yield) was obtained as a yellow solid. MS: m/z 433.1 [M+H]⁺; RT 0.663 min (Method 10). ¹H NMR (400 MHz, METANOL-d) δ ppm=8.77 (d, J=1.2 Hz, 1H), 7.81 (d, J=9.2 Hz, 1H), 7.56 (s, 1H), 7.48 (s, 1H), 6.91 (s, 1H), 6.51 (d, J=9.2 Hz, 1H), 4.88-4.85 (m, 1H), 4.15-4.10 (m, 1H), 4.02 (s, 3H), 3.86-3.83 (m, 1H), 3.42 (d, J=11.2 Hz, 1H), 3.26-3.20 (s, 1H), 3.15 (d, J=10.4 Hz, 1H), 2.46 (s, 3H), 2.38 (s, 3H), 2.28-2.23 (m, 1H), 2.20-2.15 (m, 1H).

Using the procedure described for Example 15 above, additional compounds described herein were prepared by substituting the appropriate amine starting materials in step a and b, suitable reagents and reaction conditions, obtaining compounds such as those selected from:

| Cpd ID | Data |
|---|---|
| 164 | Starting materials: 6-methoxy-2-methyl-2H-indazol-5-amine and 3- |
| | (azetidin-1-yl)pyrrolidine |
| | MS: m/z 447.2 [M + H]⁺; RT 1.13 min (Method 3) |
| | ¹H NMR (600 MHz, DMSO-d₆) δ ppm 2.06-2.12 (m, 1 H), 2.28-2.43 (m, |
| | 3 H), 3.51-3.54 (m, 1 H), 3.57-3.61 (m, 2 H), 3.70 (br d, J=6.49 Hz, 1 H), |
| | 3.95 (s, 3 H), 4.11 (s, 3 H), 4.14-4.20 (m, 4 H), 4.22 (br dd, J = 6.10, 2.67 |
| | Hz, 1 H), 6.75 (d, J=9.16 Hz, 1 H), 7.12 (s, 1 H), 7.59 (s, 1 H), 8.02 (d, J = 9.16 |
| | Hz, 1 H), 8.26 (s, 1 H), 9.42 (s, 1 H), 10.09 (br s, 1 H). |
| 165 | Starting materials: 8-fluoro-2-methylimidazo[1,2-a]pyridin-6-amine and 1- |
| | methylpiperazine |
| | MS: m/z 409.2 [M + H]⁺; RT 0.78 min (Method 3) |
| | ¹H NMR (600 MHz, DMSO-d₆) δ ppm 2.38 (s, 3 H), 2.87 (s, 3 H), 3.12- |
| | 3.24 (m, 4 H), 3.55 (br d, J = 11.44 Hz, 2 H), 4.46 (br d, J = 12.97 Hz, 2 H) |
| | 7.20 (d, J = 9.54 Hz, 1 H), 7.52 (br d, J = 12.21 Hz, 1 H), 7.66 (d, J = 0.76 Hz, 1 |
| | H), 8.02 (br s, 1 H), 8.07 (dd, J = 9.16, 0.76 Hz, 1 H), 9.16 (s, 1 H), 9.81 (br |
| | s, 1 H), 10.81 (s, 1 H). |
| 166 | Starting materials: 8-fluoro-2-methylimidazo[1,2-a]pyridin-6-amine and |
| | (R)-N,N-dimethylpyrrolidin-3-amine |
| | MS: m/z 447.2 [M + H]⁺; RT 1.14 min (Method 3) |
| | ¹H NMR (600 MHz, DMSO-d₆) δ ppm 1.99 (br s, 1 H), 2.27-2.33 (m, 2 H), |
| | 2.35 (s, 3 H), 2.44-2.50 (m, 6 H), 3.41-3.44 (m, 1 H), 3.50 (br d, J = 17.17 |
| | Hz, 1 H), 3.65-3.69 (m, 1 H), 3.82 (br d, J = 8.39 Hz, 1 H), 6.71 (d, J = 9.16 |
| | Hz, 1 H), 7.40 (dd, J = 12.97, 1.53 Hz, 1 H), 7.58 (d, J = 0.76 Hz, 1 H), 7.92- |
| | 7.96 (m, 2 H), 9.08 (d, J = 1.91 Hz, 1 H), 10.66 (s, 1 H). |
| 167 | Starting materials: 6-methoxy-2-methyl-2H-indazol-5-amine and 1-methyl- |
| | 1,7-diazaspiro[4.4]nonane |
| | MS: m/z 461.2 [M + H]⁺; RT 1.19 min (Method 3) |
| | ¹H NMR (600 MHz, DMSO-d₆) δ ppm 1.99-2.14 (m, 3 H), 2.16-2.29 (m, |
| | 2 H), 2.37 (ddd, J = 14.02, 8.49, 6.10 Hz, 1 H), 2.80 (dd, J = 17.93, 4.96 Hz, 3 |
| | H), 3.26-3.37 (m, 1 H), 3.52-3.59 (m, 2 H), 3.72-3.77 (m, 1 H), 3.91 (br |
| | d, J = 11.83 Hz, 1 H), 3.96 (s, 3 H), 4.06 (br d, J = 12.21 Hz, 1 H), 4.11 (s, 3 |
| | H), 6.75 (dd, J = 18.50, 9.35 Hz, 1 H), 7.11-7.15 (m, 1 H), 7.58 (br d, J = 11.83 |
| | Hz, 1 H), 8.04 (t, J = 8.39 Hz, 1 H), 8.23-8.34 (m, 1 H), 9.43 (d, J = 3.81 Hz, |
| | 1 H), 10.13 (br d, J = 5.34 Hz, 1 H). |
| 169 | Starting materials: 8-fluoro-2-methylimidazo[1,2-a]pyridin-6-amine and |
| | piperazine |
| | MS: m/z 395.2 [M + H]⁺; RT 0.83 min (Method 3) |
| | ¹H NMR (600 MHz, DMSO-d₆) δ ppm 2.38 (s, 3 H), 3.25 (br s, 4 H), 3.77- |
| | 3.79 (m, 4 H), 7.18 (d, J = 9.54 Hz, 1 H), 7.54 (br d, J = 12.59 Hz, 1 H), 7.66 |
| | (d, J = 0.76 Hz, 1 H), 8.03 (br s, 1 H), 8.04-8.07 (m, 1 H), 8.82 (br s, 2 H), |
| | 9.17 (s, 1 H) 10.82 (s, 1H). |
| 170 | Starting materials: 6-methoxy-2-methyl-2H-indazol-5-amine and N,N- |
| | dimethylpyrrolidin-3-amine |
| | MS: m/z 435.2 [M + H]⁺; RT 0.91 min (Method 3) |
| | ¹H NMR (600 MHz, DMSO-d₆) δ ppm 2.20-2.27 (m, 1 H), 2.47 (br dd, |
| | J = 12.02, 4.01 Hz, 1 H), 2.89 (br d, J = 4.58 Hz, 6 H), 3.45 (br s, 1 H), 3.63 |
| | (dd, J = 11.06, 6.49 Hz, 1 H), 3.73 (td, J = 9.35, 3.05 Hz, 1 H), 3.92-3.94 (m, |
| | 1 H), 3.95 (s, 3 H), 4.00-4.05 (m, 1 H), 4.11 (s, 3 H), 6.76 (d, J = 9.16 Hz, 1 |
| | H), 7.10-7.15 (m, 1 H), 7.58 (s, 1 H), 8.03 (d, J = 9.16 Hz, 1 H), 8.23-8.32 |
| | (m, 1 H), 9.42 (s, 1 H) 9.98 (br s, 1 H). |
| 171 | Starting materials: 6-methoxy-2-methylpyrazolo[1,5-a]pyridin-5-amine |
| | MS: m/z 433.2 [M + H]⁺; RT 1.10 min (Method 10) |
| | ¹H NMR (400 MHz, METHANOL-d₄) δ ppm = 8.43 (s, 1H), 8.14 (s, 1H), |
| | 7.92 (d, J = 8.8 Hz, 1H), 7.51 (s, 1H), 6.63 (d, J = 8.8 Hz, 1H), 6.29 (s, 1H), |
| | 5.05-5.02 (m, 1H), 4.20-4.15 (m, 1H), 4.03 (s, 3H), 3.90-3.81 (m, 2H), |
| | 3.73 (d, J = 12.0 Hz, 1H), 3.42-3.40 (m, 1H), 3.03-2.95 (m, 1H), 2.93- |
| | 2.85 (m, 4H), 2.41 (s, 3H) |
| 172 | Starting materials: 6-methoxy-2-methyl-2H-indazol-5-amine and (R)-3- |
| | (azetidin-1-yl)pyrrolidine |
| | MS: m/z 447.2 [M + H]⁺; RT 0.99 min (Method 3) |
| 173 | Starting materials: 6-methoxy-2-methyl-2H-indazol-5-amine and (S)-3- |
| | (azetidin-1-yl)pyrrolidine |
| | MS: m/z 447.2 [M + H]⁺; RT 0.98 min (Method 3) |
| 174 | Starting materials: tert-butyl 1,7-diazaspiro[4.4]nonane-1-carboxylate |
| | MS: m/z 461.3 [M + H]⁺; RT 1.238 min (Method 10) |
| | ¹H NMR (400 MHz, METHANOL-d₄) δ ppm = 8.79 (d, J = 1.6 Hz, 1H), 7.84 |
| | (d, J = 8.8 Hz, 1H), 7.57-7.51 (m, 2H), 6.92 (s, 1H), 6.71 (d, J = 8.8 Hz, |
| | 1H), 4.03 (s, 3H), 3.72-3.68 (m, 1H), 3.62 (d, J = 10.4 Hz, 1H), 3.53-3.50 |
| | (m, 1H), 3.35 (s, 1H), 2.90-2.85 (m, 2H), 2.38 (d, J = 6.4 Hz, 6H), 2.30- |
| | 2.20 (m, 1H), 1.94-1.89 (m, 5H). |
| 175 | Starting materials: 6-methoxy-2-methyl-2H-indazol-5-amine and 1-methyl- |
| | 1,7-diazaspiro[4.4]nonane |
| | MS: m/z 461.2 [M + H]⁺; RT 1.657 min (Method 8) |
| | ¹H NMR (400 MHz, CHLOROFORM-d) δ ppm = 9.00 (s, 1H), 8.59 (s, 1H), |
| | 8.02 (s, 1H), 7.70 (d, J = 8.8 Hz, 1H), 7.58 (s, 1H), 6.55 (d, J = 9.2 Hz, 1H), |
| | 6.22 (s, 1H), 4.00 (s, 3H), 3.80-3.70 (m, 2H), 3.58-3.50 (m, 1H), 3.38 (d, |
| | J = 10.4 Hz, 1H), 3.16-2.92 (m, 2H), 2.50 (s, 3H), 2.45 (s, 3H), 2.41- |
| | 2.32(m, 1H), 2.06-1.95 (m, 5H) |
| 259 | Starting materials: 6-methoxy-2-methylpyrazolo[1,5-a]pyridin-5-amine and |
| | 1-ethylpiperazine |
| | MS: m/z 435.2 [M + H]⁺; RT 1.900 min (Method 10) |
| 263 | Starting materials: 6-methoxy-2-methyl-2H-indazol-5-amine and 1- |
| | methylpiperazine |
| | MS: m/z 421.2 [M + H]⁺; RT 1.558 min (Method 10) |

### Example 24—Compound 176

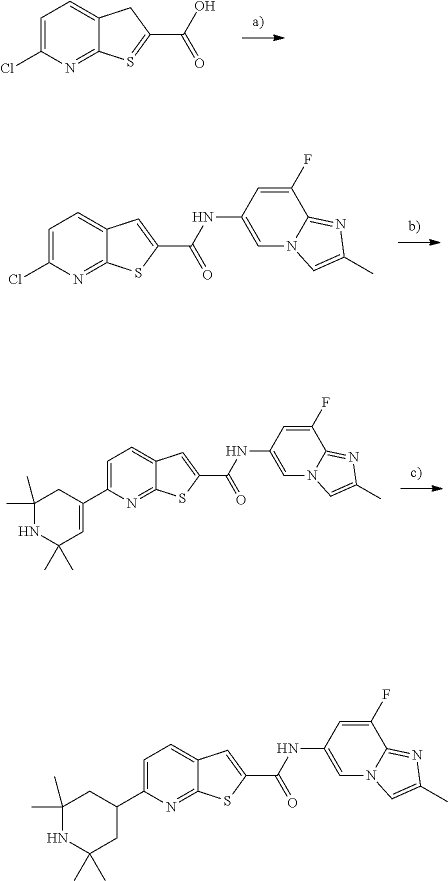

Step a: To a stirred solution of 6-chlorothieno[2,3-b]pyridine-2-carboxylic acid (250 mg, 1.2 mmol) in DMF (10 mL) was added DIPEA (454 mg, 3.51 mmol, 611 μL) and HATU (534 mg, 1.40 mmol) and 8-fluoro-2-methyl-imidazo[1,2-a]pyridin-6-amine (193.3 mg, 1.17 mmol). The reaction mixture was stirred at 20° C. for 14 h. The reaction mixture was washed with EtOAc (20 mL×3), filtered and concentrated under reduced pressure to give a residue. 6-chloro-N-(8-fluoro-2-methyl-imidazo[1,2-a]pyridin-6-yl)thieno[2,3-b]pyridine-2-carboxamide (270 mg, 739.07 μmol, 63.16% yield) was obtained as a brown solid. MS: m/z 360.8 [M+H]⁺; RT 0.648 min (Method 9)

Step b: 6-chloro-N-(8-fluoro-2-methylimidazo[1,2-a]pyridin-6-yl)thieno[2,3-b]pyridine-2-carboxamide (50 mg, 138 mmol) was dissolved in dioxane (1 mL) and water (0.3 mL). 2,2,6,6-tetramethyl-4-(4,4,5,5-tetramethyl-1,3,2-dioxaborolan-2-yl)-1,2,3,6-tetrahydropyridine (37 mg, 138 mmol) was added, followed by cesium carbonate (90 mg, 277 mmol) and PdCl₂(dppf) (8 mg, 14 mmol). The mixture was degassed with N₂ and stirred at 90° C. for 2 h. The resulting was cooled to room temperature and diluted with water, extracted with EtOAc, and concentrated. The crude was purified by acidic SCX column by acidifying with HCl methanol and releasing with 2N ammonia methanol to afford the title compound (21 mg, 0.045 mmol). MS: m/z 464.1 [M+H]⁺; RT 0.49 min (Method 4)

Step c: N-(8-fluoro-2-methylimidazo[1,2-a]pyridin-6-yl)-6-(2,2,6,6-tetramethyl-1,2,3,6-tetrahydropyridin-4-yl)thieno[2,3-b]pyridine-2-carboxamide (21 mg, 45 mmol) was dissolved in MeOH (1 mL), Ammonium formate (28 mg, 0.45 mmol) was added followed by Pd/C (10%) (5 mg, 4.5 μmol). The mixture was stirred at 60° C. for 2 h, the mixture was then cooled to room temperature and filtered on celite, washed with DCM (3×5 mL) and concentrated. The resulting was purified by RPHPLC with a basic modifier and gradient of 20-75% ACN in water to afford the title compound (3.9 mg, 8 mol) as an orange solid.

Using the procedure described for Example 15 above, additional compounds described herein were prepared by substituting the appropriate amine starting material in step a and boronic acid/ester starting material in step b, suitable reagents and reaction conditions, obtaining compounds such as those selected from:

| Cpd ID | Data |
|---|---|
| 177 | Starting materials: tert-butyl 4-(4,4,5,5-tetramethyl-1,3,2-dioxaborolan-2- |
| | y1)-2,3-dihydropyrrole-1-carboxylate |
| | MS: m/z 422.1 [M + H]⁺; RT 0.953 min (Method 10) |
| 178 | Starting materials: 1-methyl-4-(4,4,5,5-tetramethyl-1,3,2-dioxaborolan-2- |
| | yl)-3,6-dihydro-2H-pyridine |
| | MS: m/z 434.2. [M + H]⁺; RT 0.321 min (Method 9) |
| 179 | Starting materials: |
| | MS: m/z. 410.1 [M + H]⁺; RT 0.77 min (Method 4) |
| 181 | Starting materials: tert-butyl 3-(4,4,5,5-tetramethyl-1,3,2-dioxaborolan-2- |
| | yl)-2,5-dihydropyrrole-1-carboxylate |
| | MS: m/z 410.0. [M + H]⁺; RT 0.878 min (Method 10) |
| 182 | Starting materials: tert-butyl 6-(4,4,5,5-tetramethyl-1,3,2-dioxaborolan-2- |
| | yl)-2-azaspiro[3.4]oct-6-ene-2-carboxylate |
| | MS: m/z 436.1. [M + H]⁺; RT 1.21 min (Method 1) |
| 184 | Starting materials: 8-methoxy-2-methylimidazo[1,2-a]pyrazin-6-amine and |
| | 1-methyl-4-(4,4,5,5-tetramethyl-1,3,2-dioxaborolan-2-yl)-1,2,3,6- |
| | tetrahydropyridine |
| | MS: m/z 437.1 [M + H]⁺; RT 1.34 min (Method 10) |
| | ¹H NMR (400 MHz, METHANOL-d₄) δ ppm = 8.89 (s, 1H), 8.26 (d, J = 8.0 |
| | Hz, 1H), 8.18 (s, 1H), 7.72 (s, 1H), 7.42 (d, J = 8.4 Hz, 1H), 4.19 (s, 3H), |
| | 3.10-3.05 (m, 2H), 2.95-2.83 (m, 1H), 2.42 (s, 3H), 2.38 (s, 3H), 2.31-2.22 |
| | (m, 2H), 2.04-1.96 (m, 4H). |
| 185 | Starting materials: 6-methoxy-2-methyl-2H-indazol-5-amine and 1-methyl- |
| | 5-(4,4,5,5-tetramethyl-1,3,2-dioxaborolan-2-yl)-1,2,3,4-tetrahydropyridine |
| | MS: m/z 436.2 [M + H]⁺; RT 1.342 min (Method 8) |
| | ¹H NMR (400 MHz, METHANOL-d₄) δ ppm = 8.33 (s, 1H), 8.27 (d, J = 8.4 |
| | Hz, 1H), 8.08 (d, J = 18.0 Hz, 2H), 7.43 (d, J = 8.4 Hz, 1H), 7.05 (s, 1H), |
| | 4.15 (s, 3H), 4.02 (s, 3H), 3.24-3.18 (m, 2H), 3.08-3.02 (m, 1H), 2.63-2.52 |
| | (m, 1H), 2.48 (s, 3H), 2.37-2.25 (m, 1H), 2.10-2.04 (m, 1H), 1.91-1.72 (m, |
| | 3H). |
| 186 | Starting materials: 6-methoxy-2-methyl-2H-indazol-5-amine and 1-methyl- |
| | 5-(4,4,5,5-tetramethyl-1,3,2-dioxaborolan-2-yl)-1,2,3,4-tetrahydropyridine |
| | MS: m/z 436.2 [M + H]⁺; RT 1.745 min (Method 8) |
| | ¹H NMR (400 MHz, METHANOL-d₄) δ ppm = 8.33 (s, 1H), 8.26 (d, J = 8.4 |
| | Hz, 1H), 8.08 (d, J = 20.0 Hz, 2H), 7.42 (d, J = 8.0 Hz, 1H), 7.05 (s, 1H), |
| | 4.15 (s, 3H), 4.02 (s, 3H), 3.18-3.09 (m, 2H), 3.00-2.93 (m, 1H), 2.39-2.34 |
| | (m, 4H), 2.15-2.02 (m, 2H), 1.90-1.65 (m, 3H). |
| 187 | Starting materials: 2,8-dimethylimidazo[1,2-a]pyrazin-6-amine and 1- |
| | methyl-5-(4,4,5,5-tetramethyl-1,3,2-dioxaborolan-2-yl)-1,2,3,4- |
| | tetrahydropyridine |
| | MS: m/z 421.2 [M + H]⁺; RT 2.057 min (Method 10) |
| | ¹H NMR (400 MHz, CHLOROFORM-d) δ ppm = 9.04 (s, 1 H), 8.28 (s, 1 |
| | H), 8.09 (d, J = 8.0 Hz, 1 H), 7.86 (s, 1 H), 7.50 (s, 1 H), 7.32 (d, J = 8.4 Hz, |
| | 1 H), 3.34-3.19 (m, 2 H), 3.15-3.03 (m, 1 H), 2.86 (s, 3 H), 2.55-2.47 (m, |
| | 6 H), 2.26-2.16 (m, 1 H), 2.10-2.05 (m, 1 H), 1.91 (s, 2 H), 1.74-1.66 (m, |
| | 2 H). |
| | Starting materials: 2,8-dimethylimidazo[1,2-a]pyrazin-6-amine and 1- |
| | methyl-5-(4,4,5,5-tetramethyl-1,3,2-dioxaborolan-2-y1)-1,2,3,4- |
| | tetrahydropyridine |
| | MS: m/z 421.2 [M + H]⁺; RT 2.067 min (Method 10) |
| 188 | ¹H NMR (400 MHz, CHLOROFORM-d) δ ppm = 9.11 (s, 1 H), 8.28 (s, 1 |
| | H), 8.09 (d, J = 8.0 Hz, 1 H), 7.87 (s, 1 H), 7.50 (s, 1 H), 7.31 (d, J = 8.4 Hz, |
| | 1 H), 3.34-3.19 (m, 2 H), 3.15-3.03 (m, 1 H), 2.86 (s, 3 H), 2.55-2.47 (m, |
| | 6 H), 2.26-2.16 (m, 1 H), 2.10-2.05 (m, 1 H), 1.91 (s, 2 H), 1.74-1.66 (m, |
| | 2 H). |
| 265 | Starting materials: 7-fluoro-6-methoxy-2-methyl-2H-indazol-5-amine and |
| | tert-butyl 3-(4,4,5,5-tetramethyl-1,3,2-dioxaborolan-2-yl)-2,5-dihydro-1H- |
| | pyrrole-1-carboxylate |
| | MS: m/z 440.2 [M + H]⁺; RT 1.656 min (Method 8) |
| 266 | Starting materials: 7-fluoro-6-methoxy-2-methyl-2H-indazol-5-amine and |
| | tert-butyl 3-(4,4,5,5-tetramethyl-1,3,2-dioxaborolan-2-y1)-2,5-dihydro-1H- |
| | pyrrole-1-carboxylate |
| | MS: m/z 440.1 [M + H]⁺; RT 1.608 min (Method 8) |

### Example 25—Compound 210

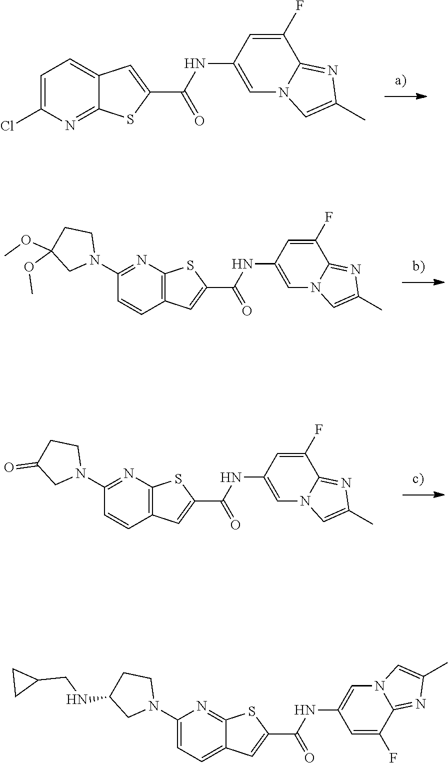

Step a: To a solution of 6-chloro-N-(8-fluoro-2-methyl-imidazo[1,2-a]pyridin-6-yl)thieno[2,3-b]pyridine-2-carboxamide (2.5 g, 6.93 mmol, 1.0 eq.) in DMF (80 mL, 0.087 M) was added 3,3-dimethoxypyrrolidine (1.82 g, 13.86 mmol, 2.0 eq.), sodium;2-methylpropan-2-olate (2.00 g, 20.79 mmol, 3.0 eq.) and [2-(2-aminophenyl)phenyl]-methylsulfonyloxy-palladium;dicyclohexyl-[2-(2,6-dimethoxyphenyl)phenyl]phosphane (1.08 g, 1.39 mmol, 0.2 eq.). Then the mixture was stirred at 130° C. for 12 h under N2 atmosphere. The residue was poured into water (100 mL) and the aqueous phase was extracted with EtOAc (100 mL×3). The combined organic phase was washed with water (100 mL×3), brine (200 mL×2), dried with anhydrous Na2SO4, filtered and concentrated. The crude material was purified by chromatography (DCM/MeOH=100/1 to 50/1, TLC: DCM/MeOH=10/1) to give 6-(3,3-dimethoxypyrrolidin-1-yl)-N-(8-fluoro-2-methylimidazo[1,2-a]pyridin-6-yl)thieno[2,3-b]pyridine-2-carboxamide (1.5 g, 3.29 mmol, 47.52% yield) as yellow solid. MS: m/z 456.1 [M+H]⁺; RT 2.067 min (Method 10)

Step b: To a solution of 6-(3,3-dimethoxypyrrolidin-1-yl)-N-(8-fluoro-2-methylimidazo[1,2-a]pyridin-6-yl)thieno[2,3-b]pyridine-2-carboxamide (1.3 g, 2.85 mmol, 1.0 eq.) in ACN (13 mL, 13 mL, 0.2 M) was added HCl (1 M, 2.6 mL, 1.0 eq.). Then the mixture was stirred at 25° C. for 2 h. The mixture was filtered and the filter cake was dried under reduced pressure. The solid was added DMSO (20 mL) and stirred at 100° C. for 1 h. The mixture was cooled to 25° C. and filtered, the filter cake was washed with EtOAc (20 mL×3) and the filter cake was concentrated under reduced pressure to give N-(8-fluoro-2-methylimidazo[1,2-a]pyridin-6-yl)-6-(3-oxopyrrolidin-1-yl)thieno[2,3-b]pyridine-2-carboxamide (734 mg, 1.68 mmol, 58.84% yield, 93.67% purity) as yellow solid. MS: m/z 410.1 [M+H]⁺; RT 2.367 min (Method 10)

Step c: To a mixture of N-(8-fluoro-2-methyl-imidazo[1,2-a]pyridin-6-yl)-6-(3-oxopyrrolidin-1-yl)thieno[2,3-b]pyridine-2-carboxamide (20 mg, 48.85 umol, 1.0 eq.) and cyclopropylmethanamine (20.84 mg, 293.09 umol, 6.0 eq.) in MeOH (4 mL, 0.012 M) was added acetic acid (14.67 mg, 244.24 umol, 5.0 eq.) in one portion at 25° C. under N2. After half an hour, add sodium;cyanoboranuide (9.21 mg, 146.54 umol, 3.0 eq.). The mixture was stirred at 25° C. for 2 hours. The mixture was further purification by pre-HPLC(Column Welch Xtimate C18 150*25 mm*5 um Condition water(FA)-ACN Begin B 2 End B 22 Gradient Time (min) 12 100% B Hold Time (min) 2 FlowRate (ml/min) 25) to give 6-(3-((cyclopropylmethyl)amino)pyrrolidin-1-yl)-N-(8-fluoro-2-methylimidazo[1,2-a]pyridin-6-yl)thieno[2,3-b]pyridine-2-carboxamide (10 mg, 21.53 umol, 44% yield). MS: m/z 465.3 [M+H]⁺; RT 0.68 min (Method 7) which was purified further by prep-SFC (Column: Chiralpak IC 50*4.6 mm 3 um, Mobile phase A: Hexane (0.1% DEA), and Phase B: IPA/MeCN=2:1, Isocratic A/B=40/60, flow rat: 1 mL/min; elution: Column Temp: 35° C.; to obtain rel-(R)-6-(3-((cyclopropylmethyl)amino)pyrrolidin-1-yl)-N-(8-fluoro-2-methylimidazo[1,2-a]pyridin-6-yl)thieno[2,3-b]pyridine-2-carboxamide.

### Section 3. Biological Assay and Data

HTT Mutant and Total HTRF iPSC Assay Protocol

The in-vitro cellular assay measures mutant and total Huntington (HTT) protein in human inducible pluripotent stem cells (iPSC) which were derived from a HTT patient with a poly-Q49 mutation. The assay measurement was performed by homogeneous time-resolved fluorescence (HTRF). The mutant HTT antibody is labeled with d-2 acceptor and recognizes an area in the poly Q region. The terbium (Tb) donor antibody recognizes a sequence at the N-terminus of the protein. The total HTT antibody was labeled with d2 acceptor and recognizes a sequence beyond the poly Q region. For each experiment run, a frozen aliquot of iPSC's was thawed from storage in liquid nitrogen and grown on Matrigel (Corning #354227) coated flasks using Complete Media ((mTeSR™1 Plus Basal Medium (STEMCELL Technologies cat #05825) supplemented with mTeSR™1 Plus (STEMCELL Technologies cat #05852) and penicillin/streptomycin (Gibco cat #10378016)) and in the presence of 10 uM Rock Inhibitor (Sigma #Y0503). The flask with cells was incubated overnight at 37° C. with 5% CO2 (Thermo) and next day the media was replaced with fresh Complete Media without Rock inhibitor and incubated for 48 hours for cell expansion at 37° C. with 5% CO2. Cells were harvested from flask using Accutase (Gibco #A1110501) and counted on Cellometer (Nexcelom Vision). A total of 10,000 cells/well were added in 30 ul volume of Complete Media with 10 uM rock inhibitor into a 384 well tissue culture plate (Perkin Elmer #NC1758152) pre-coated with Matrigel. The cell plate was centrifuged, and d cells were allowed to attach overnight at 37° C. and 5% CO2 in a high humidity incubator (Thermo Cytomat 10). The next day the cells were treated with compound. An intermediate plate was used to pre-dilute compounds in Complete Media with no rock inhibitor. Compounds were both diluted and dispensed using an ECHO (Labcyte #Echo555) into an empty 384 well PP plate (Griener #784201). A total of 60 ul of Complete Media was added per well using a multidrop Combi (Thermo #5840300). Compounds were tested in a 10 point, 3-fold titration, starting 10 uM. Media from cell plate was removed by flicking off media and plate was blotted on tissue paper. A volume of 50 uL was transferred from compound plate to the cell assay plate using an Integra (Viaflow384). Cell plate was incubated at 37° C., 5% CO2 with high humidity for 48 hours. Cell lysates were prepared by first removing media from plate and then adding 40 ul MPER lysis buffer (Thermo #78501) per well containing Protease and Phosphate inhibitor (Pierce #A32961). The plate was placed on an orbital shaker for 30 minutes at RT and an Apricot Dispenser (SPT Labtech) was used to transfer 5 ul of cell lysate into two 384 well black plates (Sigma Aldrich #CLS3821). Each plate contained either 5 ul/well of mutant or 5 ul/well of total HTT HTRF assay mix. The mutant HTT HTRF assay mix contained 2B7Ab-Tb “Donor” antibody (Thermo #CHDI-9000830)N-terminus labeled antibody at a final concentration 0.4 ng/well and MW1 (poly-Q specific) -d2 “acceptor” (Sigma #MABN2427) antibody final concentration 40 ng/well in HTRF Detection Buffer (CisBio #62SDBRDF). The total HTT HTRF assay mix contained 2B7Ab-Tb “Donor” N-terminus labeled antibody final concentration 0.4 ng/well and MAB2166-d2 (Anti-Huntingtin [1HU-4C8] mAb-d2 “acceptor” antibody with a final concentration 40 ng/well in HTRF Detection Buffer. All antibodies were labeled at Perkin Elmer. The assay plate was sealed and placed on an orbital shaker for a minute and then centrifuged for 1 minute before it was incubated at room temperature for 4 hours. The plate was read on a PHERAstar instrument (BMG LAB TECH) and HTRF ratio was calculated from (337 nm/665 nm) and (337 nm/620 nm) output. IC₅₀ values were generated from the full concentration-response curves. The curves were plotted as percent activity versus the compound concentration fitted to a variable 4-parameter logistic model.

A summary of IC₅₀ results is illustrated in Table 2, wherein “A” represents an IC₅₀ value of less than 100 nM, “B” represents an IC₅₀ value between 100 nM and 1 μM, and “C” represents an IC₅₀ value between 1 μM and 9 μM.

TABLE 2
mHTT protein lowering

| | mHTT | | |
|---|---|---|---|
| Cpd ID | IC₅₀ | Cpd ID | mHTT IC₅₀ |
| 1 | C | 48 | C |
| 2 | A | 49 | C |
| 3 | B | 50 | B |
| 4 | B | 51 | A |
| 5 | B | 52 | B |
| 6 | B | 53 | C |
| 7 | C | 54 | A |
| 9 | C | 55 | A |
| 10 | A | 56 | C |
| 11 | B | 57 | C |
| 12 | B | 58 | C |
| 13 | C | 59 | C |
| 14 | B | 60 | C |
| 15 | B | 61 | C |
| 16 | C | 62 | C |
| 17 | C | 63 | C |
| 18 | A | 64 | C |
| 19 | B | 65 | A |
| 20 | B | 66 | A |
| 21 | B | 67 | A |
| 22 | C | 68 | A |
| 23 | B | 69 | A |
| 24 | C | 70 | A |
| 25 | B | 71 | A |
| 26 | B | 72 | B |
| 27 | B | 73 | C |
| 28 | C | 74 | B |
| 29 | C | 75 | A |
| 30 | B | 76 | B |
| 31 | B | 77 | A |
| 32 | C | 78 | A |
| 33 | B | 79 | A |
| 34 | A | 80 | B |
| 35 | B | 81 | A |
| 36 | B | 82 | A |
| 37 | C | 83 | A |
| 38 | B | 84 | A |
| 39 | C | 85 | A |
| 40 | B | 86 | B |
| 41 | C | 8 | C |
| 42 | C | 87 | A |
| 43 | B | 88 | A |
| 44 | C | 89 | A |
| 45 | B | 90 | A |
| 46 | C | 91 | A |
| 47 | C | 92 | A |
| 129 | A | 93 | A |
| 130 | A | 94 | A |
| 131 | A | 95 | A |
| 132 | A | 96 | A |
| 133 | A | 97 | A |
| 134 | A | 98 | A |
| 135 | B | 99 | A |
| 136 | C | 100 | A |
| 137 | A | 101 | A |
| 138 | B | 102 | A |
| 139 | A | 103 | A |
| 140 | A | 104 | A |
| 146 | A | 105 | A |
| 147 | A | 106 | B |
| 148 | A | 107 | A |
| 149 | B | 108 | A |
| 150 | B | 109 | A |
| 151 | C | 110 | A |
| 152 | B | 111 | A |
| 153 | A | 112 | A |
| 154 | C | 113 | A |
| 155 | A | 114 | A |
| 156 | A | | |
| 157 | B | 116 | B |
| 158 | A | 117 | A |
| 159 | A | 118 | B |
| 160 | B | 119 | B |
| 161 | A | 120 | B |
| 162 | B | 121 | B |
| 163 | B | 122 | C |
| 164 | B | | |
| 165 | A | 124 | A |
| 166 | A | | |
| 167 | A | 126 | A |
| 168 | A | 127 | A |
| 169 | A | 128 | A |
| 170 | A | 176 | A |
| 171 | B | 177 | A |
| 172 | B | 178 | A |
| 173 | B | 179 | A |
| 174 | A | | |
| 175 | A | 181 | A |
| 185 | A | 182 | B |
| 186 | A | | |
| 187 | B | 184 | A |
| | | 209 | A |
| 189 | A | 210 | A |
| 190 | A | 211 | A |
| 191 | A | 212 | A |
| 192 | A | 213 | A |
| 193 | A | 214 | A |
| 194 | A | 215 | A |
| 195 | A | 216 | A |
| 196 | A | 217 | A |
| 197 | A | 218 | A |
| 198 | A | 219 | A |
| 199 | A | 220 | A |
| 200 | A | 221 | A |
| 201 | A | 222 | A |
| 202 | A | 223 | A |
| 203 | A | 224 | A |
| 204 | A | 225 | A |
| 205 | A | 226 | A |
| 206 | A | 233 | A |
| 207 | A | 234 | A |
| 208 | A | 235 | A |
| 227 | A | 236 | A |
| 228 | A | 237 | A |
| 229 | A | 238 | A |
| 230 | A | 239 | A |
| 231 | A | 240 | A |
| 232 | A | 241 | A |
| 245 | A | 242 | A |
| 247 | A | 243 | A |
| 248 | B | 244 | A |
| 249 | A | 251 | B |
| 252 | A | 264 | A |
| 250 | A | 265 | A |
| 253 | A | 266 | A |
| 254 | A | 267 | A |
| 255 | A | 259 | B |
| 246 | B | 263 | A |
| 262 | B | 269 | B |
| 180 | A | 183 | A |
| 141 | C | 256 | A |
| 142 | B | 260 | A |
| 143 | B | 261 | A |
| 144 | C | 268 | B |
| 145 | B | 257 | B |
| 258 | A | | |

What is claimed is:

1. A compound represented by Formula (I′): 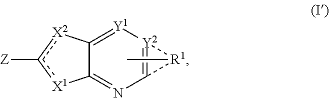
or a pharmaceutically acceptable salt thereof, wherein: [symbol]
is a single bond or double bond, provided the ring containing X₁ and X₂ is a 5-membered heteroaryl ring; 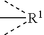
indicates that R¹ is substituted at one of two positions on the 6-membered ring to which the dash lines connect and the other position to which the dash lines connect is unsubstituted;
Z is —C(═O)NR²R³ or —NR²C(═O)R³;
X¹ is S or CH;
X² is N, O or CH;
one of Y¹ and Y² is N and the other is CH;
R¹ is 4 to 12 membered heterocyclyl, 4 to 12 membered carbocyclyl, —NR¹¹R¹², —C₁₋₆alkylene-NR¹³R¹⁴, or —OR¹⁵ wherein
said 4 to 12 membered carbocyclyl or 4 to 12-membered heterocyclyl represented by R¹ is optionally substituted with one or more R^{A}; wherein
each R^{A} is independently C₁₋₆alkyl, C₃₋₆cycloalkyl, haloC₁₋₆alkyl, —NR^{a}R^{b}, —C₁₋₃alkylene-NR^{a}R^{b}, —C₃₋₆cycloalkylene-NR^{a}R^{b}, —C(═O)R^{a}, or 4 to 6-membered saturated heterocyclyl; wherein each R^{a} and R^{b} is independently H or C₁₋₆alkyl; wherein said 4 to 6-membered saturated heterocyclyl represented by R^{A} is optionally substituted by one or more C₁₋₆alkyl;
R¹¹ is H or C₁₋₆alkyl;
R¹² is C₁₋₆alkyl, 6 to 10-membered aryl, 4 to 12-membered heterocyclyl, or 5-10 membered heteroaryl; wherein said C₁₋₆alkyl, 6 to 10-membered aryl, 4 to 12-membered heterocyclyl, or 5-10 membered heteroaryl represented by R¹² is optionally substituted by one or more R^{B}; wherein
R^{B} is halo, C₁₋₆alkyl, —NR^{a}R^{b}, 4 to 6-membered heterocyclyl, or —C₁₋₆ alkylene-4 to 6-membered heterocyclyl; wherein said 4 to 6-membered heterocyclyl represented by R^{B} is optionally substituted by one or more C₁₋₆alkyl;
R³ is H or C₁₋₆alkyl;
R¹⁴ and R¹⁵ are independently selected from H, C₁₋₆alkyl, or —C₁₋₆alkylene-4-6 membered saturated heterocyclyl;
R² is H or C₁₋₃alkyl;
R³ is 6 to 10 membered aryl or 6 to 10 member heteroaryl, wherein said 6 to 10 membered aryl and 6 to 10 member heteroaryl represented by R³ are optionally substituted by one or more R^{C}; wherein
R^{C} is halo, —CN, —OH, C₁₋₆alkyl, C₁₋₆haloalkyl, or C₁₋₆alkoxy, or two R^{C} together with the intervening atoms together form 5 to 7 membered heterocyclyl;
wherein said 5 to 7 membered heterocyclyl represented by R^{C} is optionally substituted by R^{C1}; where R^{C1} is C₁₋₃alkyl or oxo; and
wherein said heterocyclyl comprises 1-3 heteroatoms independently selected from oxygen, nitrogen, and sulfur; and said heteroaryl comprises 1-4 heteroatoms independently selected from oxygen, nitrogen, and sulfur;
provided that the compound of formula (I′) is not represented by

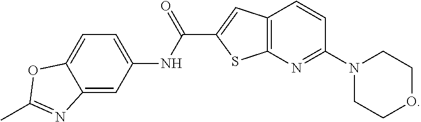 [SMILES: Cc1nc2cc(NC(=O)c3cc4ccc(N5CCOCC5)nc4s3)ccc2o1]

2. The compound of claim 1, wherein the compound is represented by Formula (I): 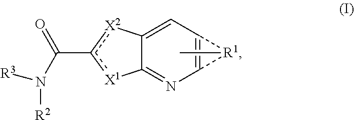
or a pharmaceutically acceptable salt thereof, wherein: [symbol]
is a single bond or double bond, provided the ring containing X₁ and X₂ is a 5-membered heteroaryl ring; 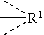
indicates that R¹ is substituted at one of two positions on the pyridyl moiety to which the dash lines connect and the other position to which the dash lines connect is unsubstituted;
X¹ is S or CH;
X² is N, O or CH;
R¹ is 4 to 12 membered heterocyclyl, —NR¹¹R¹², or —C₁₋₆alkylene-NR¹³R¹⁴, wherein
said 4 to 12-membered heterocyclyl represented by R¹ is optionally substituted with one or more R^{A}; wherein
each R^{A} is independently C₁₋₆alkyl, C₃₋₆cycloalkyl, haloC₁₋₆alkyl, —NR^{a}R^{b}, —C₁₋₃alkylene-NR^{a}R^{b}, —C₃₋₆cycloalkylene-NR^{a}R^{b}, —C(═O)R^{a}, or 4 to 6-membered saturated heterocyclyl; wherein each R^{a} and R^{b} is independently H or C₁₋₆alkyl; wherein said 4 to 6-membered saturated heterocyclyl represented by R^{A} is optionally substituted by one or more C₁₋₆alkyl;
R¹¹ is H or C₁₋₆alkyl;
R¹² is C₁₋₆alkyl, 6 to 10-membered aryl, 4 to 12-membered heterocyclyl, or 5-10 membered heteroaryl; wherein said C₁₋₆alkyl, 6 to 10-membered aryl, 4 to 12-membered heterocyclyl, or 5-10 membered heteroaryl represented by R¹² is optionally substituted by one or more R^{B}; wherein
R^{B} is C₁₋₆alkyl, —NR^{a}R^{b}, 4 to 6-membered heterocyclyl, or —C₁₋₆alkylene-4 to 6-membered heterocyclyl; wherein said 4 to 6-membered heterocyclyl represented by R^{B} is optionally substituted by one or more C₁₋₆alkyl;
R¹³ is H or C₁₋₆alkyl;
R¹⁴ is H, C₁₋₆alkyl, or —C₁₋₆alkylene-4-6 membered saturated heterocyclyl;
R² is H or C₁₋₃alkyl;
R³ is 6 to 10 membered aryl or 6 to 10 member heteroaryl, wherein said 6 to 10 membered aryl and 6 to 10 member heteroaryl represented by R³ are optionally substituted by one or more R^{C}; wherein
R^{c} is halo, —CN, —OH, C₁₋₆alkyl, C₁₋₆haloalkyl, or C₁₋₆alkoxy, or two R^{C} together with the intervening atoms together form 5 to 7 membered heterocyclyl;
wherein said 5 to 7 membered heterocyclyl represented by R^{C} is optionally substituted by R^{C1}; where R^{C1} is C₁₋₃alkyl or oxo; and
wherein said heterocyclyl comprises 1-3 heteroatoms independently selected from oxygen, nitrogen, and sulfur; and said heteroaryl comprises 1-4 heteroatoms independently selected from oxygen, nitrogen, and sulfur;
provided that the compound of formula (I) is not represented by

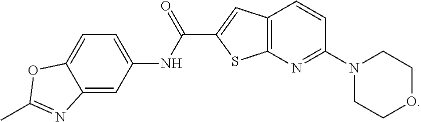 [SMILES: Cc1nc2cc(NC(=O)c3cc4ccc(N5CCOCC5)nc4s3)ccc2o1]

3. The compound of claim 1 or 2 or a pharmaceutically acceptable salt thereof, wherein the compound is represented by Formula (II):

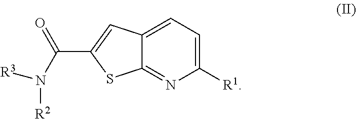

4. The compound of claim 1 or 2 or a pharmaceutically acceptable salt thereof, wherein the compound is represented by Formula (III):

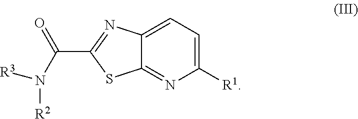

5. The compound of claim 1 or 2 or a pharmaceutically acceptable salt thereof, wherein the compound is represented by Formula (IV):

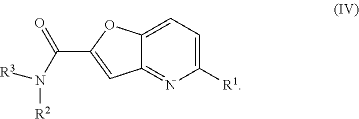

6. The compound of claim 1 or 2 or a pharmaceutically acceptable salt thereof, wherein the compound is represented by Formula (V):

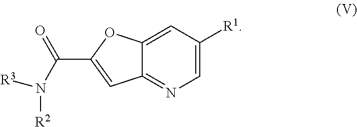

7. The compound of claim 1 or 2 or a pharmaceutically acceptable salt thereof, wherein the compound is represented by Formula (VI):

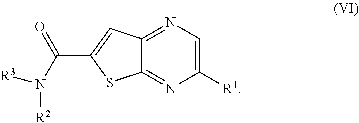

8. The compound of claim 1 or 2 or a pharmaceutically acceptable salt thereof, wherein the compound is represented by Formula (VII):

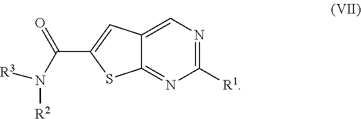

9. The compound of claim 1 or 2 or a pharmaceutically acceptable salt thereof, wherein the compound is represented by Formula (VIII):

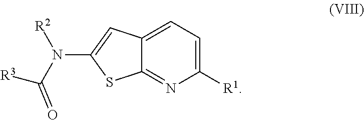

10. The compound of any one of claims 1-9 or a pharmaceutically acceptable salt thereof, wherein R² is H.

11. The compound of any one of claims 1-10 or a pharmaceutically acceptable salt thereof, wherein R¹ is a 4 to 12 membered saturated heterocyclyl.

12. The compound of any one of claims 1-11 or a pharmaceutically acceptable salt thereof,
wherein:
R¹ is a 4 to 12 membered saturated heterocyclyl comprising one or two ring N atoms, provided when said heterocyclyl comprises one ring N atom, it is then optionally substituted with —NR⁷R⁸, —C₁₋₃alkylene-NR⁷R⁸ or —C₃₋₆cycloalkylene-NR⁷R⁸ and optionally further substituted with 1 to 4 R⁹, and when said heterocyclyl comprises two ring N atoms, it is optionally substituted with 1 to 3 R⁹;
R⁷ and R⁸ are each independently H or C₁₋₆alkyl; alternatively R⁷ and R⁸ taken together with N to which they are attached forms a 4 to 6 membered heteterocycle optionally substituted with 1 to 2 C₁₋₆alkyl, wherein said 4 to 6 membered heteterocycle optionally comprises a second hetero atom selected from N and 0;
R⁹, for each occurrence, is independently selected from halo, —C(═O)R¹⁰, C₁₋₆alkyl, C₁₋₆haloalkyl, C₁₋₆alkoxyC₁₋₆alkyl, and C₃₋₆cycloalkyl; wherein said C₃₋₆cycloalkyl represented by R⁹ is optionally substituted by one or more substituents independently selected from halo and C₁₋₆alkyl; wherein R¹⁰ is H, C₁₋₃alkyl, or C₃₋₆cycloalkyl.

13. The compound of any one of claims 1-11 or a pharmaceutically acceptable salt thereof,
wherein:
R¹ is a 4 to 12 membered saturated heterocyclyl comprising one or two ring N atoms, provided when said heterocyclyl comprises one ring N atom, it is then optionally substituted with —NR⁷R⁸, —C₁₋₃alkylene-NR⁷R⁸ or —C₃₋₆cycloalkylene-NR⁷R⁸ and optionally further substituted with 1 to 2 R⁹, and when said heterocyclyl comprises two ring N atoms, it is optionally substituted with 1 to 3 R⁹;
R⁷ and R⁸ are each independently H or C₁₋₆alkyl; alternatively R⁷ and R⁸ taken together with N to which they are attached forms a 4 to 6 membered heteterocycle optionally substituted with 1 to 2 C₁₋₆alkyl, wherein said 4 to 6 membered heteterocycle optionally comprises a second hetero atom selected from N and 0;
R⁹, for each occurrence, is independently selected from halo, —C(═O)R¹⁰, C₁₋₆alkyl, C₁₋₆haloalkyl, C₁₋₆alkoxyC₁₋₆alkyl, and C₃₋₆cycloalkyl; wherein said C₃₋₆cycloalkyl represented by R⁹ is optionally substituted by one or more substituents independently selected from halo and C₁₋₆alkyl; wherein R¹⁰ is H, C₁₋₃alkyl, or C₃₋₆cycloalkyl.

14. The compound of claim 12 or a pharmaceutically acceptable salt thereof, wherein R¹ is a 4 to 12 membered saturated heterocyclyl comprising one ring N atom and is substituted with 1 to 4 R⁹.

15. The compound of claim 14 or a pharmaceutically acceptable salt thereof, wherein R¹ is selected from pyrrolidinyl, piperidinyl, azabicyclo[3.2.1]octanyl, and azaspiro[3.4]octanyl.

16. The compound of claim 14 or a pharmaceutically acceptable salt thereof, wherein R¹ is selected from:

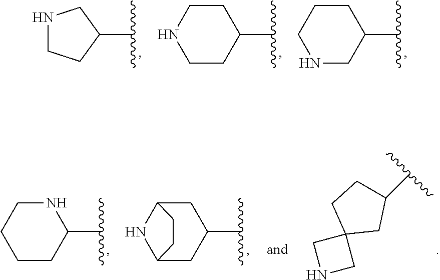

17. The compound of claim 12 or a pharmaceutically acceptable salt thereof, wherein R¹ is a 4 to 12 membered saturated heterocyclyl comprising one ring N atom and is substituted with —NR⁷R⁸, —C₁₋₃alkylene-NR⁷R⁸ or —C₃₋₆cycloalkylene-NR⁷R⁸ and optionally further substituted with 1 to 2 R⁹.

18. The compound of claim 17 or a pharmaceutically acceptable salt thereof, wherein R¹ is a 4 to 12 membered saturated heterocyclyl selected from azetidinyl, piperidinyl, pyrrolidinyl, octahydro-1H-isoindolyl, and 3-azabicyclo[3.1.0]hexanyl, each of which is substituted with —NR⁷R⁸, —C₁₋₃alkylene-NR⁷R⁸ or —C₃₋₆cycloalkylene-NR⁷R⁸ and optionally further substituted with 1 to 2 R⁹.

19. The compound of claim 17 or a pharmaceutically acceptable salt thereof, wherein R¹ is selected from 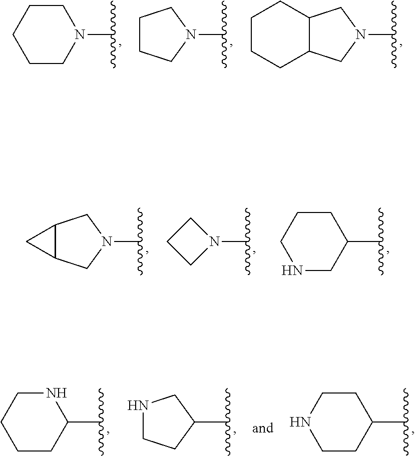
each of which is substituted with —NR⁷R⁸, —C₁₋₃alkylene-NR⁷R⁸ or —C₃₋₆cycloalkylene-NR⁷R⁸ and optionally further substituted with 1 to 2 R⁹.

20. The compound of claim 17 or a pharmaceutically acceptable salt thereof, wherein R¹ is selected from 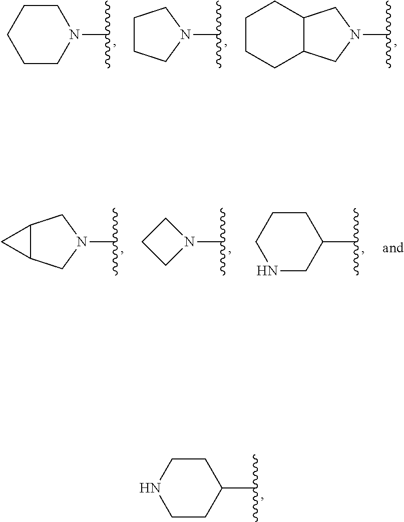
each of which is substituted with —NR⁷R⁸, —C₁₋₃alkylene-NR⁷R⁸ or —C₃₋₆cycloalkylene-NR⁷R⁸ and optionally further substituted with 1 to 2 R⁹.

21. The compound of any one of claims 1-20 or a pharmaceutically acceptable salt thereof, wherein R⁷ and R⁸ are each independently H or C₁₋₃alkyl; alternatively R⁷ and R⁸ taken together are C₂-C₄ alkylene, optionally substituted with 1 or 2 C₁₋₃alkyl.

22. The compound of any one of claims 1-21 or a pharmaceutically acceptable salt thereof, wherein R⁷ and R⁸ are each independently H, —CH₃ or —CH₂CH₃; alternatively R⁷ and R⁸ taken together are —CH₂CH₂CH₂CH₂—, —CH₂CH₂CH₂— or —CH₂C(CH₃)₂CH₂—.

23. The compound of any one of claims 17-20 or a pharmaceutically acceptable salt thereof, wherein R¹ is selected from a group consisting of 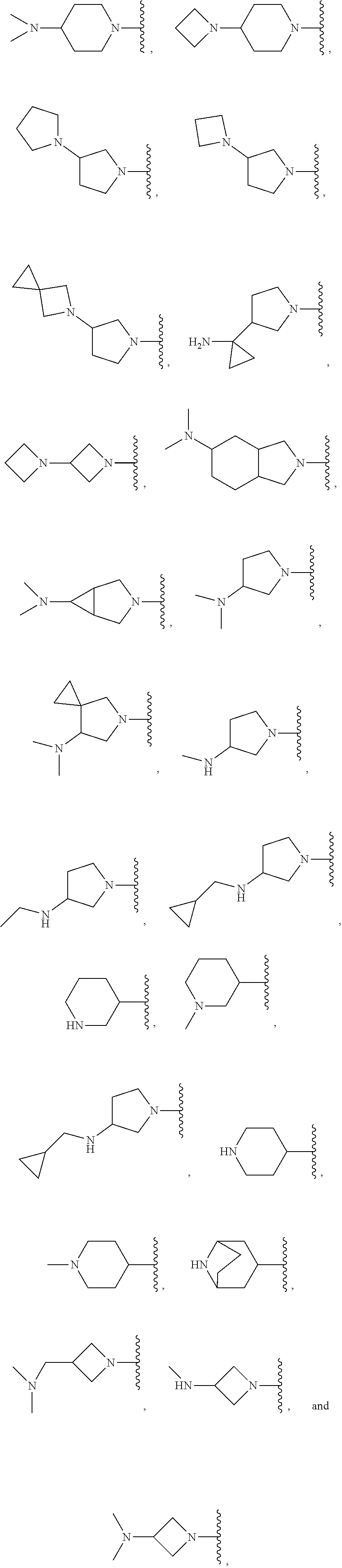
each of which is optionally further substituted with 1 to 2 R⁹.

24. The compound of any one of claims 17-19 or a pharmaceutically acceptable salt thereof, wherein R¹ is selected from a group consisting of 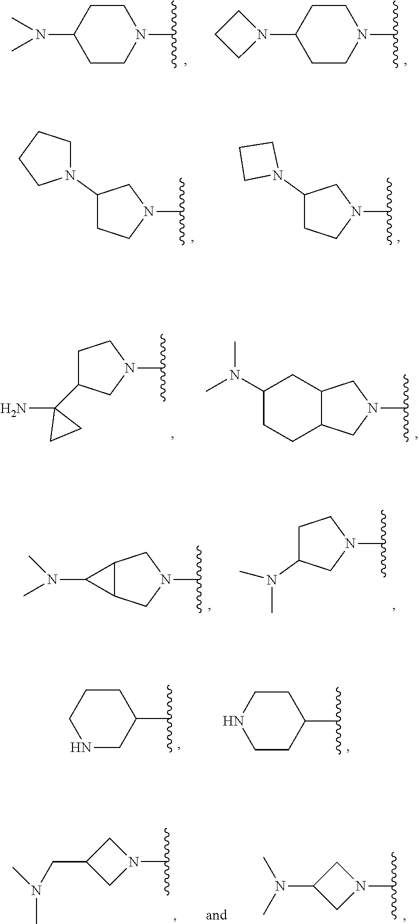
each of which is optionally further substituted with 1 to 2 R⁹.

25. The compound of claim 12 or a pharmaceutically acceptable salt thereof, wherein R¹ is a 4 to 12 membered saturated heterocyclyl comprising two ring N atoms and is optionally substituted with 1 to 3 R⁹.

26. The compound of claim 25 or a pharmaceutically acceptable salt thereof, wherein the 4 to 12 membered saturated heterocyclyl represented by R¹ is piperazinyl, 4,7-diazaspiro[2.5]octanyl, 3,9-diazaspiro[5.5]undecanyl, 1-oxa-4,9-diazaspiro[5.5]undecanyl, diazabicyclo[2.2.2]octanyl, octahydro-2H-pyrido[4,3-b][1,4]oxazinyl, octahydro-1H-pyrrolo[2,3-c]pyridinyl, 2,5-diazabicyclo[2.2.1]heptanyl, octahydropyrrolo[1,2-a]pyrazinyl, decahydro-1,6-naphthyridinyl, 1,6-diazaspiro[3.4]octanyl, 1,5-diazaspiro[3.4]octanyl, 2λ²,5-diazaspiro[3.4]octanyl, 2λ²,6-diazaspiro[3.4]octanyl, hexahydropyrrolo[3,4-c]pyrrolyl, octahydropyrrolo[3,4-c]pyrrolyl, octahydro-1H-pyrrolo[2,3-c]pyridinyl, octahydropyrrolo[3,4-b]pyrrolyl, 3,6-diazabicyclo[3.2.0]heptanyl, 1,4-diazepanyl, 2,6-diazaspiro[3.5]nonane, 2,6-diazabicyclo[3.2.0]heptanyl, or 1,7-diazaspiro[4.4]nonanyl, each of which is optionally substituted with 1 to 2 R⁹.

27. The compound of claim 25 or a pharmaceutically acceptable salt thereof, wherein the 4 to 12 membered saturated heterocyclyl represented by R¹ is piperazinyl, diazabicyclo[2.2.2]octanyl, octahydro-2H-pyrido[4,3-b][1,4]oxazinyl, octahydro-1H-pyrrolo[2,3-c]pyridinyl, 2,5-diazabicyclo[2.2.1]heptanyl, octahydropyrrolo[1,2-a]pyrazinyl, decahydro-1,6-naphthyridinyl, hexahydropyrrolo[3,4-c]pyrrolyl, octahydropyrrolo[3,4-c]pyrrolyl, octahydro-1H-pyrrolo[2,3-c]pyridinyl, octahydropyrrolo[3,4-b]pyrrolyl, 1,4-diazepanyl, or 2,6-diazaspiro[3.5]nonane, each of which is optionally substituted with 1 to 2 R⁹.

28. The compound of claim 26 or a pharmaceutically acceptable salt thereof, wherein the 4 to 12 membered saturated heterocyclyl represented by R¹ is: 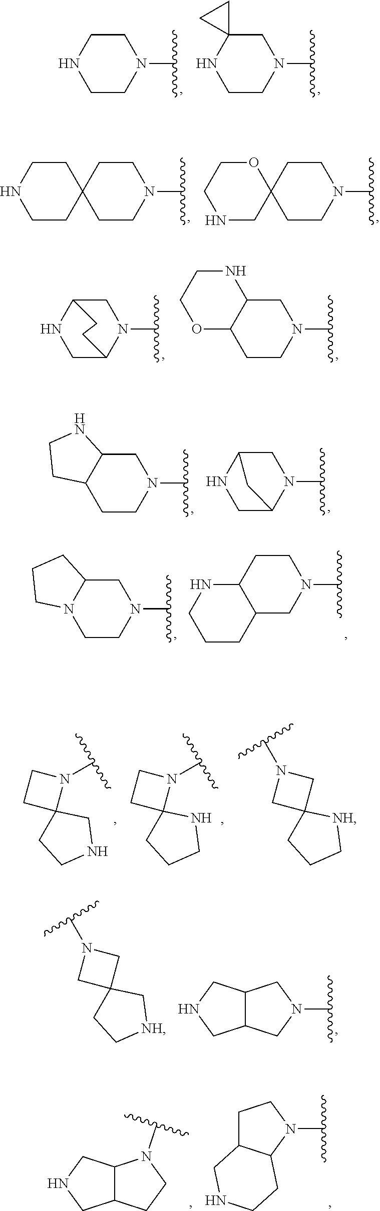 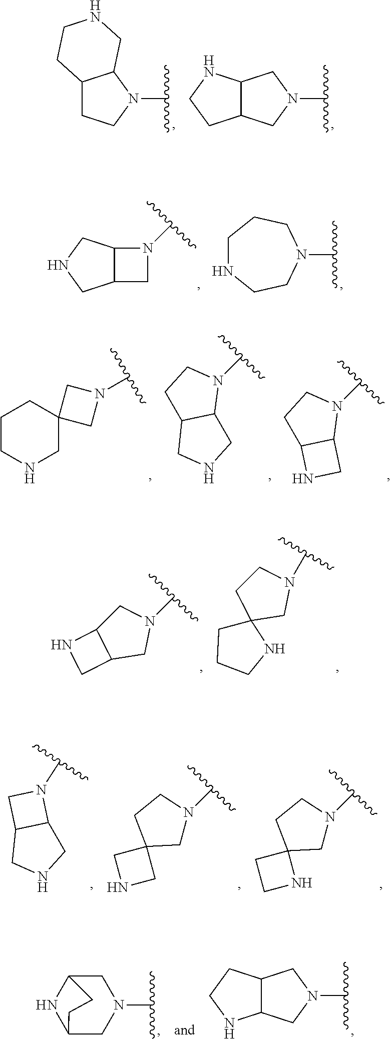
each of which is optionally substituted 1 or 3 R⁹.

29. The compound of claim 27 or a pharmaceutically acceptable salt thereof, wherein the 4 to 12 membered saturated heterocyclyl represented by R¹ is: 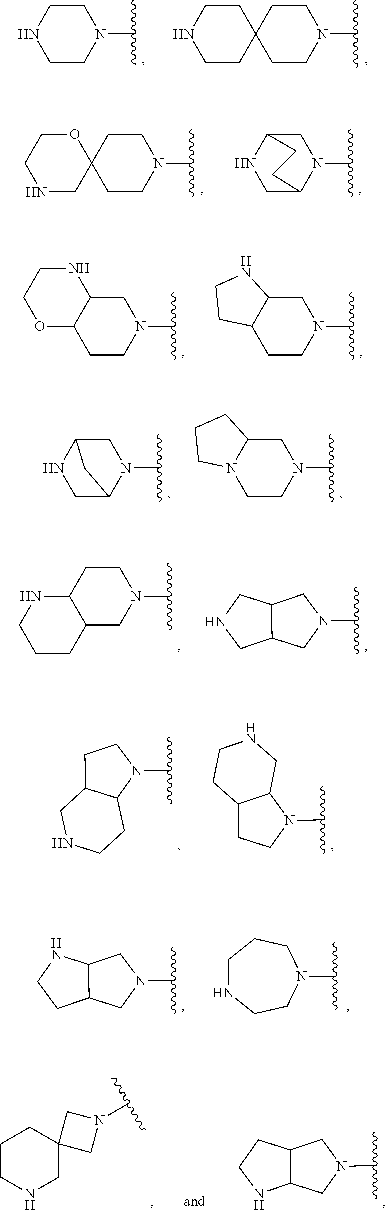
each of which is optionally substituted 1 or 3 R⁹.

30. The compound of any one of claims 1-10 or a pharmaceutically acceptable salt thereof, wherein Rt is 4 to 12 membered partially saturated heterocyclyl.

31. The compound of claim 30 or a pharmaceutically acceptable salt thereof, wherein the partially saturated heterocyclyl is 2,3,4,5-tetrahydro-1H-pyrido[2,3-e][1,4]diazepine, 1,2,3,6-tetrahydropyridinyl, 6-azabicyclo[3.1.1]hept-2-enyl, or 8-azabicyclo[3.2.1]oct-2-enyl.

32. The compound of claim 30 or a pharmaceutically acceptable salt thereof, wherein the partially saturated heterocyclyl is 2,3,4,5-tetrahydro-1H-pyrido[2,3-e][1,4]diazepine, 1,2,3,6-tetrahydropyridinyl or 8-azabicyclo[3.2.1]oct-2-enyl.

33. The compound of any one of claim 30 or 31 or a pharmaceutically acceptable salt thereof, wherein the partially saturated heterocyclyl is selected from a group consisting of: 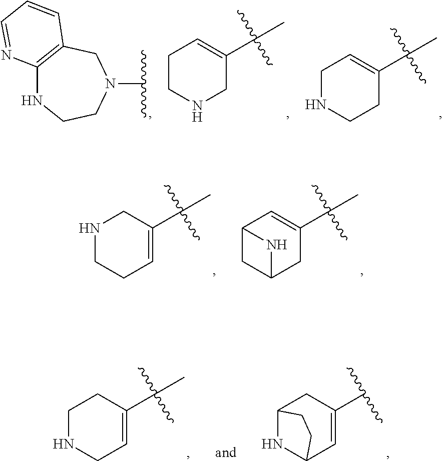
each of which is optionally substituted with 1, 2, 3 or 4 R⁹.

34. The compound of any one of claim 30 or 31 or a pharmaceutically acceptable salt thereof, wherein the partially saturated heterocyclyl is selected from a group consisting of: 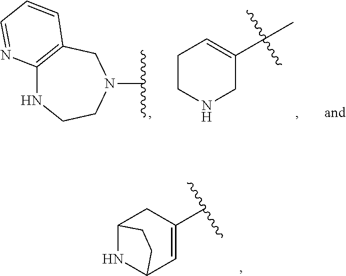
each of which is optionally substituted with 1 or 2 R⁹.

35. The compound of any one of claims 1-10 or a pharmaceutically acceptable salt thereof, wherein R¹ is 4 to 12 membered saturated or partially saturated carbocyclyl substituted with —NR⁷R⁸ and is further optionally substituted with 1 or 2 R⁹.

36. The compound of any one of claim 35 or a pharmaceutically acceptable salt thereof, wherein R¹ is cyclohexyl or cyclohexenyl, each of which is substituted with —NR⁷R⁸ and is further optionally substituted with 1 or 2 R⁹.

37. The compound of any one of claim 35 or 36 or a pharmaceutically acceptable salt thereof, wherein R¹ is selected from 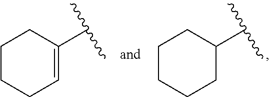 [SMILES: *C1=CCCCC1 ; *C1CCCCC1]
and each of which is substituted with —NR⁷R⁸ and is further optionally substituted with 1 or 2 R⁹.

38. The compound of any one of claims 35 to 37 or a pharmaceutically acceptable salt thereof, wherein R⁷ and R⁸ are each independently H or C₁₋₃alkyl.

39. The compound of any one of claims 35 to 37 or a pharmaceutically acceptable salt thereof, wherein R⁷ and R⁸ are each independently H or —CH₃.

40. The compound of any one of claims 1-39 or a pharmaceutically acceptable salt thereof, wherein R⁹, for each occurrence, is independently selected from halo, —C(═O)R¹⁰, C₁₋₄ alkyl, C₁₋₄haloalkyl, and C₃₋₆cycloalkyl; wherein said C₃₋₆cycloalkyl represented by R⁹ is optionally substituted by one to three substituents independently selected from F, Cl, and C₁₋₄alkyl; and R¹⁰ is H, C₁₋₂alkyl, C₃₋₄cycloalkyl.

41. The compound of any one of claims 1-39 or a pharmaceutically acceptable salt thereof, wherein R⁹, for each occurrence, is independently selected from F, —CH₃, —CH₂CH₃, —C(═O)CH₃, —CH₂CF₃, —CH(CH₃)₂, —CD₃, and cyclopropyl.

42. The compound of any one of claims 1-39 or a pharmaceutically acceptable salt thereof, wherein R⁹, for each occurrence, is independently selected from —CH₃, —C(═O)CH₃, —CH₂CF₃, —CH(CH₃)₂, and cyclopropyl.

43. The compound of any one of claims 1-10 or a pharmaceutically acceptable salt thereof, wherein:
R¹ is —NR¹¹R¹²;
R¹¹ is H or C₁₋₆alkyl;
R¹² is C₁₋₆alkyl-NR^{a}R^{b}, phenyl, 4 to 12-membered heterocyclyl comprising at least one ring N atom; wherein said phenyl represented by R¹² is substituted with —NR^{a}R^{b}, Het, or —C₁₋₃alkylene-Het, and Het is a 4 to 6-membered heterocyclyl comprising at least one ring N atom and is optionally substituted with one or two C₁₋₃alkyl; and wherein said 4 to 12-membered heterocyclyl represented by R¹² is optionally substituted by one, two, three, four or five R^{12a}; wherein each R^{12a} is independently C₁₋₃alkyl or halo.

44. The compound of any one of claims 1-10 or a pharmaceutically acceptable salt thereof, wherein:
R¹ is —NR¹¹R¹²;
R¹¹ is H or C₁₋₆alkyl;
R¹² is C₁₋₆alkyl-NR^{a}R^{b}, phenyl, 4 to 12-membered heterocyclyl comprising at least one ring N atom; wherein said phenyl represented by R¹² is substituted with —NR^{a}R^{b}, Het, or —C₁₋₃alkylene-Het, and Het is a 4 to 6-membered heterocyclyl comprising at least one ring N atom and is optionally substituted with one or two C₁₋₃alkyl; and wherein said 4 to 12-membered heterocyclyl represented by R¹² is optionally substituted by one or two C₁₋₃alkyl.

45. The compound of any one of claims 1-10 and 43 or a pharmaceutically acceptable salt thereof, wherein:
R¹ is —NR¹¹R¹²,
R¹¹ is H or —CH₃;
R¹² is selected from a group consisting of: piperidinyl, hexahydro-TH-pyrrolizinyl, octahydrocyclopenta[c]pyrrolyl, octahydroindolizinyl, isoindolinyl, phenylazetidinyl, 1,2,3,4,5-tetrahydro-1H-benzo[e][1,4]diazepinyl, benzylpyrrolidinyl, and quinuclidinyl, each of which is optionally substituted with one, two, three, four or five R^{12a}; wherein R^{12a} is C₁₋₃alkyl or halo.

46. The compound of any one of claims 43-45 or a pharmaceutically acceptable salt thereof, wherein R^{12a} is methyl or fluoro.

47. The compound of any one of claims 1-10 and 43 or a pharmaceutically acceptable salt thereof, wherein:
R¹ is —NR¹¹R¹²;
R¹¹ is H or —CH₃;
R¹² is selected from a group consisting of: hexahydro-1H-pyrrolizinyl, octahydrocyclopenta[c]pyrrolyl, octahydroindolizinyl, isoindolinyl, phenylazetidinyl, 1,2,3,4,5-tetrahydro-1H-benzo[e][1,4]diazepinyl, benzylpyrrolidinyl, and quinuclidinyl, each of which is optionally substituted with one or two independently C₁₋₂alkyl.

48. The compound of any one of claims 1-10 and 43 or a pharmaceutically acceptable salt thereof, wherein:
R¹ is —NR¹¹R¹²;
R¹¹ is H or —CH₃;
R¹² is selected from a group consisting of: 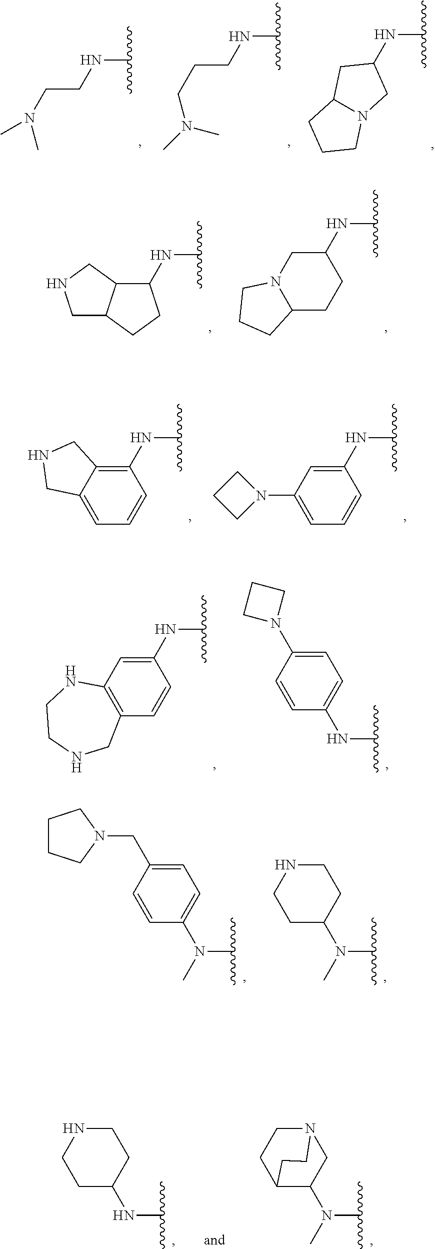
each of which is optionally substituted with one, two, three, four, or five substituents independently selected from, F, —CH₃ and —CH₂CH₃.

49. The compound of any one of claims 1-10 and 43 or a pharmaceutically acceptable salt thereof, wherein:
R¹ is —NR¹¹R¹²;
R¹¹ is H or —CH₃;
R¹² is selected from a group consisting of: 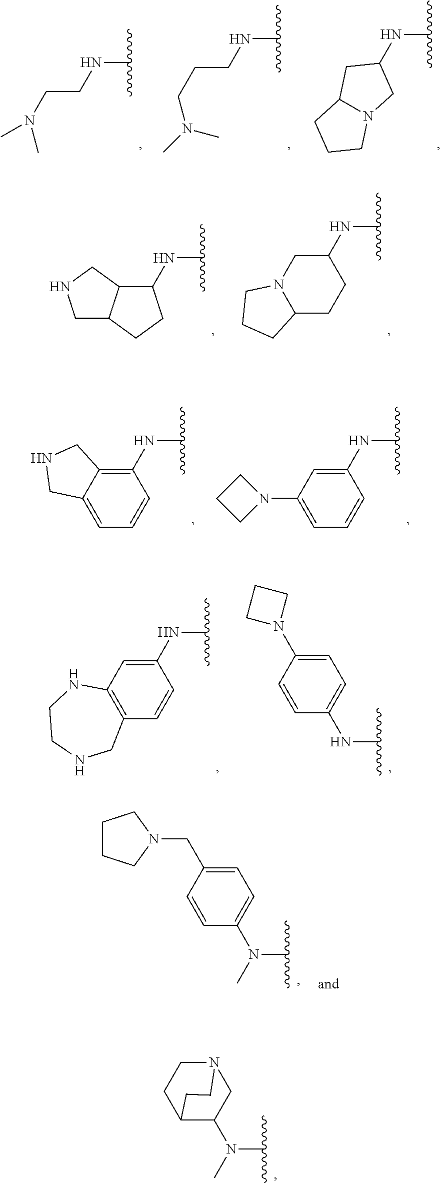
each of which is optionally substituted with one or two substituents independently selected from —CH₃ and —CH₂CH₃.

50. The compound of any one of claims 1-10 or a pharmaceutically acceptable salt thereof, wherein:
R¹ is —OR¹⁵;
R¹¹ is C₁₋₆alkyl-NR^{a}R^{b}, phenyl, 4 to 12-membered carbocyclyl, 4 to 12-membered heterocyclyl comprising at least one ring N atom; wherein said phenyl or 4 to 12-membered carbocyclyl represented by R¹¹ is substituted with —NR^{a}R^{b}, Het, or —C₁₋₃alkylene-Het, and Het is a 4 to 6-membered heterocyclyl comprising at least one ring N atom and is optionally substituted with one or two C₁₋₃alkyl; and wherein said 4 to 12-membered heterocyclyl represented by R¹⁵ is optionally substituted by one or two C₁₋₃alkyl.

51. The compound of claim 50 or a pharmaceutically acceptable salt thereof, wherein R¹⁵ is selected from piperidinyl, pyrrolidinyl, 8-azaspiro[4.5]decanyl, and 7-azaspiro[3.5]nonanyl, each of which is optionally substituted with one or two C₁₋₃alkyl or R¹ is cyclopentyl substituted with NR^{a}R^{b}; and R^{a} and R^{b} are each independently H or C₁₋₃alkyl.

52. The compound of claim 50 or a pharmaceutically acceptable salt thereof, wherein:
R¹ is —OR¹⁵;
R¹⁵ is selected from a group consisting of: 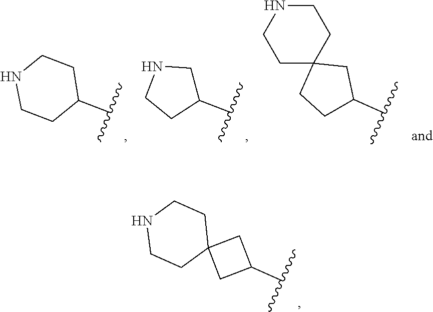
each of which is optionally substituted with one or two substituents independently selected from —CH₃ and —CH₂CH₃; or R¹⁵ is represented by

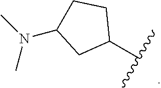 [SMILES: *C1CCC(N(C)C)C1]

53. The compound of any one of claims 1-52 or a pharmaceutically acceptable salt thereof, wherein R³ is a 9-membered bicyclic heteroaryl optionally substituted by one to three R^{C} or a phenyl fused with a 5-membered heterocyclyl optional substituted with one to three R^{C1}.

54. The compound of any one of claims 1-52 or a pharmaceutically acceptable salt thereof, wherein R³ is selected from a group consisting of indazolyl, imidazopyridinyl, imidazopyridazinyl, imidazopyrazinyl, benzothiazolyl, triazolopyrazinyl, benzooxazolyl, pyrazolopyrimidinyl, and benzothiadiazolyl, each of which is optionally substituted with one to three R^{C} or R³ is 1,3-dihydro-2H-benzo[d]imidazol-2-one or benzo[d]thiazol-2(3H)-one, each of which is optionally substituted with one or two R^{C1}.

55. The compound of claim 54 or a pharmaceutically acceptable salt thereof, wherein R³ is selected from a group consisting of: 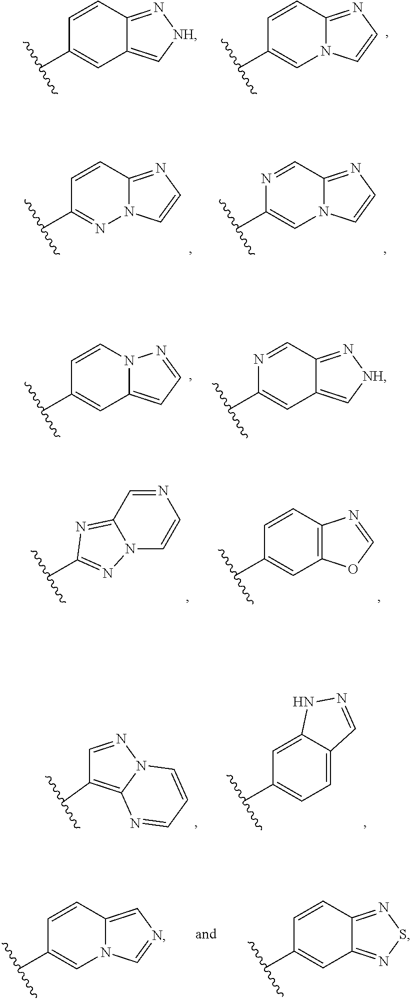
each of which is optionally substituted with one to three R^{C}; or 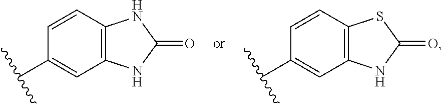 [SMILES: *c1ccc2[nH]c(=O)[nH]c2c1 ; *c1ccc2sc(=O)[nH]c2c1]
each of which is optionally substituted with one or two R^{C1}.

56. The compound of claim 54 or a pharmaceutically acceptable salt thereof, wherein R³ is selected from a group consisting of: 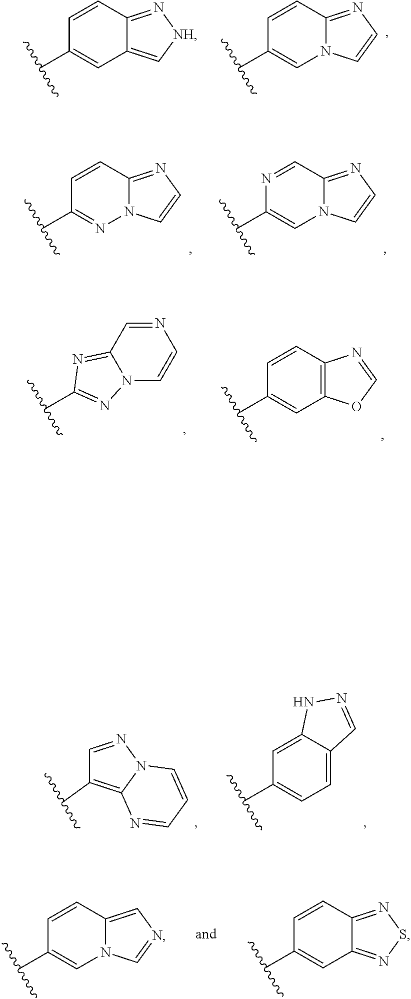
each of which is optionally substituted with one to three R^{C}; or
R³ is 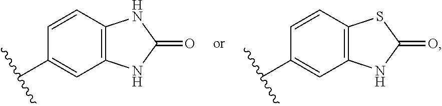 [SMILES: *c1ccc2[nH]c(=O)[nH]c2c1 ; *c1ccc2sc(=O)[nH]c2c1]
each of which is optionally substituted with one or two R^{C1}.

57. The compound of any one of claims 1-56 or a pharmaceutically acceptable salt thereof, wherein R^{c} for each occurrence is independently halo, C₁₋₃alkyl, C₁₋₂haloalkyl, or C₁₋₂ alkoxy; and R¹ for each occurrence is independently C₁₋₃alkyl.

58. The compound of claim 57 or a pharmaceutically acceptable salt thereof, wherein R^{C} for each occurrence is independently selected from —F, —CH₃, —CH(CH₃)₂, —CF₃, and —OCH₃; and R^{C1} is —CH₃.

59. The compound of claim 1, or a pharmaceutically acceptable salt thereof, wherein the compound is represented by the following Formula (IIA): 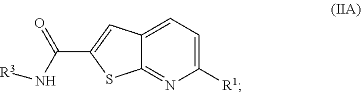
or a pharmaceutically acceptable salt thereof, wherein:
R¹ is piperazinyl, pyrrolidinyl, diazabicyclo[2.2.1]heptanyl, octahydropyrrolo[3,4-b]pyrrolyl, piperidinyl, 8-azabicyclo[3.2.1]oct-2-enyl or 1,2,3,6-tetrahydropyridinyl, wherein said piperazinyl, pyrrolidinyl, diazabicyclo[2.2.1]heptanyl, octahydropyrrolo[3,4-b]pyrrolyl, piperidinyl, 8-azabicyclo[3.2.1]oct-2-enyl or 1,2,3,6-tetrahydropyridinyl is optionally substituted with 1 to 3 R⁹ and said pyrrolidinyl is optionally substituted with —NR⁷R⁸ or —C₃₋₆cycloalkylene-NR⁷R⁸ and is further optionally substituted with 1 or 2 R⁹;
R⁷ and R⁸ are each independently H or C₁₋₄alkyl;
R⁹, for each occurrence, is independently selected from C₁₋₄alkyl and and C₃₋₆cycloalkyl; and
R³ is indazolyl, imidazopyridinyl, imidazopyrazinyl or benzooxazolyl, wherein said indazolyl, imidazopyridinyl, imidazopyrazinyl or benzooxazolyl is optionally substituted with one to two R^{C};
R^{C}, for each occurrence, is independently selected from C₁₋₄alkyl and halo.

60. The compound of claim 59, or a pharmaceutically acceptable salt thereof, wherein R¹ selected from a group consisting of 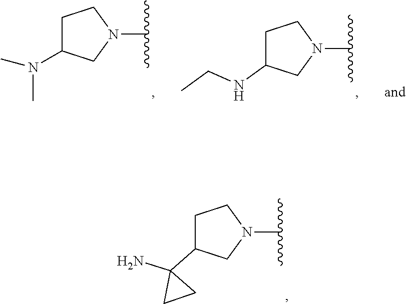
each of which is optionally substituted with 1 or 2 R⁹; or
R¹ is selected from 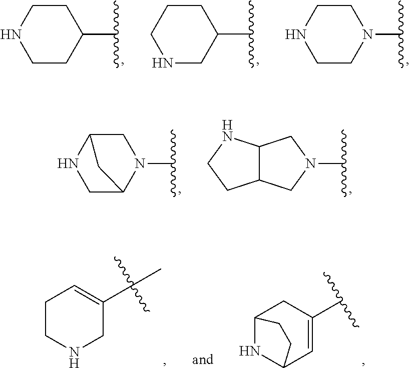
each of which is optionally substituted with 1 to 3 R⁹.

61. The compound of claim 59 or 60, or a pharmaceutically acceptable salt thereof, wherein R³ selected from a group consisting of: 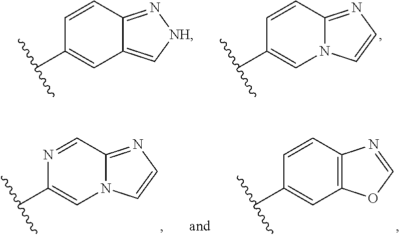 [SMILES: *c1ccc2n[nH]cc2c1 ; *c1ccc2nccn2c1 ; *c1cn2ccnc2cn1 ; *c1ccc2ncoc2c1]
each of which is is optionally substituted with one to two R^{C}.

62. The compound of any one of claims 59-61, or a pharmaceutically acceptable salt thereof, wherein R⁹, for each occurrence, is independently selected from —CH₃ and cyclopropyl.

63. The compound of any one of claims 59-62, or a pharmaceutically acceptable salt thereof, wherein R^{C}, for each occurrence, is independently selected from —CH₃ and F.

64. The compound of claim 1, or a pharmaceutically acceptable salt thereof, wherein the compound is represented by the following formula: 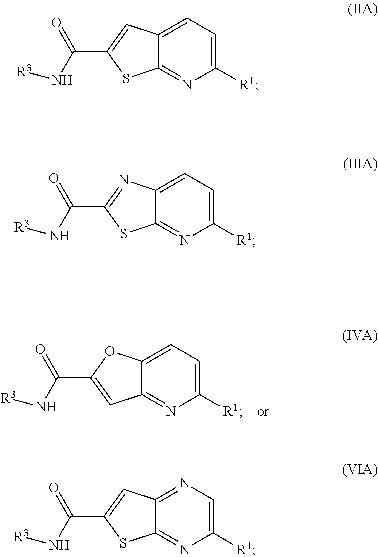
or a pharmaceutically acceptable salt thereof, wherein:
R¹ is piperazinyl, pyrrolidinyl, piperidinyl, diazasprio[4.4]nonanyl, diazabicyclo[3.2.0]heptanyl, or diazaspiro[3.4]octanyl, wherein said piperazinyl, piperidinyl, diazasprio[4.4]nonanyl, diazabicyclo[3.2.0]heptanyl, or diazaspiro[3.4]octanyl is optionally substituted with 1 to 3 R⁹ and said pyrrolidinyl is optionally substituted with —NR⁷R⁸ and is further optionally substituted with 1 or 2 R⁹;
R⁷ and R⁸ are each independently H or C₁₋₄alkyl; or R⁷ and R⁸ together with N atom from which they are attached form a 4 to 6 membered saturated monocyclic heterocyclyl;
R⁹, for each occurrence, is independently C₁₋₃alkyl; and
R³ is indazolyl, pyrazolo[1.5.a]pyridinyl, imidazopyridinyl, or imidazopyrazinyl, wherein said indazolyl, imidazopyridinyl, or imidazopyrazinyl is optionally substituted with one to two R^{C};
R^{C}, for each occurrence, is independently selected from C₁₋₃alkyl, C₁₋₃ haloalkyl, C₁₋₃alkoxy, and halo.

65. The compound of claim 64, or a pharmaceutically acceptable salt thereof, wherein R¹ is selected from a group consisting of: 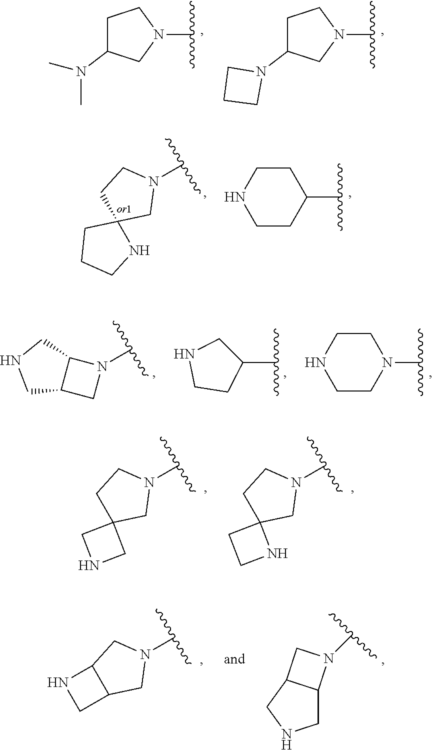
each or which is optionally substituted with 1 or 2 R⁹; and R⁹ for each occurrence is independently C₁₋₃alkyl.

66. The compound of claim 64 or 65, or a pharmaceutically acceptable salt thereof, wherein R³ is 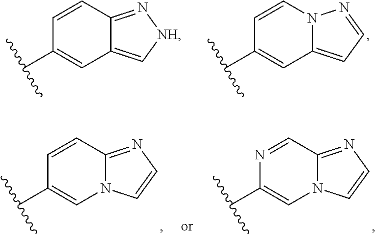 [SMILES: *c1ccc2n[nH]cc2c1 ; *c1ccn2nccc2c1 ; *c1ccc2nccn2c1 ; *c1cn2ccnc2cn1]
each of which is is optionally substituted with one to two R^{C}.

67. The compound of claim 64, 65 or 66, or a pharmaceutically acceptable salt thereof, wherein R⁹, for each occurrence, is independently selected from —CH₃ and —CH₂CH₃.

68. The compound of any one of claims 64-67, or a pharmaceutically acceptable salt thereof, wherein R^{C}, for each occurrence, is independently selected from F, —CH₃, —OCH₃, and —CHF₂.

69. The compound of claim 1 or a pharmaceutically acceptable salt thereof, wherein the compound is selected from Table 1, or a pharmaceutically acceptable salt thereof.

70. A pharmaceutical composition comprising a compound of any one of claims 1-69 or a pharmaceutically acceptable salt thereof and a pharmaceutically acceptable carrier.

71. A method of treating Huntington disease (HD) in a subject in need thereof comprising administering to the subject an effective amount of a compound of any one of claims 1-69 or a pharmaceutically acceptable salt thereof or a pharmaceutically composition of claim 70.
